(19) United States
(12) Patent Application Publication
(10) Pub. No.: US 2023/0265437 A1
(43) Pub. Date: Aug. 24, 2023

(54) RNAi Agents for Inhibiting Expression of Matrix Metalloproteinase 7 (MMP7), Compositions Thereof, and Methods of Use

(71) Applicant: Arrowhead Pharmaceuticals, Inc., Pasadena, CA (US)

(72) Inventors: Anthony Nicholas, Oregon, WI (US); Tao Pei, Middleton, WI (US); Erik W. Bush, Verona, WI (US); Tingting Yuan, Madison, WI (US)

(21) Appl. No.: 18/048,577

(22) Filed: Oct. 21, 2022

Related U.S. Application Data
(60) Provisional application No. 63/345,654, filed on May 25, 2022.
(60) Provisional application No. 63/308,289, filed on Feb. 9, 2022.
(60) Provisional application No. 63/270,849, filed on Oct. 22, 2021.

(51) Int. Cl. C12N 15/113; A61P 11/00

(52) U.S. Cl. CPC C12N 15/1137; A61P 11/00; C12N 2310/14; C12N 2310/11; C12N 2310/32

(57) ABSTRACT

Described are RNAi agents, compositions that include RNAi agents, and methods for inhibition of a matrix metallopeptidase 7 (MMP7) gene. The MMP7 RNAi agents and RNAi agent conjugates disclosed herein inhibit the expression of an MMP7 gene. Pharmaceutical compositions that include one or more MMP7 RNAi agents, optionally with one or more additional therapeutics, are also described. Delivery of the described MMP7 RNAi agents to pulmonary cells, in vivo, provides for inhibition of MMP7 gene expression, which can provide a therapeutic benefit to subjects, including human subjects, for the treatment of various diseases including pulmonary inflammation diseases such as idiopathic pulmonary fibrosis (IPF).

### CROSS REFERENCE TO RELATED APPLICATIONS

This application claims priority to U.S. Provisional Patent Application Ser. No. 63/270,849, filed on 22 Oct. 2021, U.S. Provisional Patent Application Ser. No. 63/308,289, filed on 9 Feb. 2022, and U.S. Provisional Patent Application Ser. No. 63/345,654, filed on 25 May 2022, the contents of each of which are incorporated herein by reference in their entirety.

### SEQUENCE LISTING

This application contains a Sequence Listing which has been submitted in ASCII format and is hereby incorporated by reference in its entirety. The ASCII copy is named 30673_US_SequenceListing.xml and is 3,575 kb in size.

### FIELD OF THE INVENTION

The present disclosure relates to RNA interference (RNAi) agents, e.g., double stranded RNAi agents, for inhibition of Matrix Metalloproteinase 7 (“MMP7” or “Matrilysin”) gene expression, compositions that include MMP7 RNAi agents, and methods of use thereof.

### BACKGROUND

Matrix Metalloproteinase 7 (“MMP7” or “Matrilysin”) is the smallest (28 kDa) member of the metalloproteinase (MMP) family of 24 related secreted zinc-dependent endopeptidases with diverse substrates and functions, being able to degrade components of the extracellular matrix (for example, elastin, proteoglycans, type IV collagen, fibronectin, entactin/nidogen, and the core protein of proteoglycans) as well as cleaving and modulating the activity non-extracellular matrix substrates like cytokines. (Fujishima, Shiomi et al., Arch Pathol Lab Med 134(8): 1136-1142 (2010); Craig, Zhang et al., Am J Respir Cell Mol Biol 53(5): 585-600 (2015)). These functional roles in extracellular matrix remodeling and regulation of cytokine signaling have linked members of the MMP family to common pathogenic mechanisms contributing to cancer, chronic inflammation, and fibrosis. Despite their attractiveness as drug targets, however, the development of highly selective small molecule MMP inhibitors has been challenging due to the shared structural similarity of the zinc-dependent catalytic domain among family members (Vandenbroucke and Libert, Nat Rev Drug Discov 13(12): 904-927 (2014); Fields, Cells 8(9) (2019)).

MMP7 is constitutively expressed and secreted by epithelial cells throughout the body (including the skin, lung, and glandular epithelia of the liver, intestine, pancreas, salivary gland, and reproductive tract), where it plays a role in epithelial repair (Pilcher, Wang et al., Ann N Y Acad Sci 878:12-24 (1999)). Increased MMP7 expression has been linked to pathogenic fibrosis of the lung (Rosas, Richards et al., PLoS Med 5(4): e93 (2008)), liver (Hung, Chang et al., Hepatology 50(4): 1184-1193 (2009); Roeb, Matrix Biol 68-69: 463-473 (2018); Nomden, Beljaars et al., Front Med (Lausanne) 7:617261 (2020); Irvine, Okano et al., Sci Rep 11(1):2858 (2021)), and kidney (Ke, Fan et al., Front Physiol 8:21 (2017); Zhang, Ren et al., Kidney Blood Press Res 42(3): 541-552 (2017); Tan, Li et al., JCI Insight 4(24) (2019)). MMP7 enzyme levels have been linked to pathogenic fibrosis via multiple potential mechanisms including promotion of epithelial-mesenchymal transition (EMT), extracellular matrix degradation, aberrant matrix repair, and tissue remodeling. MMP7 promotes fibrosis by cleaving E-cadherin to activate epithelial cells and proteolytically activating heparin-binding epidermal growth factor precursor (pro-HB-EGF) to release active HB-EGF, which promotes aberrant epithelial migration and human lung fibroblast proliferation (Zhang, Rice et al., Am J Respir Cell Mol Biol 24(2):123-131 (2001); McGuire, Li et al., Am J Pathol 162(6):1831-1843 (2003)). MMP7 is also known to promote fibroblast survival and resistance to apoptosis via cleavage of osteopontin and mFasL pathways (Agnihotri, Crawford et al., J Biol Chem 276(30):28261-28267 (2001); Mummler, Burgy et al., FASEB J 32(2):703-716 (2018); Nareznoi, Konikov-Rozenman et al., Cells 9(2) (2020)).

One specific type of fibrosis is idiopathic pulmonary fibrosis (IPF), a chronic lung disease that is often fatal, and for which the clinical course and rate of disease progression are relatively unpredictable (Id.). In patients with IPF, MMP7 expression is known to increase in peripheral blood, bronchoalveolar lavage fluid, and lung tissue (Zuo, Kaminski et al., Proc Natl Acad Sci USA 99(9): 6292-6297 (2002)). Serum MMP7 expression is a well validated serum biomarker for IPF, which correlates with IPF severity and progression (Song, Do et al., Chest 143(5):1422-1429 (2013); Tzouvelekis, Herazo-Maya et al., Respirology 22(3):486-493 (2017)). Consistent with its known mechanism, MMP7 modulates multiple pathways contributing to aberrant epithelial cell, fibroblast, and immune cell function in IPF. Significantly, MMP7 knockout mice are protected against bleomycin-mediated lung injury (a standard rodent model of IPF), demonstrating reduced pulmonary inflammation, fibrosis, and mortality, and thus suggesting a causative role for MMP7 in IPF (Craig, Zhang et al., Am J Respir Cell Mol Biol 53(5):585-600 (2015)).

Genome-Wide Association Studies (GWAS) have shown that gain of function MMP7 genetic variant rs11568818AA linked to IPF risk (Richards, Park et al., Am J Physiol Lung Cell Mol Physiol 302(8):L746-754 (2012)). Further, in such GWAS studies it is described that MMP7 genetic variants are associated with multiple cancer diseases and sclerosis (Moreno-Ortiz, Gutierrez-Angulo et al., Genet Mol Res 13(2):3537-3544 (2014); Fu, Chien et al., Anticancer Res 40(2):695-702 (2020)).

### SUMMARY

There exists a need for novel RNA interference (RNAi) agents (termed RNAi agents, RNAi triggers, or triggers), e.g., double stranded RNAi agents, that are able to selectively and efficiently inhibit the expression of a MMP7 gene, including for use as a therapeutic or medicament. Further, there exists a need for compositions of novel MMP7-specific RNAi agents for the treatment of diseases or disorders associated with pathological inflammation (such as IPF) and/or disorders that can be mediated at least in part by a reduction in MMP7 gene expression.

The nucleotide sequences and chemical modifications of the MMP7 RNAi agents disclosed herein, as well as their combination with certain specific targeting ligands suitable for selectively and efficiently delivering the MMP7 RNAi agents to relevant pulmonary cells in vivo, differ from what is previously disclosed or known in the art. The MMP7 RNAi agents disclosed herein provide for highly potent and efficient inhibition of the expression of a MMP7 gene.

In general, the present disclosure features MMP7 gene-specific RNAi agents, compositions that include MMP7 RNAi agents, and methods for inhibiting expression of a MMP7 gene in vitro and/or in vivo using the MMP7 RNAi agents and compositions that include MMP7 RNAi agents described herein. The MMP7 RNAi agents described herein are able to selectively and efficiently decrease expression of a MMP7 gene, and thereby decrease the amount of MMP7 available which is believed to be pro-fibrotic through potentially multiple mechanisms including by cleaving E-cadherin to activate epithelial cells and proteolytically activating heparin-binding epidermal growth factor precursor (pro-HB-EGF) to release active HB-EGF, which promotes aberrant epithelial migration and human lung fibroblast proliferation, and well as cleaving and activating other pro-fibrotic substrates such as osteopontin and membrane bound Fas ligand (mFasL).

The described MMP7 RNAi agents can be used in methods for therapeutic treatment (including preventative or prophylactic treatment) of symptoms and diseases including, but not limited to, idiopathic pulmonary fibrosis (IPF), asthma, various other types of fibrosis, chronic inflammation, interstitial lung diseases (ILD), infectious disease (for example, SARS-COV-2), acute lung injury (for example, acute respiratory distress syndrome (ARDS)), pulmonary hypertension, various cancers, nonalcoholic fatty liver disease (NAFLD), nonalcoholic steatohepatitis (NASH), fatty liver disease, biliary atresia, and chronic kidney disease (CKD).

In one aspect, the disclosure features RNAi agents for inhibiting expression of a MMP7 gene, wherein the RNAi agent includes a sense strand (also referred to as a passenger strand) and an antisense strand (also referred to as a guide strand). The sense strand and the antisense strand can be partially, substantially, or fully complementary to each other. The length of the RNAi agent sense strands described herein each can be 12 to 49 nucleotides in length. The length of the RNAi agent antisense strands described herein each can be 18 to 30 nucleotides in length. In some embodiments, the sense and antisense strands are independently 18 to 26 nucleotides in length. The sense and antisense strands can be either the same length or different lengths. In some embodiments, the sense and antisense strands are independently 21 to 26 nucleotides in length. In some embodiments, the sense and antisense strands are independently 21 to 24 nucleotides in length. In some embodiments, both the sense strand and the antisense strand are 21 nucleotides in length. In some embodiments, the antisense strands are independently 18, 19, 20, 21, 22, 23, 24, 25, 26, 27, 28, 29, or 30 nucleotides in length. In some embodiments, the sense strands are independently 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. The RNAi agents described herein, upon delivery to a cell expressing MMP7 such as a pulmonary cell, inhibit the expression of one or more MMP7 gene variants in vivo and/or in vitro.

The MMP7 RNAi agents disclosed herein target a human MMP7 gene (see, e.g., SEQ ID NO:1). In some embodiments, the MMP7 RNAi agents disclosed herein target a portion of an MMP7 gene having the sequence of any of the sequences disclosed in Table 1.

In another aspect, the disclosure features compositions, including pharmaceutical compositions, that include one or more of the disclosed MMP7 RNAi agents that are able to selectively and efficiently decrease expression of an MMP7 gene. The compositions that include one or more MMP7 RNAi agents described herein can be administered to a subject, such as a human or animal subject, for the treatment (including prophylactic treatment or inhibition) of symptoms and diseases including, but not limited to, various pulmonary diseases including idiopathic pulmonary fibrosis (IPF), asthma, various other types of fibrosis, chronic inflammation, interstitial lung diseases (ILD), infectious disease (for example, SARS-COV-2), acute lung injury (for example, acute respiratory distress syndrome (ARDS)), pulmonary hypertension, various cancers, nonalcoholic fatty liver disease (NAFLD), nonalcoholic steatohepatitis (NASH), fatty liver disease, biliary atresia, and chronic kidney disease (CKD).

Examples of MMP7 RNAi agent sense strands and antisense strands that can be used in a MMP7 RNAi agent are provided in Tables 3, 4, 5, and 6. Examples of MMP7 RNAi agent duplexes are provided in Tables 7A, 7B, 8, 9, and 10. Examples of 19-nucleotide core stretch sequences that may consist of or may be included in the sense strands and antisense strands of certain MMP7 RNAi agents disclosed herein, are provided in Table 2.

In another aspect, the disclosure features methods for delivering MMP7 RNAi agents to epithelial cells in a subject, such as a mammal, in vivo. Also described herein are compositions for use in such methods. In some embodiments, disclosed herein are methods for delivering MMP7 RNAi agents to pulmonary cells (epithelial cells, macrophages, smooth muscle, endothelial cells) to a subject in vivo. In some embodiments, the subject is a human subject.

The methods disclosed herein include the administration of one or more MMP7 RNAi agents to a subject, e.g., a human or animal subject, by any suitable means known in the art. The pharmaceutical compositions disclosed herein that include one or more MMP7 RNAi agents can be administered in a number of ways depending upon whether local or systemic treatment is desired. Administration can be, but is not limited to, for example, intravenous, intraarterial, subcutaneous, intraperitoneal, subdermal (e.g., via an implanted device), and intraparenchymal administration. In some embodiments, the pharmaceutical compositions described herein are administered by inhalation (such as dry powder inhalation or aerosol inhalation), intranasal administration, intratracheal administration, or oropharyngeal aspiration administration.

In some embodiments, it is desired that the MMP7 RNAi agents described herein inhibit the expression of an MMP7 gene in the pulmonary epitheliur, for which the administration is by inhalation (e.g., by an inhaler device, such as a metered-dose inhaler, or a nebulizer such as a jet or vibrating mesh nebulizer, or a soft mist inhaler).

The one or more MMP7 RNAi agents can be delivered to target cells or tissues using any oligonucleotide delivery technology known in the art. In some embodiments, a MMP7 RNAi agent is delivered to cells or tissues by covalently linking the RNAi agent to a targeting group. In some embodiments, the targeting group can include a cell receptor ligand, such as an integrin targeting ligand. Integrins are a family of transmembrane receptors that facilitate cell-extracellular matrix (ECM) adhesion. In particular, integrin alpha-v-beta-6 (αvβ6) is an epithelial-specific integrin that is known to be a receptor for ECM proteins and the TGF-beta latency-associated peptide (LAP), and is expressed in various cells and tissues. Integrin αvβ6 is known to be highly upregulated in injured pulmonary epithelium. In some embodiments, the MMP7 RNAi agents described herein are linked to an integrin targeting ligand that has affinity for integrin αvβ6. As referred to herein, an “αvβ6 integrin targeting ligand” is a compound that has affinity for integrin αvβ6, which can be utilized as a ligand to facilitate the targeting and delivery of an RNAi agent to which it is attached to the desired cells and/or tissues (i.e., to cells expressing integrin αvβ6). In some embodiments, multiple αvβ6 integrin targeting ligands or clusters of αvβ6 integrin targeting ligands are linked to a MMP7 RNAi agent. In some embodiments, the MMP7 RNAi agent-αvβ6 integrin targeting ligand conjugates are selectively internalized by lung epithelial cells, either through receptor-mediated endocytosis or by other means.

Examples of targeting groups useful for delivering MMP7 RNAi agents that include αvβ6 integrin targeting ligands are disclosed, for example, in International Patent Application Publication No. WO 2018/085415 and International Patent Application Publication No. WO 2019/089765, the contents of each of which are incorporated by reference herein in their entirety.

A targeting group can be linked to the 3′ or 5′ end of a sense strand or an antisense strand of a MMP7 RNAi agent. In some embodiments, a targeting group is linked to the 3′ or 5′ end of the sense strand. In some embodiments, a targeting group is linked to the 5′ end of the sense strand. In some embodiments, a targeting group is linked internally to a nucleotide on the sense strand and/or the antisense strand of the RNAi agent. In some embodiments, one or more targeting ligands are linked internally to one or more nucleotides on the sense strand of the RNAi agent. In some embodiments, a targeting group is linked to the RNAi agent via a linker.

In another aspect, the disclosure features compositions that include one or more MMP7 RNAi agents that have the duplex structures disclosed in Tables 7A, 7B, 8, 9, and 10.

The use of MMP7 RNAi agents provides methods for therapeutic (including prophylactic) treatment of diseases or disorders for which a reduction in MMP7 can provide a therapeutic benefit. The MMP7 RNAi agents disclosed herein can be used to treat various pulmonary diseases various pulmonary diseases including idiopathic pulmonary fibrosis (IPF), asthma, various other types of fibrosis, chronic inflammation, interstitial lung diseases (ILD), infectious disease (for example, SARS-COV-2), acute lung injury (for example, acute respiratory distress syndrome (ARDS)), pulmonary hypertension, various cancers, nonalcoholic fatty liver disease (NAFLD), nonalcoholic steatohepatitis (NASH), fatty liver disease, biliary atresia, and chronic kidney disease (CKD). In some embodiments, the MMP7 RNAi agents disclosed herein can be used to treat a pulmonary inflammatory disease or condition. MMP7 RNAi agents can be used to treat, for example, IPF or other types of pulmonary fibrosis. Such methods of treatment include administration of a MMP7 RNAi agent to a human being or animal for which a reduction in MMP7 levels is desired.

As used herein, the terms “oligonucleotide” and “polynucleotide” mean a polymer of linked nucleosides each of which can be independently modified or unmodified.

As used herein, an “RNAi agent” (also referred to as an “RNAi trigger”) means a composition that contains an RNA or RNA-like (e.g., chemically modified RNA) oligonucleotide molecule that is capable of degrading or inhibiting (e.g., degrades or inhibits under appropriate conditions) translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short (or small) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the mRNA being targeted (i.e. MMP7 mRNA). RNAi agents can include one or more modified nucleotides and/or one or more non-phosphodiester linkages.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or otherwise suitable in vivo or in vitro conditions)) and form a duplex or double helical structure under certain standard conditions with an oligonucleotide that includes the second nucleotide sequence. The person of ordinary skill in the art would be able to select the set of conditions most appropriate for a hybridization test. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary,” “fully complementary,” “partially complementary,” and “substantially complementary” are used with respect to the nucleobase or nucleotide matching between the sense strand and the antisense strand of an RNAi agent, or between the antisense strand of an RNAi agent and a sequence of an MMP7 mRNA.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition or reduction of the number, severity, and/or frequency of one or more symptoms of a disease in a subject.

As used herein, the phrase “introducing into a cell,” when referring to an RNAi agent, means functionally delivering the RNAi agent into a cell. The phrase “functional delivery,” means delivering the RNAi agent to the cell in a manner that enables the RNAi agent to have the expected biological activity, e.g., sequence-specific inhibition of gene expression.

Unless stated otherwise, use of the symbol

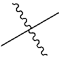

as used herein means that any group or groups may be linked thereto that is in accordance with the scope of the inventions described herein.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art. Correspondingly, compounds described herein with labile protons or basic atoms should also be understood to represent salt forms of the corresponding compound. Compounds described herein may be in a free acid, free base, or salt form. Pharmaceutically acceptable salts of the compounds described herein should be understood to be within the scope of the invention.

As used herein, the term “linked” or “conjugated” when referring to the connection between two compounds or molecules means that two compounds or molecules are joined by a covalent bond. Unless stated, the terms “linked” and “conjugated” as used herein may refer to the connection between a first compound and a second compound either with or without any intervening atoms or groups of atoms.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other objects, features, aspects, and advantages of the invention will be apparent from the following detailed description, accompanying figures, and from the claims.

### BRIEF DESCRIPTION OF THE DRAWINGS

FIG. 1. Chemical structure representation of the tridentate αvβ6 epithelial cell targeting ligand referred to herein as Tri-SM6.1-αvβ6-(TA14).

FIG. 2. Chemical structure representation of the peptide αvβ6 epithelial cell targeting ligand referred to herein as αvβ6-pep1.

FIG. 3. MTT colorimetric assay for cell metabolic activity, demonstrating little cytotoxic effect of MMP7 RNAi agent, as described in Example 24.

FIG. 4A. Schematic diagram of the modified sense and antisense strands of the MMP7 RNAi agent conjugate having the structure of AC001651 (see, e.g., Tables 8 and 10), having a tridentate αvβ6 epithelial cell targeting ligand linked at the 5′ end of the sense strand.

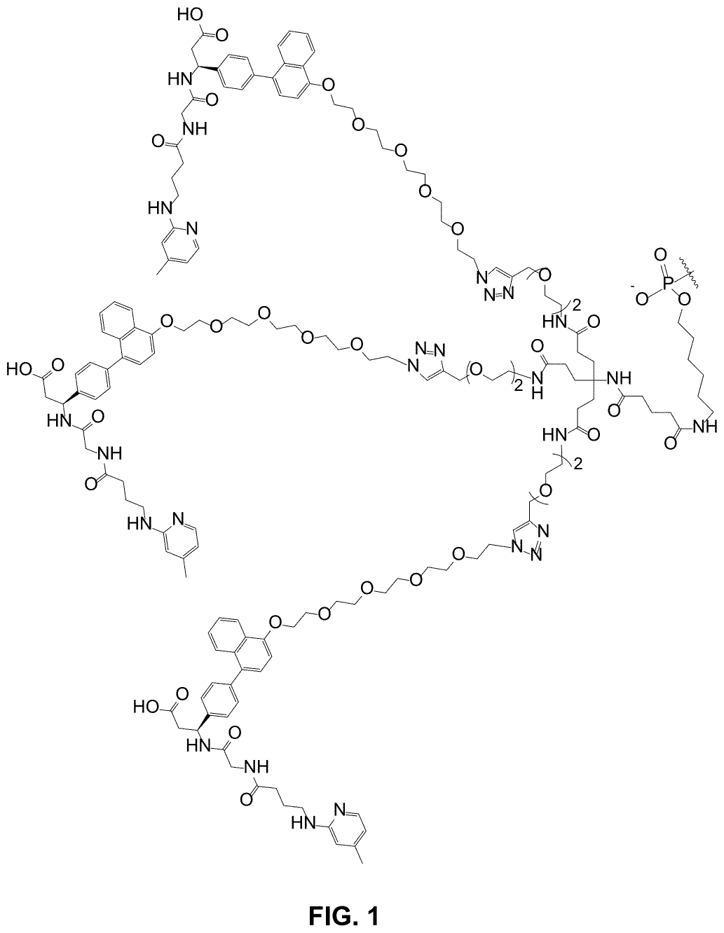

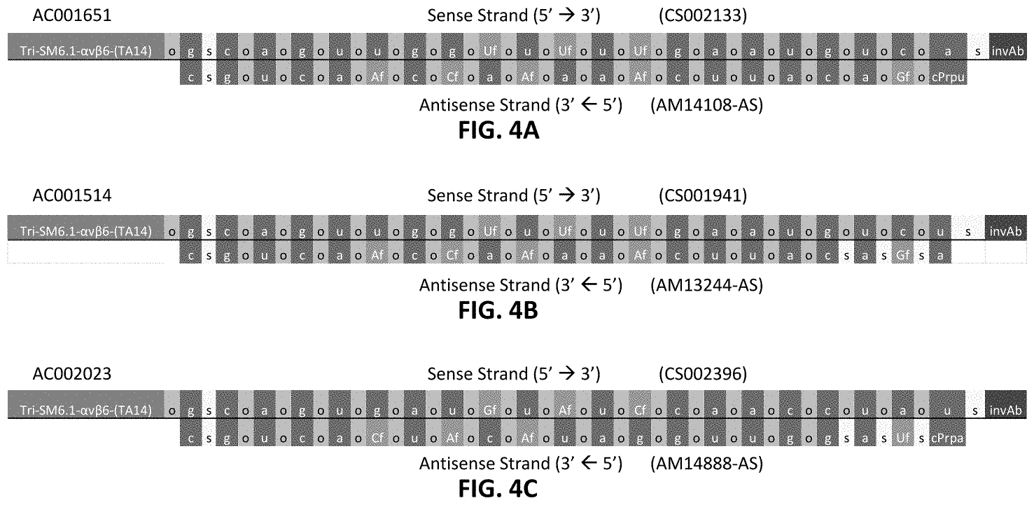

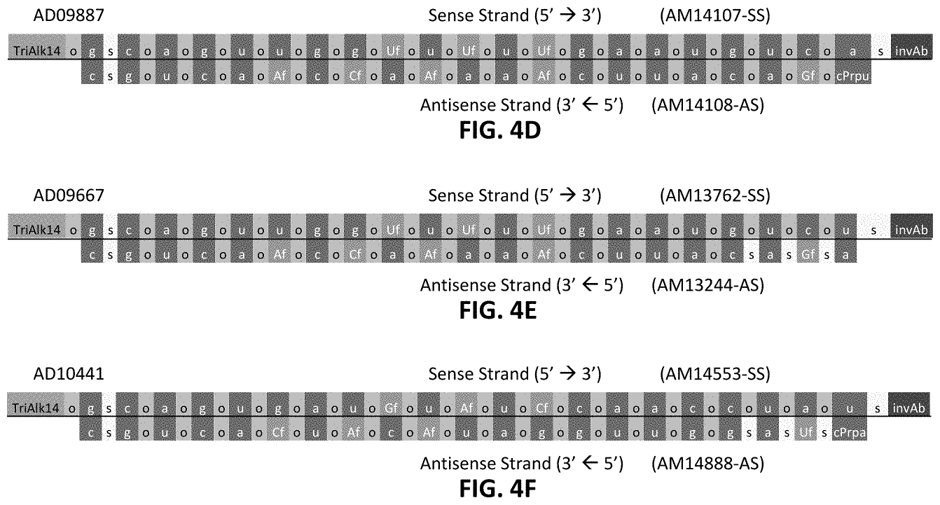

The following abbreviations are used in FIGS. 4A to 4F: a, c, g, and u are 2′-O-methyl modified nucleotides; Af, Cf, Gf, and Uf are 2′-fluoro modified nucleotides; o is a phosphodiester linkage; s is a phosphorothioate linkage; invAb is an inverted abasic residue (see, e.g., Table 11); cPrpu is a 5′-cyclopropyl phosphonate-2′-O-methyluridine modified nucleotide (see, e.g., Table 11); cPrpa is a 5′-cyclopropyl phosphonate-2′-O-methyladenosine modified nucleotide (see, e.g., Table 11); Tri-SM6.1-αvβ6-(TA14) is the tridentate αvβ6 epithelial cell targeting ligand having the structure shown in FIG. 1; and (TriAlk14) is the linking group as shown in Table 11, which is suitable for subsequent conjugation to targeting ligands (See also, Example 1 herein).

FIG. 4B. Schematic diagram of the modified sense and antisense strands of the MMP7 RNAi agent conjugate having the structure of AC001514 (see, e.g., Tables 8 and 10), having a tridentate αvβ6 epithelial cell targeting ligand linked at the 5′ end of the sense strand.

FIG. 4C. Schematic diagram of the modified sense and antisense strands of the MMP7 RNAi agent conjugate having the structure of AC0002023 (see, e.g., Tables 8 and 10), having a tridentate αvβ6 epithelial cell targeting ligand linked at the 5′ end of the sense strand.

FIG. 4D. Schematic diagram of the modified sense and antisense strands of the MMP7 RNAi agent duplex having the structure of AD09887 (see, e.g., Tables 8 and 10), having a (TriAlk14) linker at the 5′ end of the sense strand.

FIG. 4E. Schematic diagram of the modified sense and antisense strands of the MMP7 RNAi agent duplex having the structure of AD09667 (see, e.g., Tables 8 and 10), having a (TriAlk14) linker at the 5′ end of the sense strand.

FIG. 4F. Schematic diagram of the modified sense and antisense strands of the MMP7 RNAi agent duplex having the structure of AD10441 (see, e.g., Tables 8 and 10), having a (TriAlk14) linker at the 5′ end of the sense strand.

### DETAILED DESCRIPTION

### RNAi Agents

Described herein are RNAi agents for inhibiting expression of the MMP7 gene (referred to herein as MMP7 RNAi agents or MMP7 RNAi triggers). Each MMP7 RNAi agent disclosed herein comprises a sense strand and an antisense strand. The sense strand can be 12 to 49 nucleotides in length. In some embodiments, the sense strand is 12 to 49 nucleotides in length. The antisense strand can be 18 to 49 nucleotides in length. The sense and antisense strands can be either the same length or they can be different lengths. In some embodiments, the sense and antisense strands are each independently 18 to 27 nucleotides in length. In some embodiments, both the sense and antisense strands are each 21-26 nucleotides in length. In some embodiments, the sense and antisense strands are each 21-24 nucleotides in length. In some embodiments, the sense and antisense strands are each independently 19-21 nucleotides in length. In some embodiments, the sense strand is about 19 nucleotides in length while the antisense strand is about 21 nucleotides in length. In some embodiments, the sense strand is about 21 nucleotides in length while the antisense strand is about 23 nucleotides in length. In some embodiments, a sense strand is 23 nucleotides in length and an antisense strand is 21 nucleotides in length. In some embodiments, both the sense and antisense strands are each 21 nucleotides in length. In some embodiments, the RNAi agent sense strands are each independently 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. In some embodiments, the RNAi agent antisense strands are each independently 18, 19, 20, 21, 22, 23, 24, 25, 26, 27, 28, 29, or 30 nucleotides in length. In some embodiments, the RNAi agent is double stranded and has a duplex length of about 12, 13, 14, 15, 16, 17, 18, 19, 20, 21, 22, 23 or 24 nucleotides. In some embodiments, the RNAi agent is double stranded and has a duplex length of 19, 20, 21, 22, or 23 nucleotides.

Examples of nucleotide sequences used in forming MMP7 RNAi agents are provided in Tables 2, 3, 4, 5, 6, and 10. Examples of RNAi agent duplexes, that include the sense strand and antisense strand sequences in Tables 2, 3, 4, 5, 6, are shown in Tables 7A, 7B, 8, 9, and 10.

In some embodiments, the region of perfect, substantial, or partial complementarity between the sense strand and the antisense strand is 16-26 (e.g., 16, 17, 18, 19, 20, 21, 22, 23, 24, 25, or 26) nucleotides in length and occurs at or near the 5′ end of the antisense strand (e.g., this region may be separated from the 5′ end of the antisense strand by 0, 1, 2, 3, or 4 nucleotides that are not perfectly, substantially, or partially complementary).

A sense strand of the MMP7 RNAi agents described herein includes at least 12 consecutive nucleotides that have at least 85% identity to a core stretch sequence (also referred to herein as a “core stretch” or “core sequence”) of the same number of nucleotides in an MMP7 mRNA. In some embodiments, a sense strand core stretch sequence is 100% (perfectly) complementary or at least about 85% (substantially) complementary to a core stretch sequence in the antisense strand, and thus the sense strand core stretch sequence is typically perfectly identical or at least about 85% identical to a nucleotide sequence of the same length (sometimes referred to, e.g., as a target sequence) present in the MMP7 mRNA target. In some embodiments, this sense strand core stretch is 16, 17, 18, 19, 20, 21, 22, or 23 nucleotides in length. In some embodiments, this sense strand core stretch is 17 nucleotides in length. In some embodiments, this sense strand core stretch is 19 nucleotides in length. In some embodiments, this sense strand core stretch is 21 nucleotides in length.

An antisense strand of a MMP7 RNAi agent described herein includes at least 15 consecutive nucleotides that have at least 85% complementarity to a core stretch of the same number of nucleotides in an MMP7 mRNA and to a core stretch of the same number of nucleotides in the corresponding sense strand. In some embodiments, an antisense strand core stretch is 100% (perfectly) complementary or at least about 85% (substantially) complementary to a nucleotide sequence (e.g., target sequence) of the same length present in the MMP7 mRNA target. In some embodiments, this antisense strand core stretch is 17, 18, 19, 20, 21, 22, or 23 nucleotides in length. In some embodiments, this antisense strand core stretch is 19 nucleotides in length. In some embodiments, this antisense strand core stretch is 17 nucleotides in length. A sense strand core stretch sequence can be the same length as a corresponding antisense core sequence or it can be a different length.

The MMP7 RNAi agent sense and antisense strands anneal to form a duplex. A sense strand and an antisense strand of a MMP7 RNAi agent can be partially, substantially, or fully complementary to each other. Within the complementary duplex region, the sense strand core stretch sequence is at least 85% complementary or 100% complementary to the antisense core stretch sequence. In some embodiments, the sense strand core stretch sequence contains a sequence of at least 16, at least 17, at least 18, at least 19, at least 20, at least 21, at least 22, or at least 23 nucleotides that is at least 85% or 100% complementary to a corresponding 16, 17, 18, 19, 20, 21, 22, or 23 nucleotide sequence of the antisense strand core stretch sequence (i.e., the sense and antisense core stretch sequences of a MMP7 RNAi agent have a region of at least 16, at least 17, at least 18, at least 19, at least 20, at least 21, at least 22, or at least 23 nucleotides that is at least 85% base paired or 100% base paired.)

In some embodiments, the antisense strand of a MMP7 RNAi agent disclosed herein differs by 0, 1, 2, or 3 nucleotides from any of the antisense strand sequences in Table 2 or Table 3. In some embodiments, the sense strand of a MMP7 RNAi agent disclosed herein differs by 0, 1, 2, or 3 nucleotides from any of the sense strand sequences in Table 2, Table 4, Table 5, Table 6, or Table 10.

In some embodiments, the sense strand and/or the antisense strand can optionally and independently contain an additional 1, 2, 3, 4, 5, or 6 nucleotides (extension) at the 3′ end, the 5′ end, or both the 3′ and 5′ ends of the core stretch sequences. The antisense strand additional nucleotides, if present, may or may not be complementary to the corresponding sequence in the MMP7 mRNA. The sense strand additional nucleotides, if present, may or may not be identical to the corresponding sequence in the MMP7 mRNA. The antisense strand additional nucleotides, if present, may or may not be complementary to the corresponding sense strand's additional nucleotides, if present.

As used herein, an extension comprises 1, 2, 3, 4, 5, or 6 nucleotides at the 5′ and/or 3′ end of the sense strand core stretch sequence and/or antisense strand core stretch sequence. The extension nucleotides on a sense strand may or may not be complementary to nucleotides, either core stretch sequence nucleotides or extension nucleotides, in the corresponding antisense strand. Conversely, the extension nucleotides on an antisense strand may or may not be complementary to nucleotides, either core stretch nucleotides or extension nucleotides, in the corresponding sense strand. In some embodiments, both the sense strand and the antisense strand of an RNAi agent contain 3′ and 5′ extensions. In some embodiments, one or more of the 3′ extension nucleotides of one strand base pairs with one or more 5′ extension nucleotides of the other strand. In other embodiments, one or more of 3′ extension nucleotides of one strand do not base pair with one or more 5′ extension nucleotides of the other strand. In some embodiments, a MMP7 RNAi agent has an antisense strand having a 3′ extension and a sense strand having a 5′ extension. In some embodiments, the extension nucleotide(s) are unpaired and form an overhang. As used herein, an “overhang” refers to a stretch of one or more unpaired nucleotides located at a terminal end of either the sense strand or the antisense strand that does not form part of the hybridized or duplexed portion of an RNAi agent disclosed herein (See, e.g., U.S. Pat. No. 8,362,231).

In some embodiments, a MMP7 RNAi agent comprises an antisense strand having a 3′ extension of 1, 2, 3, 4, 5, or 6 nucleotides in length. In other embodiments, a MMP7 RNAi agent comprises an antisense strand having a 3′ extension of 1, 2, or 3 nucleotides in length. In some embodiments, one or more of the antisense strand extension nucleotides comprise nucleotides that are complementary to the corresponding MMP7 mRNA sequence. In some embodiments, one or more of the antisense strand extension nucleotides comprise nucleotides that are not complementary to the corresponding MMP7 mRNA sequence.

In some embodiments, a MMP7 RNAi agent comprises a sense strand having a 3′ extension of 1, 2, 3, 4, or 5 nucleotides in length. In some embodiments, one or more of the sense strand extension nucleotides comprises adenosine, uracil, or thymidine nucleotides, AT dinucleotide, or nucleotides that correspond to or are the identical to nucleotides in the MMP7 mRNA sequence. In some embodiments, the 3′ sense strand extension includes or consists of one of the following sequences, but is not limited to: T, UT, TT, UU, UUT, TTT, or TTTT (each listed 5′ to 3′).

A sense strand can have a 3′ extension and/or a 5′ extension. In some embodiments, a MMP7 RNAi agent comprises a sense strand having a 5′ extension of 1, 2, 3, 4, 5, or 6 nucleotides in length. In some embodiments, one or more of the sense strand extension nucleotides comprise nucleotides that correspond to or are identical to nucleotides in the MMP7 mRNA sequence.

Examples of sequences used in forming MMP7 RNAi agents are provided in Tables 2, 3, 4, 5, 6, and 10. In some embodiments, a MMP7 RNAi agent antisense strand includes a sequence of any of the sequences in Tables 2, 3, or 10. In certain embodiments, a MMP7 RNAi agent antisense strand comprises or consists of any one of the modified sequences in Table 3. In some embodiments, a MMP7 RNAi agent antisense strand includes the sequence of nucleotides (from 5′ end→3′ end) 1-17, 2-15, 2-17, 1-18, 2-18, 1-19, 2-19, 1-20, 2-20, 1-21, or 2-21, of any of the sequences in Tables 2 or 3. In some embodiments, a MMP7 RNAi agent sense strand includes the sequence of any of the sequences in Tables 2, 4, 5, or 6. In some embodiments, a MMP7 RNAi agent sense strand includes the sequence of nucleotides (from 5′ end→3′ end) 1-18, 1-19, 1-20, 1-21, 2-19, 2-20, 2-21, 3-20, 3-21, or 4-21 of any of the sequences in Tables 2, 4, 5, or 6. In certain embodiments, a MMP7 RNAi agent sense strand comprises or consists of a modified sequence of any one of the modified sequences in Table 4, 5, 6, or 10.

In some embodiments, the sense and antisense strands of the RNAi agents described herein contain the same number of nucleotides. In some embodiments, the sense and antisense strands of the RNAi agents described herein contain different numbers of nucleotides. In some embodiments, the sense strand 5′ end and the antisense strand 3′ end of an RNAi agent form a blunt end. In some embodiments, the sense strand 3′ end and the antisense strand 5′ end of an RNAi agent form a blunt end. In some embodiments, both ends of an RNAi agent form blunt ends. In some embodiments, neither end of an RNAi agent is blunt-ended. As used herein a “blunt end” refers to an end of a double stranded RNAi agent in which the terminal nucleotides of the two annealed strands are complementary (form a complementary base-pair).

In some embodiments, the sense strand 5′ end and the antisense strand 3′ end of an RNAi agent form a frayed end. In some embodiments, the sense strand 3′ end and the antisense strand 5′ end of an RNAi agent form a frayed end. In some embodiments, both ends of an RNAi agent form a frayed end. In some embodiments, neither end of an RNAi agent is a frayed end. As used herein a frayed end refers to an end of a double stranded RNAi agent in which the terminal nucleotides of the two annealed strands form a pair (i.e., do not form an overhang) but are not complementary (i.e. form a non-complementary pair). In some embodiments, one or more unpaired nucleotides at the end of one strand of a double stranded RNAi agent form an overhang. The unpaired nucleotides may be on the sense strand or the antisense strand, creating either 3′ or 5′ overhangs. In some embodiments, the RNAi agent contains: a blunt end and a frayed end, a blunt end and 5′ overhang end, a blunt end and a 3′ overhang end, a frayed end and a 5′ overhang end, a frayed end and a 3′ overhang end, two 5′ overhang ends, two 3′ overhang ends, a 5′ overhang end and a 3′ overhang end, two frayed ends, or two blunt ends. Typically, when present, overhangs are located at the 3′ terminal ends of the sense strand, the antisense strand, or both the sense strand and the antisense strand.

The MMP7 RNAi agents disclosed herein may also be comprised of one or more modified nucleotides. In some embodiments, substantially all of the nucleotides of the sense strand and substantially all of the nucleotides of the antisense strand of the MMP7 RNAi agent are modified nucleotides. The MMP7 RNAi agents disclosed herein may further be comprised of one or more modified internucleoside linkages, e.g., one or more phosphorothioate linkages. In some embodiments, a MMP7 RNAi agent contains one or more modified nucleotides and one or more modified internucleoside linkages. In some embodiments, a 2′-modified nucleotide is combined with modified internucleoside linkage.

In some embodiments, a MMP7 RNAi agent is prepared or provided as a salt, mixed salt, or a free-acid. In some embodiments, a MMP7 RNAi agent is prepared as a pharmaceutically acceptable salt. In some embodiments, a MMP7 RNAi agent is prepared as a pharmaceutically acceptable sodium salt. Such forms that are well known in the art are within the scope of the inventions disclosed herein.

### Modified Nucleotides

Modified nucleotides, when used in various oligonucleotide constructs, can preserve activity of the compound in cells while at the same time increasing the serum stability of these compounds, and can also minimize the possibility of activating interferon activity in humans upon administration of the oligonucleotide construct.

In some embodiments, a MMP7 RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides, 2′-modified nucleotides, inverted nucleotides, modified nucleobase-comprising nucleotides, bridged nucleotides, peptide nucleic acids (PNAs), 2′,3′-seco nucleotide mimics (unlocked nucleobase analogues), locked nucleotides, 3′-O-methoxy (2′ internucleoside linked) nucleotides, 2′-F-Arabino nucleotides, 5′-Methyl-2′-fluoro nucleotides, morpholino nucleotides (modified nucleotides with a morpholine ring), nucleotides where the typical 5-membered sugar ring of the nucleotide has been modified, vinyl phosphonate deoxyribonucleotides, vinyl phosphonate containing nucleotides, and cyclopropyl phosphonate containing nucleotides. 2′-modified nucleotides (i.e., a nucleotide with a group other than a hydroxyl group at the 2′ position of the five-membered sugar ring) include, but are not limited to, 2′-O-methyl nucleotides (also referred to as 2′-methoxy nucleotides), 2′-fluoro nucleotides (also referred to herein as 2′-deoxy-2′-fluoro nucleotides), 2′-deoxy nucleotides, 2′-methoxyethyl (2′-O-2-methoxylethyl) nucleotides (also referred to as 2′-MOE), 2′-amino nucleotides, 2′-halo nucleotides, and 2′-alkyl nucleotides. It is not necessary for all positions in a given compound to be uniformly modified. Conversely, more than one modification can be incorporated in a single MMP7 RNAi agent or even in a single nucleotide thereof. The MMP7 RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrinidines, 6-azapyrimidines and N-2, N-6 and O-6 substituted purines, (e.g., 2-aninopropyladenine, 5-propynyluracil, or 5-propynylcytosine), 5-methylcytosine (5-me-C), 5-hydroxymethyl cytosine, inosine, xanthine, hypoxanthine, 2-aninoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulflydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-mnethyladenine, 8-azaguanine and 8-azaadenine, 7-deazaguanine, 7-deazaadenine, 3-deazaguanine, and 3-deazaadenine.

In some embodiments, the 5′ and/or 3′ end of the antisense strand can include abasic residues (Ab), which can also be referred to as an “abasic site” or “abasic nucleotide.” An abasic residue (Ab) is a nucleotide or nucleoside that lacks a nucleobase at the 1′ position of the sugar moiety. (See, e.g., U.S. Pat. No. 5,998,203). In some embodiments, an abasic residue can be placed internally in a nucleotide sequence. In some embodiments, Ab or AbAb can be added to the 3′ end of the antisense strand. In some embodiments, the 5′ end of the sense strand can include one or more additional abasic residues (e.g., (Ab) or (AbAb)). In some embodiments, UUAb, UAb, or Ab are added to the 3′ end of the sense strand. In some embodiments, an abasic (deoxyribose) residue can be replaced with a ribitol (abasic ribose) residue.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense sense strand wherein substantially all of the nucleotides present are modified nucleotides is an antisense strand having two or fewer (i.e., 0, 1, or 2) nucleotides in the antisense strand being unmodified ribonucleotides. In some embodiments, one or more nucleotides of an RNAi agent is an unmodified ribonucleotide. Chemical structures for certain modified nucleotides are set forth in Table 11 herein.

### Modified Internucleoside Linkages

In some embodiments, one or more nucleotides of a MMP7 RNAi agent are linked by non-standard linkages or backbones (i.e., modified internucleoside linkages or modified backbones). Modified internucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of a MMP7 RNAi agent can contain 1, 2, 3, 4, 5, or 6 phosphorothioate linkages, an antisense strand of a MMP7 RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of a MMP7 RNAi agent can contain 1, 2, 3, or 4 phosphorothioate linkages, an antisense strand of a MMP7 RNAi agent can contain 1, 2, 3, or 4 phosphorothioate linkages, or both the sense strand and the antisense strand independently can contain 1, 2, 3, or 4 phosphorothioate linkages.

In some embodiments, a MMP7 RNAi agent sense strand contains at least two phosphorothioate internucleoside linkages. In some embodiments, the phosphorothioate internucleoside linkages are between the nucleotides at positions 1-3 from the 3′ end of the sense strand. In some embodiments, one phosphorothioate internucleoside linkage is at the 5′ end of the sense strand nucleotide sequence, and another phosphorothioate linkage is at the 3′ end of the sense strand nucleotide sequence. In some embodiments, two phosphorothioate internucleoside linkage are located at the 5′ end of the sense strand, and another phosphorothioate linkage is at the 3′ end of the sense strand. In some embodiments, the sense strand does not include any phosphorothioate internucleoside linkages between the nucleotides, but contains one, two, or three phosphorothioate linkages between the terminal nucleotides on both the 5′ and 3′ ends and the optionally present inverted abasic residue terminal caps. In some embodiments, the targeting ligand is linked to the sense strand via a phosphorothioate linkage.

In some embodiments, a MMP7 RNAi agent antisense strand contains four phosphorothioate internucleoside linkages. In some embodiments, the four phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, three phosphorothioate internucleoside linkages are located between positions 1-4 from the 5′ end of the antisense strand, and a fourth phosphorothioate internucleoside linkage is located between positions 20-21 from the 5′ end of the antisense strand. In some embodiments, a MMP7 RNAi agent contains at least three or four phosphorothioate internucleoside linkages in the antisense strand.

### Capping Residues or Moieties

In some embodiments, the sense strand may include one or more capping residues or moieties, sometimes referred to in the art as a “cap,” a “terminal cap,” or a “capping residue.” As used herein, a “capping residue” is anon-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some embodiments, inverted abasic residues (invAb) (also referred to in the art as “inverted abasic sites”) are added as capping residues (see Table 11). (See, e.g., F. Czaudema, Nucleic Acids Res., 2003, 31(11), 2705-16). Capping residues are generally known in the art, and include, for example, inverted abasic residues as well as carbon chains such as a terminal C₃H₇ (propyl), C₆H₁₃ (hexyl), or C₁₂H₂₅ (dodecyl) groups. In some embodiments, a capping residue is present at either the 5′ terminal end, the 3′ terminal end, or both the 5′ and 3′ terminal ends of the sense strand. In some embodiments, the 5′ end and/or the 3′ end of the sense strand may include more than one inverted abasic deoxyribose moiety as a capping residue.

In some embodiments, one or more inverted abasic residues (invAb) are added to the 3′ end of the sense strand. In some embodiments, one or more inverted abasic residues (invAb) are added to the 5′ end of the sense strand. In some embodiments, one or more inverted abasic residues or inverted abasic sites are inserted between the targeting ligand and the nucleotide sequence of the sense strand of the RNAi agent. In some embodiments, the inclusion of one or more inverted abasic residues or inverted abasic sites at or near the terminal end or terminal ends of the sense strand of an RNAi agent allows for enhanced activity or other desired properties of an RNAi agent.

In some embodiments, one or more inverted abasic residues (invAb) are added to the 5′ end of the sense strand. In some embodiments, one or more inverted abasic residues can be inserted between the targeting ligand and the nucleotide sequence of the sense strand of the RNAi agent. The inverted abasic residues may be linked via phosphate, phosphorothioate (e.g., shown herein as (invAb)s)), or other internucleoside linkages. In some embodiments, the inclusion of one or more inverted abasic residues at or near the terminal end or terminal ends of the sense strand of an RNAi agent may allow for enhanced activity or other desired properties of an RNAi agent. In some embodiments, an inverted abasic (deoxyribose) residue can be replaced with an inverted ribitol (abasic ribose) residue. In some embodiments, the 3′ end of the antisense strand core stretch sequence, or the 3′ end of the antisense strand sequence, may include an inverted abasic residue. The chemical structures for inverted abasic deoxyribose residues are shown in Table 11 below.

### MMP7 RNAi Agents

The MMP7 RNAi agents disclosed herein are designed to target specific positions on a MMP7 gene (e.g., SEQ ID NO:1 (NM_002423.5)). As defined herein, an antisense strand sequence is designed to target a MMP7 gene at a given position on the gene when the 5′ terminal nucleobase of the antisense strand is aligned with a position that is 21 nucleotides downstream (towards the 3′ end) from the position on the gene when base pairing to the gene. For example, as illustrated in Tables 1 and 2 herein, an antisense strand sequence designed to target a MMP7 gene at position 303 requires that when base pairing to the gene, the 5′ terminal nucleobase of the antisense strand is aligned with position 323 of a MMP7 gene.

As provided herein, a MMP7 RNAi agent does not require that the nucleobase at position 1 (5′→3′) of the antisense strand be complementary to the gene, provided that there is at least 85% complementarity (e.g., at least 85, 86, 87, 88, 89, 90, 91, 92, 93, 94, 95, 96, 97, 98, 99, or 100% complementarity) of the antisense strand and the gene across a core stretch sequence of at least 16 consecutive nucleotides. For example, for a MMP7 RNAi agent disclosed herein that is designed to target position 303 of a MMP7 gene, the 5′ terminal nucleobase of the antisense strand of the of the MMP7 RNAi agent must be aligned with position 323 of the gene; however, the 5′ terminal nucleobase of the antisense strand may be, but is not required to be, complementary to position 323 of a MMP7 gene, provided that there is at least 85% complementarity (e.g., at least 85, 86, 87, 88, 89, 90, 91, 92, 93, 94, 95, 96, 97, 98, 99, or 100% complementarity) of the antisense strand and the gene transcript across a core stretch sequence of at least 16 consecutive nucleotides. As shown by, among other things, the various examples disclosed herein, the specific site of binding of the gene by the antisense strand of the MMP7 RNAi agent (e.g., whether the MMP7 RNAi agent is designed to target a MMP7 gene at position 303, at position 418, at position 971, or at some other position) is an important factor to the level of inhibition achieved by the MMP7 RNAi agent. (See, e.g., Kamola et al., The siRNA Non-seed Region and Its Target Sequences are Auxiliary Determinants of Off-Target Effects, PLOS Computational Biology, 11(12), FIG. 1 (2015)).

In some embodiments, the MMP7 RNAi agents disclosed herein target a MMP7 gene at or near the positions of the MMP7 sequence shown in Table 1. In some embodiments, the antisense strand of a MMP7 RNAi agent disclosed herein includes a core stretch sequence that is fully, substantially, or at least partially complementary to a target MMP7 19-mer sequence disclosed in Table 1.

TABLE 1
MMP7 19-mer mRNA Target Sequences (taken from homo sapiens matrix
metallopeptidase 7 (MMP7) transcript, GenBank NM_002423.5 (SEQ ID NO: 1))

| | MMP7 19-mer | Corresponding | Targeted Gene |
|---|---|---|---|
| SEQ ID | Target Sequences | Positions of Sequence | Position (as |
| No. | (5′→3′) | on SEQ ID NO: 1 | referred to herein) |
| 2 | AUGUGGAGUGCCAGAUGUU | 305-323 | 303 |
| 3 | GUGGAGUGCCAGAUGUUGC | 307-325 | 305 |
| 4 | UGGAGUGCCAGAUGUUGCA | 308-326 | 306 |
| 5 | GAGUGCCAGAUGUUGCAGA | 310-328 | 308 |
| 6 | AGUGCCAGAUGUUGCAGAA | 311-329 | 309 |
| 7 | GUGCCAGAUGUUGCAGAAU | 312-330 | 310 |
| 8 | UGCCAGAUGUUGCAGAAUA | 313-331 | 311 |
| 9 | CAUUUGAUGGGCCAGGAAA | 550-568 | 548 |
| 10 | UGAUGGGCCAGGAAACACG | 554-572 | 552 |
| 11 | GGCUCAGGACUAUCUCAAG | 149-167 | 147 |
| 12 | CUCAAGAGAUUUUAUCUCU | 162-180 | 160 |
| 13 | AGAGAUUUUAUCUCUAUGA | 166-184 | 164 |
| 14 | GAUUUUAUCUCUAUGACUC | 169-187 | 167 |
| 15 | UGUGGAGUGCCAGAUGUUA | 306-324 | 304 |
| 16 | AGUGCCAGAUGUUGCAGAA | 311-329 | 309 |
| 17 | CAGAUGUUGCAGAAUACUC | 316-334 | 314 |
| 18 | UUGCAGAAUACUCACUAUU | 322-340 | 320 |
| 19 | UGCAGAAUACUCACUAUUU | 323-341 | 321 |
| 20 | UACAGUGGAUCGAUUAGUG | 416-434 | 414 |
| 21 | ACAGUGGAUCGAUUAGUGU | 417-435 | 415 |
| 22 | GUGGAUCGAUUAGUGUCAA | 420-438 | 418 |
| 23 | GAGAUGCUCACUUCGAUGA | 610-628 | 608 |
| 24 | AUUAACUUCCUGUAUGCUA | 663-681 | 661 |
| 25 | AGUGAUGUAUCCAACCUAU | 737-755 | 735 |
| 26 | GUAUCCAACCUAUGGAAAU | 743-461 | 741 |
| 27 | AAGGCAUUCAGAAACUAUA | 802-820 | 800 |
| 28 | GUUGCACAAUCAGAAUUGA | 902-920 | 900 |
| 29 | UUGCACAAUCAGAAUUGAU | 903-921 | 901 |
| 30 | CAAUCAGAAUUGAUAAGCA | 908-926 | 906 |
| 31 | AAUCAGAAUUGAUAAGCAA | 909-927 | 907 |
| 32 | GUCACCCUUUUUUAUUGCA | 955-973 | 953 |
| 33 | GCAGUUGGUUUUUGAAUGU | 971-989 | 969 |
| 34 | AGUUGGUUUUUGAAUGUCU | 973-991 | 971 |
| 35 | GUUGGUUUUUGAAUGUCUU | 974-992 | 972 |
| 36 | CUCCUUUUAAGGAUAAACU | 996-1014 | 994 |

Homo sapiens matrix metallopeptidase 7 (MMP7), GenBank NM_002423.5 (SEQ ID NO:1), gene transcript (1119 bases):

| | |
|---|---|
| 1 | accaaatcaa ccataggtcc aagaacaatt gtctctggac ggcagctatg cgactcaccg |
| 61 | tgctgtgtgc tgtgtgcctg ctgcctggca gcctggccct gccgctgcct caggaggcgg |
| 121 | gaggcatgag tgagctacag tgggaacagg ctcaggacta tctcaagaga ttttatctct |
| 181 | atgactcaga aacaaaaaat gccaacagtt tagaagccaa actcaaggag atgcaaaaat |
| 241 | tctttggcct acctataact ggaatgttaa actcccgcgt catagaaata atgcagaagc |
| 301 | ccagatgtgg agtgccagat gttgcagaat actcactatt tccaaatagc ccaaaatgga |
| 361 | cttccaaagt ggtcacctac aggatcgtat catatactcg agacttaccg catattacag |
| 421 | tggatcgatt agtgtcaaag gctttaaaca tgtggggcaa agagatcccc ctgcatttca |
| 481 | ggaaagttgt atggggaact gctgacatca tgattggctt tgcgcgagga gctcatgggg |
| 541 | actcctaccc atttgatggg ccaggaaaca cgctggctca tgcctttgcg cctgggacag |
| 601 | gtctcggagg agatgctcac ttcgatgagg atgaacgctg gacggatggt agcagtctag |
| 661 | ggattaactt cctgtatgct gcaactcatg aacttggcca ttctttgggt atgggacatt |
| 721 | cctctgatcc taatgcagtg atgtatccaa cctatggaaa tggagatccc caaaatttta |
| 781 | aactttccca ggatgatatt aaaggcattc agaaactata tggaaagaga agtaattcaa |
| 841 | gaaagaaata gaaacttcag gcagaacatc cattcattca ttcattggat tgtatatcat |
| 901 | tgttgcacaa tcagaattga taagcactgt tcctccactc catttagcaa ttatgtcacc |
| 961 | cttttttatt gcagttggtt tttgaatgtc tttcactcct tttaaggata aactccttta |
| 1021 | tggtgtgact gtgtcttatt catctatact tgcagtgggt agatgtcaat aaatgttaca |
| 1081 | tacacaaata aataaaatgt ttattccatg gtaaattta |

In some embodiments, a MMP7 RNAi agent includes an antisense strand wherein position 19 of the antisense strand (5′→3′) is capable of forming a base pair with position 1 of a 19-mer target sequence disclosed in Table 1. In some embodiments, a MMP7 agent includes an antisense strand wherein position 1 of the antisense strand (5′→3′) is capable of forming a base pair with position 19 of a 19-mer target sequence disclosed in Table 1.

In some embodiments, a MMP7 agent includes an antisense strand wherein position 2 of the antisense strand (5′→3′) is capable of forming a base pair with position 18 of a 19-mer target sequence disclosed in Table 1. In some embodiments, a MMP7 agent includes an antisense strand wherein positions 2 through 18 of the antisense strand (5′→3′) are capable of forming base pairs with each of the respective complementary bases located at positions 18 through 2 of the 19-mer target sequence disclosed in Table 1.

For the RNAi agents disclosed herein, the nucleotide at position 1 of the antisense strand (from 5′ end→3′ end) can be perfectly complementary to a MMP7 gene, or can be non-complementary to a MMP7 gene. In some embodiments, the nucleotide at position 1 of the antisense strand (from 5′ end→3′ end) is a U, A, or dT. In some embodiments, the nucleotide at position 1 of the antisense strand (from 5′ end→3′ end) forms an A:U or U:A base pair with the sense strand.

In some embodiments, a MMP7 RNAi agent antisense strand comprises the sequence of nucleotides (from 5′ end→3′ end) 2-18 or 2-19 of any of the antisense strand sequences in Table 2 or Table 3. In some embodiments, a MMP7 RNAi sense strand comprises the sequence of nucleotides (from 5′ end→3′ end) 1-17, 1-18, or 2-18 of any of the sense strand sequences in Table 2, Table 4, Table 5, or Table 6.

In some embodiments, a MMP7 RNAi agent is comprised of (i) an antisense strand comprising the sequence of nucleotides (from 5′ end→3′ end) 2-18 or 2-19 of any of the antisense strand sequences in Table 2 or Table 3, and (ii) a sense strand comprising the sequence of nucleotides (from 5′ end→3′ end) 1-17 or 1-18 of any of the sense strand sequences in Table 2, Table 4, Table 5, or Table 6.

In some embodiments, the MMP7 RNAi agents include core 19-mer nucleotide sequences shown in the following Table 2.

TABLE 2
MMP7 RNAi Agent Antisense Strand and Sense Strand Core Stretch Base Sequences
(N = any nucleobase; I = inosine (hypoxanthine nucleobase)

| | | | | Corresponding | |
|---|---|---|---|---|---|
| | Antisense Strand Base | | Sense Strand Base | Positions of | |
| | Sequence (5′ → 3′) | | Sequence (5′ → 3′) | Identified | Targeted |
| SEQ ID | (Shown as an Unmodified | SEQ ID | (Shown as an Unmodified | Sequence on | Gene |
| NO:. | Nucleotide Sequence) | NO:. | Nucleotide Sequence) | SEQ ID NO: 1 | Position |
| 37 | AACAUCUGGCACUCCACAU | 187 | AUGUGGAGUGCCAGAUGUU | 305-323 | 303 |
| 38 | UACAUCUGGCACUCCACAU | 188 | AUGUGGAGUGCCAGAUGUA | 305-323 | 303 |
| 39 | NACAUCUGGCACUCCACAU | 189 | AUGUGGAGUGCCAGAUGUN | 305-323 | 303 |
| 40 | NACAUCUGGCACUCCACAN | 190 | NUGUGGAGUGCCAGAUGUN | 305-323 | 303 |
| 41 | UCAACAUCUGGCACUCCAC | 191 | GUGGAGUGCCAGAUGUUGA | 307-325 | 305 |
| 42 | ACAACAUCUGGCACUCCAC | 192 | GUGGAGUGCCAGAUGUUGU | 307-325 | 305 |
| 43 | GCAACAUCUGGCACUCCAC | 193 | GUGGAGUGCCAGAUGUUGC | 307-325 | 305 |
| 44 | NCAACAUCUGGCACUCCAC | 194 | GUGGAGUGCCAGAUGUUGN | 307-325 | 305 |
| 45 | NCAACAUCUGGCACUCCAN | 195 | NUGGAGUGCCAGAUGUUGN | 307-325 | 305 |
| 46 | UGCAACAUCUGGCACUCCA | 196 | UGGAGUGCCAGAUGUUGCA | 308-326 | 306 |
| 47 | AGCAACAUCUGGCACUCCA | 197 | UGGAGUGCCAGAUGUUGCU | 308-326 | 306 |
| 48 | NGCAACAUCUGGCACUCCA | 198 | UGGAGUGCCAGAUGUUGCN | 308-326 | 306 |
| 49 | NGCAACAUCUGGCACUCCN | 199 | NGGAGUGCCAGAUGUUGCN | 308-326 | 306 |
| 50 | UCUGCAACAUCUGGCACUC | 200 | GAGUGCCAGAUGUUGCAGA | 310-328 | 308 |
| 51 | ACUGCAACAUCUGGCACUC | 201 | GAGUGCCAGAUGUUGCAGU | 310-328 | 308 |
| 52 | NCUGCAACAUCUGGCACUC | 202 | GAGUGCCAGAUGUUGCAGN | 310-328 | 308 |
| 53 | NCUGCAACAUCUGGCACUN | 203 | NAGUGCCAGAUGUUGCAGN | 310-328 | 308 |
| 54 | UUCUGCAACAUCUGGCACU | 204 | AGUGCCAGAUGUUGCAGAA | 311-329 | 309 |
| 55 | AUCUGCAACAUCUGGCACU | 205 | AGUGCCAGAUGUUGCAGAU | 311-329 | 309 |
| 56 | NUCUGCAACAUCUGGCACU | 206 | AGUGCCAGAUGUUGCAGAN | 311-329 | 309 |
| 57 | NUCUGCAACAUCUGGCACN | 207 | NGUGCCAGAUGUUGCAGAN | 311-329 | 309 |
| 58 | AUUCUGCAACAUCUGGCAC | 208 | GUGCCAGAUGUUGCAGAAU | 312-330 | 310 |
| 59 | UUUCUGCAACAUCUGGCAC | 209 | GUGCCAGAUGUUGCAGAAA | 312-330 | 310 |
| 60 | NUUCUGCAACAUCUGGCAC | 210 | GUGCCAGAUGUUGCAGAAN | 312-330 | 310 |
| 61 | NUUCUGCAACAUCUGGCAN | 211 | NUGCCAGAUGUUGCAGAAN | 312-330 | 310 |
| 62 | UAUUCUGCAACAUCUGGCA | 212 | UGCCAGAUGUUGCAGAAUA | 313-331 | 311 |
| 63 | AAUUCUGCAACAUCUGGCA | 213 | UGCCAGAUGUUGCAGAAUU | 313-331 | 311 |
| 64 | NAUUCUGCAACAUCUGGCA | 214 | UGCCAGAUGUUGCAGAAUN | 313-331 | 311 |
| 65 | NAUUCUGCAACAUCUGGCN | 215 | NGCCAGAUGUUGCAGAAUN | 313-331 | 311 |
| 66 | UUUCCUGGCCCAUCAAAUG | 216 | CAUUUGAUGGGCCAGGAAA | 550-568 | 548 |
| 67 | AUUCCUGGCCCAUCAAAUG | 217 | CAUUUGAUGGGCCAGGAAU | 550-568 | 548 |
| 68 | NUUCCUGGCCCAUCAAAUG | 218 | CAUUUGAUGGGCCAGGAAN | 550-568 | 548 |
| 69 | NUUCCUGGCCCAUCAAAUN | 219 | NAUUUGAUGGGCCAGGAAN | 550-568 | 548 |
| 70 | UGUGUUUCCUGGCCCAUCA | 220 | UGAUGGGCCAGGAAACACA | 554-572 | 552 |
| 71 | AGUGUUUCCUGGCCCAUCA | 221 | UGAUGGGCCAGGAAACACU | 554-572 | 552 |
| 72 | CGUGUUUCCUGGCCCAUCA | 222 | UGAUGGGCCAGGAAACACG | 554-572 | 552 |
| 73 | NGUGUUUCCUGGCCCAUCA | 223 | UGAUGGGCCAGGAAACACN | 554-572 | 552 |
| 74 | NGUGUUUCCUGGCCCAUCN | 224 | NGAUGGGCCAGGAAACACN | 554-572 | 552 |
| 75 | UUUGAGAUAGUCCUGAGCC | 225 | GGCUCAGGACUAUCUCAAA | 149-167 | 147 |
| 76 | AUUGAGAUAGUCCUGAGCC | 226 | GGCUCAGGACUAUCUCAAU | 149-167 | 147 |
| 77 | CUUGAGAUAGUCCUGAGCC | 227 | GGCUCAGGACUAUCUCAAG | 149-167 | 147 |
| 78 | NUUGAGAUAGUCCUGAGCC | 228 | GGCUCAGGACUAUCUCAAN | 149-167 | 147 |
| 79 | NUUGAGAUAGUCCUGAGCN | 229 | NGCUCAGGACUAUCUCAAN | 149-167 | 147 |
| 80 | AGAGAUAAAAUCUCUUGAG | 230 | CUCAAGAGAUUUUAUCUCU | 162-180 | 160 |
| 81 | UGAGAUAAAAUCUCUUGAG | 231 | CUCAAGAGAUUUUAUCUCA | 162-180 | 160 |
| 82 | NGAGAUAAAAUCUCUUGAG | 232 | CUCAAGAGAUUUUAUCUCN | 162-180 | 160 |
| 83 | NGAGAUAAAAUCUCUUGAN | 233 | NUCAAGAGAUUUUAUCUCN | 162-180 | 160 |
| 84 | UCAUAGAGAUAAAAUCUCU | 234 | AGAGAUUUUAUCUCUAUGA | 166-184 | 164 |
| 85 | ACAUAGAGAUAAAAUCUCU | 235 | AGAGAUUUUAUCUCUAUGU | 166-184 | 164 |
| 86 | NCAUAGAGAUAAAAUCUCU | 236 | AGAGAUUUUAUCUCUAUGN | 166-184 | 164 |
| 87 | NCAUAGAGAUAAAAUCUCN | 237 | NGAGAUUUUAUCUCUAUGN | 166-184 | 164 |
| 88 | UAGUCAUAGAGAUAAAAUC | 238 | GAUUUUAUCUCUAUGACUA | 169-187 | 167 |
| 89 | AAGUCAUAGAGAUAAAAUC | 239 | GAUUUUAUCUCUAUGACUU | 169-187 | 167 |
| 90 | GAGUCAUAGAGAUAAAAUC | 240 | GAUUUUAUCUCUAUGACUC | 169-187 | 167 |
| 91 | NAGUCAUAGAGAUAAAAUC | 241 | GAUUUUAUCUCUAUGACUN | 169-187 | 167 |
| 92 | NAGUCAUAGAGAUAAAAUN | 242 | NAUUUUAUCUCUAUGACUN | 169-187 | 167 |
| 93 | UAACAUCUGGCACUCCACA | 243 | UGUGGAGUGCCAGAUGUUA | 306-324 | 304 |
| 94 | AAACAUCUGGCACUCCACA | 244 | UGUGGAGUGCCAGAUGUUU | 306-324 | 304 |
| 95 | NAACAUCUGGCACUCCACA | 245 | UGUGGAGUGCCAGAUGUUN | 306-324 | 304 |
| 96 | NAACAUCUGGCACUCCACN | 246 | NGUGGAGUGCCAGAUGUUN | 306-324 | 304 |
| 97 | UUCUGCAACAUCUGGCACU | 247 | AGUGCCAGAUGUUGCAGAA | 311-329 | 309 |
| 98 | AUCUGCAACAUCUGGCACU | 248 | AGUGCCAGAUGUUGCAGAU | 311-329 | 309 |
| 99 | NUCUGCAACAUCUGGCACU | 249 | AGUGCCAGAUGUUGCAGAN | 311-329 | 309 |
| 100 | NUCUGCAACAUCUGGCACN | 250 | NGUGCCAGAUGUUGCAGAN | 311-329 | 309 |
| 101 | UAGUAUUCUGCAACAUCUG | 251 | CAGAUGUUGCAGAAUACUA | 316-334 | 314 |
| 102 | AAGUAUUCUGCAACAUCUG | 252 | CAGAUGUUGCAGAAUACUU | 316-334 | 314 |
| 103 | GAGUAUUCUGCAACAUCUG | 253 | CAGAUGUUGCAGAAUACUC | 316-334 | 314 |
| 104 | NAGUAUUCUGCAACAUCUG | 254 | CAGAUGUUGCAGAAUACUN | 316-334 | 314 |
| 105 | NAGUAUUCUGCAACAUCUN | 255 | NAGAUGUUGCAGAAUACUN | 316-334 | 314 |
| 106 | AAUAGUGAGUAUUCUGCAA | 256 | UUGCAGAAUACUCACUAUU | 322-340 | 320 |
| 107 | UAUAGUGAGUAUUCUGCAA | 257 | UUGCAGAAUACUCACUAUA | 322-340 | 320 |
| 108 | NAUAGUGAGUAUUCUGCAA | 258 | UUGCAGAAUACUCACUAUN | 322-340 | 320 |
| 109 | NAUAGUGAGUAUUCUGCAN | 259 | NUGCAGAAUACUCACUAUN | 322-340 | 320 |
| 110 | AAAUAGUGAGUAUUCUGCA | 260 | UGCAGAAUACUCACUAUUU | 323-341 | 321 |
| 111 | UAAUAGUGAGUAUUCUGCA | 261 | UGCAGAAUACUCACUAUUA | 323-341 | 321 |
| 112 | NAAUAGUGAGUAUUCUGCA | 262 | UGCAGAAUACUCACUAUUN | 323-341 | 321 |
| 113 | NAAUAGUGAGUAUUCUGCN | 263 | NGCAGAAUACUCACUAUUN | 323-341 | 321 |
| 114 | UACUAAUCGAUCCACUGUA | 264 | UACAGUGGAUCGAUUAGUA | 416-434 | 414 |
| 115 | AACUAAUCGAUCCACUGUA | 265 | UACAGUGGAUCGAUUAGUU | 416-434 | 414 |
| 116 | CACUAAUCGAUCCACUGUA | 266 | UACAGUGGAUCGAUUAGUG | 416-434 | 414 |
| 117 | NACUAAUCGAUCCACUGUA | 267 | UACAGUGGAUCGAUUAGUN | 416-434 | 414 |
| 118 | NACUAAUCGAUCCACUGUN | 268 | NACAGUGGAUCGAUUAGUN | 416-434 | 414 |
| 119 | ACACUAAUCGAUCCACUGU | 269 | ACAGUGGAUCGAUUAGUGU | 417-435 | 415 |
| 120 | UCACUAAUCGAUCCACUGU | 270 | ACAGUGGAUCGAUUAGUGA | 417-435 | 415 |
| 121 | NCACUAAUCGAUCCACUGU | 271 | ACAGUGGAUCGAUUAGUGN | 417-435 | 415 |
| 122 | NCACUAAUCGAUCCACUGN | 272 | NCAGUGGAUCGAUUAGUGN | 417-435 | 415 |
| 123 | UUGACACUAAUCGAUCCAC | 273 | GUGGAUCGAUUAGUGUCAA | 420-438 | 418 |
| 124 | AUGACACUAAUCGAUCCAC | 274 | GUGGAUCGAUUAGUGUCAU | 420-438 | 418 |
| 125 | NUGACACUAAUCGAUCCAC | 275 | GUGGAUCGAUUAGUGUCAN | 420-438 | 418 |
| 126 | NUGACACUAAUCGAUCCAN | 276 | NUGGAUCGAUUAGUGUCAN | 420-438 | 418 |
| 123 | UUGACACUAAUCGAUCCAC | 277 | GUGGAUCGAUUAGUIUCAA | 420-438 | 418 |
| 128 | AUGACACUAAUCGAUCCAC | 278 | GUGGAUCGAUUAGUIUCAU | 420-438 | 418 |
| 129 | NUGACACUAAUCGAUCCAC | 279 | GUGGAUCGAUUAGUIUCAN | 420-438 | 418 |
| 130 | NUGACACUAAUCGAUCCAN | 280 | NUGGAUCGAUUAGUIUCAN | 420-438 | 418 |
| 131 | UCAUCGAAGUGAGCAUCUC | 281 | GAGAUGCUCACUUCGAUGA | 610-628 | 608 |
| 132 | ACAUCGAAGUGAGCAUCUC | 282 | GAGAUGCUCACUUCGAUGU | 610-628 | 608 |
| 133 | NCAUCGAAGUGAGCAUCUC | 283 | GAGAUGCUCACUUCGAUGN | 610-628 | 608 |
| 134 | NCAUCGAAGUGAGCAUCUN | 284 | NAGAUGCUCACUUCGAUGN | 610-628 | 608 |
| 135 | UAGCAUACAGGAAGUUAAU | 285 | AUUAACUUCCUGUAUGCUA | 663-681 | 661 |
| 136 | AAGCAUACAGGAAGUUAAU | 286 | AUUAACUUCCUGUAUGCUU | 663-681 | 661 |
| 137 | NAGCAUACAGGAAGUUAAU | 287 | AUUAACUUCCUGUAUGCUN | 663-681 | 661 |
| 138 | NAGCAUACAGGAAGUUAAN | 288 | NUUAACUUCCUGUAUGCUN | 663-681 | 661 |
| 139 | AUAGGUUGGAUACAUCACU | 289 | AGUGAUGUAUCCAACCUAU | 737-755 | 735 |
| 140 | UUAGGUUGGAUACAUCACU | 290 | AGUGAUGUAUCCAACCUAA | 737-755 | 735 |
| 141 | NUAGGUUGGAUACAUCACU | 291 | AGUGAUGUAUCCAACCUAN | 737-755 | 735 |
| 142 | NUAGGUUGGAUACAUCACN | 292 | NGUGAUGUAUCCAACCUAN | 737-755 | 735 |
| 143 | AUUUCCAUAGGUUGGAUAC | 293 | GUAUCCAACCUAUGGAAAU | 743-461 | 741 |
| 144 | UUUUCCAUAGGUUGGAUAC | 294 | GUAUCCAACCUAUGGAAAA | 743-461 | 741 |
| 145 | NUUUCCAUAGGUUGGAUAC | 295 | GUAUCCAACCUAUGGAAAN | 743-461 | 741 |
| 146 | NUUUCCAUAGGUUGGAUAN | 296 | NUAUCCAACCUAUGGAAAN | 743-461 | 741 |
| 147 | UAUAGUUUCUGAAUGCCUU | 297 | AAGGCAUUCAGAAACUAUA | 802-820 | 800 |
| 148 | AAUAGUUUCUGAAUGCCUU | 298 | AAGGCAUUCAGAAACUAUU | 802-820 | 800 |
| 149 | NAUAGUUUCUGAAUGCCUU | 299 | AAGGCAUUCAGAAACUAUN | 802-820 | 800 |
| 150 | NAUAGUUUCUGAAUGCCUN | 300 | NAGGCAUUCAGAAACUAUN | 802-820 | 800 |
| 151 | UCAAUUCUGAUUGUGCAAC | 301 | GUUGCACAAUCAGAAUUGA | 902-920 | 900 |
| 152 | ACAAUUCUGAUUGUGCAAC | 302 | GUUGCACAAUCAGAAUUGU | 902-920 | 900 |
| 153 | NCAAUUCUGAUUGUGCAAC | 303 | GUUGCACAAUCAGAAUUGN | 902-920 | 900 |
| 154 | NCAAUUCUGAUUGUGCAAN | 304 | NUUGCACAAUCAGAAUUGN | 902-920 | 900 |
| 155 | AUCAAUUCUGAUUGUGCAA | 305 | UUGCACAAUCAGAAUUGAU | 903-921 | 901 |
| 156 | UUCAAUUCUGAUUGUGCAA | 306 | UUGCACAAUCAGAAUUGAA | 903-921 | 901 |
| 157 | NUCAAUUCUGAUUGUGCAA | 307 | UUGCACAAUCAGAAUUGAN | 903-921 | 901 |
| 158 | NUCAAUUCUGAUUGUGCAN | 308 | NUGCACAAUCAGAAUUGAN | 903-921 | 901 |
| 159 | UGCUUAUCAAUUCUGAUUG | 309 | CAAUCAGAAUUGAUAAGCA | 908-926 | 906 |
| 160 | AGCUUAUCAAUUCUGAUUG | 310 | CAAUCAGAAUUGAUAAGCU | 908-926 | 906 |
| 161 | NGCUUAUCAAUUCUGAUUG | 311 | CAAUCAGAAUUGAUAAGCN | 908-926 | 906 |
| 162 | NGCUUAUCAAUUCUGAUUN | 312 | NAAUCAGAAUUGAUAAGCN | 908-926 | 906 |
| 163 | UUGCUUAUCAAUUCUGAUU | 313 | AAUCAGAAUUGAUAAGCAA | 909-927 | 907 |
| 164 | AUGCUUAUCAAUUCUGAUU | 314 | AAUCAGAAUUGAUAAGCAU | 909-927 | 907 |
| 165 | NUGCUUAUCAAUUCUGAUU | 315 | AAUCAGAAUUGAUAAGCAN | 909-927 | 907 |
| 166 | NUGCUUAUCAAUUCUGAUN | 316 | NAUCAGAAUUGAUAAGCAN | 909-927 | 907 |
| 167 | UGCAAUAAAAAAGGGUGAC | 317 | GUCACCCUUUUUUAUUGCA | 955-973 | 953 |
| 168 | AGCAAUAAAAAAGGGUGAC | 318 | GUCACCCUUUUUUAUUGCU | 955-973 | 953 |
| 169 | NGCAAUAAAAAAGGGUGAC | 319 | GUCACCCUUUUUUAUUGCN | 955-973 | 953 |
| 170 | NGCAAUAAAAAAGGGUGAN | 320 | NUCACCCUUUUUUAUUGCN | 955-973 | 953 |
| 171 | ACAUUCAAAAACCAACUGC | 321 | GCAGUUGGUUUUUGAAUGU | 971-989 | 969 |
| 172 | UCAUUCAAAAACCAACUGC | 322 | GCAGUUGGUUUUUGAAUGA | 971-989 | 969 |
| 173 | NCAUUCAAAAACCAACUGC | 323 | GCAGUUGGUUUUUGAAUGN | 971-989 | 969 |
| 174 | NCAUUCAAAAACCAACUGN | 324 | NCAGUUGGUUUUUGAAUGN | 971-989 | 969 |
| 175 | AGACAUUCAAAAACCAACU | 325 | AGUUGGUUUUUGAAUGUCU | 973-991 | 971 |
| 176 | UGACAUUCAAAAACCAACU | 326 | AGUUGGUUUUUGAAUGUCA | 973-991 | 971 |
| 177 | NGACAUUCAAAAACCAACU | 327 | AGUUGGUUUUUGAAUGUCN | 973-991 | 971 |
| 178 | NGACAUUCAAAAACCAACN | 328 | NGUUGGUUUUUGAAUGUCN | 973-991 | 971 |
| 179 | AAGACAUUCAAAAACCAAC | 329 | GUUGGUUUUUGAAUGUCUU | 974-992 | 972 |
| 180 | UAGACAUUCAAAAACCAAC | 330 | GUUGGUUUUUGAAUGUCUA | 974-992 | 972 |
| 181 | NAGACAUUCAAAAACCAAC | 331 | GUUGGUUUUUGAAUGUCUN | 974-992 | 972 |
| 182 | NAGACAUUCAAAAACCAAN | 332 | NUUGGUUUUUGAAUGUCUN | 974-992 | 972 |
| 183 | AGUUUAUCCUUAAAAGGAG | 333 | CUCCUUUUAAGGAUAAACU | 996-1014 | 994 |
| 184 | UGUUUAUCCUUAAAAGGAG | 334 | CUCCUUUUAAGGAUAAACA | 996-1014 | 994 |
| 185 | NGUUUAUCCUUAAAAGGAG | 335 | CUCCUUUUAAGGAUAAACN | 996-1014 | 994 |
| 186 | NGUUUAUCCUUAAAAGGAN | 336 | NUCCUUUUAAGGAUAAACN | 996-1014 | 994 |

The MMP7 RNAi agent sense strands and antisense strands that comprise or consist of the nucleotide sequences in Table 2 can be modified nucleotides or unmodified nucleotides. In some embodiments, the MMP7 RNAi agents having the sense and antisense strand sequences that comprise or consist of any of the nucleotide sequences in Table 2 are all or substantially all modified nucleotides.

In some embodiments, the antisense strand of a MMP7 RNAi agent disclosed herein differs by 0, 1, 2, or 3 nucleotides from any of the antisense strand sequences in Table 2. In some embodiments, the sense strand of a MMP7 RNAi agent disclosed herein differs by 0, 1, 2, or 3 nucleotides from any of the sense strand sequences in Table 2.

As used herein, each N listed in a sequence disclosed in Table 2 may be independently selected from any and all nucleobases (including those found on both modified and unmodified nucleotides). In some embodiments, an N nucleotide listed in a sequence disclosed in Table 2 has a nucleobase that is complementary to the N nucleotide at the corresponding position on the other strand. In some embodiments, an N nucleotide listed in a sequence disclosed in Table 2 has a nucleobase that is not complementary to the N nucleotide at the corresponding position on the other strand. In some embodiments, an N nucleotide listed in a sequence disclosed in Table 2 has a nucleobase that is the same as the N nucleotide at the corresponding position on the other strand. In some embodiments, an N nucleotide listed in a sequence disclosed in Table 2 has a nucleobase that is different from the N nucleotide at the corresponding position on the other strand.

Certain modified MMP7 RNAi agent sense and antisense strands are provided in Table 3, Table 4, Table 5, Table 6, and Table 10. Certain modified MMP7 RNAi agent antisense strands, as well as their underlying unmodified nucleobase sequences, are provided in Table 3. Certain modified MMP7 RNAi agent sense strands, as well as their underlying unmodified nucleobase sequences, are provided in Tables 4, 5, and 6. In forming MMP7 RNAi agents, each of the nucleotides in each of the underlying base sequences listed in Tables 3, 4, 5, and 6, as well as in Table 2, above, can be a modified nucleotide.

The MMP7 RNAi agents described herein are formed by annealing an antisense strand with a sense strand. A sense strand containing a sequence listed in Table 2, Table 4, Table 5, or Table 6 can be hybridized to any antisense strand containing a sequence listed in Table 2 or Table 3, provided the two sequences have a region of at least 85% complementarity over a contiguous 16, 17, 18, 19, 20, or 21 nucleotide sequence.

In some embodiments, a MMP7 RNAi agent antisense strand comprises a nucleotide sequence of any of the sequences in Table 2 or Table 3.

In some embodiments, a MMP7 RNAi agent comprises or consists of a duplex having the nucleobase sequences of the sense strand and the antisense strand of any of the sequences in Table 2, Table 3, Table 4, Table 5, Table 6, or Table 10.

Examples of antisense strands containing modified nucleotides are provided in Table 3. Examples of sense strands containing modified nucleotides are provided in Tables 4, 5 and 6.

As used in Tables 3, 4, 5, 6, and 10, the following notations are used to indicate modified nucleotides, targeting groups, and linking groups:
A=adenosine-3′-phosphate
C=cytidine-3′-phosphate
G=guanosine-3′-phosphate
U=uridine-3′-phosphate
I=inosine-3′-phosphate
a=2′-O-methyladenosine-3′-phosphate
as =2′-O-methyladenosine-3′-phosphorothioate
c=2′-O-methylcytidine-3′-phosphate
cs=2′-O-methylcytidine-3′-phosphorothioate
g=2′-O-methylguanosine-3′-phosphate
gs=2′-O-methylguanosine-3′-phosphorothioate
i=2′-O-methylinosine-3′-phosphate
is =2′-O-methylinosine-3′-phosphorothioate
t=2′-O-methyl-5-methyluridine-3′-phosphate
ts=2′-O-methyl-5-methyluridine-3′-phosphorothioate
u=2′-O-methyluridine-3′-phosphate
us=2′-O-methyluridine-3′-phosphorothioate
Af=2′-fluoroadenosine-3′-phosphate
Afs=2′-fluoroadenosine-3′-phosporothioate
Cf=2′-fluorocytidine-3′-phosphate
Cfs=2′-fluorocytidine-3′-phosphorothioate
Gf=2′-fluoroguanosine-3′-phosphate
Gfs=2′-fluoroguanosine-3′-phosphorothioate
Tf=2′-fluoro-5′-methyluridine-3′-phosphate
Tfs=2′-fluoro-5′-methyluridine-3′-phosphorothioate
Uf=2′-fluorouridine-3′-phosphate
Ufs=2′-fluorouridine-3′-phosphorothioate
dT=2′-deoxythymidine-3′-phosphate
A_{UNA}=2′,3′-seco-adenosine-3′-phosphate
A_{UNAS}=2′,3′-seco-adenosine-3′-phosphorothioate
C_{UNA}=2′,3′-seco-cytidine-3′-phosphate
C_{UNAS}=2′,3′-seco-cytidine-3′-phosphorothioate
G_{UNA}=2′,3′-seco-guanosine-3′-phosphate
G_{UNAS}=2′,3′-seco-guanosine-3′-phosphorothioate
U_{UNA}=2′,3′-seco-uridine-3′-phosphate
U_{UNAS}=2′,3′-seco-uridine-3′-phosphorothioate
a_2N=see Table 11
a_2Ns=see Table 11
(invAb)=inverted abasic deoxyribonucleotide-5′-phosphate, see Table 11
(invAb)s=inverted abasic deoxyribonucleotide-5′-phosphorothioate, see Table 11
s=phosphorothioate linkage
p=terminal phosphate (as synthesized)
vpdN=vinyl phosphonate deoxyribonucleotide
cPrpa=5′-cyclopropyl phosphonate-2′-O-methyladenosine-3′-phosphate (see Table 11)
cPrpas=5′-cyclopropyl phosphonate-2′-O-methyladenosine-3′-phosphorothioate (see Table 11)
cPrpu=5′-cyclopropyl phosphonate-2′-O-methyluridine-3′-phosphate (see Table 11)
cPrpus=5′-cyclopropyl phosphonate-2′-O-methyluridine-3′-phosphorothioate (see Table 11)
(Alk-SS-C6)=see Table 11
(C6-SS-Alk)=see Table 11
(C6-SS-C6)=see Table 11
(6-SS-6)=see Table 11
(C6-SS-Alk-Me)=see Table 11
(NH2-C6)=see Table 11
(TriAlk14)=see Table 11
(TriAlk14)s=see Table 11
-C6-=see Table 11
-C6s-=see Table 11
-L6-C6-=see Table 11
-L6-C6s-=see Table 11
-Alk-cyHex-=see Table 11
Alk-cyHexs-=see Table 11
(TA14)=see Table 11 (structure of (TriAlk14)s after conjugation)
(TA14)s=see Table 11 (structure of (TriAlk14)s after conjugation)

As the person of ordinary skill in the art would readily understand, unless otherwise indicated by the sequence (such as, for example, by a phosphorothioate linkage “s”), when present in an oligonucleotide, the nucleotide monomers are mutually linked by 5′-3′-phosphodiester bonds. As the person of ordinary skill in the art would clearly understand, the inclusion of a phosphorothioate linkage as shown in the modified nucleotide sequences disclosed herein replaces the phosphodiester linkage typically present in oligonucleotides. Further, the person of ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo. Additionally, for the embodiments disclosed herein, when viewing the respective strand 5′→3′, the inverted abasic residues are inserted such that the 3′ position of the deoxyribose is linked at the 3′ end of the preceding monomer on the respective strand (see, e.g., Table 11). Moreover, as the person of ordinary skill would readily understand and appreciate, while the phosphorothioate chemical structures depicted herein typically show the anion on the sulfur atom, the inventions disclosed herein encompass all phosphorothioate tautomers (e.g., where the sulfur atom has a double-bond and the anion is on an oxygen atom). Unless expressly indicated otherwise herein, such understandings of the person of ordinary skill in the art are used when describing the MMP7 RNAi agents and compositions of MMP7 RNAi agents disclosed herein.

Certain examples of targeting groups and linking groups used with the MMP7 RNAi agents disclosed herein are included in the chemical structures provided below in Table 11. Each sense strand and/or antisense strand can have any targeting groups or linking groups listed herein, as well as other targeting or linking groups, conjugated to the 5′ and/or 3′ end of the sequence.

TABLE 3
MMP7 RNAi Agent Antisense Strand Sequences

| | | | Underlying Base | |
|---|---|---|---|---|
| | | | Sequence (5′ → 3′) | |
| AS | Modified Antisense | SEQ ID | (Shown as an Unmodified | SEQ ID |
| Strand ID | Strand (5′ → 3′) | NO. | Nucleotide Sequence) | NO. |
| AM08523-AS | asAfscsAfuCfuGfgCfaCfuCfcAfcAfuCfsu | 337 | AACAUCUGGCACUCCACAUCU | 629 |
| AM08525-AS | asAfscsAfuCfuGfgCfaCfuCfcAfcAfuCfsg | 338 | AACAUCUGGCACUCCACAUCG | 630 |
| AM08527-AS | usCfsasAfcAfuCfuGfgCfaCfuCfcAfcAfsu | 339 | UCAACAUCUGGCACUCCACAU | 631 |
| AM08529-AS | usCfsasAfcAfuCfuGfgCfaCfuCfcAfcAfsg | 340 | UCAACAUCUGGCACUCCACAG | 632 |
| AM08531-AS | usGfscsAfaCfaUfcUfgGfcAfcUfcCfaCfsa | 341 | UGCAACAUCUGGCACUCCACA | 633 |
| AM08533-AS | usGfscsAfaCfaUfcUfgGfcAfcUfcCfaCfsg | 342 | UGCAACAUCUGGCACUCCACG | 634 |
| AM08535-AS | usCfsusGfcAfaCfaUfcUfgGfcAfcUfcCfsa | 343 | UCUGCAACAUCUGGCACUCCA | 635 |
| AM08537-AS | usCfsusGfcAfaCfaUfcUfgGfcAfcUfcCfsg | 344 | UCUGCAACAUCUGGCACUCCG | 636 |
| AM08539-AS | usUfscsUfgCfaAfcAfuCfuGfgCfaCfuCfsc | 345 | UUCUGCAACAUCUGGCACUCC | 637 |
| AM08541-AS | asUfsusCfuGfcAfaCfaUfcUfgGfcAfcUfsc | 346 | AUUCUGCAACAUCUGGCACUC | 638 |
| AM08543-AS | usAfsusUfcUfgCfaAfcAfuCfuGfgCfaCfsu | 347 | UAUUCUGCAACAUCUGGCACU | 639 |
| AM08545-AS | usAfsusUfcUfgCfaAfcAfuCfuGfgCfaCfsg | 348 | UAUUCUGCAACAUCUGGCACG | 640 |
| AM08547-AS | usUfsusCfcUfgGfcCfcAfuCfaAfaUfgGfsg | 349 | UUUCCUGGCCCAUCAAAUGGG | 641 |
| AM08549-AS | usGfsusGfuUfuCfcUfgGfcCfcAfuCfaAfsg | 350 | UGUGUUUCCUGGCCCAUCAAG | 642 |
| AM08789-AS | usCfsusUfcUfuUfgUfuUfuAfgAfgUfcGfsu | 351 | UCUUCUUUGUUUUAGAGUCGU | 643 |
| AM08791-AS | asAfsgsAfaCfuUfcUfgCfaUfuUfcCfcUfsc | 352 | AAGAACUUCUGCAUUUCCCUC | 644 |
| AM08793-AS | usUfsgsAfuCfuAfcUfaAfgAfaCfcGfaGfsg | 353 | UUGAUCUACUAAGAACCGAGG | 645 |
| AM08797-AS | asCfsasAfaCfaGfgAfaGfuUfcAfcUfcCfsu | 354 | ACAAACAGGAAGUUCACUCCU | 646 |
| AM08801-AS | usAfscsAfgUfaCfaAfgGfaAfgAfaAfgGfsa | 355 | UACAGUACAAGGAAGAAAGGA | 647 |
| AM08803-AS | usGfsasCfaUfuUfaUfuGfcUfgGfuGfuCfsu | 356 | UGACAUUUAUUGCUGGUGUCU | 648 |
| AM08805-AS | usGfsasCfaUfuUfaUfuGfcUfgGfuGfuCfsg | 357 | UGACAUUUAUUGCUGGUGUCG | 649 |
| AM08807-AS | usGfsasCfaUfuUfaUfuGfcUfgAfuGfuCfsg | 358 | UGACAUUUAUUGCUGAUGUCG | 650 |
| AM12367-AS | usUfsusGfaGfaUfaGfuCfcUfgAfgCfcUfsg | 359 | UUUGAGAUAGUCCUGAGCCUG | 651 |
| AM12369-AS | asGfsasGfaUfaAfaAfuCfuCfuUfgAfgAfsc | 360 | AGAGAUAAAAUCUCUUGAGAC | 652 |
| AM12371-AS | usCfsasUfaGfaGfaUfaAfaAfuCfuCfuUfsg | 361 | UCAUAGAGAUAAAAUCUCUUG | 653 |
| AM12373-AS | usAfsgsUfcAfuAfgAfgAfuAfaAfaUfcUfsc | 362 | UAGUCAUAGAGAUAAAAUCUC | 654 |
| AM12375-AS | usAfsasCfaUfcUfgGfcAfcUfcCfaCfaUfsc | 363 | UAACAUCUGGCACUCCACAUC | 655 |
| AM12378-AS | usAfsgsUfaUfuCfuGfcAfaCfaUfcUfgGfsc | 364 | UAGUAUUCUGCAACAUCUGGC | 656 |
| AM12380-AS | asAfsusAfgUfgAfgUfaUfuCfuGfcAfaCfsc | 365 | AAUAGUGAGUAUUCUGCAACC | 657 |
| AM12382-AS | asAfsasUfaGfuGfaGfuAfuUfcUfgCfaAfsc | 366 | AAAUAGUGAGUAUUCUGCAAC | 658 |
| AM12384-AS | usAfscsUfaAfuCfgAfuCfcAfcUfgUfaAfsc | 367 | UACUAAUCGAUCCACUGUAAC | 659 |
| AM12386-AS | asCfsasCfuAfaUfcGfaUfcCfaCfuGfuAfsc | 368 | ACACUAAUCGAUCCACUGUAC | 660 |
| AM12388-AS | usUfsgsAfcAfcUfaAfuCfgAfuCfcAfcUfsg | 369 | UUGACACUAAUCGAUCCACUG | 661 |
| AM12390-AS | usCfsasUfcGfaAfgUfgAfgCfaUfcUfcCfsu | 370 | UCAUCGAAGUGAGCAUCUCCU | 662 |
| AM12392-AS | usAfsgsCfaUfaCfaGfgAfaGfuUfaAfuCfsc | 371 | UAGCAUACAGGAAGUUAAUCC | 663 |
| AM12394-AS | asUfsasGfgUfuGfgAfuAfcAfuCfaCfuGfsc | 372 | AUAGGUUGGAUACAUCACUGC | 664 |
| AM12396-AS | asUfsusUfcCfaUfaGfgUfuGfgAfuAfcAfsc | 373 | AUUUCCAUAGGUUGGAUACAC | 665 |
| AM12398-AS | usCfscsUfuUfaAfuAfuCfaUfcCfuGfgGfsa | 374 | UCCUUUAAUAUCAUCCUGGGA | 666 |
| AM12400-AS | usAfsusAfgUfuUfcUfgAfaUfgCfcUfuUfsc | 375 | UAUAGUUUCUGAAUGCCUUUC | 667 |
| AM12402-AS | usCfsasAfuUfcUfgAfuUfgUfgCfaAfcAfsc | 376 | UCAAUUCUGAUUGUGCAACAC | 668 |
| AM12404-AS | asUfscsAfaUfuCfuGfaUfuGfuGfcAfaCfsc | 377 | AUCAAUUCUGAUUGUGCAACC | 669 |
| AM12406-AS | usGfscsUfuAfuCfaAfuUfcUfgAfuUfgUfsg | 378 | UGCUUAUCAAUUCUGAUUGUG | 670 |
| AM12408-AS | usUfsgsCfuUfaUfcAfaUfuCfuGfaUfuGfsc | 379 | UUGCUUAUCAAUUCUGAUUGC | 671 |
| AM12410-AS | usGfscsAfaUfaAfaAfaAfgGfgUfgAfcAfsc | 380 | UGCAAUAAAAAAGGGUGACAC | 672 |
| AM12412-AS | asCfsasUfuCfaAfaAfaCfcAfaCfuGfcAfsc | 381 | ACAUUCAAAAACCAACUGCAC | 673 |
| AM12414-AS | asGfsasCfaUfuCfaAfaAfaCfcAfaCfuGfsc | 382 | AGACAUUCAAAAACCAACUGC | 674 |
| AM12416-AS | asAfsgsAfcAfuUfcAfaAfaAfcCfaAfcUfsg | 383 | AAGACAUUCAAAAACCAACUG | 675 |
| AM12418-AS | asGfsusUfuAfuCfcUfuAfaAfaGfgAfgUfsg | 384 | AGUUUAUCCUUAAAAGGAGUG | 676 |
| AM12875-AS | cPrpusAfsasCfaUfcUfgGfcAfcUfcCfaCfaUfsc | 385 | UAACAUCUGGCACUCCACAUC | 655 |
| AM12877-AS | cPrpuAfacaucuggcAfcUfcCfacausc | 386 | UAACAUCUGGCACUCCACAUC | 655 |
| AM13076-AS | usCfsasAfuUfcUfgAfuUfgUfgCfaAfcAfsg | 387 | UCAAUUCUGAUUGUGCAACAG | 677 |
| AM13077-AS | cPrpusCfsasAfuUfcUfgAfuUfgUfgCfaAfcAfsc | 388 | UCAAUUCUGAUUGUGCAACAC | 668 |
| AM13079-AS | usCfsasauucugauUfgUfgCfaacasc | 389 | UCAAUUCUGAUUGUGCAACAC | 668 |
| AM13081-AS | usCfsasAfuucugauUfgUfgCfaacasc | 390 | UCAAUUCUGAUUGUGCAACAC | 668 |
| AM13082-AS | usCfsasAfuUfcugauUfgUfgCfaacasc | 391 | UCAAUUCUGAUUGUGCAACAC | 668 |
| AM13083-AS | usCfsasAfuUfcugauUfgUfgCfaAfcasc | 392 | UCAAUUCUGAUUGUGCAACAC | 668 |
| AM13212-AS | usUfsgsacacUfaAfuCfgAfuCfcacusg | 393 | UUGACACUAAUCGAUCCACUG | 661 |
| AM13215-AS | usCfscsuuuaAfuAfuCfaUfcCfugggsa | 394 | UCCUUUAAUAUCAUCCUGGGA | 666 |
| AM13241-AS | asGfsascauucaaaAfaCfcAfacugsc | 395 | AGACAUUCAAAAACCAACUGC | 674 |
| AM13244-AS | asGfsascauucAfaaAfaCfcAfacugsc | 396 | AGACAUUCAAAAACCAACUGC | 674 |
| AM13245-AS | asGfsascauuCfAfaaAfaCfcAfacugsc | 397 | AGACAUUCAAAAACCAACUGC | 674 |
| AM13246-AS | asUfsasgguuggauAfcAfuCfacugsc | 398 | AUAGGUUGGAUACAUCACUGC | 664 |
| AM13247-AS | asUfsasgguUUNAggauAfcAfuCfacugsc | 399 | AUAGGUUGGAUACAUCACUGC | 664 |
| AM13400-AS | usAfsgsuauucugcAfaCfaUfcuggsc | 400 | UAGUAUUCUGCAACAUCUGGC | 656 |
| AM13402-AS | usAfsgsuauUUNAcugcAfaCfaUfcuggsc | 401 | UAGUAUUCUGCAACAUCUGGO | 656 |
| AM13403-AS | usAfsgsuaUUNAUfcugcAfaCfaUfcuggsc | 402 | UAGUAUUCUGCAACAUCUGGC | 656 |
| AM13405-AS | asGfsusuuauccuuAfaAfaGfgagusg | 403 | AGUUUAUCCUUAAAAGGAGUG | 676 |
| AM13407-AS | asGfsusUfuauccuuAfaAfaGfgagusg | 404 | AGUUUAUCCUUAAAAGGAGUG | 676 |
| AM13409-AS | asGfsusUfuauccuuAfaAfaGfgagusc | 405 | AGUUUAUCCUUAAAAGGAGUC | 678 |
| AM14108-AS | cPrpuGfacauucAfaaAfaCfcAfacugsc | 406 | UGACAUUCAAAAACCAACUGC | 679 |
| AM14109-AS | cPrpuUfgacacUfaAfuCfgAfuCfcacusg | 407 | UUGACACUAAUCGAUCCACUG | 661 |
| AM14110-AS | cPrpasGfsasCfaUfuCfaAfaAfaCfcAfaCfuGfsc | 408 | AGACAUUCAAAAACCAACUGC | 674 |
| AM14112-AS | cPrpusGfsasCfaUfuCfaAfaAfaCfcAfaCfuGfsc | 409 | UGACAUUCAAAAACCAACUGC | 679 |
| AM14113-AS | cPrpuGfaCfaUfuCfaAfaAfaCfcAfaCfuGfsc | 410 | UGACAUUCAAAAACCAACUGC | 679 |
| AM14117-AS | cPrpusUfsgsAfcAfcUfaAfuCfgAfuCfcAfcUfsg | 411 | UUGACACUAAUCGAUCCACUG | 661 |
| AM14118-AS | cPrpuUfgAfcAfcUfaAfuCfgAfuCfcAfcUfsg | 412 | UUGACACUAAUCGAUCCACUG | 661 |
| AM14119-AS | cPrpusUfsgsacacUfaAfuCfgAfuCfcacusg | 413 | UUGACACUAAUCGAUCCACUG | 661 |
| AM14121-AS | usAfsascaucuggcAfcUfcCfacausc | 414 | UAACAUCUGGCACUCCACAUC | 655 |
| AM14122-AS | cPrpusAfsascaucuggcAfcUfcCfacausc | 415 | UAACAUCUGGCACUCCACAUC | 655 |
| AM14124-AS | cPrpusUfsasGfgUfuGfgAfuAfcAfuCfaCfuGfsc | 416 | UUAGGUUGGAUACAUCACUGC | 680 |
| AM14125-AS | usUfsasGfgUfuggauAfcAfuCfaCfugsc | 417 | UUAGGUUGGAUACAUCACUGC | 680 |
| AM14126-AS | cPrpusUfsasGfgUfuggauAfcAfuCfaCfugsc | 418 | UUAGGUUGGAUACAUCACUGC | 680 |
| AM14127-AS | cPrpuUfaGfgUfuggauAfcAfuCfaCfugsc | 419 | UUAGGUUGGAUACAUCACUGC | 680 |
| AM14129-AS | cPrpusGfsusUfuauccuuAfaAfaGfgagusg | 420 | UGUUUAUCCUUAAAAGGAGUG | 681 |
| AM14130-AS | cPrpuGfuUfuauccuuAfaAfaGfgagusg | 421 | UGUUUAUCCUUAAAAGGAGUG | 681 |
| AM14131-AS | cPrpusGfsusUfuAfuCfcUfuAfaAfaGfgAfgUfsg | 422 | UGUUUAUCCUUAAAAGGAGUG | 681 |
| AM14114-AS | cPrpasGfsascauucAfaaAfaCfcAfacugsc | 423 | AGACAUUCAAAAACCAACUGC | 674 |
| AM14116-AS | cPrpusGfsascauucAfaaAfaCfcAfacugsc | 424 | UGACAUUCAAAAACCAACUGC | 679 |
| AM14651-AS | usUfsasGfgUfuGfgAfuAfcAfuCfaCfuGfsc | 425 | UUAGGUUGGAUACAUCACUGC | 680 |
| AM14652-AS | cPrpasUfsasGfgUfuGfgAfuAfcAfuCfaCfuGfsc | 426 | AUAGGUUGGAUACAUCACUGC | 664 |
| AM14653-AS | asUfsagguuggauAfcAfuCfacusgsc | 427 | AUAGGUUGGAUACAUCACUGC | 664 |
| AM14654-AS | cPrpasUfsagguuggauAfcAfuCfacusgsc | 428 | AUAGGUUGGAUACAUCACUGC | 664 |
| AM14655-AS | cPrpasUfsaGfguuggauAfcAfuCfacusgsc | 429 | AUAGGUUGGAUACAUCACUGC | 664 |
| AM14656-AS | cPrpaUfaGfguuggauAfcAfuCfacusgsc | 430 | AUAGGUUGGAUACAUCACUGC | 664 |
| AM14657-AS | cPrpasUfsagGfuuggauAfcAfuCfacusgsc | 431 | AUAGGUUGGAUACAUCACUGC | 664 |
| AM14658-AS | cPrpasUfsagguUfggauAfcAfuCfacusgsc | 432 | AUAGGUUGGAUACAUCACUGC | 664 |
| AM14659-AS | cPrpasUfsagguugGfauAfcAfuCfacusgsc | 433 | AUAGGUUGGAUACAUCACUGC | 664 |
| AM14888-AS | cPrpasUfsasgguuggauAfcAfuCfacugsc | 434 | AUAGGUUGGAUACAUCACUGC | 664 |
| AM14889-AS | cPrpaUfagguuggauAfcAfuCfacugsc | 435 | AUAGGUUGGAUACAUCACUGC | 664 |
| AM15155-AS | cPrpaGfacauucAfaaAfaCfcAfacugsc | 436 | AGACAUUCAAAAACCAACUGC | 674 |

TABLE 4
MMP7 Agent Sense Strand Sequences
(Shown Without Linkers, Conjugates, or Capping Moieties)

| | | | Underlying Base | |
|---|---|---|---|---|
| | | | Sequence (5′ → 3′) | |
| | Modified Sense | SEQ ID | (Shown as an Unmodified | SEQ ID |
| Strand ID | Strand (5′ → 3′) | NO. | Nucleotide Sequence) | NO. |
| AM08522-SS-NL | asgauguggAfGfUfgccagauguu | 458 | AGAUGUGGAGUGCCAGAUGUU | 682 |
| AM08524-SS-NL | csgauguggAfGfUfgccagauguu | 459 | CGAUGUGGAGUGCCAGAUGUU | 683 |
| AM08526-SS-NL | asuguggagUfGfCfcagauguuga | 460 | AUGUGGAGUGCCAGAUGUUGA | 684 |
| AM08528-SS-NL | csuguggagUfGfCfcagauguuga | 461 | CUGUGGAGUGCCAGAUGUUGA | 685 |
| AM08530-SS-NL | usguggaguGfCfCfagauguugca | 462 | UGUGGAGUGCCAGAUGUUGCA | 686 |
| AM08532-SS-NL | csguggaguGfCfCfagauguugca | 463 | CGUGGAGUGCCAGAUGUUGCA | 687 |
| AM08534-SS-NL | usggagugcCfAfGfauguugcaga | 464 | UGGAGUGCCAGAUGUUGCAGA | 688 |
| AM08536-SS-NL | csggagugcCfAfGfauguugcaga | 465 | CGGAGUGCCAGAUGUUGCAGA | 689 |
| AM08538-SS-NL | gsgagugccAfGfAfuguugcagaa | 466 | GGAGUGCCAGAUGUUGCAGAA | 690 |
| AM08540-SS-NL | gsagugccaGfAfUfguugcagaau | 467 | GAGUGCCAGAUGUUGCAGAAU | 691 |
| AM08542-SS-NL | asgugccagAfUfGfuugcagaaua | 468 | AGUGCCAGAUGUUGCAGAAUA | 692 |
| AM08544-SS-NL | csgugccagAfUfGfuugcagaaua | 469 | CGUGCCAGAUGUUGCAGAAUA | 693 |
| AM08546-SS-NL | csccauuugAfUfGfggccaggaaa | 470 | CCCAUUUGAUGGGCCAGGAAA | 694 |
| AM08548-SS-NL | csuugauggGfCfCfaggaaacaca | 471 | CUUGAUGGGCCAGGAAACACA | 695 |
| AM12366-SS-NL | caggcucaGfGfAfcuaucucaaa | 472 | CAGGCUCAGGACUAUCUCAAA | 696 |
| AM12368-SS-NL | gucucaagAfGfAfuuuuaucucu | 473 | GUCUCAAGAGAUUUUAUCUCU | 697 |
| AM12370-SS-NL | ca_2NagagauUfUfUfaucucuauga | 474 | CAAGAGAUUUUAUCUCUAUGA | 698 |
| AM12372-SS-NL | gagauuuuAfUfCfucuaugacua | 475 | GAGAUUUUAUCUCUAUGACUA | 699 |
| AM12374-SS-NL | gauguggaGfUfGfccagauguua | 476 | GAUGUGGAGUGCCAGAUGUUA | 700 |
| AM12376-SS-NL | ggagugccAfGfAfuguuicagaa | 477 | GGAGUGCCAGAUGUUICAGAA | 701 |
| AM12377-SS-NL | gccagaugUfUfGfcagaauacua | 478 | GCCAGAUGUUGCAGAAUACUA | 702 |
| AM12379-SS-NL | gguugcagAfAfUfacucacuauu | 479 | GGUUGCAGAAUACUCACUAUU | 703 |
| AM12381-SS-NL | sguugcagaAfUfAfcucacuauuu | 480 | GUUGCAGAAUACUCACUAUUU | 704 |
| AM12383-SS-NL | guuacaguGfGfAfucgauuagua | 481 | GUUACAGUGGAUCGAUUAGUA | 705 |
| AM12385-SS-NL | guacagugGfAfUfcgauuaiugu | 482 | GUACAGUGGAUCGAUUAIUGU | 706 |
| AM12387-SS-NL | caguggauCfGfAfuuaguiucaa | 483 | CAGUGGAUCGAUUAGUIUCAA | 707 |
| AM12389-SS-NL | aggagaugCfUfCfacuuciauga | 484 | AGGAGAUGCUCACUUCIAUGA | 708 |
| AM12391-SS-NL | ggauuaacUfUfCfcuguaugcua | 485 | GGAUUAACUUCCUGUAUGCUA | 709 |
| AM12393-SS-NL | gcagugauGfUfAfuccaaccuau | 486 | GCAGUGAUGUAUCCAACCUAU | 710 |
| AM12395-SS-NL | guguauccAfAfCfcuauggaaau | 487 | GUGUAUCCAACCUAUGGAAAU | 711 |
| AM12397-SS-NL | ucccaggaUfGfAfuauuaaagga | 488 | UCCCAGGAUGAUAUUAAAGGA | 712 |
| AM12399-SS-NL | gaaaggcaUfUfCfagaaacuaua | 489 | GAAAGGCAUUCAGAAACUAUA | 713 |
| AM12401-SS-NL | guguugcaCfAfAfucagaauuga | 490 | GUGUUGCACAAUCAGAAUUGA | 714 |
| AM12403-SS-NL | gguugcacAfAfUfcagaauugau | 491 | GGUUGCACAAUCAGAAUUGAU | 715 |
| AM12405-SS-NL | cacaaucaGfAfAfuugauaagca | 492 | CACAAUCAGAAUUGAUAAGCA | 716 |
| AM12407-SS-NL | gcaaucagAfAfUfugauaagcaa | 493 | GCAAUCAGAAUUGAUAAGCAA | 717 |
| AM12409-SS-NL | gugucaccCfUfUfuuuuauugca | 494 | GUGUCACCCUUUUUUAUUGCA | 718 |
| AM12411-SS-NL | gugcaguuGfGfUfuuuugaaugu | 495 | GUGCAGUUGGUUUUUGAAUGU | 719 |
| AM12413-SS-NL | gcaguuggUfUfUfuugaaugucu | 496 | GCAGUUGGUUUUUGAAUGUCU | 720 |
| AM12415-SS-NL | caguugguUfUfUfugaaugucuu | 497 | CAGUUGGUUUUUGAAUGUCUU | 721 |
| AM12417-SS-NL | cacuccuuUfUfAfaggauaaacu | 498 | CACUCCUUUUAAGGAUAAACU | 722 |
| AM12874-SS-NL | gsauguggaGfUfGfccagauguua | 499 | GAUGUGGAGUGCCAGAUGUUA | 700 |
| AM12876-SS-NL | gsauguggaGfuGfcCfagauguua | 500 | GAUGUGGAGUGCCAGAUGUUA | 700 |
| AM13075-SS-NL | cuguugcaCfAfAfucagaauuga | 501 | CUGUUGCACAAUCAGAAUUGA | 723 |
| AM13078-SS-NL | guguugCfaCfaAfucagaauuga | 502 | GUGUUGCACAAUCAGAAUUGA | 714 |
| AM13080-SS-NL | guguugcaCfaAfuCfagaauuga | 503 | GUGUUGCACAAUCAGAAUUGA | 714 |
| AM13084-SS-NL | guguugcaCfaAfucagaauuga | 504 | GUGUUGCACAAUCAGAAUUGA | 714 |
| AM13210-SS-NL | caguggauCfGfAfuuagugucaa | 505 | CAGUGGAUCGAUUAGUGUCAA | 724 |
| AM13211-SS-NL | caguggAfuCfgAfuuagugucaa | 506 | CAGUGGAUCGAUUAGUGUCAA | 724 |
| AM13213-SS-NL | caguggauCfgAfuUfagugucaa | 507 | CAGUGGAUCGAUUAGUGUCAA | 724 |
| AM13214-SS-NL | caguggauCfgAfuUfaguiucaa | 508 | CAGUGGAUCGAUUAGUIUCAA | 707 |
| AM13216-SS-NL | ucccaggaUfGfAfuAfuuaaagga | 509 | UCCCAGGAUGAUAUUAAAGGA | 712 |
| AM13217-SS-NL | ucccaggaUfgAfuAfuuaaagga | 510 | UCCCAGGAUGAUAUUAAAGGA | 712 |
| AM13218-SS-NL | ucccaggaUfGfAfua_2Nuuaaagga | 511 | UCCCAGGAUGAUAUUAAAGGA | 712 |
| AM13242-SS-NL | gcaguuggUfuUfuUfgaaugucu | 512 | GCAGUUGGUUUUUGAAUGUCU | 720 |
| AM13243-SS-NL | gcaguuggUfUfUfuUfgaaugucu | 513 | GCAGUUGGUUUUUGAAUGUCU | 720 |
| AM13248-SS-NL | gcagugauGfuAfUfccaaccuau | 514 | GCAGUGAUGUAUCCAACCUAU | 710 |
| AM13249-SS-NL | gcagugAfuGfuAfuccaaccuau | 515 | GCAGUGAUGUAUCCAACCUAU | 710 |
| AM13250-SS-NL | gcagugauGfuAfuCfcaaccuau | 516 | GCAGUGAUGUAUCCAACCUAU | 710 |
| AM13399-SS-NL | gccagaugUfuGfcAfgaauacua | 517 | GCCAGAUGUUGCAGAAUACUA | 702 |
| AM13401-SS-NL | gccagaUfgUfuGfcagaauacua | 518 | GCCAGAUGUUGCAGAAUACUA | 702 |
| AM13404-SS-NL | cacuccUfuUfuAfaggauaaacu | 519 | CACUCCUUUUAAGGAUAAACU | 722 |
| AM13406-SS-NL | cacuccuuUfuAfAfggauaaacu | 520 | CACUCCUUUUAAGGAUAAACU | 722 |
| AM13408-SS-NL | gacuccuuUfUfAfaggauaaacu | 521 | GACUCCUUUUAAGGAUAAACU | 725 |
| AM13762-SS-NL | gscaguuggUfuUfuUfgaaugucu | 522 | GCAGUUGGUUUUUGAAUGUCU | 720 |
| AM13763-SS-NL | gscaguuggUfUfUfuugaaugucu | 523 | GCAGUUGGUUUUUGAAUGUCU | 720 |
| AM13764-SS-NL | csaguggauCfgAfuUfagugucaa | 524 | CAGUGGAUCGAUUAGUGUCAA | 724 |
| AM13765-SS-NL | csaguggauCfgAfuUfaguiucaa | 525 | CAGUGGAUCGAUUAGUIUCAA | 707 |
| AM14107-SS-NL | gscaguuggUfuUfuUfgaauguca | 526 | GCAGUUGGUUUUUGAAUGUCA | 726 |
| AM14111-SS-NL | gcaguuggUfUfUfuugaauguca | 527 | GCAGUUGGUUUUUGAAUGUCA | 726 |
| AM14120-SS-NL | gauguggaGfuGfcCfagauguua | 528 | GAUGUGGAGUGCCAGAUGUUA | 700 |
| AM14123-SS-NL | gcagugauGfUfAfuccaaccuaa | 529 | GCAGUGAUGUAUCCAACCUAA | 727 |
| AM14128-SS-NL | cacuccuuUfUfAfaggauaaaca | 530 | CACUCCUUUUAAGGAUAAACA | 728 |
| AM14115-SS-NL | gcaguuggUfuUfuUfgaauguca | 531 | GCAGUUGGUUUUUGAAUGUCA | 726 |
| AM14553-SS-NL | gscagugauGfuAfuCfcaaccuau | 532 | GCAGUGAUGUAUCCAACCUAU | 710 |
| AM14890-SS-NL | gscsaguuggUfuUfuUfgaaugucsu | 533 | GCAGUUGGUUUUUGAAUGUCU | 720 |
| AM14892-SS-NL | gsCfsasguuggUfuuUfuGfaAfugucsu | 534 | GCAGUUGGUUUUUGAAUGUCU | 720 |
| AM15418-SS-NL | gscguuggUfuUfuUfgaauguca | 535 | GCGUUGGUUUUUGAAUGUCA | 729 |
| AM15419-SS-NL | gscaguugggUfuUfuUfgaauguca | 536 | GCAGUUGGGUUUUUGAAUGUCA | 730 |
| AM15420-SS-NL | gscaguuggUfuUfuUfgaauguca | 526 | GCAGUUGGUUUUUGAAUGUCA | 726 |
| AM16013-SS-NL | gscaguuggUfuUfuUfgaauguca | 526 | GCAGUUGGUUUUUGAAUGUCA | 726 |

a_2N = 2-aminoadenosine nucleotide;
I = hypoxanthine (inosine) nucleotide

TABLE 5
MMP7 Agent Sense Strand Sequences (Shown With Linkers and
and Capping Groups (see Table 11 for structure information.))

| | | | Underlying Base | |
|---|---|---|---|---|
| | | | Sequence (5′ → 3′) | |
| | | SEQ | (Shown as an Unmodified | SEQ |
| Strand ID | Modified Sense Strand (5′ → 3′) | ID NO. | Nucleotide Sequence) | ID NO. |
| AM08522-SS | (NH2-C6)asgauguggAfGfUfgccagauguus(invAb) | 539 | AGAUGUGGAGUGCCAGAUGUU | 682 |
| AM08524-SS | (NH2-C6)csgauguggAfGfUfgccagauguus(invAb) | 540 | CGAUGUGGAGUGCCAGAUGUU | 683 |
| AM08526-SS | (NH2-C6)asuguggagUfGfCfcagauguugas(invAb) | 541 | AUGUGGAGUGCCAGAUGUUGA | 684 |
| AM08528-SS | (NH2-C6)csuguggagUfGfCfcagauguugas(invAb) | 542 | CUGUGGAGUGCCAGAUGUUGA | 685 |
| AM08530-SS | (NH2-C6)usguggaguGfCfCfagauguugcas(invAb) | 543 | UGUGGAGUGCCAGAUGUUGCA | 686 |
| AM08532-SS | (NH2-C6)csguggaguGfCfCfagauguugcas(invAb) | 544 | CGUGGAGUGCCAGAUGUUGCA | 687 |
| AM08534-SS | (NH2-C6)usggagugcCfAfGfauguugcagas(invAb) | 545 | UGGAGUGCCAGAUGUUGCAGA | 688 |
| AM08536-SS | (NH2-C6)csggagugcCfAfGfauguugcagas(invAb) | 546 | CGGAGUGCCAGAUGUUGCAGA | 689 |
| AM08538-SS | (NH2-C6)gsgagugccAfGfAfuguugcagaas(invAb) | 547 | GGAGUGCCAGAUGUUGCAGAA | 690 |
| AM08540-SS | (NH2-C6)gsagugccaGfAfUfguugcagaaus(invAb) | 548 | GAGUGCCAGAUGUUGCAGAAU | 691 |
| AM08542-SS | (NH2-C6)asgugccagAfUfGfuugcagaauas(invAb) | 549 | AGUGCCAGAUGUUGCAGAAUA | 692 |
| AM08544-SS | (NH2-C6)csgugccagAfUfGfuugcagaauas(invAb) | 550 | CGUGCCAGAUGUUGCAGAAUA | 693 |
| AM08546-SS | (NH2-C6)csccauuugAfUfGfggccaggaaas(invAb) | 551 | CCCAUUUGAUGGGCCAGGAAA | 694 |
| AM08548-SS | (NH2-C6)csuugauggGfCfCfaggaaacacas(invAb) | 552 | CUUGAUGGGCCAGGAAACACA | 695 |
| AM12366-SS | (NAG37)s(invAb)scaggcucaGfGfAfcuaucucaaas(invAb) | 553 | CAGGCUCAGGACUAUCUCAAA | 696 |
| AM12368-SS | (NAG37)s(invAb)sgucucaagAfGfAfuuuuaucucus(invAb) | 554 | GUCUCAAGAGAUUUUAUCUCU | 697 |
| AM12370-SS | (NAG37)s(invAb)sca_2NagagauUfUfUfaucucuaugas(invAb) | 555 | CAAGAGAUUUUAUCUCUAUGA | 698 |
| AM12372-SS | (NAG37)s(invAb)sgagauuuuAfUfCfucuaugacuas(invAb) | 556 | GAGAUUUUAUCUCUAUGACUA | 699 |
| AM12374-SS | (NAG37)s(invAb)sgauguggaGfUfGfccagauguuas(invAb) | 557 | GAUGUGGAGUGCCAGAUGUUA | 700 |
| AM12376-SS | (NAG37)s(invAb)sggagugccAfGfAfuguuicagaas(invAb) | 558 | GGAGUGCCAGAUGUUICAGAA | 701 |
| AM12377-SS | (NAG37)s(invAb)sgccagaugUfUfGfcagaauacuas(invAb) | 559 | GCCAGAUGUUGCAGAAUACUA | 702 |
| AM12379-SS | (NAG37)s(invAb)sgguugcagAfAfUfacucacuauus(invAb) | 560 | GGUUGCAGAAUACUCACUAUU | 703 |
| AM12381-SS | (NAG37)s(invAb)sguugcagaAfUfAfcucacuauuus(invAb) | 561 | GUUGCAGAAUACUCACUAUUU | 704 |
| AM12383-SS | (NAG37)s(invAb)sguuacaguGfGfAfucgauuaguas(invAb) | 562 | GUUACAGUGGAUCGAUUAGUA | 705 |
| AM12385-SS | (NAG37)s(invAb)sguacagugGfAfUfcgauuaiugus(invAb) | 563 | GUACAGUGGAUCGAUUAIUGU | 706 |
| AM12387-SS | (NAG37)s(invAb)scaguggauCfGfAfuuaguiucaas(invAb) | 564 | CAGUGGAUCGAUUAGUIUCAA | 707 |
| AM12389-SS | (NAG37)s(invAb)saggagaugCfUfCfacuuciaugas(invAb) | 565 | AGGAGAUGCUCACUUCIAUGA | 708 |
| AM12391-SS | (NAG37)s(invAb)sggauuaacUfUfCfcuguaugcuas(invAb) | 566 | GGAUUAACUUCCUGUAUGCUA | 709 |
| AM12393-SS | (NAG37)s(invAb)sgcagugauGfUfAfuccaaccuaus(invAb) | 567 | GCAGUGAUGUAUCCAACCUAU | 710 |
| AM12395-SS | (NAG37)s(invAb)sguguauccAfAfCfcuauggaaaus(invAb) | 568 | GUGUAUCCAACCUAUGGAAAU | 711 |
| AM12397-SS | (NAG37)s(invAb)succcaggaUfGfAfuauuaaaggas(invAb) | 569 | UCCCAGGAUGAUAUUAAAGGA | 712 |
| AM12399-SS | (NAG37)s(invAb)sgaaaggcaUfUfCfagaaacuauas(invAb) | 570 | GAAAGGCAUUCAGAAACUAUA | 713 |
| AM12401-SS | (NAG37)s(invAb)sguguugcaCfAfAfucagaauugas(invAb) | 571 | GUGUUGCACAAUCAGAAUUGA | 714 |
| AM12403-SS | (NAG37)s(invAb)sgguugcacAfAfUfcagaauugaus(invAb) | 572 | GGUUGCACAAUCAGAAUUGAU | 715 |
| AM12405-SS | (NAG37)s(invAb)scacaaucaGfAfAfuugauaagcas(invAb) | 573 | CACAAUCAGAAUUGAUAAGCA | 716 |
| AM12407-SS | (NAG37)s(invAb)sgcaaucagAfAfUfugauaagcaas(invAb) | 574 | GCAAUCAGAAUUGAUAAGCAA | 717 |
| AM12409-SS | (NAG37)s(invAb)sgugucaccCfUfUfuuuuauugcas(invAb) | 575 | GUGUCACCCUUUUUUAUUGCA | 718 |
| AM12411-SS | (NAG37)s(invAb)sgugcaguuGfGfUfuuuugaaugus(invAb) | 576 | GUGCAGUUGGUUUUUGAAUGU | 719 |
| AM12413-SS | (NAG37)s(invAb)sgcaguuggUfUfUfuugaaugucus(invAb) | 577 | GCAGUUGGUUUUUGAAUGUCU | 720 |
| AM12415-SS | (NAG37)s(invAb)scaguugguUfUfUfugaaugucuus(invAb) | 578 | CAGUUGGUUUUUGAAUGUCUU | 721 |
| AM12417-SS | (NAG37)s(invAb)scacuccuuUfUfAfaggauaaacus(invAb) | 579 | CACUCCUUUUAAGGAUAAACU | 722 |
| AM12874-SS | (TriAlk14)gsauguggaGfUfGfccagauguuas(invAb) | 580 | GAUGUGGAGUGCCAGAUGUUA | 700 |
| AM12876-SS | (TriAlk14)gsauguggaGfuGfcCfagauguuas(invAb) | 581 | GAUGUGGAGUGCCAGAUGUUA | 700 |
| AM13075-SS | (NAG37)s(invAb)scuguugcaCfAfAfucagaauugas(invAb) | 582 | CUGUUGCACAAUCAGAAUUGA | 723 |
| AM13078-SS | (NAG37)s(invAb)sguguugCfaCfaAfucagaauugas(invAb) | 583 | GUGUUGCACAAUCAGAAUUGA | 714 |
| AM13080-SS | (NAG37)s(invAb)sguguugcaCfaAfuCfagaauugas(invAb) | 584 | GUGUUGCACAAUCAGAAUUGA | 714 |
| AM13084-SS | (NAG37)s(invAb)sguguugcaCfaAfucagaauugas(invAb) | 585 | GUGUUGCACAAUCAGAAUUGA | 714 |
| AM13210-SS | (NAG37)s(invAb)scaguggauCfGfAfuuagugucaas(invAb) | 586 | CAGUGGAUCGAUUAGUGUCAA | 724 |
| AM13211-SS | (NAG37)s(invAb)scaguggAfuCfgAfuuagugucaas(invAb) | 587 | CAGUGGAUCGAUUAGUGUCAA | 724 |
| AM13213-SS | (NAG37)s(invAb)scaguggauCfgAfuUfagugucaas(invAb) | 588 | CAGUGGAUCGAUUAGUGUCAA | 724 |
| AM13214-SS | (NAG37)s(invAb)scaguggauCfgAfuUfaguiucaas(invAb) | 589 | CAGUGGAUCGAUUAGUIUCAA | 707 |
| AM13216-SS | (NAG37)s(invAb)succcaggaUfGfAfuAfuuaaaggas(invAb) | 590 | UCCCAGGAUGAUAUUAAAGGA | 712 |
| AM13217-SS | (NAG37)s(invAb)succcaggaUfgAfuAfuuaaaggas(invAb) | 591 | UCCCAGGAUGAUAUUAAAGGA | 712 |
| AM13218-SS | (NAG37)s(invAb)succcaggaUfGfAfua_2Nuuaaaggas(invAb) | 592 | UCCCAGGAUGAUAUUAAAGGA | 712 |
| AM13242-SS | (NAG37)s(invAb)sgcaguuggUfuUfuUfgaaugucus(invAb) | 593 | GCAGUUGGUUUUUGAAUGUCU | 720 |
| AM13243-SS | (NAG37)s(invAb)sgcaguuggUfUfUfuUfgaaugucus(invAb) | 594 | GCAGUUGGUUUUUGAAUGUCU | 720 |
| AM13248-SS | (NAG37)s(invAb)sgcagugauGfuAfUfccaaccuaus(invAb) | 595 | GCAGUGAUGUAUCCAACCUAU | 710 |
| AM13249-SS | (NAG37)s(invAb)sgcagugAfuGfuAfuccaaccuaus(invAb) | 596 | GCAGUGAUGUAUCCAACCUAU | 710 |
| AM13250-SS | (NAG37)s(invAb)sgcagugauGfuAfuCfcaaccuaus(invAb) | 597 | GCAGUGAUGUAUCCAACCUAU | 710 |
| AM13399-SS | (NAG37)s(invAb)sgccagaugUfuGfcAfgaauacuas(invAb) | 598 | GCCAGAUGUUGCAGAAUACUA | 702 |
| AM13401-SS | (NAG37)s(invAb)sgccagaUfgUfuGfcagaauacuas(invAb) | 599 | GCCAGAUGUUGCAGAAUACUA | 702 |
| AM13404-SS | (NAG37)s(invAb)scacuccUfuUfuAfaggauaaacus(invAb) | 600 | CACUCCUUUUAAGGAUAAACU | 722 |
| AM13406-SS | (NAG37)s(invAb)scacuccuuUfuAfAfggauaaacus(invAb) | 601 | CACUCCUUUUAAGGAUAAACU | 722 |
| AM13408-SS | (NAG37)s(invAb)sgacuccuuUfUfAfaggauaaacus(invAb) | 602 | GACUCCUUUUAAGGAUAAACU | 725 |
| AM13762-SS | (TriAlk14)gscaguuggUfuUfuUfgaaugucus(invAb) | 603 | GCAGUUGGUUUUUGAAUGUCU | 720 |
| AM13763-SS | (TriAlk14)gscaguuggUfUfUfuugaaugucus(invAb) | 604 | GCAGUUGGUUUUUGAAUGUCU | 720 |
| AM13764-SS | (TriAlk14)csaguggauCfgAfuUfagugucaas(invAb) | 605 | CAGUGGAUCGAUUAGUGUCAA | 724 |
| AM13765-SS | (TriAlk14)csaguggauCfgAfuUfaguiucaas(invAb) | 606 | CAGUGGAUCGAUUAGUIUCAA | 707 |
| AM14107-SS | (TriAlk14)gscaguuggUfuUfuUfgaaugucas(invAb) | 607 | GCAGUUGGUUUUUGAAUGUCA | 726 |
| AM14111-SS | (NAG37)s(invAb)sgcaguuggUfUfUfuugaaugucas(invAb) | 608 | GCAGUUGGUUUUUGAAUGUCA | 726 |
| AM14120-SS | (NAG37)s(invAb)sgauguggaGfuGfcCfagauguuas(invAb) | 609 | GAUGUGGAGUGCCAGAUGUUA | 700 |
| AM14123-SS | (NAG37)s(invAb)sgcagugauGfUfAfuccaaccuaas(invAb) | 610 | GCAGUGAUGUAUCCAACCUAA | 727 |
| AM14128-SS | (NAG37)s(invAb)scacuccuuUfUfAfaggauaaacas(invAb) | 611 | CACUCCUUUUAAGGAUAAACA | 728 |
| AM14115-SS | (NAG37)s(invAb)sgcaguuggUfuUfuUfgaaugucas(invAb) | 612 | GCAGUUGGUUUUUGAAUGUCA | 726 |
| AM14553-SS | (TriAlk14)gscagugauGfuAfuCfcaaccuaus(invAb) | 613 | GCAGUGAUGUAUCCAACCUAU | 710 |
| AM14890-SS | (invAb)sgscsaguuggUfuUfuUfgaaugucsu | 614 | GCAGUUGGUUUUUGAAUGUCU | 720 |
| AM15418-SS | (TriAlk14)gscguuggUfuUfuUfgaaugucas(invAb) | 615 | GCGUUGGUUUUUGAAUGUCA | 729 |
| AM15419-SS | (TriAlk14)gscaguugggUfuUfuUfgaaugucas(invAb) | 616 | GCAGUUGGGUUUUUGAAUGUCA | 730 |
| AM15420-SS | (TriAlk14)gscaguuggUfuUfuUfgaauguca(invAb) | 617 | GCAGUUGGUUUUUGAAUGUCA | 726 |

a_2N = 2-aminoadenosine nucleotide;
I = hypoxanthine (inosine) nucleotide

TABLE 6
MMP7 Agent Sense Strand Sequences (Shown with Targeting Ligand Conjugate.
The structure of αvβ6-SM6.1 is shown in Table 11,
and the structure of Tri-SM6.1-αvβ6-(TA14) is shown in FIG. 1.)

| | | | Corresponding |
|---|---|---|---|
| | | | Sense Strand |
| | | | AM Number |
| | | | Without Linker |
| Strand | | SEQ | or Conjugate |
| ID | Modified Sense Strand (5′ → 3′) | ID NO. | (See Table 4) |
| CS001585 | Tri-SM6.1-αvβ6-(TA14)-gsauguggaGfUfGfccagauguuas(invAb) | 618 | AM12874-SS |
| CS001588 | Tri-SM6.1-αvβ6-(TA14)-gsauguggaGfuGfcCfagauguuas(invAb) | 619 | AM12876-SS |
| CS001941 | Tri-SM6.1-αvβ6-(TA14)-gscaguuggUfuUfuUfgaaugucus(invAb) | 620 | AM13762-SS |
| CS001944 | Tri-SM6.1-αvβ6-(TA14)-gscaguuggUfUfUfuugaaugucus(invAb) | 621 | AM13763-SS |
| CS001945 | Tri-SM6.1-αvβ6-(TA14)-csaguggauCfgAfuUfagugucaas(invAb) | 622 | AM13764-SS |
| CS001947 | Tri-SM6.1-αvβ6-(TA14)-csaguggauCfgAfuUfaguiucaas(invAb) | 623 | AM13765-SS |
| CS002396 | Tri-SM6.1-αvβ6-(TA14)-gscagugauGfuAfuCfcaaccuaus(invAb) | 624 | AM14553-SS |
| CS002805 | Tri-SM6.1-αvβ6-(TA14)-gscguuggUfuUfuUfgaaugucas(invAb) | 625 | AM15418-SS |
| CS002806 | Tri-SM6.1-αvβ6-(TA14)-gscaguugggUfuUfuUfgaaugucas(invAb) | 626 | AM15419-SS |
| CS002807 | Tri-SM6.1-αvβ6-(TA14)-gscaguuggUfuUfuUfgaauguca(invAb) | 627 | AM15420-SS |
| CS002133 | Tri-SM6.1-αvβ6-(TA14)-gscaguuggUfuUfuUfgaaugucas(invAb) | 628 | AM14107-SS |

The MMP7 RNAi agents disclosed herein are formed by annealing an antisense strand with a sense strand. A sense strand containing a sequence listed in Table 2, Table 4, Table 5, or Table 6 can be hybridized to any antisense strand containing a sequence listed in Table 2 or Table 3, provided the two sequences have a region of at least 85% complementarity over a contiguous 16, 17, 18, 19, 20, or 21 nucleotide sequence.

As shown in Table 5 above, certain of the example MMP7 RNAi agent nucleotide sequences are shown to further include reactive linking groups at one or both of the 5′ terminal end and the 3′ terminal end of the sense strand. For example, many of the MMP7 RNAi agent sense strand sequences shown in Table 5 above have a (TriAlk14) linking group at the 5′ end of the nucleotide sequence. Other linking groups, such as an (NH2-C6) linking group or a (6-SS-6) or (C6-SS-C6) linking group, may be present as well or alternatively in certain embodiments. Such reactive linking groups are positioned to facilitate the linking of targeting ligands, targeting groups, and/or PK/PD modulators to the MMP7 RNAi agents disclosed herein. Linking or conjugation reactions are well known in the art and provide for formation of covalent linkages between two molecules or reactants. Suitable conjugation reactions for use in the scope of the inventions herein include, but are not limited to, amide coupling reaction, Michael addition reaction, hydrazone formation reaction, inverse-demand Diels-Alder cycloaddition reaction, oxime ligation, and Copper (I)-catalyzed or strain-promoted azide-alkyne cycloaddition reaction cycloaddition reaction.

In some embodiments, targeting ligands, such as the integrin targeting ligands shown in the examples and figures disclosed herein, can be synthesized as activated esters, such as tetrafluorophenyl (TFP) esters, which can be displaced by a reactive amino group (e.g., NH₂-C₆) to attach the targeting ligand to the MMP7 RNAi agents disclosed herein. In some embodiments, targeting ligands are synthesized as azides, which can be conjugated to a propargyl (e.g., TriAlk14) or DBCO group, for example, via Copper (I)-catalyzed or strain-promoted azide-alkyne cycloaddition reaction.

Additionally, certain of the nucleotide sequences can be synthesized with a dT nucleotide at the 3′ terminal end of the sense strand, followed by (3′→5′) a linker (e.g., C6-SS-C6). The linker can, in some embodiments, facilitate the linkage to additional components, such as, for example, a PK/PD modulator or one or more targeting ligands. As described herein, the disulfide bond of C6-SS-C6 is first reduced, removing the dT from the molecule, which can then facilitate the conjugation of the desired PK/PD modulator. The terminal dT nucleotide therefore is not a part of the fully conjugated construct.

In some embodiments, the antisense strand of a MMP7 RNAi agent disclosed herein differs by 0, 1, 2, or 3 nucleotides from any of the antisense strand sequences in Table 3 or Table 10. In some embodiments, the sense strand of a MMP7 RNAi agent disclosed herein differs by 0, 1, 2, or 3 nucleotides from any of the sense strand sequences in Table 4, Table 5, Table 6, or Table 10.

In some embodiments, a MMP7 RNAi agent antisense strand comprises a nucleotide sequence of any of the sequences in Table 2 or Table 3. In some embodiments, a MMP7 RNAi agent antisense strand comprises the sequence of nucleotides (from 5′ end→3′ end) 1-17, 2-17, 1-18, 2-18, 1-19, 2-19, 1-20, 2-20, 1-21, 2-21, 1-22, 2-22, 1-23, 2-23, 1-24, or 2-24 of any of the sequences in Table 2, Table 3, or Table 10. In certain embodiments, a MMP7 RNAi agent antisense strand comprises or consists of a modified sequence of any one of the modified sequences in Table 3 or Table 10.

In some embodiments, a MMP7 RNAi agent sense strand comprises the nucleotide sequence of any of the sequences in Table 2 or Table 4. In some embodiments, a MMP7 RNAi agent sense strand comprises the sequence of nucleotides (from 5′ end→3′ end) 1-17, 2-17, 3-17, 4-17, 1-18, 2-18, 3-18, 4-18, 1-19, 2-19, 3-19, 4-19, 1-20, 2-20, 3-20, 4-20, 1-21, 2-21, 3-21, 4-21, 1-22, 2-22, 3-22, 4-22, 1-23, 2-23, 3-23, 4-23, 1-24, 2-24, 3-24, or 4-24, of any of the sequences in Table 2, Table 4, Table 5, Table 6, or Table 10. In certain embodiments, a MMP7 RNAi agent sense strand comprises or consists of a modified sequence of any one of the modified sequences in Table 3 or Table 10.

For the RNAi agents disclosed herein, the nucleotide at position 1 of the antisense strand (from 5′ end→3′ end) can be perfectly complementary to a MMP7 gene, or can be non-complementary to a MMP7 gene. In some embodiments, the nucleotide at position 1 of the antisense strand (from 5′ end→3′ end) is a U, A, or dT (or a modified version of U, A or dT). In some embodiments, the nucleotide at position 1 of the antisense strand (from 5′ end→3′ end) forms an A:U or U:A base pair with the sense strand.

In some embodiments, a MMP7 RNAi agent antisense strand comprises the sequence of nucleotides (from 5′ end→3′ end) 2-18 or 2-19 of any of the antisense strand sequences in Table 2, Table 3, or Table 10. In some embodiments, a MMP7 RNAi sense strand comprises the sequence of nucleotides (from 5′ end→3′ end) 1-17 or 1-18 of any of the sense strand sequences in Table 2, Table 4, Table 5, Table 6, or Table 10.

In some embodiments, a MMP7 RNAi agent includes (i) an antisense strand comprising the sequence of nucleotides (from 5′ end→3′ end) 2-18 or 2-19 of any of the antisense strand sequences in Table 2, Table 3, or Table 10, and (ii) a sense strand comprising the sequence of nucleotides (from 5′ end→3′ end) 1-17 or 1-18 of any of the sense strand sequences in Table 2, Table 4, Table 5, Table 6, or Table 10.

A sense strand containing a sequence listed in Table 2 or Table 4 can be hybridized to any antisense strand containing a sequence listed in Table 2 or Table 3 provided the two sequences have a region of at least 85% complementarity over a contiguous 16, 17, 18, 19, 20, or 21 nucleotide sequence. In some embodiments, the MMP7 RNAi agent has a sense strand consisting of the modified sequence of any of the modified sequences in Table 4, Table 5, Table 6, or Table 10, and an antisense strand consisting of the modified sequence of any of the modified sequences in Table 3 or Table 10. Certain representative sequence pairings are exemplified by the Duplex ID Nos. shown in Tables 7A, 7B, 8, and 9.

In some embodiments, a MMP7 RNAi agent comprises, consists of, or consists essentially of a duplex represented by any one of the Duplex ID Nos. presented herein. In some embodiments, a MMP7 RNAi agent consists of any of the Duplex ID Nos. presented herein. In some embodiments, a MMP7 RNAi agent comprises the sense strand and antisense strand nucleotide sequences of any of the Duplex ID Nos. presented herein. In some embodiments, a MMP7 RNAi agent comprises the sense strand and antisense strand nucleotide sequences of any of the Duplex ID Nos. presented herein and a targeting group, linking group, and/or other non-nucleotide group wherein the targeting group, linking group, and/or other non-nucleotide group is covalently linked (i.e., conjugated) to the sense strand or the antisense strand. In some embodiments, a MMP7 RNAi agent includes the sense strand and antisense strand modified nucleotide sequences of any of the Duplex ID Nos. presented herein. In some embodiments, a MMP7 RNAi agent comprises the sense strand and antisense strand modified nucleotide sequences of any of the Duplex ID Nos. presented herein and a targeting group, linking group, and/or other non-nucleotide group, wherein the targeting group, linking group, and/or other non-nucleotide group is covalently linked to the sense strand or the antisense strand.

In some embodiments, a MMP7 RNAi agent comprises an antisense strand and a sense strand having the nucleotide sequences of any of the antisense strand/sense strand duplexes of Tables 2, 7A, 7B, 8, 9, or 10, and comprises a targeting group. In some embodiments, a MMP7 RNAi agent comprises an antisense strand and a sense strand having the nucleotide sequences of any of the antisense strand/sense strand duplexes of Tables 2, 7A, 7B, 8, 9, or 10, and comprises one or more αvβ6 integrin targeting ligands.

In some embodiments, a MMP7 RNAi agent comprises an antisense strand and a sense strand having the nucleotide sequences of any of the antisense strand/sense strand duplexes of Tables 2, 7A, 7B, 8, 9, or 10, and comprises a targeting group that is an integrin targeting ligand. In some embodiments, a MMP7 RNAi agent comprises an antisense strand and a sense strand having the nucleotide sequences of any of the antisense strand/sense strand duplexes of Tables 2, 7A, 7B, 8, 9, or 10, and comprises one or more αvβ6 integrin targeting ligands or clusters of αvβ6 integrin targeting ligands (e.g., a tridentate αvβ6 integrin targeting ligand).

In some embodiments, a MMP7 RNAi agent comprises an antisense strand and a sense strand having the modified nucleotide sequences of any of the antisense strand/sense strand duplexes of Tables 7A, 7B, 8, 9, and 10.

In some embodiments, a MMP7 RNAi agent comprises an antisense strand and a sense strand having the modified nucleotide sequences of any of the antisense strand/sense strand duplexes of Tables 7A, 7B, 8, 9, and 10, and comprises an integrin targeting ligand.

In some embodiments, a MMP7 RNAi agent comprises, consists of, or consists essentially of any of the duplexes of Tables 7A, 7B, 8, 9, and 10.

TABLE 7A
MMP7 RNAi Agent Duplexes with Corresponding Sense and Antisense
Strand ID Numbers and Sequence ID numbers for the modified and unmodified nucleotide
sequences. (Shown without Linking Agents or Conjugates)

| | AS | | | SS | |
|---|---|---|---|---|---|
| | modified | AS | | modified | SS |
| | SEQ ID | unmodified | | SEQ ID | unmodified |
| AS ID | NO: | SEQ ID NO: | SS ID | NO: | SEQ ID NO: |
| AM08523-AS | 337 | 629 | AM08522-SS-NL | 458 | 682 |
| AM08525-AS | 338 | 630 | AM08524-SS-NL | 459 | 683 |
| AM08527-AS | 339 | 631 | AM08526-SS-NL | 460 | 684 |
| AM08529-AS | 340 | 632 | AM08528-SS-NL | 461 | 685 |
| AM08531-AS | 341 | 633 | AM08530-SS-NL | 462 | 686 |
| AM08533-AS | 342 | 634 | AM08532-SS-NL | 463 | 687 |
| AM08535-AS | 343 | 635 | AM08534-SS-NL | 464 | 688 |
| AM08537-AS | 344 | 636 | AM08536-SS-NL | 465 | 689 |
| AM08539-AS | 345 | 637 | AM08538-SS-NL | 466 | 690 |
| AM08541-AS | 346 | 638 | AM08540-SS-NL | 467 | 691 |
| AM08543-AS | 347 | 639 | AM08542-SS-NL | 468 | 692 |
| AM08545-AS | 348 | 640 | AM08544-SS-NL | 469 | 693 |
| AM08547-AS | 349 | 641 | AM08546-SS-NL | 470 | 694 |
| AM08549-AS | 350 | 642 | AM08548-SS-NL | 471 | 695 |
| AM12367-AS | 359 | 651 | AM12366-SS-NL | 472 | 696 |
| AM12369-AS | 360 | 652 | AM12368-SS-NL | 473 | 697 |
| AM12371-AS | 361 | 653 | AM12370-SS-NL | 474 | 698 |
| AM12373-AS | 362 | 654 | AM12372-SS-NL | 475 | 699 |
| AM12375-AS | 363 | 655 | AM12374-SS-NL | 476 | 700 |
| AM08539-AS | 345 | 637 | AM12376-SS-NL | 477 | 701 |
| AM12378-AS | 364 | 656 | AM12377-SS-NL | 478 | 702 |
| AM12380-AS | 365 | 657 | AM12379-SS-NL | 479 | 703 |
| AM12382-AS | 366 | 658 | AM12381-SS-NL | 480 | 704 |
| AM12384-AS | 367 | 659 | AM12383-SS-NL | 481 | 705 |
| AM12386-AS | 368 | 660 | AM12385-SS-NL | 482 | 706 |
| AM12388-AS | 369 | 661 | AM12387-SS-NL | 483 | 707 |
| AM12390-AS | 370 | 662 | AM12389-SS-NL | 484 | 708 |
| AM12392-AS | 371 | 663 | AM12391-SS-NL | 485 | 709 |
| AM12394-AS | 372 | 664 | AM12393-SS-NL | 486 | 710 |
| AM12396-AS | 373 | 665 | AM12395-SS-NL | 487 | 711 |
| AM12398-AS | 374 | 666 | AM12397-SS-NL | 488 | 712 |
| AM12400-AS | 375 | 667 | AM12399-SS-NL | 489 | 713 |
| AM12402-AS | 376 | 668 | AM12401-SS-NL | 490 | 714 |
| AM12404-AS | 377 | 669 | AM12403-SS-NL | 491 | 715 |
| AM12406-AS | 378 | 670 | AM12405-SS-NL | 492 | 716 |
| AM12408-AS | 379 | 671 | AM12407-SS-NL | 493 | 717 |
| AM12410-AS | 380 | 672 | AM12409-SS-NL | 494 | 718 |
| AM12412-AS | 381 | 673 | AM12411-SS-NL | 495 | 719 |
| AM12414-AS | 382 | 674 | AM12413-SS-NL | 496 | 720 |
| AM12416-AS | 383 | 675 | AM12415-SS-NL | 497 | 721 |
| AM12418-AS | 384 | 676 | AM12417-SS-NL | 498 | 722 |
| AM12375-AS | 363 | 655 | AM12874-SS-NL | 499 | 700 |
| AM12875-AS | 385 | 655 | AM12874-SS-NL | 499 | 700 |
| AM12877-AS | 386 | 655 | AM12876-SS-NL | 500 | 700 |
| AM13076-AS | 387 | 677 | AM13075-SS-NL | 501 | 723 |
| AM13077-AS | 388 | 668 | AM12401-SS-NL | 490 | 714 |
| AM13079-AS | 389 | 668 | AM13078-SS-NL | 502 | 714 |
| AM13079-AS | 389 | 668 | AM13080-SS-NL | 503 | 714 |
| AM13081-AS | 390 | 668 | AM13080-SS-NL | 503 | 714 |
| AM13082-AS | 391 | 668 | AM13080-SS-NL | 503 | 714 |
| AM13083-AS | 392 | 668 | AM13080-SS-NL | 503 | 714 |
| AM13083-AS | 392 | 668 | AM13084-SS-NL | 504 | 714 |
| AM13083-AS | 392 | 668 | AM13078-SS-NL | 502 | 714 |
| AM12388-AS | 369 | 661 | AM13210-SS-NL | 505 | 724 |
| AM13212-AS | 393 | 661 | AM13211-SS-NL | 506 | 724 |
| AM13212-AS | 393 | 661 | AM13213-SS-NL | 507 | 724 |
| AM13212-AS | 393 | 661 | AM13214-SS-NL | 508 | 707 |
| AM13215-AS | 394 | 666 | AM12397-SS-NL | 488 | 712 |
| AM13215-AS | 394 | 666 | AM13216-SS-NL | 509 | 712 |
| AM13215-AS | 394 | 666 | AM13217-SS-NL | 510 | 712 |
| AM13215-AS | 394 | 666 | AM13218-SS-NL | 511 | 712 |
| AM13241-AS | 395 | 674 | AM12413-SS-NL | 496 | 720 |
| AM13241-AS | 395 | 674 | AM13242-SS-NL | 512 | 720 |
| AM13244-AS | 396 | 674 | AM13243-SS-NL | 513 | 720 |
| AM13244-AS | 396 | 674 | AM13242-SS-NL | 512 | 720 |
| AM13245-AS | 397 | 674 | AM13243-SS-NL | 513 | 720 |
| AM13246-AS | 398 | 664 | AM12393-SS-NL | 486 | 710 |
| AM13247-AS | 399 | 664 | AM12393-SS-NL | 486 | 710 |
| AM13246-AS | 398 | 664 | AM13248-SS-NL | 514 | 710 |
| AM13246-AS | 398 | 664 | AM13249-SS-NL | 515 | 710 |
| AM13246-AS | 398 | 664 | AM13250-SS-NL | 516 | 710 |
| AM13400-AS | 400 | 656 | AM13399-SS-NL | 517 | 702 |
| AM13400-AS | 400 | 656 | AM13401-SS-NL | 518 | 702 |
| AM13402-AS | 401 | 656 | AM13399-SS-NL | 517 | 702 |
| AM13403-AS | 402 | 656 | AM13399-SS-NL | 517 | 702 |
| AM13405-AS | 403 | 676 | AM13404-SS-NL | 519 | 722 |
| AM13405-AS | 403 | 676 | AM13406-SS-NL | 520 | 722 |
| AM13407-AS | 404 | 676 | AM12417-SS-NL | 498 | 722 |
| AM13409-AS | 405 | 678 | AM13408-SS-NL | 521 | 725 |
| AM13241-AS | 395 | 674 | AM13762-SS-NL | 522 | 720 |
| AM13244-AS | 396 | 674 | AM13762-SS-NL | 522 | 720 |
| AM13241-AS | 395 | 674 | AM13763-SS-NL | 523 | 720 |
| AM13212-AS | 393 | 661 | AM13764-SS-NL | 524 | 724 |
| AM13212-AS | 393 | 661 | AM13765-SS-NL | 525 | 707 |
| AM14108-AS | 406 | 679 | AM14107-SS-NL | 526 | 726 |
| AM14109-AS | 407 | 661 | AM13764-SS-NL | 524 | 724 |
| AM14110-AS | 408 | 674 | AM12413-SS-NL | 496 | 720 |
| AM14112-AS | 409 | 679 | AM14111-SS-NL | 527 | 726 |
| AM14113-AS | 410 | 679 | AM14111-SS-NL | 527 | 726 |
| AM14117-AS | 411 | 661 | AM13210-SS-NL | 505 | 724 |
| AM14118-AS | 412 | 661 | AM13210-SS-NL | 505 | 724 |
| AM14119-AS | 413 | 661 | AM13213-SS-NL | 507 | 724 |
| AM14109-AS | 407 | 661 | AM13213-SS-NL | 507 | 724 |
| AM12875-AS | 385 | 655 | AM12374-SS-NL | 476 | 700 |
| AM14121-AS | 414 | 655 | AM14120-SS-NL | 528 | 700 |
| AM14122-AS | 415 | 655 | AM14120-SS-NL | 528 | 700 |
| AM12877-AS | 386 | 655 | AM14120-SS-NL | 528 | 700 |
| AM14124-AS | 416 | 680 | AM14123-SS-NL | 529 | 727 |
| AM14125-AS | 417 | 680 | AM14123-SS-NL | 529 | 727 |
| AM14126-AS | 418 | 680 | AM14123-SS-NL | 529 | 727 |
| AM14127-AS | 419 | 680 | AM14123-SS-NL | 529 | 727 |
| AM14129-AS | 420 | 681 | AM14128-SS-NL | 530 | 728 |
| AM14130-AS | 421 | 681 | AM14128-SS-NL | 530 | 728 |
| AM14131-AS | 422 | 681 | AM14128-SS-NL | 530 | 728 |
| AM14114-AS | 423 | 674 | AM13242-SS-NL | 512 | 720 |
| AM14116-AS | 424 | 679 | AM14115-SS-NL | 531 | 726 |
| AM14108-AS | 406 | 679 | AM14115-SS-NL | 531 | 726 |
| AM13246-AS | 398 | 664 | AM14553-SS-NL | 532 | 710 |
| AM14651-AS | 425 | 680 | AM14123-SS-NL | 526 | 727 |
| AM14652-AS | 426 | 664 | AM12393-SS-NL | 486 | 710 |
| AM14653-AS | 427 | 664 | AM13250-SS-NL | 516 | 710 |
| AM14654-AS | 428 | 664 | AM13250-SS-NL | 516 | 710 |
| AM14655-AS | 429 | 664 | AM13250-SS-NL | 516 | 710 |
| AM14656-AS | 430 | 664 | AM13250-SS-NL | 516 | 710 |
| AM14657-AS | 431 | 664 | AM13250-SS-NL | 516 | 710 |
| AM14658-AS | 432 | 664 | AM13250-SS-NL | 516 | 710 |
| AM14659-AS | 433 | 664 | AM13250-SS-NL | 516 | 710 |
| AM14888-AS | 434 | 664 | AM13250-SS-NL | 516 | 710 |
| AM14889-AS | 435 | 664 | AM13250-SS-NL | 516 | 710 |
| AM14888-AS | 434 | 664 | AM14553-SS-NL | 532 | 710 |
| AM14889-AS | 435 | 664 | AM14553-SS-NL | 532 | 710 |
| AM14116-AS | 424 | 679 | AM14107-SS-NL | 526 | 726 |
| AM15155-AS | 436 | 674 | AM13762-SS-NL | 522 | 720 |
| AM14114-AS | 423 | 674 | AM13762-SS-NL | 522 | 720 |
| AM14108-AS | 406 | 679 | AM15418-SS-NL | 535 | 729 |
| AM14108-AS | 406 | 679 | AM15419-SS-NL | 536 | 730 |
| AM14108-AS | 406 | 679 | AM15420-SS-NL | 537 | 726 |
| AM14655-AS | 429 | 664 | AM14553-SS-NL | 532 | 710 |
| AM14657-AS | 431 | 664 | AM14553-SS-NL | 532 | 710 |
| AM14108-AS | 406 | 679 | AM16013-SS-NL | 538 | 726 |
| AM14116-AS | 424 | 679 | AM16013-SS-NL | 538 | 726 |

TABLE 7B
MMP7 RNAi Agent Duplexes with Corresponding Sense and Antisense
Strand ID Numbers and Sequence ID numbers for the modified and unmodified nucleotide
sequences.

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD06348 | AM08523-AS | 337 | 629 | AM08522-SS | 539 | 682 |
| AD06349 | AM08525-AS | 338 | 630 | AM08524-SS | 540 | 683 |
| AD06350 | AM08527-AS | 339 | 631 | AM08526-SS | 541 | 684 |
| AD06351 | AM08529-AS | 340 | 632 | AM08528-SS | 542 | 685 |
| AD06352 | AM08531-AS | 341 | 633 | AM08530-SS | 543 | 686 |
| AD06353 | AM08533-AS | 342 | 634 | AM08532-SS | 544 | 687 |
| AD06354 | AM08535-AS | 343 | 635 | AM08534-SS | 545 | 688 |
| AD06355 | AM08537-AS | 344 | 636 | AM08536-SS | 546 | 689 |
| AD06356 | AM08539-AS | 345 | 637 | AM08538-SS | 547 | 690 |
| AD06357 | AM08541-AS | 346 | 638 | AM08540-SS | 548 | 691 |
| AD06358 | AM08543-AS | 347 | 639 | AM08542-SS | 549 | 692 |
| AD06359 | AM08545-AS | 348 | 640 | AM08544-SS | 550 | 693 |
| AD06360 | AM08547-AS | 349 | 641 | AM08546-SS | 551 | 694 |
| AD06361 | AM08549-AS | 350 | 642 | AM08548-SS | 552 | 695 |
| AD08797 | AM12367-AS | 359 | 651 | AM12366-SS | 553 | 696 |
| AD08798 | AM12369-AS | 360 | 652 | AM12368-SS | 554 | 697 |
| AD08799 | AM12371-AS | 361 | 653 | AM12370-SS | 555 | 698 |
| AD08800 | AM12373-AS | 362 | 654 | AM12372-SS | 556 | 699 |
| AD08801 | AM12375-AS | 363 | 655 | AM12374-SS | 557 | 700 |
| AD08802 | AM08539-AS | 345 | 637 | AM12376-SS | 558 | 701 |
| AD08803 | AM12378-AS | 364 | 656 | AM12377-SS | 559 | 702 |
| AD08804 | AM12380-AS | 365 | 657 | AM12379-SS | 560 | 703 |
| AD08805 | AM12382-AS | 366 | 658 | AM12381-SS | 561 | 704 |
| AD08806 | AM12384-AS | 367 | 659 | AM12383-SS | 562 | 705 |
| AD08807 | AM12386-AS | 368 | 660 | AM12385-SS | 563 | 706 |
| AD08808 | AM12388-AS | 369 | 661 | AM12387-SS | 564 | 707 |
| AD08809 | AM12390-AS | 370 | 662 | AM12389-SS | 565 | 708 |
| AD08810 | AM12392-AS | 371 | 663 | AM12391-SS | 566 | 709 |
| AD08811 | AM12394-AS | 372 | 664 | AM12393-SS | 567 | 710 |
| AD08812 | AM12396-AS | 373 | 665 | AM12395-SS | 568 | 711 |
| AD08813 | AM12398-AS | 374 | 666 | AM12397-SS | 569 | 712 |
| AD08814 | AM12400-AS | 375 | 667 | AM12399-SS | 570 | 713 |
| AD08815 | AM12402-AS | 376 | 668 | AM12401-SS | 571 | 714 |
| AD08816 | AM12404-AS | 377 | 669 | AM12403-SS | 572 | 715 |
| AD08817 | AM12406-AS | 378 | 670 | AM12405-SS | 573 | 716 |
| AD08818 | AM12408-AS | 379 | 671 | AM12407-SS | 574 | 717 |
| AD08819 | AM12410-AS | 380 | 672 | AM12409-SS | 575 | 718 |
| AD08820 | AM12412-AS | 381 | 673 | AM12411-SS | 576 | 719 |
| AD08821 | AM12414-AS | 382 | 674 | AM12413-SS | 577 | 720 |
| AD08822 | AM12416-AS | 383 | 675 | AM12415-SS | 578 | 721 |
| AD08823 | AM12418-AS | 384 | 676 | AM12417-SS | 579 | 722 |
| AD09128 | AM12375-AS | 363 | 655 | AM12874-SS | 580 | 700 |
| AD09129 | AM12875-AS | 385 | 655 | AM12874-SS | 580 | 700 |
| AD09130 | AM12877-AS | 386 | 655 | AM12876-SS | 581 | 700 |
| AD09242 | AM13076-AS | 387 | 677 | AM13075-SS | 582 | 723 |
| AD09243 | AM13077-AS | 388 | 668 | AM12401-SS | 571 | 714 |
| AD09244 | AM13079-AS | 389 | 668 | AM13078-SS | 583 | 714 |
| AD09245 | AM13079-AS | 389 | 668 | AM13080-SS | 584 | 714 |
| AD09246 | AM13081-AS | 390 | 668 | AM13080-SS | 584 | 714 |
| AD09247 | AM13082-AS | 391 | 668 | AM13080-SS | 584 | 714 |
| AD09248 | AM13083-AS | 392 | 668 | AM13080-SS | 584 | 714 |
| AD09249 | AM13083-AS | 392 | 668 | AM13084-SS | 585 | 714 |
| AD09250 | AM13083-AS | 392 | 668 | AM13078-SS | 583 | 714 |
| AD09330 | AM12388-AS | 369 | 661 | AM13210-SS | 586 | 724 |
| AD09331 | AM13212-AS | 393 | 661 | AM13211-SS | 587 | 724 |
| AD09332 | AM13212-AS | 393 | 661 | AM13213-SS | 588 | 724 |
| AD09333 | AM13212-AS | 393 | 661 | AM13214-SS | 589 | 707 |
| AD09334 | AM13215-AS | 394 | 666 | AM12397-SS | 569 | 712 |
| AD09335 | AM13215-AS | 394 | 666 | AM13216-SS | 590 | 712 |
| AD09336 | AM13215-AS | 394 | 666 | AM13217-SS | 591 | 712 |
| AD09337 | AM13215-AS | 394 | 666 | AM13218-SS | 592 | 712 |
| AD09349 | AM13241-AS | 395 | 674 | AM12413-SS | 577 | 720 |
| AD09350 | AM13241-AS | 395 | 674 | AM13242-SS | 593 | 720 |
| AD09351 | AM13244-AS | 396 | 674 | AM13243-SS | 594 | 720 |
| AD09352 | AM13244-AS | 396 | 674 | AM13242-SS | 593 | 720 |
| AD09353 | AM13245-AS | 397 | 674 | AM13243-SS | 594 | 720 |
| AD09354 | AM13246-AS | 398 | 664 | AM12393-SS | 567 | 710 |
| AD09355 | AM13247-AS | 399 | 664 | AM12393-SS | 567 | 710 |
| AD09356 | AM13246-AS | 398 | 664 | AM13248-SS | 595 | 710 |
| AD09357 | AM13246-AS | 398 | 664 | AM13249-SS | 596 | 710 |
| AD09358 | AM13246-AS | 398 | 664 | AM13250-SS | 597 | 710 |
| AD09441 | AM13400-AS | 400 | 656 | AM13399-SS | 598 | 702 |
| AD09442 | AM13400-AS | 400 | 656 | AM13401-SS | 599 | 702 |
| AD09443 | AM13402-AS | 401 | 656 | AM13399-SS | 598 | 702 |
| AD09444 | AM13403-AS | 402 | 656 | AM13399-SS | 598 | 702 |
| AD09445 | AM13405-AS | 403 | 676 | AM13404-SS | 600 | 722 |
| AD09446 | AM13405-AS | 403 | 676 | AM13406-SS | 601 | 722 |
| AD09447 | AM13407-AS | 404 | 676 | AM12417-SS | 579 | 722 |
| AD09448 | AM13409-AS | 405 | 678 | AM13408-SS | 602 | 725 |
| AD09666 | AM13241-AS | 395 | 674 | AM13762-SS | 603 | 720 |
| AD09667 | AM13244-AS | 396 | 674 | AM13762-SS | 603 | 720 |
| AD09668 | AM13241-AS | 395 | 674 | AM13763-SS | 604 | 720 |
| AD09669 | AM13212-AS | 393 | 661 | AM13764-SS | 605 | 724 |
| AD09670 | AM13212-AS | 393 | 661 | AM13765-SS | 606 | 707 |
| AD09887 | AM14108-AS | 406 | 679 | AM14107-SS | 607 | 726 |
| AD09888 | AM14109-AS | 407 | 661 | AM13764-SS | 605 | 724 |
| AD09889 | AM14110-AS | 408 | 674 | AM12413-SS | 577 | 720 |
| AD09890 | AM14112-AS | 409 | 679 | AM14111-SS | 608 | 726 |
| AD09891 | AM14113-AS | 410 | 679 | AM14111-SS | 608 | 726 |
| AD09892 | AM14117-AS | 411 | 661 | AM13210-SS | 586 | 724 |
| AD09893 | AM14118-AS | 412 | 661 | AM13210-SS | 586 | 724 |
| AD09894 | AM14119-AS | 413 | 661 | AM13213-SS | 588 | 724 |
| AD09895 | AM14109-AS | 407 | 661 | AM13213-SS | 588 | 724 |
| AD09896 | AM12875-AS | 385 | 655 | AM12374-SS | 557 | 700 |
| AD09897 | AM14121-AS | 414 | 655 | AM14120-SS | 609 | 700 |
| AD09898 | AM14122-AS | 415 | 655 | AM14120-SS | 609 | 700 |
| AD09899 | AM12877-AS | 386 | 655 | AM14120-SS | 609 | 700 |
| AD09900 | AM14124-AS | 416 | 680 | AM14123-SS | 610 | 727 |
| AD09901 | AM14125-AS | 417 | 680 | AM14123-SS | 610 | 727 |
| AD09902 | AM14126-AS | 418 | 680 | AM14123-SS | 610 | 727 |
| AD09903 | AM14127-AS | 419 | 680 | AM14123-SS | 610 | 727 |
| AD09904 | AM14129-AS | 420 | 681 | AM14128-SS | 611 | 728 |
| AD09905 | AM14130-AS | 421 | 681 | AM14128-SS | 611 | 728 |
| AD09906 | AM14131-AS | 422 | 681 | AM14128-SS | 611 | 728 |
| AD09907 | AM14114-AS | 423 | 674 | AM13242-SS | 593 | 720 |
| AD09908 | AM14116-AS | 424 | 679 | AM14115-SS | 612 | 726 |
| AD09909 | AM14108-AS | 406 | 679 | AM14115-SS | 612 | 726 |
| AD10212 | AM13246-AS | 398 | 664 | AM14553-SS | 613 | 710 |
| AD10284 | AM14651-AS | 425 | 680 | AM14123-SS | 610 | 727 |
| AD10285 | AM14652-AS | 426 | 664 | AM12393-SS | 567 | 710 |
| AD10286 | AM14653-AS | 427 | 664 | AM13250-SS | 597 | 710 |
| AD10287 | AM14654-AS | 428 | 664 | AM13250-SS | 597 | 710 |
| AD10288 | AM14655-AS | 429 | 664 | AM13250-SS | 597 | 710 |
| AD10289 | AM14656-AS | 430 | 664 | AM13250-SS | 597 | 710 |
| AD10290 | AM14657-AS | 431 | 664 | AM13250-SS | 597 | 710 |
| AD10291 | AM14658-AS | 432 | 664 | AM13250-SS | 597 | 710 |
| AD10292 | AM14659-AS | 433 | 664 | AM13250-SS | 597 | 710 |
| AD10438 | AM14888-AS | 434 | 664 | AM13250-SS | 597 | 710 |
| AD10439 | AM14889-AS | 435 | 664 | AM13250-SS | 597 | 710 |
| AD10441 | AM14888-AS | 434 | 664 | AM14553-SS | 613 | 710 |
| AD10442 | AM14889-AS | 435 | 664 | AM14553-SS | 613 | 710 |
| AD10637 | AM14116-AS | 424 | 679 | AM14107-SS | 607 | 726 |
| AD10638 | AM15155-AS | 436 | 674 | AM13762-SS | 603 | 720 |
| AD10639 | AM14114-AS | 423 | 674 | AM13762-SS | 603 | 720 |
| AD10815 | AM14108-AS | 406 | 679 | AM15418-SS | 615 | 729 |
| AD10816 | AM14108-AS | 406 | 679 | AM15419-SS | 616 | 730 |
| AD10817 | AM14108-AS | 406 | 679 | AM15420-SS | 617 | 726 |
| AD10879 | AM14655-AS | 429 | 664 | AM14553-SS | 613 | 710 |
| AD10880 | AM14657-AS | 431 | 664 | AM14553-SS | 613 | 710 |

TABLE 8
MMP7 RNAi Agent Duplexes with Corresponding Sense and Antisense
Strand ID Numbers and Sequence ID numbers for the modified and unmodified nucleotide
sequences. (Shown with Targeting Ligand Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AC001271 | AM12375-AS | 363 | 655 | CS001585 | 618 | 700 |
| AC001272 | AM12875-AS | 385 | 655 | CS001585 | 618 | 700 |
| AC001273 | AM12877-AS | 386 | 655 | CS001588 | 619 | 700 |
| AC001513 | AM13241-AS | 395 | 674 | CS001941 | 620 | 720 |
| AC001514 | AM13244-AS | 396 | 674 | CS001941 | 620 | 720 |
| AC001515 | AM13241-AS | 395 | 674 | CS001944 | 621 | 720 |
| AC001516 | AM13212-AS | 393 | 661 | CS001945 | 622 | 724 |
| AC001517 | AM13212-AS | 393 | 661 | CS001947 | 623 | 707 |
| AC001651 | AM14108-AS | 406 | 679 | CS002133 | 628 | 726 |
| AC001652 | AM14109-AS | 407 | 661 | CS001945 | 622 | 724 |
| AC001875 | AM13246-AS | 398 | 664 | CS002396 | 624 | 710 |
| AC002023 | AM14888-AS | 434 | 664 | CS002396 | 624 | 710 |
| AC002024 | AM14889-AS | 435 | 664 | CS002396 | 624 | 710 |
| AC002085 | AM14116-AS | 424 | 679 | CS002133 | 628 | 726 |
| AC002086 | AM15155-AS | 436 | 674 | CS001941 | 620 | 720 |
| AC002087 | AM14114-AS | 423 | 674 | CS001941 | 620 | 720 |
| AC002217 | AM14655-AS | 429 | 664 | CS002396 | 624 | 710 |
| AC002218 | AM14657-AS | 431 | 664 | CS002396 | 624 | 710 |
| AC002219 | AM14108-AS | 406 | 679 | CS002805 | 625 | 729 |
| AC002220 | AM14108-AS | 406 | 679 | CS002806 | 626 | 730 |
| AC002221 | AM14108-AS | 406 | 679 | CS002807 | 627 | 726 |

TABLE 9
Conjugate Duplex ID Numbers Referencing Position
Targeted On MMP7 (MMP7) Gene

| | | | | Targeted |
|---|---|---|---|---|
| | | | | MMP7 |
| | | | | Gene |
| | | | | Position |
| | | | | (Of SEQ |
| Duplex | AS ID | SS ID | Duplex | ID NO: 1) |
| AC001271 | AM12375-AS | CS001585 | AD09128 | 304 |
| AC001272 | AM12875-AS | CS001585 | AD09129 | 304 |
| AC001273 | AM12877-AS | CS001588 | AD09130 | 304 |
| AC001513 | AM13241-AS | CS001941 | AD09666 | 971 |
| AC001514 | AM13244-AS | CS001941 | AD09667 | 971 |
| AC001515 | AM13241-AS | CS001944 | AD09668 | 971 |
| AC001516 | AM13212-AS | CS001945 | AD09669 | 418 |
| AC001517 | AM13212-AS | CS001947 | AD09670 | 418 |
| AC001651 | AM14108-AS | CS002133 | AD09887 | 971 |
| AC001652 | AM14109-AS | CS001945 | AD09888 | 418 |
| AC001875 | AM13246-AS | CS002396 | AD10212 | 735 |
| AC002023 | AM14888-AS | CS002396 | AD10441 | 735 |
| AC002024 | AM14889-AS | CS002396 | AD10442 | 735 |
| AC002085 | AM14116-AS | CS002133 | AD10637 | 971 |
| AC002086 | AM15155-AS | CS001941 | AD10638 | 971 |
| AC002087 | AM14114-AS | CS001941 | AD10639 | 971 |
| AC002217 | AM14655-AS | CS002396 | AD10879 | 735 |
| AC002218 | AM14657-AS | CS002396 | AD10880 | 735 |
| AC002219 | AM14108-AS | CS002805 | AD10815 | 971 |
| AC002220 | AM14108-AS | CS002806 | AD10816 | 971 |
| AC002221 | AM14108-AS | CS002807 | AD10817 | 971 |

TABLE 10
Conjugate ID Numbers With Chemically Modified Antisense
and Sense Strands (including Linkers and Conjugates)

| | Sense Strand (Fully | | | |
|---|---|---|---|---|
| AC ID | Modified with Conjugated | SEQ ID | | SEQ ID |
| Number | Targeting Ligand)(5′ → 3′) | NO: | Antisense Strand (5′ → 3′) | NO: |
| AC001271 | Tri-SM6.1-αvβ6-(TA14) | 618 | usAfsasCfaUfcUfgGfcAfcUfcC | 363 |
| | gsauguggaGfUfGfccagauguuas(invAb) | | faCfaUfsc | |
| AC001272 | Tri-SM6.1-αvβ6-(TA14) | 618 | cPrpusAfsasCfaUfcUfgGfcAfc | 385 |
| | gsauguggaGfUfGfccagauguuas(invAb) | | UfcCfaCfaUfsc | |
| AC001273 | Tri-SM6.1-αvβ6-(TA14) | 619 | cPrpuAfacaucuggcAfcUfcCfac | 386 |
| | gsauguggaGfuGfcCfagauguuas(invAb) | | ausc | |
| AC001513 | Tri-SM6.1-αvβ6-(TA14) | 620 | asGfsascauucaaaAfaCfcAfacu | 395 |
| | gscaguuggUfuUfuUfgaaugucus(invAb) | | gsc | |
| AC001514 | Tri-SM6.1-αvβ6-(TA14) | 620 | asGfsascauucAfaaAfaCfcAfac | 396 |
| | gscaguuggUfuUfuUfgaaugucus(invAb) | | ugsc | |
| AC001515 | Tri-SM6.1-αvβ6-(TA14) | 621 | asGfsascauucaaaAfaCfcAfacu | 395 |
| | gscaguuggUfUfUfuugaaugucus(invAb) | | gsc | |
| AC001516 | Tri-SM6.1-αvβ6-(TA14) | 622 | usUfsgsacacUfaAfuCfgAfuCfc | 393 |
| | csaguggauCfgAfuUfagugucaas(invAb) | | acusg | |
| AC001517 | Tri-SM6.1-αvβ6-(TA14) | 623 | usUfsgsacacUfaAfuCfgAfuCfc | 393 |
| | csaguggauCfgAfuUfaguiucaas(invAb) | | acusg | |
| AC001875 | Tri-SM6.1-αvβ6-(TA14) | 624 | asUfsasgguuggauAfcAfuCfacu | 398 |
| | gscagugauGfuAfuCfcaaccuaus(invAb) | | gsc | |
| AC002217 | Tri-SM6.1-αvβ6-(TA14) | 624 | cPrpasUfsaGfguuggauAfcAfuC | 429 |
| | gscagugauGfuAfuCfcaaccuaus(invAb) | | facusgsc | |
| AC002218 | Tri-SM6.1-αvβ6-(TA14) | 624 | cPrpasUfsagGfuuggauAfcAfuC | 431 |
| | gscagugauGfuAfuCfcaaccuaus(invAb) | | facusgsc | |
| AC002219 | Tri-SM6.1-αvβ6-(TA14) | 625 | cPrpuGfacauucAfaaAfaCfcAfa | 406 |
| | gscguuggUfuUfuUfgaaugucas(invAb) | | cugsc | |
| AC002220 | Tri-SM6.1-αvβ6-(TA14) | 626 | cPrpuGfacauucAfaaAfaCfcAfa | 406 |
| | gscaguugggUfuUfuUfgaaugucas(invAb) | | cugsc | |
| AC002221 | Tri-SM6.1-αvβ6-(TA14) | 627 | cPrpuGfacauucAfaaAfaCfcAfa | 406 |
| | gscaguuggUfuUfuUfgaauguca(invAb) | | cugsc | |
| AC001651 | Tri-SM6.1-αvβ6-(TA14) | 628 | cPrpuGfacauucAfaaAfaCfcAfa | 406 |
| | gscaguuggUfuUfuUfgaaugucas(invAb) | | cugsc | |
| AC001652 | Tri-SM6.1-αvβ6-(TA14) | 622 | cPrpuUfgacacUfaAfuCfgAfuCf | 407 |
| | csaguggauCfgAfuUfagugucaas(invAb) | | cacusg | |
| AC002085 | Tri-SM6.1-αvβ6-(TA14) | 628 | cPrpusGfsascauucAfaaAfaCfc | 424 |
| | gscaguuggUfuUfuUfgaaugucas(invAb) | | Afacugsc | |
| AC002086 | Tri-SM6.1-αvβ6-(TA14) | 620 | cPrpaGfacauucAfaaAfaCfcAfa | 436 |
| | gscaguuggUfuUfuUfgaaugucus(invAb) | | cugsc | |
| AC002087 | Tri-SM6.1-αvβ6-(TA14) | 620 | cPrpasGfsascauucAfaaAfaCfc | 423 |
| | gscaguuggUfuUfuUfgaaugucus(invAb) | | Afacugsc | |
| AC002023 | Tri-SM6.1-αvβ6-(TA14) | 624 | cPrpasUfsasgguuggauAfcAfuC | 434 |
| | gscagugauGfuAfuCfcaaccuaus(invAb) | | facugsc | |
| AC002024 | Tri-SM6.1-αvβ6-(TA14) | 624 | cPrpaUfagguuggauAfcAfuCfac | 435 |
| | gscagugauGfuAfuCfcaaccuaus(invAb) | | ugsc | |

In some embodiments, a MMP7 RNAi agent is prepared or provided as a salt, mixed salt, or a free-acid. In some embodiments, a MMP7 RNAi agent is prepared or provided as a pharmaceutically acceptable salt. In some embodiments, a MMP7 RNAi agent is prepared or provided as a pharmaceutically acceptable sodium or potassium salt The RNAi agents described herein, upon delivery to a cell expressing an MMP7 gene, inhibit or knockdown expression of one or more MMP7 genes in vivo and/or in vitro.

### Targeting Groups, Linking Groups, Pharmacokinetic/Pharmacodynamic (PK/PD) Modulators, and Delivery Vehicles

In some embodiments, a MMP7 RNAi agent contains or is conjugated to one or more non-nucleotide groups including, but not limited to, a targeting group, a linking group, a pharmacokinetic/pharmacodynamic (PK/PD) modulator, a delivery polymer, or a delivery vehicle. The non-nucleotide group can enhance targeting, delivery, or attachment of the RNAi agent. The non-nucleotide group can be covalently linked to the 3′ and/or 5′ end of either the sense strand and/or the antisense strand. In some embodiments, a MMP7 RNAi agent contains a non-nucleotide group linked to the 3′ and/or 5′ end of the sense strand. In some embodiments, a non-nucleotide group is linked to the 5′ end of a MMP7 RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

Targeting groups or targeting moieties enhance the pharmacokinetic or biodistribution properties of a conjugate or RNAi agent to which they are attached to improve cell-specific (including, in some cases, organ specific) distribution and cell-specific (or organ specific) uptake of the conjugate or RNAi agent. A targeting group can be monovalent, divalent, trivalent, tetravalent, or have higher valency for the target to which it is directed. Representative targeting groups include, without limitation, compounds with affinity to cell surface molecule, cell receptor ligands, hapten, antibodies, monoclonal antibodies, antibody fragments, and antibody mimics with affinity to cell surface molecules. In some embodiments, a targeting group is linked to an RNAi agent using a linker, such as a PEG linker or one, two, or three abasic and/or ribitol (abasic ribose) residues, which in some instances can serve as linkers.

A targeting group, with or without a linker, can be attached to the 5′ or 3′ end of any of the sense and/or antisense strands disclosed in Tables 2, 3, 4, 5, 6, and 10. A linker, with or without a targeting group, can be attached to the 5′ or 3′ end of any of the sense and/or antisense strands disclosed in Tables 2, 3, 4, 5, 6, and 10.

The MMP7 RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

For example, in some embodiments, the MMP7 RNAi agents disclosed herein are synthesized having an NH₂-C₆ group at the 5′-terminus of the sense strand of the RNAi agent. The terminal amino group subsequently can be reacted to form a conjugate with, for example, a group that includes an αvβ6 integrin targeting ligand. In some embodiments, the MMP7 RNAi agents disclosed herein are synthesized having one or more alkyne groups at the 5′-terminus of the sense strand of the RNAi agent. The terminal alkyne group(s) can subsequently be reacted to form a conjugate with, for example, a group that includes an αvβ6 integrin targeting ligand.

In some embodiments, a targeting group comprises an integrin targeting ligand. In some embodiments, an integrin targeting ligand is an αvβ6 integrin targeting ligand. The use of an αvβ6 integrin targeting ligand facilitates cell-specific targeting to cells having αvβ6 on its respective surface, and binding of the integrin targeting ligand can facilitate entry of the therapeutic agent, such as an RNAi agent, to which it is linked, into cells such as epithelial cells, including pulmonary epithelial cells and renal epithelial cells. Integrin targeting ligands can be monomeric or monovalent (e.g., having a single integrin targeting moiety) or multimeric or multivalent (e.g., having multiple integrin targeting moieties). The targeting group can be attached to the 3′ and/or 5′ end of the RNAi oligonucleotide using methods known in the art. The preparation of targeting groups, such as αvβ6 integrin targeting ligands, is described, for example, in International Patent Application Publication No. WO 2018/085415 and in International Patent Application Publication No. WO 2019/089765, the contents of each of which are incorporated herein in its entirety.

In some embodiments, targeting groups are linked to the MMP7 RNAi agents without the use of an additional linker. In some embodiments, the targeting group is designed having a linker readily present to facilitate the linkage to a MMP7 RNAi agent. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

In some embodiments, a linking group is conjugated to the RNAi agent. The linking group facilitates covalent linkage of the agent to a targeting group, pharmacokinetic modulator, delivery polymer, or delivery vehicle. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include but are not limited to: C6-SS-C6, 6-SS-6, reactive groups such a primary amines (e.g., NH2-C6) and alkynes, alkyl groups, abasic residues/nucleotides, amino acids, tri-alkyne functionalized groups, ribitol, and/or PEG groups. Examples of certain linking groups are provided in Table 11.

A linker or linking group is a connection between two atoms that links one chemical group (such as an RNAi agent) or segment of interest to another chemical group (such as a targeting group, pharmacokinetic modulator, or delivery polymer) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description. In some embodiments, a MMP7 RNAi agent is conjugated to a polyethylene glycol (PEG) moiety, or to a hydrophobic group having 12 or more carbon atoms, such as a cholesterol or palmitoyl group.

In some embodiments, a MMP7 RNAi agent is linked to one or more pharmacokinetic/pharmacodynamic (PK/PD) modulators. PK/PD modulators can increase circulation time of the conjugated drug and/or increase the activity of the RNAi agent through improved cell receptor binding, improved cellular uptake, and/or other means. Various PK/PD modulators suitable for use with RNAi agents are known in the art. In some embodiments, the PK/PD modulatory can be cholesterol or cholesteryl derivatives, or in some circumstances a PK/PD modulator can be comprised of alkyl groups, alkenyl groups, alkynyl groups, aryl groups, aralkyl groups, aralkenyl groups, or aralkynyl groups, each of which may be linear, branched, cyclic, and/or substituted or unsubstituted. In some embodiments, the location of attachment for these moieties is at the 5′ or 3′ end of the sense strand, at the 2′ position of the ribose ring of any given nucleotide of the sense strand, and/or attached to the phosphate or phosphorothioate backbone at any position of the sense strand.

Any of the MMP7 RNAi agent nucleotide sequences listed in Tables 2, 3, 4, 5, 6, and 10, whether modified or unmodified, can contain 3′ and/or 5′ targeting group(s), linking group(s), and/or PK/PD modulator(s). Any of the MMP7 RNAi agent sequences listed in Tables 3, 4, 5, 6, and 10, or are otherwise described herein, which contain a 3′ or 5′ targeting group, linking group, and/or PK/PD modulator can alternatively contain no 3′ or 5′ targeting group, linking group, or PK/PD modulator, or can contain a different 3′ or 5′ targeting group, linking group, or pharmacokinetic modulator including, but not limited to, those depicted in Table 11. Any of the MMP7 RNAi agent duplexes listed in Tables 7A, 7B, 8, 9 and 10, whether modified or unmodified, can further comprise a targeting group or linking group, including, but not limited to, those depicted in Table 11, and the targeting group or linking group can be attached to the 3′ or 5′ terminus of either the sense strand or the antisense strand of the MMP7 RNAi agent duplex.

Examples of certain modified nucleotides, capping moieties, and linking groups are provided in Table 11.

TABLE 11
Structures Representing Various Modified Nucleotides, Capping Moieties, and
Linking Groups (wherein [symbol] indicates the point of connection)

| | |
|---|---|
| 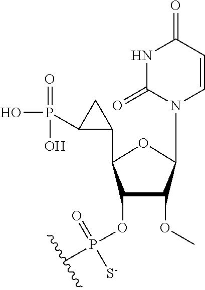 | 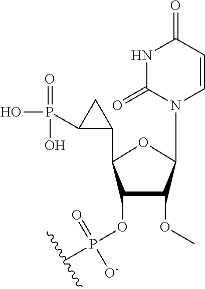 |
| cPrpus | cPrpu |
| 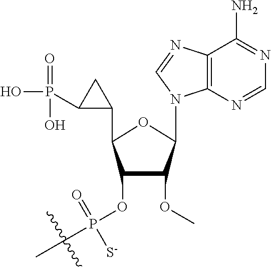 | 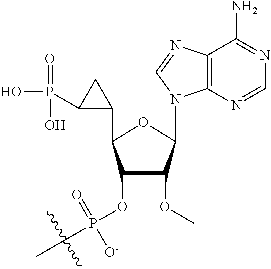 |
| cPrpas | cPrpa |
| 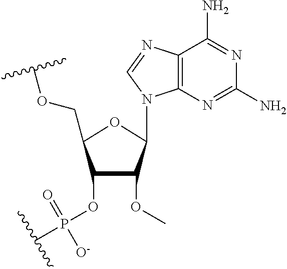 | 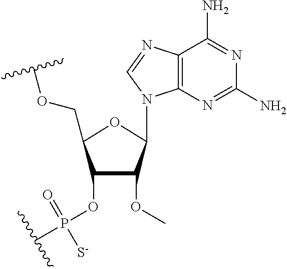 |
| a_2N | a_2Ns |
| When positioned internally: | When positioned internally: |
| linkage towards 5′ end | linkage towards 5′ end |
| 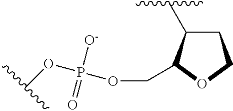 | 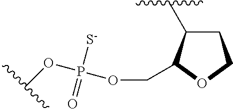 |
| linkage towards 3′ end | linkage towards 3′ end |
| (invAb) | (invAb)s |

When positioned at the 3′ terminal end:
linkage towards 5′ end

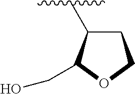

(invAb)
When positioned at the 3′ terminal end:

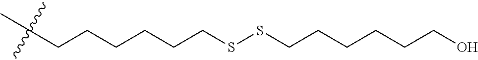 [SMILES: *CCCCCCSSCCCCCCO]

linkage towards 5′ end
(C6-SS-C6)
When positioned internally:
linkage towards 5′ end

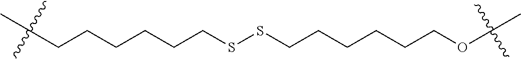 [SMILES: *CCCCCCSSCCCCCCO*]

linkage towards 3′ end
(C6-SS-C6)
When positioned at the 3′ terminal end:

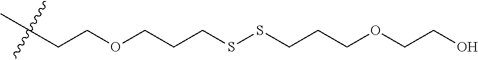 [SMILES: *CCOCCCSSCCCOCCO]

linkage towards 5′ end
(6-SS-6)
When positioned internally:
linkage towards 5′ end

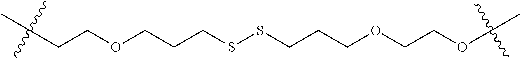 [SMILES: *CCOCCCSSCCCOCCO*]

linkage towards 3′ end
(6-SS-6)

| | |
|---|---|
| 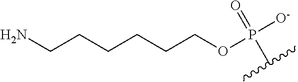 [SMILES: *P(=O)([O-])OCCCCCCN] | 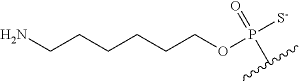 [SMILES: *P(=O)([S-])OCCCCCCN] |
| (NH2-C6) | (NH2-C6)s |
| 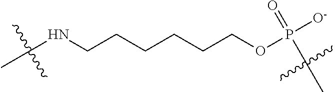 [SMILES: *NCCCCCCOP(*)(=O)[O-]] | 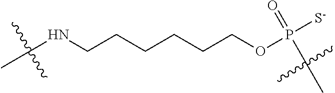 [SMILES: *NCCCCCCOP(*)(=O)[S-]] |
| -C6- | -C6s- |

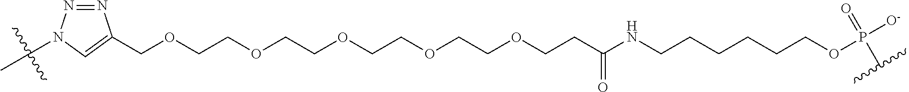 [SMILES: *n1cc(COCCOCCOCCOCCOCCC(=O)NCCCCCCOP(*)(=O)[O-])nn1]

-L6-C6-

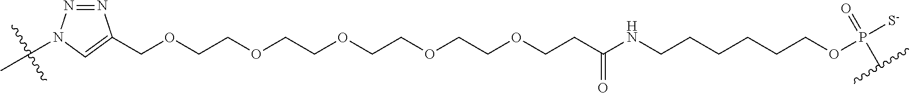 [SMILES: *n1cc(COCCOCCOCCOCCOCCC(=O)NCCCCCCOP(*)(=O)[S-])nn1]

-L6-C6s-

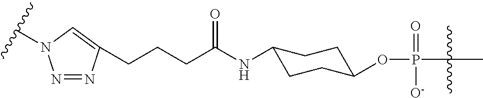 [SMILES: *n1cc(CCCC(=O)NC2CCC(OP(*)(=O)[O-])CC2)nn1]

-Alk-cyHex-

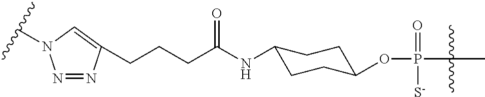 [SMILES: *n1cc(CCCC(=O)NC2CCC(OP(*)(=O)[S-])CC2)nn1]

-Alk-cyHexs-

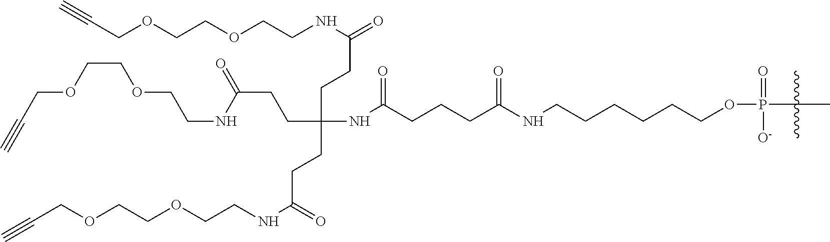 [SMILES: *P(=O)([O-])OCCCCCCNC(=O)CCCC(=O)NC(CCC(=O)NCCOCCOCC#C)(CCC(=O)NCCOCCOCC#C)CCC(=O)NCCOCCOCC#C]

(TriAlk14)

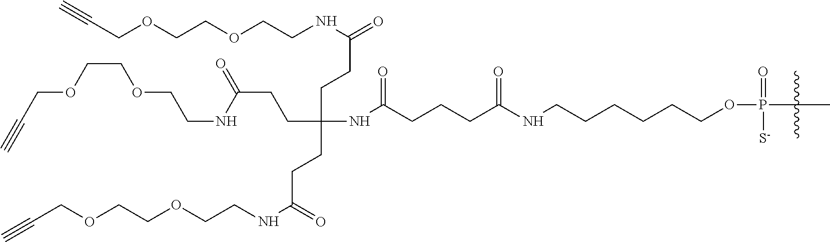 [SMILES: *P(=O)([S-])OCCCCCCNC(=O)CCCC(=O)NC(CCC(=O)NCCOCCOCC#C)(CCC(=O)NCCOCCOCC#C)CCC(=O)NCCOCCOCC#C]

(TriAlk14)s

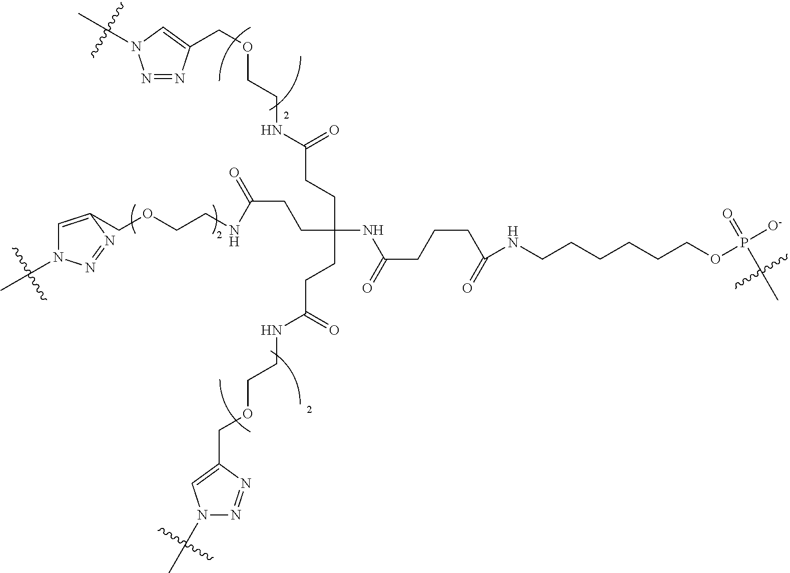

(TA14)

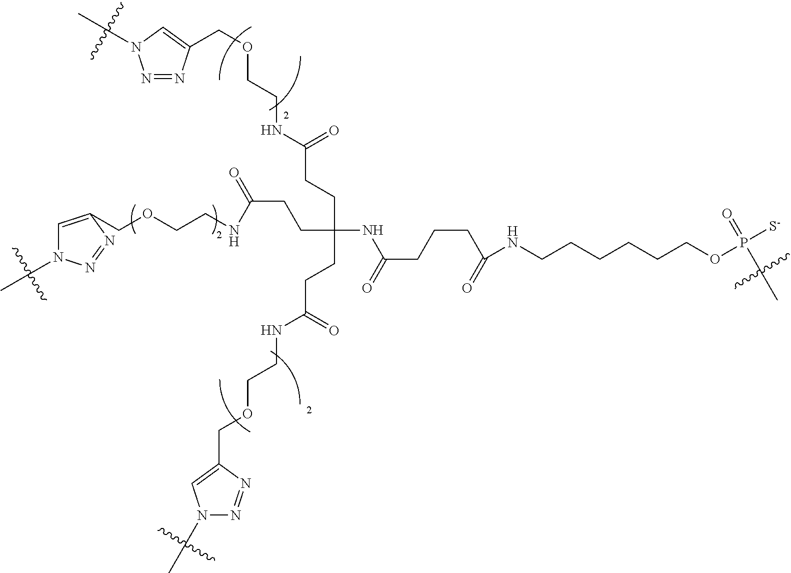

(TA14)s

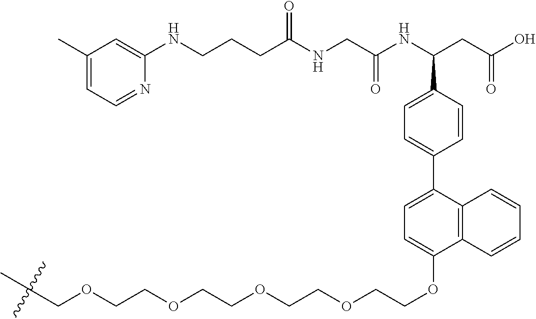 [SMILES: *COCCOCCOCCOCCOc1ccc(-c2ccc([C@H](CC(=O)O)NC(=O)CNC(=O)CCCNc3cc(C)ccn3)cc2)c2ccccc12]

SM6.1-ανβ6

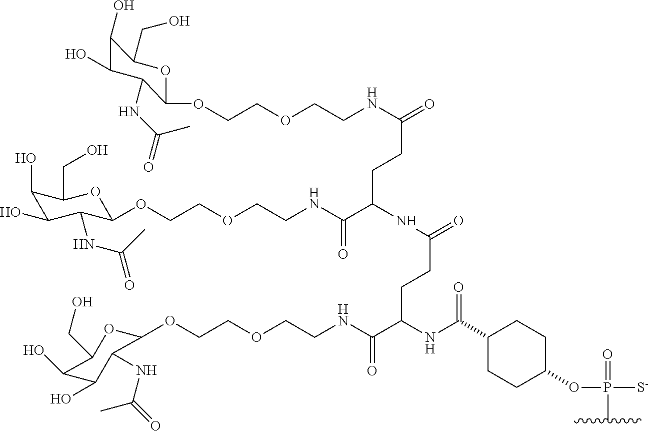

(NAG37)s

Alternatively, other linking groups known in the art may be used. In many instances, linking groups can be commercially acquired or alternatively, are incorporated into commercially available nucleotide phosphoramidites. (See, e.g., International Patent Application Publication No. WO 2019/161213, which is incorporated herein by reference in its entirety).

In some embodiments, a MMP7 RNAi agent is delivered without being conjugated to a targeting ligand or pharmacokinetic/pharmacodynamic (PK/PD) modulator (referred to as being “naked” or a “naked RNAi agent”).

In some embodiments, a MMP7 RNAi agent is conjugated to a targeting group, a linking group, a PK modulator, and/or another non-nucleotide group to facilitate delivery of the MMP7 RNAi agent to the cell or tissue of choice, for example, to an epithelial cell in vivo. In some embodiments, a MMP7 RNAi agent is conjugated to a targeting group wherein the targeting group includes an integrin targeting ligand. In some embodiments, the integrin targeting ligand is an αvβ6 integrin targeting ligand. In some embodiments, a targeting group includes one or more αvβ6 integrin targeting ligands.

In some embodiments, a delivery vehicle may be used to deliver an RNAi agent to a cell or tissue. A delivery vehicle is a compound that improves delivery of the RNAi agent to a cell or tissue. A delivery vehicle can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents can be combined with lipids, nanoparticles, polymers, liposomes, micelles, DPCs or other delivery systems available in the art for nucleic acid delivery. The RNAi agents can also be chemically conjugated to targeting groups, lipids (including, but not limited to cholesteryl and cholesteryl derivatives), encapsulating in nanoparticles, liposomes, micelles, conjugating to polymers or DPCs (see, for example WO 2000/053722, WO 2008/022309, WO 2011/104169, and WO 2012/083185, WO 2013/032829, WO 2013/158141, each of which is incorporated herein by reference), by iontophoresis, or by incorporation into other delivery vehicles or systems available in the art such as hydrogels, cyclodextrins, biodegradable nanocapsules, bioadhesive microspheres, or proteinaceous vectors. In some embodiments the RNAi agents can be conjugated to antibodies having affinity for pulmonary epithelial cells. In some embodiments, the RNAi agents can be linked to targeting ligands that have affinity for pulmonary epithelial cells or receptors present on pulmonary epithelial cells.

### Pharmaceutical Compositions and Formulations

The MMP7 RNAi agents disclosed herein can be prepared as pharmaceutical compositions (alternatively referred to as pharmaceutical formulations or medicaments). The pharmaceutical compositions disclosed herein include at least one MMP7 RNAi agent. These pharmaceutical compositions are particularly useful in the inhibition of the expression of MMP7 mRNA in a target cell, a group of cells, a tissue, or an organism. The pharmaceutical compositions can be used to treat a subject having a disease, disorder, or condition that would benefit from reduction in the level of the target mRNA, or inhibition in expression of the target gene. The pharmaceutical compositions can be used to treat a subject at risk of developing a disease or disorder that would benefit from reduction of the level of the target mRNA or an inhibition in expression the target gene. In one embodiment, the method includes administering a MMP7 RNAi agent linked to a targeting ligand as described herein, to a subject to be treated. In some embodiments, one or more pharmaceutically acceptable excipients (including vehicles, carriers, diluents, and/or delivery polymers) are added to the pharmaceutical compositions that include a MMP7 RNAi agent, thereby forming a pharmaceutical formulation or medicament suitable for in vivo delivery to a subject, including a human.

The pharmaceutical compositions that include a MMP7 RNAi agent and methods disclosed herein decrease the level of the target mRNA in a cell, group of cells, group of cells, tissue, organ, or subject, including by administering to the subject a therapeutically effective amount of a herein described MMP7 RNAi agent, thereby inhibiting the expression of MMP7 mRNA in the subject. In some embodiments, the subject has been previously identified or diagnosed as having a disease or disorder that can be mediated at least in part by a reduction in MMP7 expression. In some embodiments, the subject has been previously diagnosed with having one or more pulmonary diseases such as idiopathic pulmonary fibrosis (IPF), asthma (including severe asthma), acute respiratory distress syndrome, lung cancer, chronic inflammation, interstitial lung diseases (ILD), or another type of fibrosis. In some embodiments, the subject has been previously diagnosed with having IPF.

Embodiments of the present disclosure include pharmaceutical compositions for delivering a MMP7 RNAi agent to a pulmonary epithelial cell in vivo. Such pharmaceutical compositions can include, for example, a MMP7 RNAi agent conjugated to a targeting group that comprises an integrin targeting ligand. In some embodiments, the integrin targeting ligand is comprised of an αvβ6 integrin ligand.

In some embodiments, the described pharmaceutical compositions including a MMP7 RNAi agent are used for treating or managing clinical presentations in a subject that would benefit from the inhibition of expression of MMP7. In some embodiments, a therapeutically or prophylactically effective amount of one or more of pharmaceutical compositions is administered to a subject in need of such treatment. In some embodiments, administration of any of the disclosed MMP7 RNAi agents can be used to decrease the number, severity, and/or frequency of symptoms of a disease in a subject.

In some embodiments, the described MMP7 RNAi agents are optionally combined with one or more additional (i.e., second, third, etc.) therapeutics. A second therapeutic can be another MMP7 RNAi agent (e.g., a MMP7 RNAi agent that targets a different sequence within a MMP7 gene). In some embodiments, a second therapeutic can be an RNAi agent that targets the MMP7 gene. An additional therapeutic can also be a small molecule drug, antibody, antibody fragment, and/or aptamer. The MMP7 RNAi agents, with or without the one or more additional therapeutics, can be combined with one or more excipients to form pharmaceutical compositions.

The described pharmaceutical compositions that include a MMP7 RNAi agent can be used to treat at least one symptom in a subject having a disease or disorder that would benefit from reduction or inhibition in expression of MMP7 mRNA. In some embodiments, the subject is administered a therapeutically effective amount of one or more pharmaceutical compositions that include a MMP7 RNAi agent thereby treating the symptom. In other embodiments, the subject is administered aprophylactically effective amount of one or more MMP7 RNAi agents, thereby preventing or inhibiting the at least one symptom.

In some embodiments, one or more of the described MMP7 RNAi agents are administered to a mammal in a pharmaceutically acceptable carrier or diluent. In some embodiments, the mammal is a human.

The route of administration is the path by which a MMP7 RNAi agent is brought into contact with the body. In general, methods of administering drugs, oligonucleotides, and nucleic acids, for treatment of a mammal are well known in the art and can be applied to administration of the compositions described herein. The MMP7 RNAi agents disclosed herein can be administered via any suitable route in a preparation appropriately tailored to the particular route. Thus, in some embodiments, the herein described pharmaceutical compositions are administered via inhalation, intranasal administration, intratracheal administration, or oropharyngeal aspiration administration. In some embodiments, the pharmaceutical compositions can be administered by injection, for example, intravenously, intramuscularly, intracutaneously, subcutaneously, intraarticularly, intraocularly, or intraperitoneally, or topically.

The pharmaceutical compositions including a MMP7 RNAi agent described herein can be delivered to a cell, group of cells, tissue, or subject using oligonucleotide delivery technologies known in the art. In general, any suitable method recognized in the art for delivering a nucleic acid molecule (in vitro or in vivo) can be adapted for use with the compositions described herein. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In some embodiments, the compositions are administered via inhalation, intranasal administration, oropharyngeal aspiration administration, or intratracheal administration.

For example, in some embodiments, it is desired that the MMP7 RNAi agents described herein inhibit the expression of an MMP7 gene in the pulmonary epithelium, for which administration via inhalation (e.g., by an inhaler device, such as a metered-dose inhaler, or a nebulizer such as a jet or vibrating mesh nebulizer, or a soft mist inhaler) is particularly suitable and advantageous

In some embodiments, the pharmaceutical compositions described herein comprise one or more pharmaceutically acceptable excipients. The pharmaceutical compositions described herein are formulated for administration to a subject.

As used herein, a pharmaceutical composition includes a pharmacologically effective amount of at least one of the described therapeutic compounds and one or more pharmaceutically acceptable excipients. Pharmaceutically acceptable excipients (excipients) are substances other than the Active Pharmaceutical Ingredient (API, therapeutic product, e.g., MMP7 RNAi agent) that are intentionally included in the drug delivery system. Excipients do not exert or are not intended to exert a therapeutic effect at the intended dosage. Excipients can act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during stoMMP7 or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, detergents, dextran, dextrose, diluents, disintegrants, emulsifiers, extenders, fillers, flavors, glidants, humectants, lubricants, oils, polymers, preservatives, saline, salts, solvents, sugars, surfactants, suspending agents, sustained release matrices, sweeteners, thickening agents, tonicity agents, vehicles, water-repelling agents, and wetting agents.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor® EL™ (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

Formulations suitable for inhalation administration can be prepared by incorporating the active compound in the desired amount in an appropriate solvent, followed by sterile filtration. In general, formulations for inhalation administration are sterile solutions at physiological pH and have low viscosity (<5 cP). Salts may be added to the formulation to balance tonicity. In some cases, surfactants or co-solvents can be added to increase active compound solubility and improve aerosol characteristics. In some cases, excipients can be added to control viscosity in order to ensure size and distribution of nebulized droplets.

In some embodiments, pharmaceutical formulations that include the MMP7 RNAi agents disclosed herein suitable for inhalation administration can be prepared in water for injection (sterile water), or an aqueous sodium phosphate buffer (for example, the MMP7 RNAi agent formulated in 0.5 mM sodium phosphate monobasic, 0.5 mM sodium phosphate dibasic, in water).

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The MMP7 RNAi agents can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). It is also envisioned that cells, tissues, or isolated organs that express or comprise the herein defined RNAi agents may be used as “pharmaceutical compositions.” As used herein, “pharmacologically effective amount,” “therapeutically effective amount,” or simply “effective amount” refers to that amount of an RNAi agent to produce a pharmacological, therapeutic, or preventive result.

In some embodiments, the methods disclosed herein further comprise the step of administering a second therapeutic or treatment in addition to administering an RNAi agent disclosed herein. In some embodiments, the second therapeutic is another MMP7 RNAi agent (e.g., a MMP7 RNAi agent that targets a different sequence within the MMP7 target). In other embodiments, the second therapeutic can be a small molecule drug, an antibody, an antibody fragment, and/or an aptamer.

In some embodiments, described herein are compositions that include a combination or cocktail of at least two MMP7 RNAi agents having different sequences. In some embodiments, the two or more MMP7 RNAi agents are each separately and independently linked to targeting groups. In some embodiments, the two or more MMP7 RNAi agents are each linked to targeting groups that include or consist of integrin targeting ligands. In some embodiments, the two or more MMP7 RNAi agents are each linked to targeting groups that include or consist of αvβ6 integrin targeting ligands.

Described herein are compositions for delivery of MMP7 RNAi agents to pulmonary epithelial cells. Furthermore, compositions for delivery of MMP7 RNAi agents to cells, including renal epithelial cells and/or epithelial cells in the GI or reproductive tract and/or and ocular surface epithelial cells in the eye, in vivo, are generally described herein.

Generally, an effective amount of a MMP7 RNAi agent disclosed herein will be in the range of from about 0.0001 to about 20 mg/kg of body weight/deposited dose, e.g., from about 0.001 to about 5 mg/kg of body weight/deposited dose. In some embodiments, an effective amount of a MMP7 RNAi agent will be in the range of from about 0.01 mg/kg to about 3.0 mg/kg of body weight per deposited dose. In some embodiments, an effective amount of a MMP7 RNAi agent will be in the range of from about 0.03 mg/kg to about 2.0 mg/kg of body weight per deposited dose. In some embodiments, an effective amount of a MMP7 RNAi agent will be in the range of from about 0.01 to about 1.0 mg/kg of deposited dose per body weight. In some embodiments, an effective amount of a MMP7 RNAi agent will be in the range of from about 0.50 to about 1.0 mg/kg of deposited dose per body weight. Calculating the pulmonary deposited dose (PDD) is done in accordance with methods known in the art. (See Wolff R. K., Dorato M. A., Toxicologic Testing of Inhaled Pharmaceutical Aerosols, Crit Rev Toxicol., 1993; 23(4):343-369; Tepper et al., International J. Toxicology, 2016, vol. 35(4):376-392). The amount administered will also likely depend on such variables as the overall health status of the patient, the relative biological efficacy of the compound delivered, the formulation of the drug, the presence and types of excipients in the formulation, and the route of administration. Also, it is to be understood that the initial dosage administered can be increased beyond the above upper level to rapidly achieve the desired blood-level or tissue level, or the initial dosage can be smaller than the optimum. In some embodiments, a dose is administered daily. In some embodiments, a dose is administered weekly. In further embodiments, a dose is administered bi-weekly, tri-weekly, once monthly, or once quarterly (i.e., once every three months).

For treatment of disease or for formation of a medicament or composition for treatment of a disease, the pharmaceutical compositions described herein including a MMP7 RNAi agent can be combined with an excipient or with a second therapeutic agent or treatment including, but not limited to: a second or other RNAi agent, a small molecule drug, an antibody, an antibody fragment, peptide, and/or an aptamer.

The described MMP7 RNAi agents, when added to pharmaceutically acceptable excipients or adjuvants, can be packaged into kits, containers, packs, or dispensers. The pharmaceutical compositions described herein can be packaged in dry powder or aerosol inhalers, other metered-dose inhalers, nebulizers, pre-filled syringes, or vials.

### Methods of Treatment and Inhibition of MMP7 Expression

The MMP7 RNAi agents disclosed herein can be used to treat a subject (e.g., a human or other mammal) having a disease or disorder that would benefit from administration of the RNAi agent. In some embodiments, the RNAi agents disclosed herein can be used to treat a subject (e.g., a human) that would benefit from a reduction and/or inhibition in expression of MMP7 mRNA and/or a reduction in MMP7 enzyme levels.

In some embodiments, the RNAi agents disclosed herein can be used to treat a subject (e.g., a human) having a disease or disorder for which the subject would benefit from reduction in MMP7 enzyme levels, including but not limited to, idiopathic pulmonary fibrosis (IPF), asthma, various other types of fibrosis, chronic inflammation, interstitial lung diseases (ILD), infectious disease (for example, SARS-COV-2), acute lung injury (for example, acute respiratory distress syndrome (ARDS)), pulmonary hypertension, various cancers, nonalcoholic fatty liver disease (NAFLD), nonalcoholic steatohepatitis (NASH), fatty liver disease, biliary atresia, and chronic kidney disease (CKD). In some embodiments the disease is IPF. In some embodiments the subject has been previously diagnosed with having IPF, asthma, ILD, ARDS, or another type of fibrosis. Treatment of a subject can include therapeutic and/or prophylactic treatment. The subject is administered a therapeutically effective amount of any one or more MMP7 RNAi agents described herein. The subject can be a human, patient, or human patient. The subject may be an adult, adolescent, child, or infant. Administration of a pharmaceutical composition described herein can be to a human being or animal.

Increased membrane MMP7 enzyme levels are known to contribute to aberrant epithelial cell, fibroblast, and immune cell function and have been linked to fibrosis particularly in pulmonary tissues and cells. In some embodiments, the described MMP7 RNAi agents are used to treat at least one symptom mediated at least in part by a reduction in MMP7 enzyme levels, in a subject. The subject is administered a therapeutically effective amount of any one or more of the described MMP7 RNAi agents. In some embodiments, the subject is administered a prophylactically effective amount of any one or more of the described RNAi agents, thereby treating the subject by preventing or inhibiting the at least one symptom.

In certain embodiments, the present disclosure provides methods for treatment of diseases, disorders, conditions, or pathological states mediated at least in part by MMP7 gene expression, in a patient in need thereof, wherein the methods include administering to the patient any of the MMP7 RNAi agents described herein.

In some embodiments, the MMP7 RNAi agents are used to treat or manage a clinical presentation or pathological state in a subject, wherein the clinical presentation or pathological state is mediated at least in part by a reduction in MMP7 expression. The subject is administered a therapeutically effective amount of one or more of the MMP7 RNAi agents or MMP7 RNAi agent-containing compositions described herein. In some embodiments, the method comprises administering a composition comprising a MMP7 RNAi agent described herein to a subject to be treated.

In a further aspect, the disclosure features methods of treatment (including prophylactic or preventative treatment) of diseases or symptoms that may be addressed by a reduction in MMP7 enzyme levels, the methods comprising administering to a subject in need thereof a MMP7 RNAi agent that includes an antisense strand comprising the sequence of any of the sequences in Table 2, Table 3, or Table 10. Also described herein are compositions for use in such methods.

The described MMP7 RNAi agents and/or compositions that include MMP7 RNAi agents can be used in methods for therapeutic treatment of disease or conditions caused by enhanced or elevated MMP7 enzyme levels. Such methods include administration of a MMP7 RNAi agent as described herein to a subject, e.g., a human or animal subject.

In another aspect, the disclosure provides methods for the treatment (including prophylactic treatment) of a pathological state (such as a condition or disease) mediated at least in part by MMP7 expression, wherein the methods include administering to a subject a therapeutically effective amount of an RNAi agent that includes an antisense strand comprising the sequence of any of the sequences in Table 2, Table 3, or Table 10.

In some embodiments, methods for inhibiting expression of an MMP7 gene are disclosed herein, wherein the methods include administering to a cell an RNAi agent that includes an antisense strand comprising the sequence of any of the sequences in Table 2, Table 3, or Table 10.

In some embodiments, methods for the treatment (including prophylactic treatment) of a pathological state mediated at least in part by MMP7 expression are disclosed herein, wherein the methods include administering to a subject a therapeutically effective amount of an RNAi agent that includes a sense strand comprising the sequence of any of the sequences in Table 2, Table 4, Table 5, Table 6, or Table 10.

In some embodiments, methods for inhibiting expression of an MMP7 gene are disclosed herein, wherein the methods comprise administering to a cell an RNAi agent that includes a sense strand comprising the sequence of any of the sequences in Table 2, Table 4, Table 5, Table 6, or Table 10.

In some embodiments, methods for the treatment (including prophylactic treatment) of a pathological state mediated at least in part by MMP7 expression are disclosed herein, wherein the methods include administering to a subject a therapeutically effective amount of an RNAi agent that includes a sense strand comprising the sequence of any of the sequences in Table 4, Table 5. Table 6, or Table 10, and an antisense strand comprising the sequence of any of the sequences in Table 3 or Table 10.

In some embodiments, methods for inhibiting expression of a MMP7 gene are disclosed herein, wherein the methods include administering to a cell an RNAi agent that includes a sense strand comprising the sequence of any of the sequences in Table 4, Table 5, Table 6, or Table 10, and an antisense strand comprising the sequence of any of the sequences in Table 3 or Table 10.

In some embodiments, methods of inhibiting expression of a MMP7 gene are disclosed herein, wherein the methods include administering to a subject a MMP7 RNAi agent that includes a sense strand consisting of the nucleobase sequence of any of the sequences in Table 4, Table 5, Table 6, or Table 10, and the antisense strand consisting of the nucleobase sequence of any of the sequences in Table 3 or Table 10. In other embodiments, disclosed herein are methods of inhibiting expression of a MMP7 gene, wherein the methods include administering to a subject a MMP7 RNAi agent that includes a sense strand consisting of the modified sequence ofany of the modified sequences in Table 4, Table 5, Table 6, or Table 10, and the antisense strand consisting of the modified sequence of any of the modified sequences in Table 3 or Table 10.

In some embodiments, methods for inhibiting expression of an MMP7 gene in a cell are disclosed herein, wherein the methods include administering one or more MMP7 RNAi agents comprising a duplex structure of one of the duplexes set forth in Tables 7A, 7B, 8, 9, and 10.

In some embodiments, the MMP7 gene expression level and/or MMP7 mRNA level in certain pulmonary epithelial cells of subject to whom a described MMP7 RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject's respective level prior to being administered the MMP7 RNAi agent or to a different subject not receiving the MMP7 RNAi agent. In some embodiments, the MMP7 enzyme levels in certain epithelial cells or circulating MMP7 enzyme levels of a subject to whom a described MMP7 RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the MMP7 RNAi agent or to a different subject not receiving the MMP7 RNAi agent. The gene expression level, enzyme or protein level, and/or mRNA level in the subject may be reduced in a cell, group of cells, serum, and/or tissue of the subject. In some embodiments, the MMP7 enzyme levels in certain subject to whom a described MMP7 RNAi agent has been administered is reduced by at least about 30%, 35%, 40%, 45%, 50%, 55%, 60%, 65%, 70%, 75%, 80%, 85%, 90%, 95%, or 98% relative to the subject prior to being administered the MMP7 RNAi agent or to a subject not receiving the MMP7 RNAi agent.

A reduction in gene expression, mRNA, and enzyme or protein levels can be assessed by any methods known in the art. Reduction or decrease in MMP7 enzyme levels or MMP7 mRNA levels are sometimes collectively referred to herein as a decrease in, reduction of, or inhibition of MMP7 gene expression. The Examples set forth herein illustrate known methods for assessing inhibition of MMP7.

### Cells, Tissues, Organs, and Non-Human Organisms

Cells, tissues, organs, and non-human organisms that include at least one of the MMP7 RNAi agents described herein are contemplated. The cell, tissue, organ, or non-human organism is made by delivering the RNAi agent to the cell, tissue, organ, or non-human organism.

### Additional Illustrative Embodiments

Provided here are certain additional illustrative embodiments of the disclosed technology. These embodiments are illustrative only and do not limit the scope of the present disclosure or of the claims attached hereto.
Embodiment 1. An RNAi agent for inhibiting expression of a matrix metallopeptidase 7 gene, comprising:
   an antisense strand comprising at least 17 contiguous nucleotides differing by 0 or 1 nucleotides from any one of the sequences provided in Table 2 or Table 3; and
   a sense strand comprising a nucleotide sequence that is at least partially complementary to the antisense strand.
Embodiment 2. The RNAi agent of embodiment 1, wherein the antisense strand comprises nucleotides 2-18 of any one of the sequences provided in Table 2 or Table 3.
Embodiment 3. The RNAi agent of embodiment 1 or embodiment 2, wherein the sense strand comprises a nucleotide sequence of at least 17 contiguous nucleotides differing by 0 or 1 nucleotides from any one of the sequences provided in Table 2 or Table 4, and wherein the sense strand has a region of at least 85% complementarity over the 17 contiguous nucleotides to the antisense strand.
Embodiment 4. The RNAi agent of any one of embodiments 1-3, wherein at least one nucleotide of the MMP7 RNAi agent is a modified nucleotide or includes a modified internucleoside linkage.
Embodiment 5. The RNAi agent of any one of embodiments 1-4, wherein all or substantially all of the nucleotides are modified nucleotides.
Embodiment 6. The RNAi agent of any one of embodiments 4-5, wherein the modified nucleotide is selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.
Embodiment 7. The RNAi agent of embodiment 5, wherein all or substantially all of the nucleotides are modified with 2′-O-methyl nucleotides, 2′-fluoro nucleotides, or combinations thereof.
Embodiment 8. The RNAi agent of any one of embodiments 1-7, wherein the antisense strand comprises the nucleotide sequence of any one of the modified sequences provided in Table 3.
Embodiment 9. The RNAi agent of any one of embodiments 1-8, wherein the sense strand comprises the nucleotide sequence of any one of the modified sequences provided in Table 4.
Embodiment 10. The RNAi agent of embodiment 1, wherein the antisense strand comprises the nucleotide sequence of any one of the modified sequences provided in Table 3 and the sense strand comprises the nucleotide sequence of any one of the modified sequences provided in Table 4.
Embodiment 11. The RNAi agent of any one of embodiments 1-10, wherein the sense strand is between 18 and 30 nucleotides in length, and the antisense strand is between 18 and 30 nucleotides in length.
Embodiment 12. The RNAi agent of embodiment 11, wherein the sense strand and the antisense strand are each between 18 and 27 nucleotides in length.
Embodiment 13. The RNAi agent of embodiment 12, wherein the sense strand and the antisense strand are each between 18 and 24 nucleotides in length.
Embodiment 14. The RNAi agent of embodiment 13, wherein the sense strand and the antisense strand are each 21 nucleotides in length.
Embodiment 15. The RNAi agent of embodiment 14, wherein the RNAi agent has two blunt ends.
Embodiment 16. The RNAi agent of any one of embodiments 1-15, wherein the sense strand comprises one or two terminal caps.
Embodiment 17. The RNAi agent of any one of embodiments 1-16, wherein the sense strand comprises one or two inverted abasic residues.
Embodiment 18. The RNAi agent of embodiment 1, wherein the RNAi agent is comprised of a sense strand and an antisense strand that form a duplex having the structure of any one of the duplexes in Table 7A, Table 7B, Table 8, Table 9, or Table 10.
Embodiment 19. The RNAi agent of embodiment 18, wherein all or substantially all of the nucleotides are modified nucleotides.
Embodiment 20. The RNAi agent of embodiment 1, comprising an antisense strand that consists of, consists essentially of, or comprises a nucleotide sequence that differs by 0 or 1 nucleotides from one of the following nucleotide sequences (5′→3′):

(SEQ ID NO: 175)
AGACAUUCAAAAACCAACU;
(SEQ ID NO: 123)
UUGACACUAAUCGAUCCAC;
(SEQ ID NO: 176)
UGACAUUCAAAAACCAACU;
(SEQ ID NO: 674)
AGACAUUCAAAAACCAACUGC;
(SEQ ID NO: 661)
UUGACACUAAUCGAUCCACUG;
or
(SEQ ID NO: 679)
UGACAUUCAAAAACCAACUGC.

Embodiment 21. The RNAi agent of embodiment 20, wherein the sense strand consists of, consists essentially of, or comprises a nucleotide sequence that differs by 0 or 1 nucleotides from one of the following nucleotide sequences (5′→3′):

(SEQ ID NO: 325)
AGUUGGUUUUUGAAUGUCU;
(SEQ ID NO: 273)
GUGGAUCGAUUAGUGUCAA;
(SEQ ID NO: 326)
AGUUGGUUUUUGAAUGUCA;
(SEQ ID NO: 720)
GCAGUUGGUUUUUGAAUGUCU;
(SEQ ID NO: 724)
CAGUGGAUCGAUUAGUGUCAA;
or
(SEQ ID NO: 726)
GCAGUUGGUUUUUGAAUGUCA.

Embodiment 22. The RNAi agent of embodiment 20 or 21, wherein all or substantially all of the nucleotides are modified nucleotides.
Embodiment 23. The RNAi agent of embodiment 1, comprising an antisense strand that comprises, consists of, or consists essentially of a modified nucleotide sequence that differs by 0 or 1 nucleotides from one of the following nucleotide sequences (5′→3′):

(SEQ ID NO: 396)
asGfsascauucAfaaAfaCfcAfacugsc;
(SEQ ID NO: 393)
usUfsgsacacUfaAfuCfgAfuCfcacusg;
or
(SEQ ID NO: 406)
cPrpuGfacauucAfaaAfaCfcAfacugsc;

wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; cPrpu represents a 5′-cyclopropyl phosphonate-2′-O-methyl uridine; s represents a phosphorothioate linkage; and wherein all or substantially all of the nucleotides on the sense strand are modified nucleotides.
Embodiment 24. The RNAi agent of embodiment 1, wherein the sense strand comprises, consists of, or consists essentially of a modified nucleotide sequence that differs by 0 or 1 nucleotides from one of the following nucleotide sequences (5′→3′):

(SEQ ID NO: 522)
gscaguuggUfuUfuUfgaaugucu;
(SEQ ID NO: 524)
csaguggauCfgAfuUfagugucaa;
(SEQ ID NO: 526)
gscaguuggUfuUfuUfgaauguca;

wherein a, c, g, i, and u represent 2′-O-methyl adenosine, 2′-O-methyl cytidine, 2′-O-methyl guanosine, 2′-O-methyl inosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; and s represents a phosphorothioate linkage; and wherein all or substantially all of the nucleotides on the antisense strand are modified nucleotides.
Embodiment 25. The RNAi agent of any one of embodiments 20-24, wherein the sense strand further includes inverted abasic residues at the 3′ terminal end of the nucleotide sequence, at the 5′ end of the nucleotide sequence, or at both.
Embodiment 26. The RNAi agent of any one of embodiments 1-25, wherein the RNAi agent is linked to a targeting ligand.
Embodiment 27. The RNAi agent of embodiment 26, wherein the targeting ligand has affinity for a cell receptor expressed on an epithelial cell.
Embodiment 28. The RNAi agent of embodiment 27, wherein the targeting ligand comprises an integrin targeting ligand.
Embodiment 29. The RNAi agent of embodiment 28, wherein the integrin targeting ligand is an αvβ6 integrin targeting ligand.
Embodiment 30. The RNAi agent of embodiment 29, wherein the targeting ligand comprises the structure:

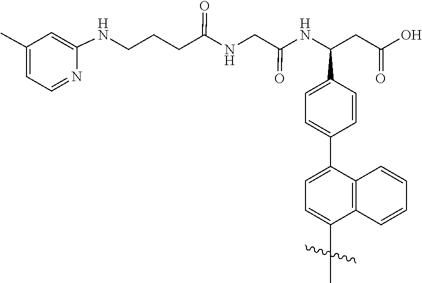 [SMILES: *c1ccc(-c2ccc([C@H](CC(=O)O)NC(=O)CNC(=O)CCCNc3cc(C)ccn3)cc2)c2ccccc12]

or a pharmaceutically acceptable salt thereof,

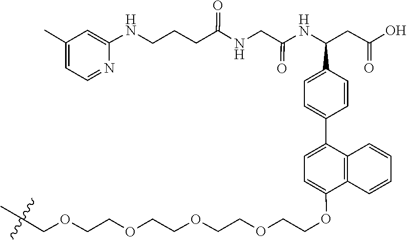 [SMILES: *COCCOCCOCCOCCOc1ccc(-c2ccc([C@H](CC(=O)O)NC(=O)CNC(=O)CCCNc3cc(C)ccn3)cc2)c2ccccc12]

or a pharmaceutically acceptable salt thereof,
wherein [symbol] indicates the point of connection to the RNAi agent.
Embodiment 31. The RNAi agent of any one of embodiments 26-29, wherein the targeting ligand has a structure selected from the group consisting of:

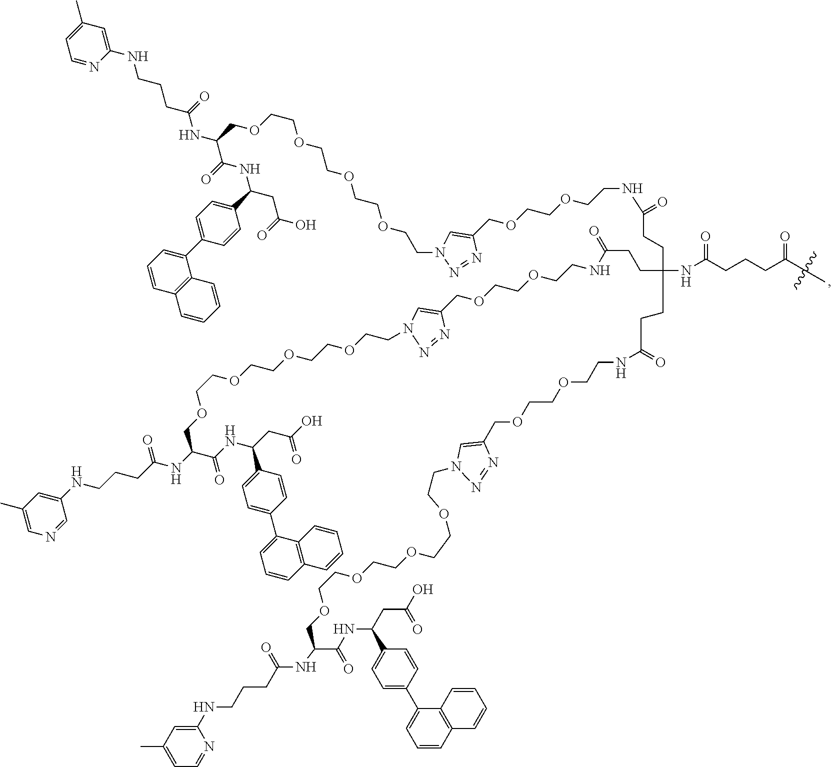 [SMILES: *C(=O)CCCC(=O)NC(CCC(=O)NCCOCCOCc1cn(CCOCCOCCOCCOC[C@H](NC(=O)CCCNc2cncc(C)c2)C(=O)N[C@@H](CC(=O)O)c2ccc(-c3cccc4ccccc34)cc2)nn1)(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1]

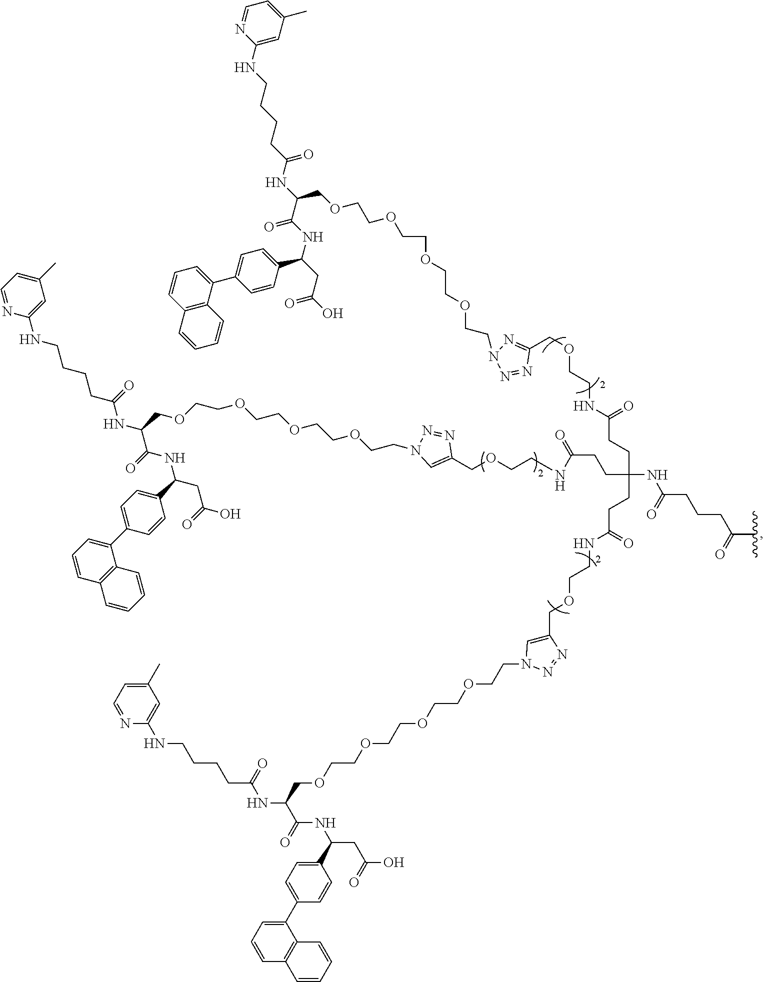

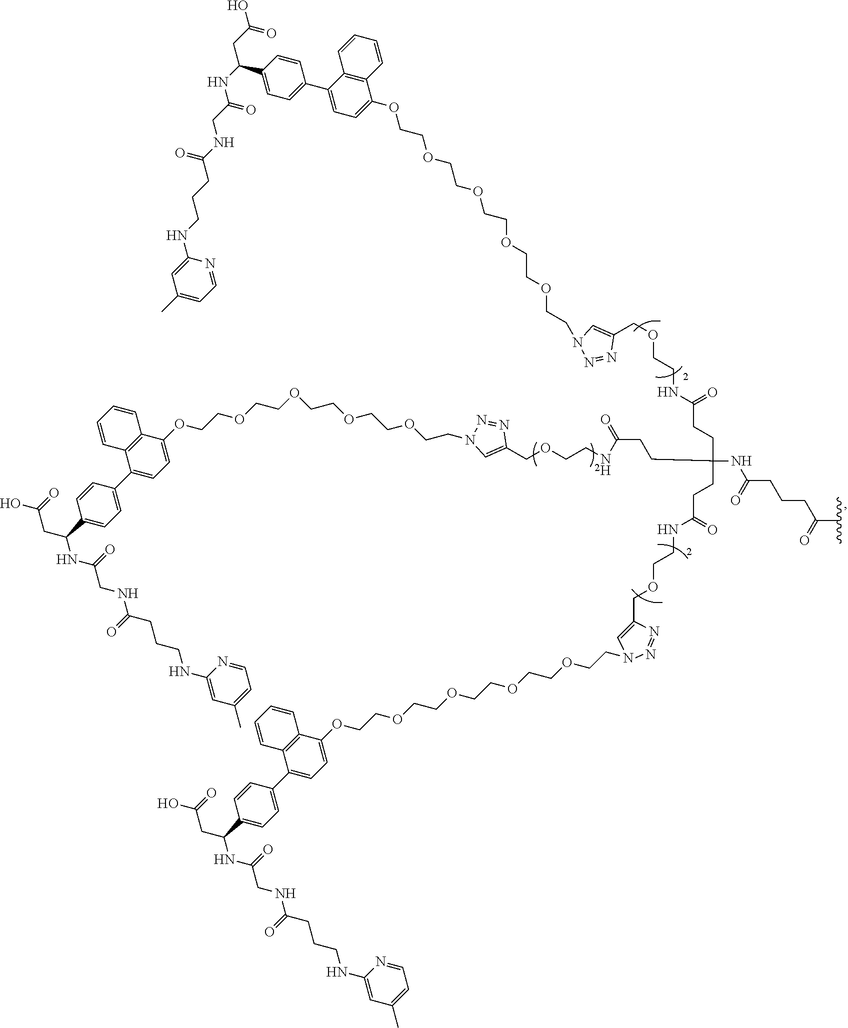

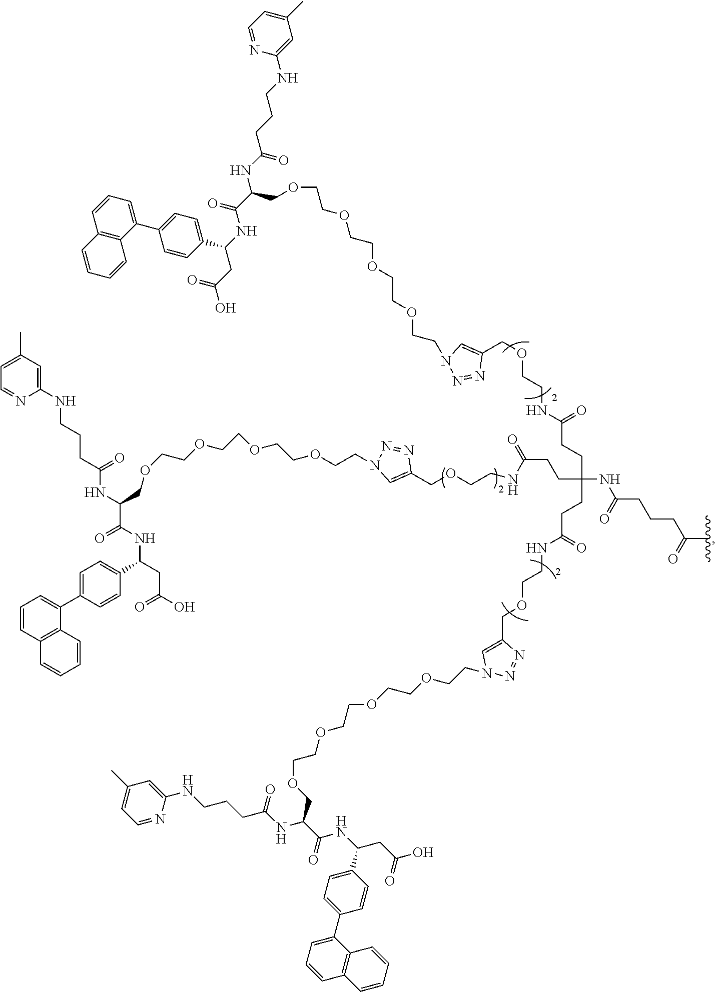

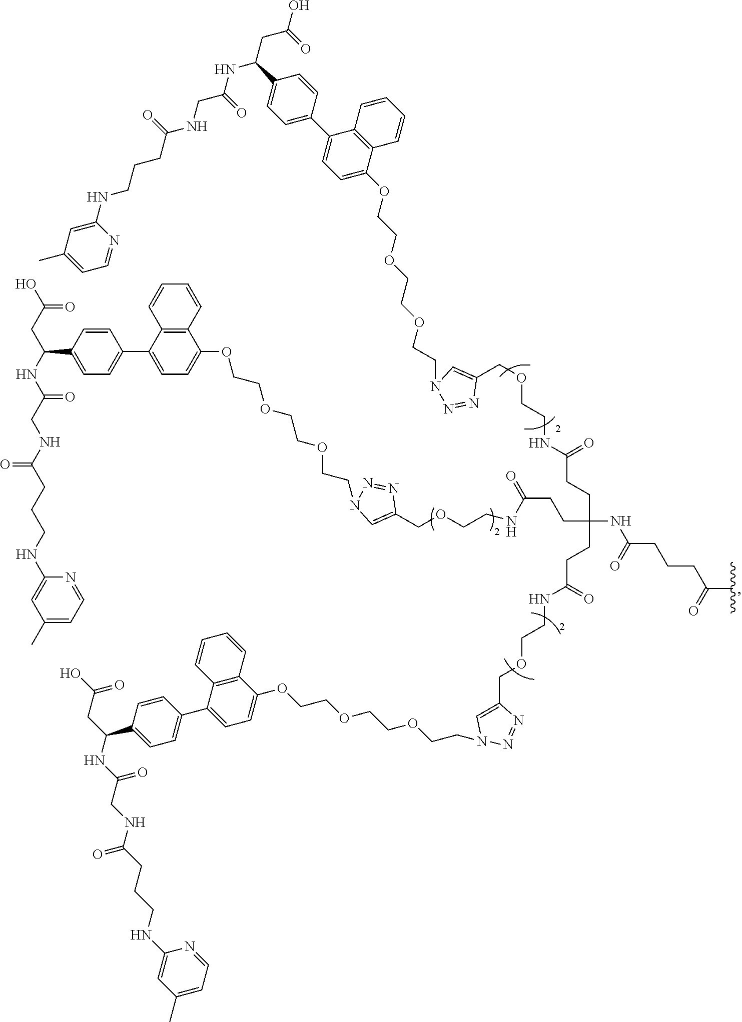

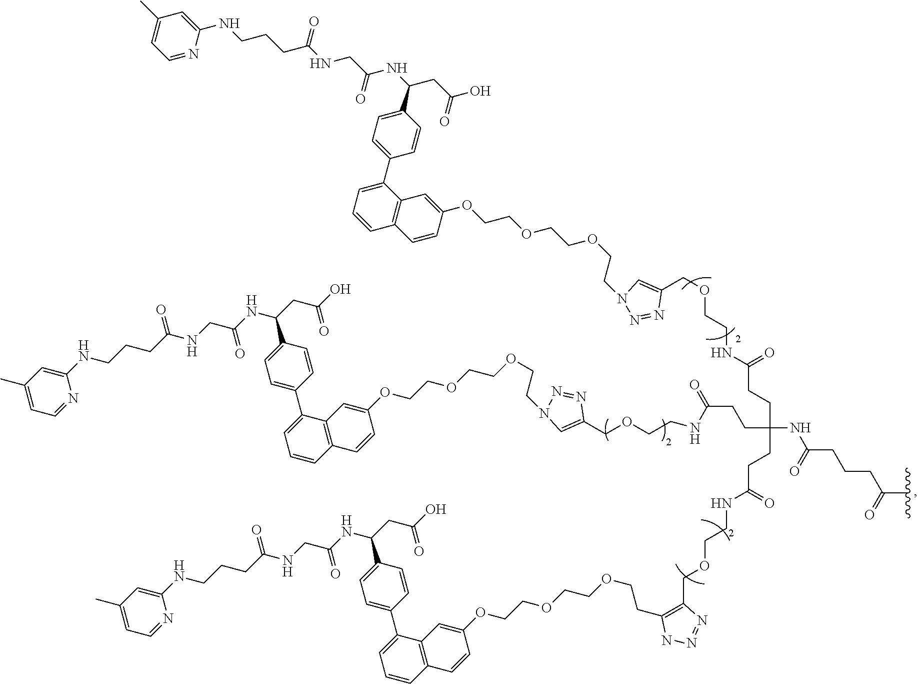

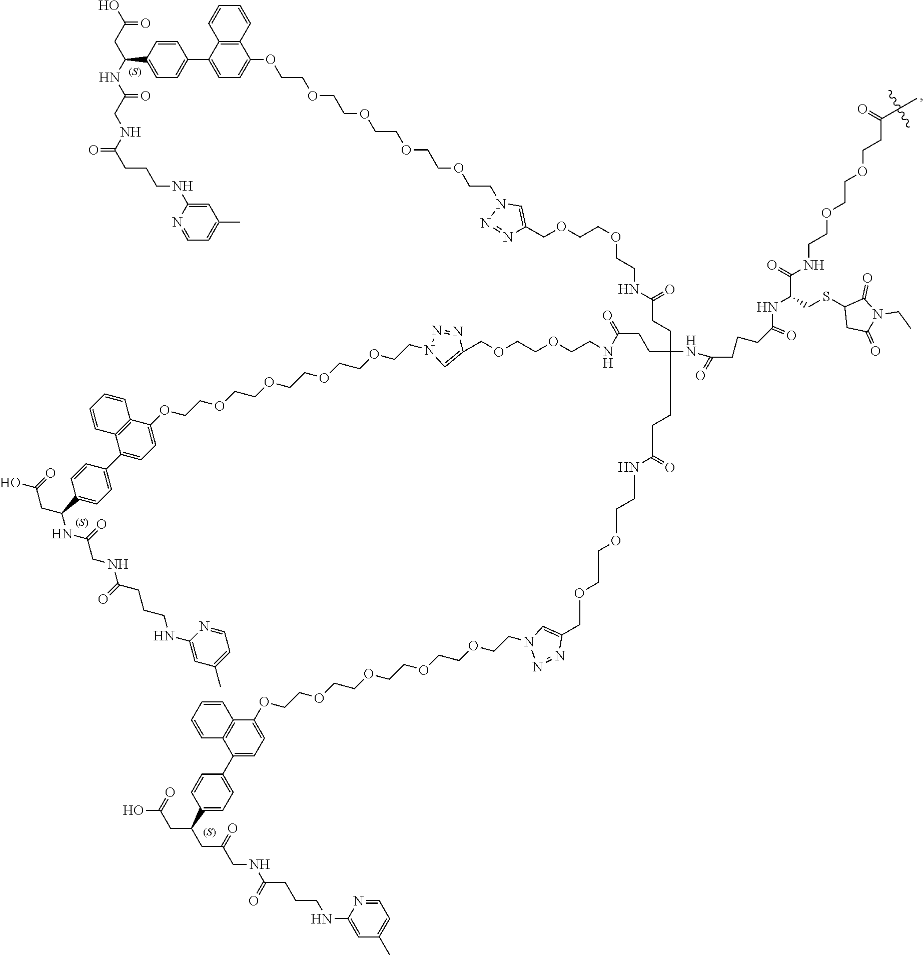

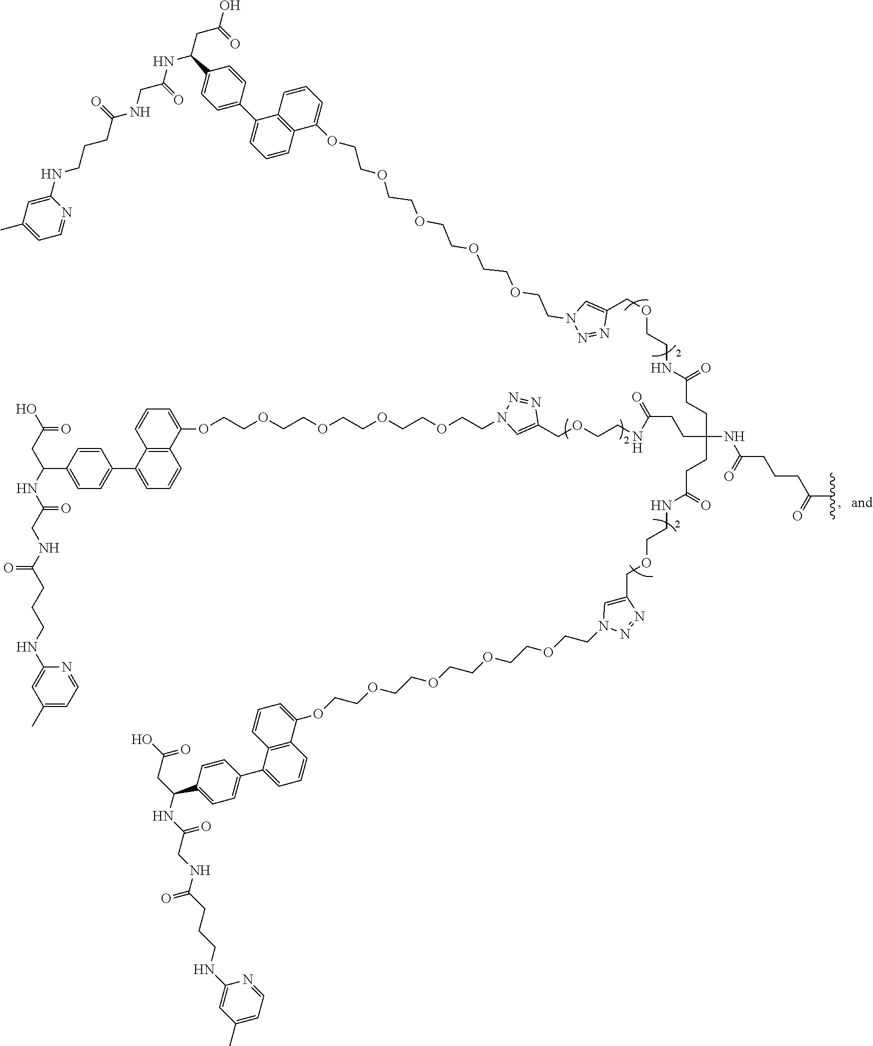

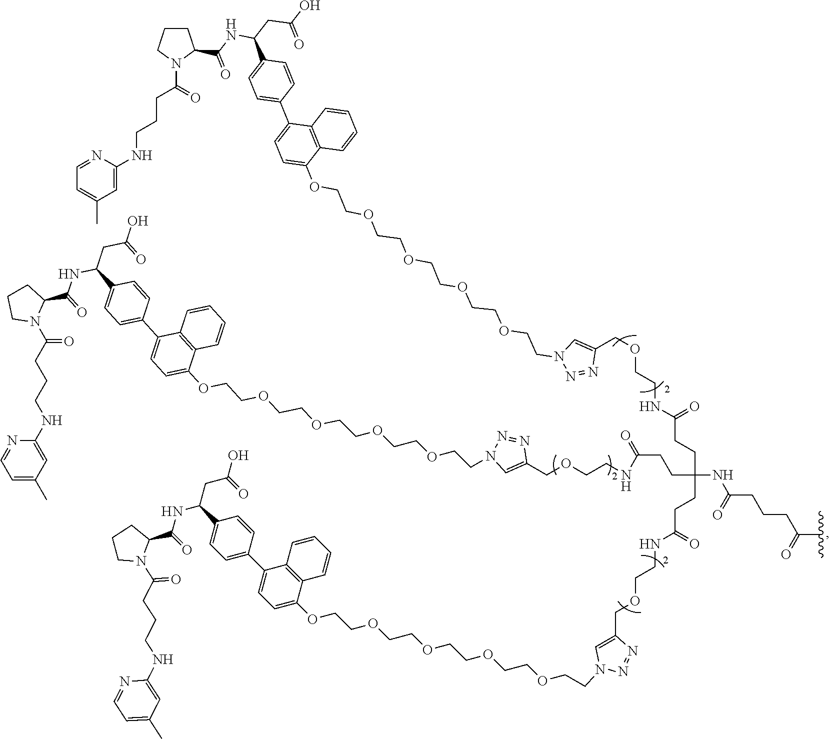

wherein [symbol] indicates the point of connection to the RNAi agent.
Embodiment 32. The RNAi agent of embodiment 31, wherein RNAi agent is conjugated to a targeting ligand having the following structure:

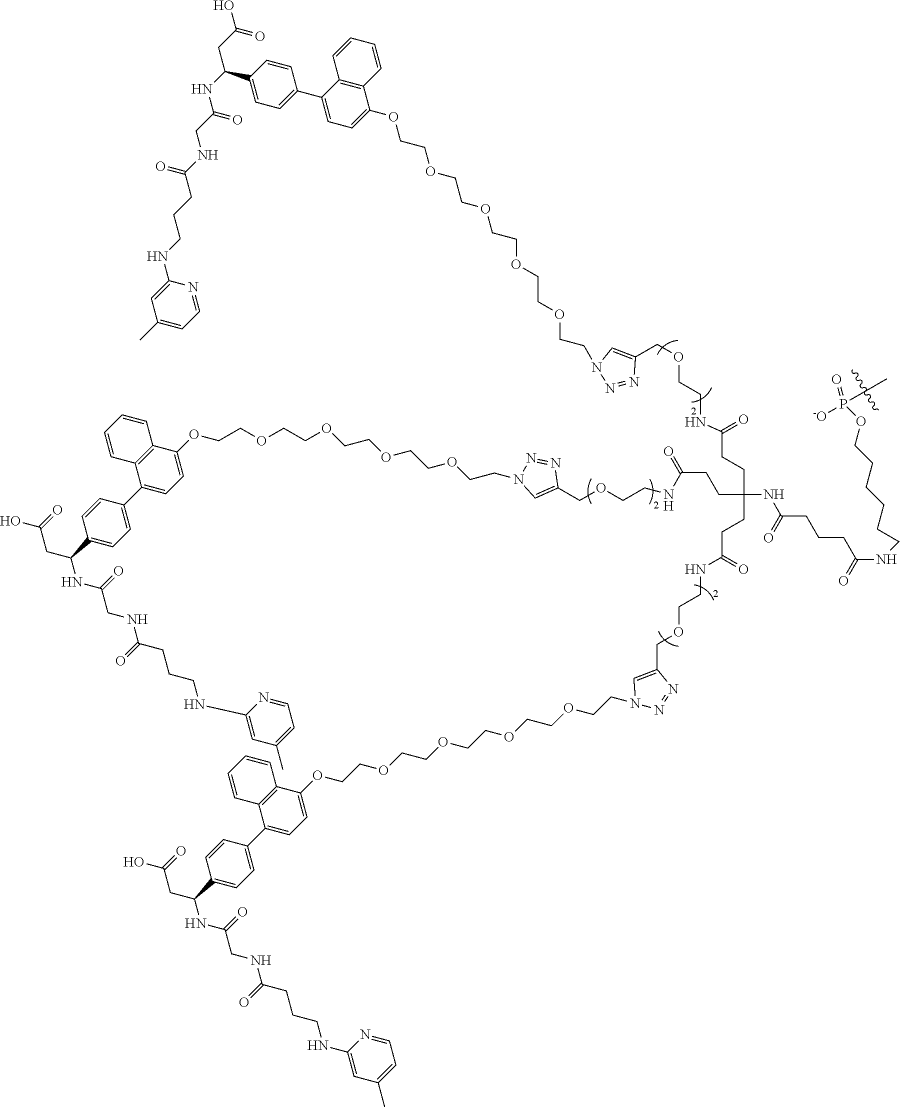

Embodiment 33. The RNAi agent of any one of embodiments 26-29, wherein the targeting ligand has the following structure:

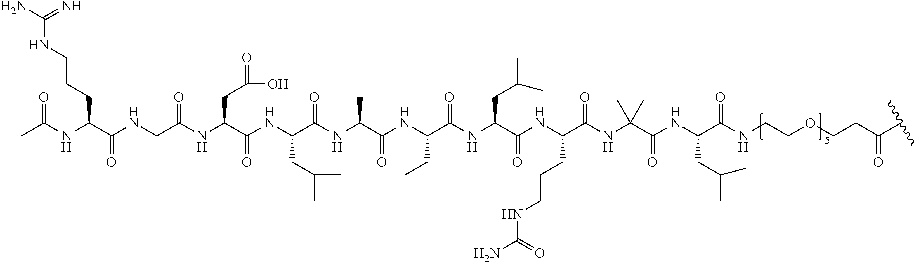

Embodiment 34. The RNAi agent of any one of embodiments 26-33, wherein the targeting ligand is conjugated to the sense strand.
Embodiment 35. The RNAi agent of embodiment 34, wherein the targeting ligand is conjugated to the 5′ terminal end of the sense strand.
Embodiment 36. A composition comprising the RNAi agent of any one of embodiments 1-35, wherein the composition further comprises a pharmaceutically acceptable excipient.
Embodiment 37. The composition of embodiment 36, further comprising a second RNAi agent capable of inhibiting the expression of matrix metallopeptidase 7 gene expression.
Embodiment 38. The composition of any one of embodiments 36-37, further comprising one or more additional therapeutics.
Embodiment 39. The composition of any one of embodiments 36-38, wherein the composition is formulated for administration by inhalation.
Embodiment 40. The composition of embodiment 39, wherein the composition is delivered by a metered-dose inhaler, jet nebulizer, vibrating mesh nebulizer, or soft mist inhaler.
Embodiment 41. The composition of any of embodiments 36-40, wherein the RNAi agent is a sodium salt.
Embodiment 42. The composition of any of embodiments 36-41, wherein the pharmaceutically acceptable excipient is water for injection.
Embodiment 43. The composition of any of embodiments 36-41, wherein the pharmaceutically acceptable excipient is a buffered saline solution.
Embodiment 44. A method for inhibiting expression of a MMP7 gene in a cell, the method comprising introducing into a cell an effective amount of an RNAi agent of any one of embodiments 1-33 or the composition of any one of embodiments 36-43.
Embodiment 45. The method of embodiment 44, wherein the cell is within a subject.
Embodiment 46. The method of embodiment 45, wherein the subject is a human subject.
Embodiment 47. The method of any one of embodiments 44-46, wherein following the administration of the RNAi agent the matrix metallopeptidase 7 gene expression is inhibited by at least about 30%.
Embodiment 48. A method of treating one or more symptoms or diseases associated with enhanced or elevated membrane MMP7 activity levels, the method comprising administering to a human subject in need thereof a therapeutically effective amount of the composition of any one of embodiments 36-43.
Embodiment 49. The method of embodiment 48, wherein the disease is a respiratory or pulmonary disease.
Embodiment 50. The method of embodiment 48, wherein the disease is selected from the group consisting of: idiopathic pulmonary fibrosis (IPF), another type of pulmonary fibrosis, asthma, chronic inflammation, interstitial lung diseases (ILD), SARS-COV-2 or another type of infectious disease in the airway, acute respiratory distress syndrome (ARDS) or another type of acute lung injury, pulmonary hypertension, lung cancer, nonalcoholic fatty liver disease (NAFLD), nonalcoholic steatohepatitis (NASH), fatty liver disease, biliary atresia, and chronic kidney disease (CKD).
Embodiment 51. The method of embodiment 50, wherein the disease is idiopathic pulmonary fibrosis (IPF).
Embodiment 54. The method of any one of embodiments 44-51, wherein the RNAi agent is administered at a deposited dose of about 0.01 mg/kg to about 5.0 mg/kg of body weight of the subject.
Embodiment 55. The method of any one of embodiments 44-54, wherein the RNAi agent is administered at a deposited dose of about 0.03 mg/kg to about 2.0 mg/kg of body weight of the subject.
Embodiment 56. The method of any one of embodiments 44-55, wherein the RNAi agent is administered in two or more doses.
Embodiment 57. Use of the RNAi agent of any one of embodiments 1-35, for the treatment of a disease, disorder, or symptom that is mediated at least in part by membrane MMP7 activity and/or MMP7 gene expression.
Embodiment 58. Use of the composition according to any one of embodiments 36-43, for the treatment of a disease, disorder, or symptom that is mediated at least in part by matrix metallopeptidase 7 activity and/or matrix metallopeptidase 7 gene expression.
Embodiment 59. Use of the composition according to any one of embodiments 36-43, for the manufacture of a medicament for treatment of a disease, disorder, or symptom that is mediated at least in part by matrix metallopeptidase 7 and/or matrix metallopeptidase 7 gene expression.
Embodiment 60. The use of any one of embodiments 57-59, wherein the disease is pulmonary inflammation.
Embodiment 61. A method of making an RNAi agent of any one of embodiments 1-35, comprising annealing a sense strand and an antisense strand to form a double-stranded ribonucleic acid molecule.
Embodiment 62. The method of embodiment 61, wherein the sense strand comprises a targeting ligand.
Embodiment 63. The method of embodiment 62, comprising conjugating a targeting ligand to the sense strand.
The above provided embodiments and items are now illustrated with the following, non-limiting examples.

### EXAMPLES

### Example 1. Synthesis of MMP7 RNAi Agents

MMP7 RNAi agent duplexes disclosed herein were synthesized in accordance with the following:

### A. Synthesis

The sense and antisense strands of the MMP7 RNAi agents were synthesized according to phosphoramidite technology on solid phase used in oligonucleotide synthesis. Depending on the scale, a MerMade96E® (Bioautomation), a MerMade12® (Bioautomation), or an OP Pilot 100 (GE Healthcare) was used. Syntheses were performed on a solid support made of controlled pore glass (CPG, 500 Å or 600 Å, obtained from Prime Synthesis, Aston, Pa., USA). All RNA and 2′-modified RNA phosphoramidites were purchased from Thermo Fisher Scientific (Milwaukee, Wis., USA). Specifically, the 2′-O-methyl phosphoramidites that were used included the following: (5′-O-dimethoxytrityl-N⁶-(benzoyl)-2′-O-methyl-adenosine-3′-O-(2-cyanoethyl-N,N-diisopropylamino) phosphoramidite, 5′-O-dimethoxy-trityl-N⁴-(acetyl)-2′-O-methyl-cytidine-3′-O-(2-cyanoethyl-N,N-diisopropyl-amino) phosphoramidite, (5′-O-dimethoxytrityl-N²-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino) phosphoramidite. The 2′-deoxy-2′-fluoro-phosphoramidites carried the same protecting groups as the 2′-O-methyl RNA amidites. 5′-dimethoxytrityl-2′-O-methyl-inosine-3′-O-(2-cyanoethyl-N,N-diisopropylamino) phosphoramidites were purchased from Glen Research (Virginia). The inverted abasic (3′-O-dimethoxytrityl-2′-deoxyribose-5′-O-(2-cyanoethyl-N,N-diisopropylamino) phosphoramidites were purchased from ChemGenes (Wilmington, Mass., USA). The following UNA phosphoramidites were used: 5′-(4,4′-Dimethoxytrityl)-N6-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-diiso-propyl)]-phosphoramidite. TFA aminolink phosphoramidites were also commercially purchased (ThermoFisher). Linker L6 was purchased as propargyl-PEG5-NHS from BroadPharm (catalog #BP-20907) and coupled to the NH₂-C₆ group from an aminolink phosphoramidite to form -L6-C6-, using standard coupling conditions. The linker Alk-cyHex was similarly commercially purchased from Lumiprobe (alkyne phosphoramidite, 5′-terminal) as a propargyl-containing compound phosphoramidite compound to form the linker -Alk-cyHex-. In each case, phosphorothioate linkages were introduced as specified using the conditions set forth herein. The cyclopropyl phosphonate phosphoramidites were synthesized in accordance with International Patent Application Publication No. WO 2017/214112 (see also Altenhofer et. al., Chem. Communications (Royal Soc. Chem.), 57(55):6808-6811 (July 2021)).

Tri-alkyne-containing phosphoramidites were dissolved in anhydrous dichloromethane or anhydrous acetonitrile (50 mM), while all other amidites were dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 minutes (RNA), 90 seconds (2′ O-Me), and 60 seconds (2′ F). In order to introduce phosphorothioate linkages, a 100 mM solution of 3-phenyl 1,2,4-dithiazoline-5-one (POS, obtained from PolyOrg, Inc., Leominster, Mass., USA) in anhydrous acetonitrile was employed.

Alternatively, tri-alkyne moieties were introduced post-synthetically (see section E, below). For this route, the sense strand was functionalized with a 5′ and/or 3′ terminal nucleotide containing a primary amine. TFA aminolink phosphoramidite was dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 minutes (RNA), 90 seconds (2′ O-Me), and 60 seconds (2′ F). In order to introduce phosphorothioate linkages, a 100 mM solution of 3-phenyl 1,2,4-dithiazoline-5-one (POS, obtained from PolyOrg, Inc., Leominster, Mass., USA) in anhydrous acetonitrile was employed.

### B. Cleavage and Deprotection of Support Bound Oligomer

After finalization of the solid phase synthesis, the dried solid support was treated with a 1:1 volume solution of 40 wt. % methylamine in water and 28% to 31% ammonium hydroxide solution (Aldrich) for 1.5 hours at 30° C. The solution was evaporated and the solid residue was reconstituted in water (see below).

### C. Purification

Crude oligomers were purified by anionic exchange HPLC using a TSKgel SuperQ-5PW 13 μm column and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 16/40 column packed with Sephadex G-25 fine with a running buffer of 100 mM ammonium bicarbonate, pH 6.7 and 20% Acetonitrile or filtered water. Alternatively, pooled fractions were desalted and exchanged into an appropriate buffer or solvent system via tangential flow filtration.

### D. Annealing

Complementary strands were mixed by combining equimolar RNA solutions (sense and antisense) in 1×PBS (Phosphate-Buffered Saline, 1×, Corning, Cellgro) to form the RNAi agents. Some RNAi agents were lyophilized and stored at −15 to −25° C. Duplex concentration was determined by measuring the solution absorbance on a UV-Vis spectrometer in 1×PBS. The solution absorbance at 260 nm was then multiplied by a conversion factor (0.050 mg/(mL-cm)) and the dilution factor to determine the duplex concentration.

### E. Conjugation of Tri-Alkyne Linker

In some embodiments a tri-alkyne linker is conjugated to the sense strand of the RNAi agent on resin as a phosphoramidite (see Example 1G for the synthesis of an example tri-alkyne linker phosphoramidite and Example 1A for the conjugation of the phosphoramidite.). In other embodiments, a tri-alkyne linker may be conjugated to the sense strand following cleavage from the resin, described as follows: either prior to or after annealing, in some embodiments, the 5′ or 3′ amine functionalized sense strand is conjugated to a tri-alkyne linker. An example tri-alkyne linker structure that can be used in forming the constructs disclosed herein is as follows:

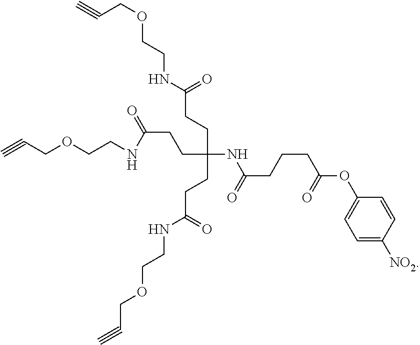 [SMILES: C#CCOCCNC(=O)CCC(CCC(=O)NCCOCC#C)(CCC(=O)NCCOCC#C)NC(=O)CCCC(=O)Oc1ccc([N+](=O)[O-])cc1]

To conjugate the tri-alkyne linker to the annealed duplex, amine-functionalized duplex was dissolved in 90% DMSO/10% H₂O, at ˜50-70 mg/mL. 40 equivalents triethylamine was added, followed by 3 equivalents tri-alkyne-PNP. Once complete, the conjugate was precipitated twice in a solvent system of 1× phosphate buffered saline/acetonitrile (1:14 ratio), and dried.

### F. Synthesis of Targeting Ligand SM6.1

### ((S)-3-(4-(4-((14-azido-3,6,9,12-tetraoxatetradecyl)oxy)naphthalen-1-yl)phenyl)-3-(2-(4-((4-methylpyridin-2-yl)amino)butanamido)acetamido)propanoic acid)

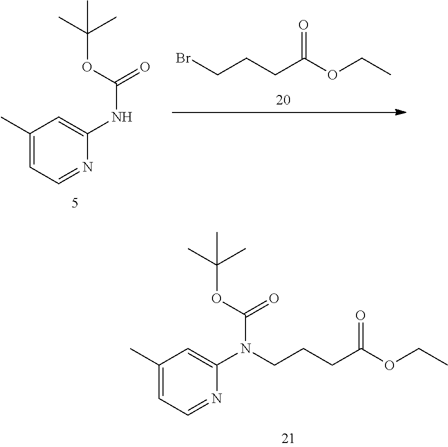

Compound 5 (tert-Butyl(4-methylpyridin-2-yl)carbamate) (0.501 g, 2.406 mmol, 1 equiv.) was dissolved in DMF (17 mL). To the mixture was added NaH (0.116 mg, 3.01 mmol, 1.25 eq, 60% dispersion in oil) The mixture stirred for 10 min before adding Compound 20 (Ethyl 4-Bromobutyrate (0.745 g, 3.82 mmol, 0.547 mL)) (Sigma 167118). After 3 hours the reaction was quenched with ethanol (18 mL) and concentrated. The concentrate was dissolved in DCM (50 mL) and washed with saturated aq. NaCl solution (1×50 mL), dried over Na₂SO₄, filtered and concentrated. The product was purified on silica column, gradient 0-5% Methanol in DCM.

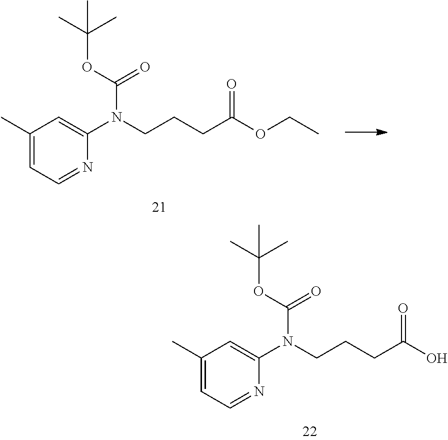

Compound 21 was dissolved (0.80 g, 2.378 mmol) in 100 mL of Acetone:0.1 M NaOH [1:1]. The reaction was monitored by TLC (5% ethyl acetate in hexane). The organics were concentrated away, and the residue was acidified to pH 3-4 with 0.3 M Citric Acid (40 mL). The product was extracted with DCM (3×75 mL). The organics were pooled, dried over Na₂SO₄, filtered and concentrated. The product was used without further purification.

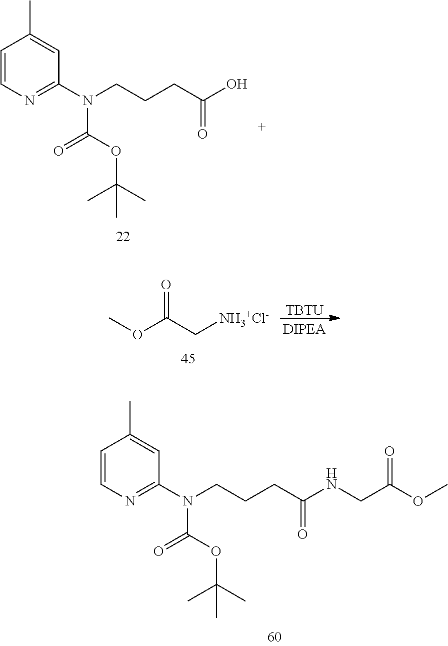

To a solution of Compound 22 (1.1 g, 3.95 mmol, 1 equiv.), Compound 45 (595 mg, 4.74 mmol, 1.2 equiv.), and TBTU (1.52 g, 4.74 mmol, 1.2 equiv.) in anhydrous DMF (10 mL) was added diisopropylethylamine (2.06 mL, 11.85 mmol, 3 equiv.) at 0° C. The reaction mixture was warmed to room temperature and stirred 3 hours. The reaction was quenched by saturated NaHCO₃ solution (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase. LC-MS: calculated [M+H]+ 366.20, found 367.

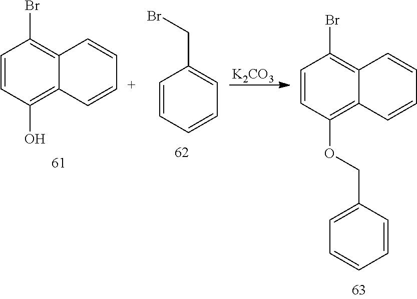

To a solution of compound 61 (2 g, 8.96 mmol, 1 equiv.), and compound 62 (2.13 mL, 17.93 mmol, 2 equiv.) in anhydrous DMF (10 mL) was added K₂CO₃ (2.48 g, 17.93 mmol, 2 equiv.) at 0° C. The reaction mixture was warmed to room temperature and stirred overnight. The reaction was quenched by water (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase.

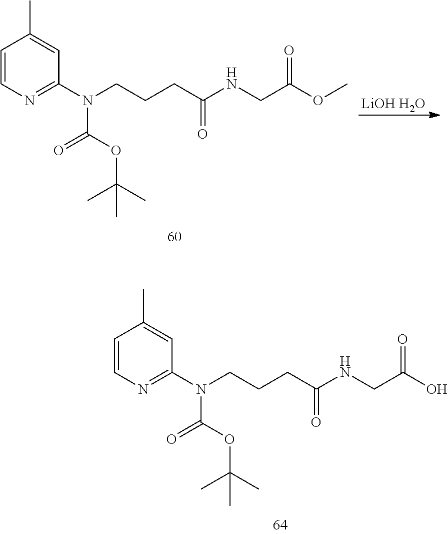

To a solution of compound 60 (1.77 g, 4.84 mmol, 1 equiv.) in THF (5 mL) and H₂O (5 mL) was added lithium hydroxide monohydrate (0.61 g, 14.53 mmol, 3 equiv.) portion-wise at 0° C. The reaction mixture was warmed to room temperature. After stirring at room temperature for 3 hours, the reaction mixture was acidified by HCl (6 N) to pH 3.0. The aqueous phase was extracted with ethyl acetate (3×20 mL) and the organic layer was combined, dried over Na₂SO₄, and concentrated. LC-MS: calculated [M+H]+ 352.18, found 352.

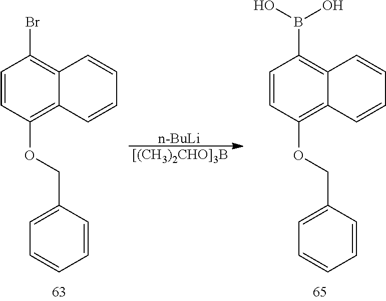

To a solution of compound 63 (1.88 g, 6.0 mmol, 1.0 equiv.) in anhydrous THF (20 mL) was added n-BuLi in hexane (3.6 mL, 9.0 mmol, 1.5 equiv.) drop-wise at −78° C. The reaction was kept at −78° C. for another 1 hour. Triisopropylborate (2.08 mL, 9.0 mmol, 1.5 equiv.) was then added into the mixture at −78° C. The reaction was then warmed up to room temperature and stirred for another 1 hour. The reaction was quenched by saturated NH₄Cl solution (20 mL) and the pH was adjusted to 3. The aqueous phase was extracted with EtOAc (3×20 mL) and the organic phase was combined, dried over Na₂SO₄, and concentrated.

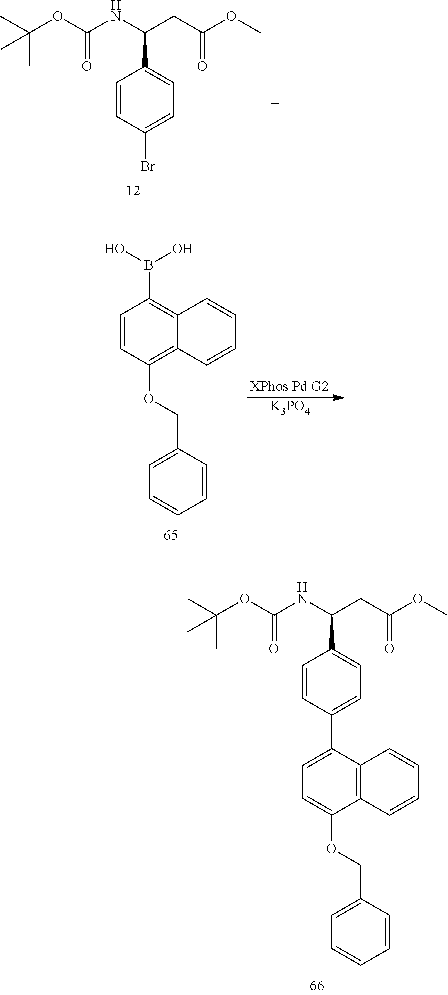

Compound 12 (300 mg, 0.837 mmol, 1.0 equiv.), Compound 65 (349 mg, 1.256 mmol, 1.5 equiv.), XPhos Pd G2 (13 mg, 0.0167 mmol, 0.02 equiv.), and K₃PO₄ (355 mg, 1.675 mmol, 2.0 equiv.) were mixed in a round-bottom flask. The flask was sealed with a screw-cap septum, and then evacuated and backfilled with nitrogen (this process was repeated a total of 3 times). Then, THF (8 mL) and water (2 mL) were added via syringe. The mixture was bubbled with nitrogen for 20 min and the reaction was kept at room temperature for overnight. The reaction was quenched with water (10 mL), and the aqueous phase was extracted with ethyl acetate (3×10 mL). The organic phase was dried over Na₂SO₄, concentrated, and purified via CombiFlash® using silica gel as the stationary phase and was eluted with 15% EtOAc in hexane. LC-MS: calculated [M+H]+ 512.24, found 512.56.

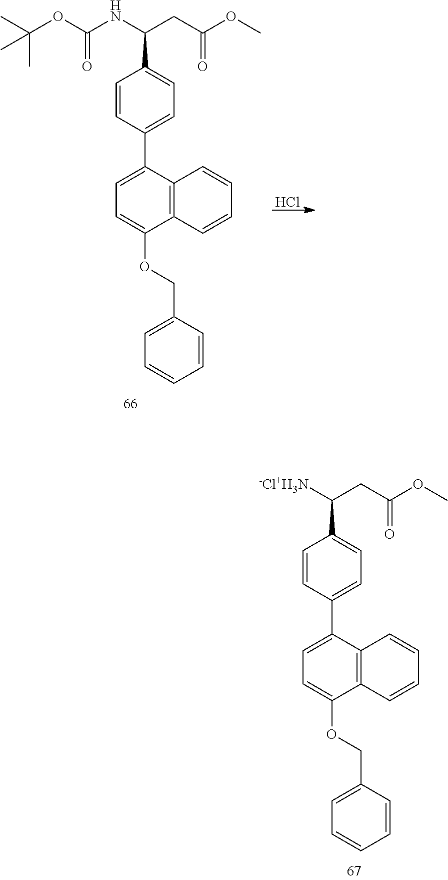

Compound 66 (858 mg, 1.677 mmol, 1.0 equiv.) was cooled by ice bath. HCl in dioxane (8.4 mL, 33.54 mmol, 20 equiv.) was added into the flask. The reaction was warmed to room temperature and stirred for another 1 hr. The solvent was removed by rotary evaporator and the product was directly used without further purification. LC-MS: calculated [M+H]+ 412.18, found 412.46.

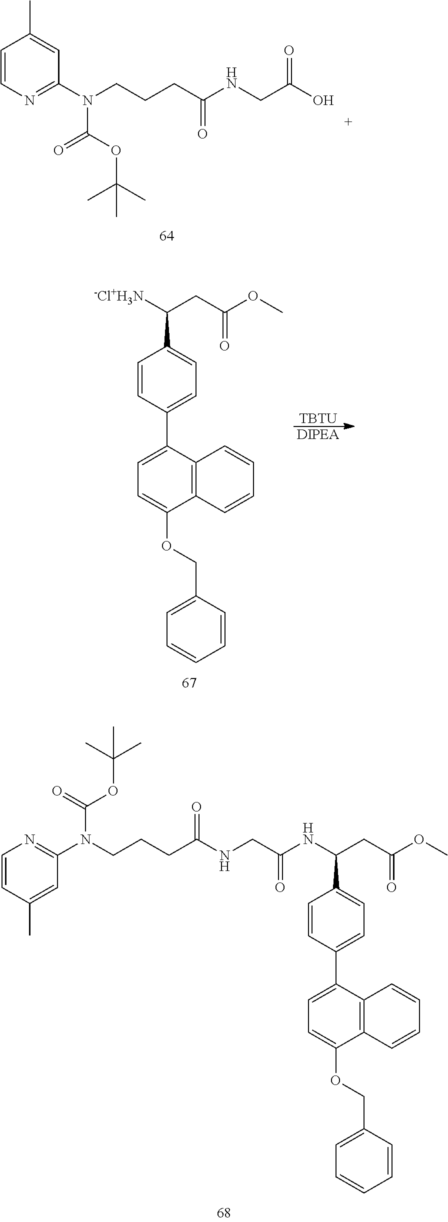

To a solution of compound 64 (500 mg, 1.423 mmol, 1 equiv.), compound 67 (669 mg, 1.494 mmol, 1.05 equiv.), and TBTU (548 mg, 0.492 mmol, 1.2 equiv.) in anhydrous DMF (15 mL) was added diisopropylethylamine (0.744 mL, 4.268 mmol, 3 equiv.) at 0° C. The reaction mixture was warmed to room temperature and stirred for another 1 hr. The reaction was quenched by saturated NaHCO₃ aqueous solution (10 mL) and the product was extracted with ethyl acetate (3×20 mL). The organic phase was combined, dried over Na₂SO₄, and concentrated. The product was purified by CombiFlash® using silica gel as the stationary phase and was eluted with 3-4% methanol in DCM. The yield was 96.23%. LC-MS: calculated [M+H]+ 745.35, found 746.08.

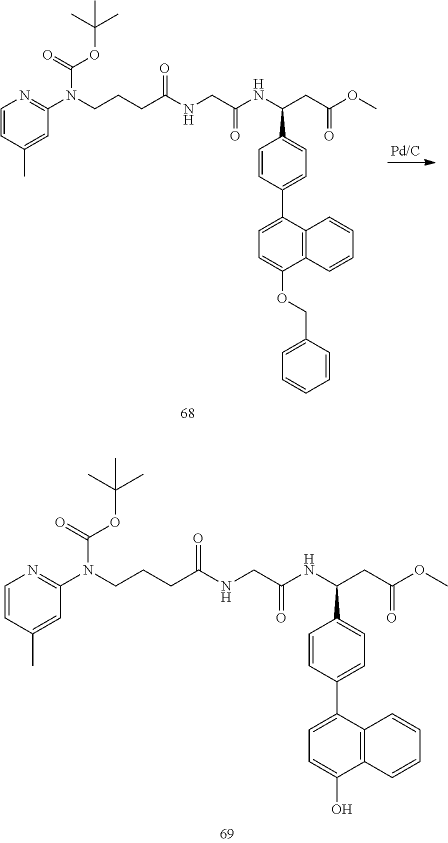 [SMILES: COC(=O)C[C@H](NC(=O)CNC(=O)CCCN(C(=O)OC(C)(C)C)c1cc(C)ccn1)c1ccc(-c2ccc(OCc3ccccc3)c3ccccc23)cc1 ; COC(=O)C[C@H](NC(=O)CNC(=O)CCCN(C(=O)OC(C)(C)C)c1cc(C)ccn1)c1ccc(-c2ccc(O)c3ccccc23)cc1]

To a solution of compound 68 (1.02 g, 1.369 mmol, 1 equiv.) in ethyl acetate (10 mL) was added 10% Pd/C (0.15 g, 50% H₂O) at room temperature. The reaction mixture was warmed to room temperature and the reaction was monitored by LC-MS. The reaction was kept at room temperature overnight. The solids were filtered through Celite® and the solvent was removed by rotary evaporator. The product was directly used without further purification. LC-MS: [M+H]+ 655.31, found 655.87.

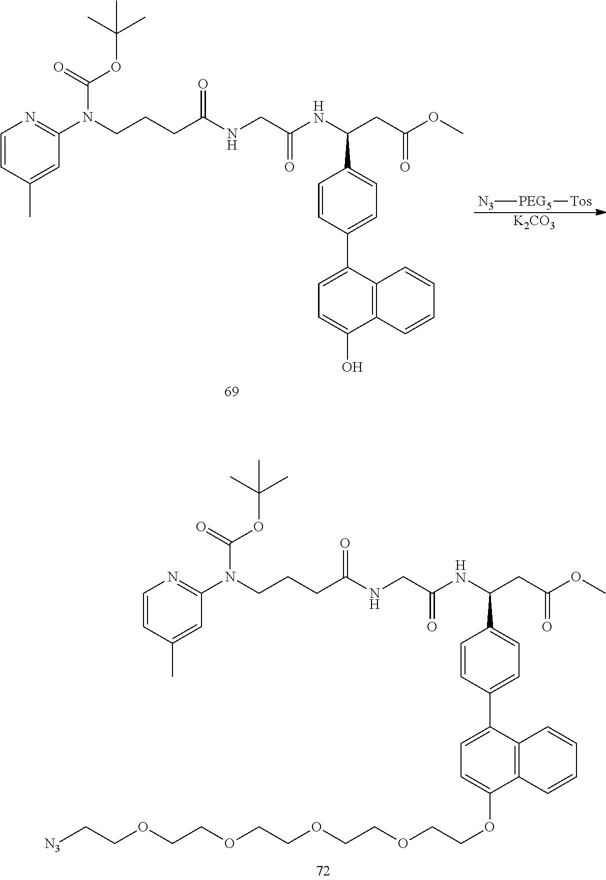

To a solution of compound 69 (100 mg, 0.152 mmol, 1 equiv.) and azido-PEG5-OTs (128 mg, 0.305 mmol, 2 equiv.) in anhydrous DMF (2 mL) was added K₂CO₃ (42 mg, 0.305 mmol, 2 equiv.) at 0° C. The reaction mixture was stirred for 6 hours at 80° C. The reaction was quenched by saturated NaHCO₃ solution and the aqueous layer was extracted with ethyl acetate (3×10 mL). The organic phase was combined, dried over Na₂SO₄, and concentrated. LC-MS: calculated [M+H]+ 900.40, found 901.46.

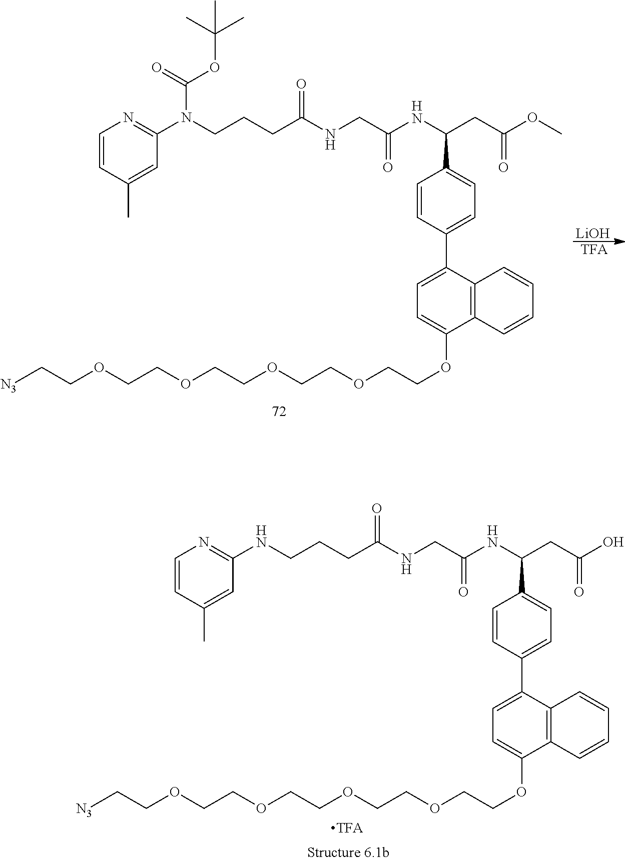

To a solution of compound 72 (59 mg, 0.0656 mmol, 1.0 equiv.) in THF (2 mL) and water (2 mL) was added lithium hydroxide (5 mg, 0.197 mmol, 3.0 equiv.) at room temperature. The mixture was stirred at room temperature for another 1 hr. The pH was adjusted to 3.0 by HCl (6N) and the aqueous phase was extracted with EtOAc (3×10 mL). The organic phase was combined, dried over Na₂SO₄, and concentrated. TFA (0.5 mL) and DCM (0.5 mL) was added into the residue and the mixture was stirred at room temperature for another 3 hr. The solvent was removed by rotary evaporator. LC-MS: calculated [M+H]+ 786.37, found 786.95.

### G. Synthesis of TriAlk 14

TriAlk14 and (TriAlk14)s as shown in Table 11, above, may be synthesized using the synthetic route shown below. Compound 14 may be added to the sense strand as a phosphoramidite using standard oligonucleotide synthesis techniques, or compound 22 may be conjugated to the sense strand comprising an amine in an amide coupling reaction.

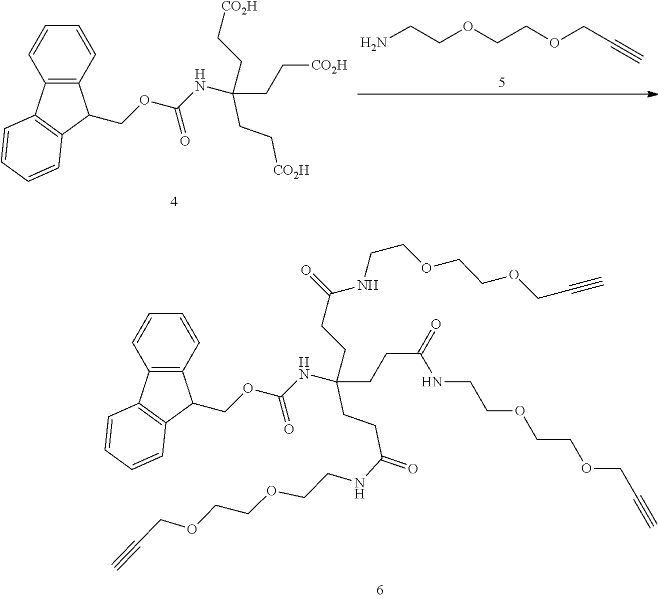

To a 3-L jacketed reactor was added 500 mL DCM and 4 (75.0 g, 0.16 mol). The internal temperature of the reaction was cooled to 0° C. and TBTU (170.0 g, 0.53 mol) was added. The suspension was then treated with the amine 5 (75.5 g, 0.53 mol) dropwise keeping the internal temperature less than 5° C. The reaction was then treated with DIPEA (72.3 g, 0.56 mol) slowly, keeping the internal temperature less than 5° C. After the addition was complete, the reaction was warmed up to 23° C. over 1 hour, and allowed to stir for 3 hours. A 10% kicker charge of all three reagents were added and allowed to stir an additional 3 hours. The reaction was deemed complete when <1% of 4 remained. The reaction mixture was washed with saturated ammonium chloride solution (2×500 mL) and once with saturated sodium bicarbonate solution (500 mL). The organic layer was then dried over sodium sulfate and concentrated to an oil. The mass of the crude oil was 188 g which contained 72% 6 by QNMR. The crude oil was carried to the next step. Calculated mass for C₄₆H₆₀N₄O₁₁=845.0 m/z. Found [M+H]=846.0.

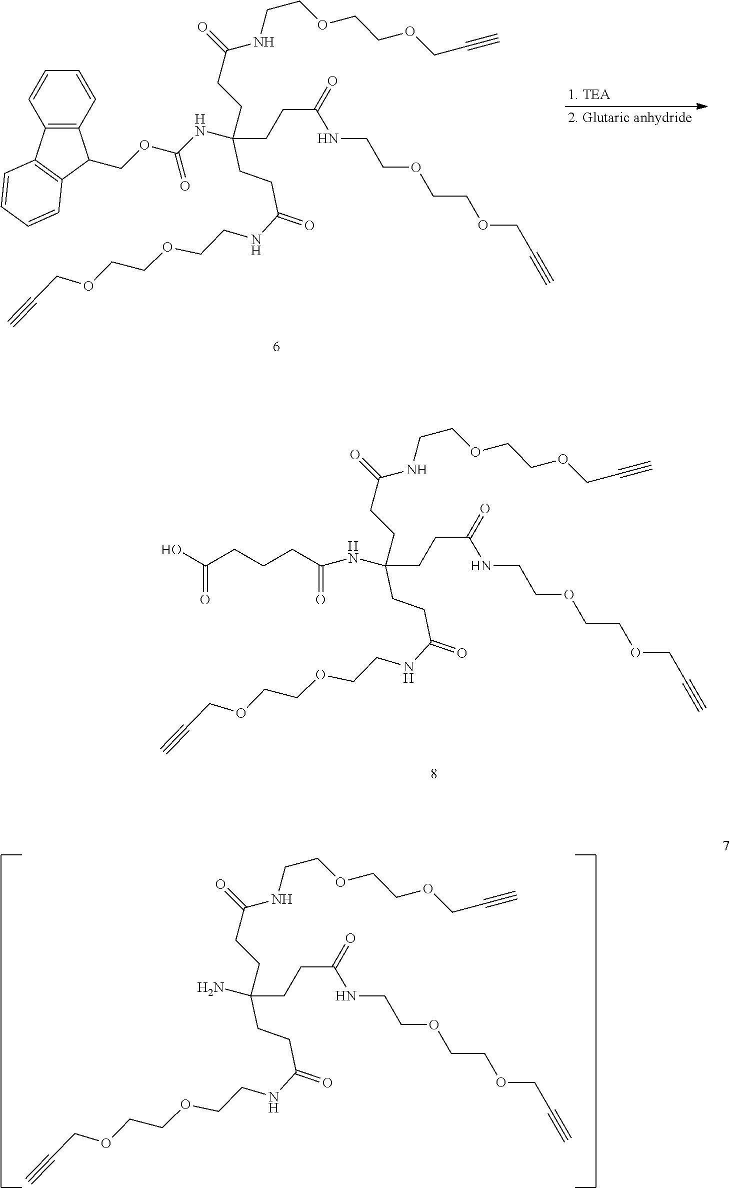

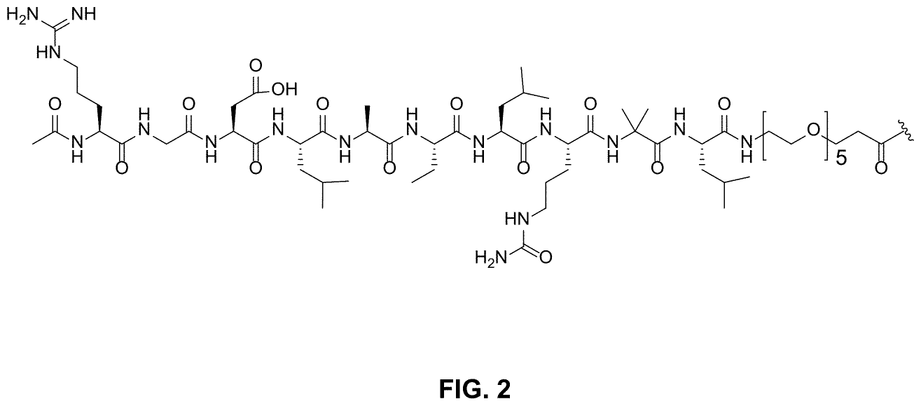

The 121.2 g of crude oil containing 72 wt % compound 6 (86.0 g, 0.10 mol) was dissolved in DMF (344 mL) and treated with TEA (86 mL, 20 v/v %), keeping the internal temperature below 23° C. The formation of dibenzofulvene (DBF) relative to the consumption of Fmoc-amine 6 was monitored via HPLC method 1 (FIG. 2) and the reaction was complete within 10 hours. To the solution was added glutaric anhydride (12.8 g, 0.11 mol) and the intermediate amine 7 was converted to compound 8 within 2 hours. Upon completion, the DMF and TEA were removed at 30° C. under reduced pressure resulting in 100 g of a crude oil. Due to the high solubility of compound 7 in water, an aqueous workup could not be used, and chromatography is the only way to remove DBF, TMU, and glutaric anhydride. The crude oil (75 g) was purified on a Teledyne ISCO Combi-Flash® purification system in three portions. The crude oil (25 g) was loaded onto a 330 g silica column and eluted from 0-20% methanol/DCM over 30 minutes resulting in 42 g of compound 8 (54% yield over 3 steps). Calculated mass for C₃₆H₅₅N₄O₁₂=736.4 m/z. Found [M+H]=737.0.

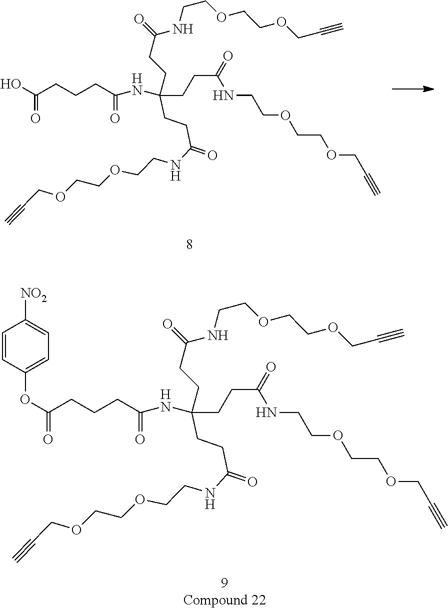 [SMILES: C#CCOCCOCCNC(=O)CCC(CCC(=O)NCCOCCOCC#C)(CCC(=O)NCCOCCOCC#C)NC(=O)CCCC(=O)O ; C#CCOCCOCCNC(=O)CCC(CCC(=O)NCCOCCOCC#C)(CCC(=O)NCCOCCOCC#C)NC(=O)CCCC(=O)Oc1ccc([N+](=O)[O-])cc1]

Compound 8 (42.0 g, 0.057 mol) was co-stripped with 10 volumes of acetonitrile prior to use to remove any residual methanol from chromatography solvents. The oil was redissolved in DMF (210 mL) and cooled to 0° C. The solution was treated with 4-nitrophenol (8.7 g, 0.063 moL) followed by EDC-hydrochloride (12.0 g, 0.063 mol) and found to reach completion within 10 hours. The solution was cooled to 0° C. and 10 volumes ethyl acetate was added followed by 10 volumes saturated ammonium chloride solution, keeping the internal temperature below 15° C. The layers were allowed to separate and the ethyl acetate layer was washed with brine. The combined aqueous layers were extracted twice with 5 volumes ethyl acetate. The combined organic layers were dried over sodium sulfate and concentrated to an oil. The crude oil (55 g) was purified on a Teledyne ISCO Combi-Flash® purification system in three portions. The crude oil (25 g) was loaded onto a 330 g silica column and eluted from 0-10% methanol/DCM over 30 minutes resulting in 22 g of pure 9 (Compound 22) (50% yield). Calculated mass for C₄₂H₅₉N₅O₁₄=857.4 m/z. Found [M+H]=858.0.

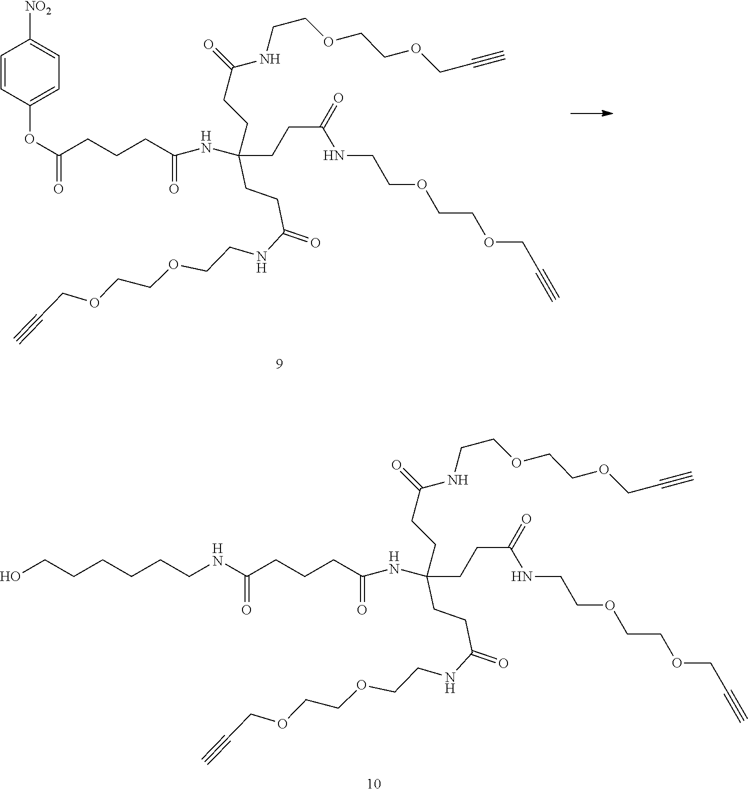 [SMILES: C#CCOCCOCCNC(=O)CCC(CCC(=O)NCCOCCOCC#C)(CCC(=O)NCCOCCOCC#C)NC(=O)CCCC(=O)Oc1ccc([N+](=O)[O-])cc1 ; C#CCOCCOCCNC(=O)CCC(CCC(=O)NCCOCCOCC#C)(CCC(=O)NCCOCCOCC#C)NC(=O)CCCC(=O)NCCCCCCO]

A solution of ester 9 (49.0 g, 57.1 mmol) and 6-amino-1-hexanol (7.36 g, 6.28 mmol) in dichloromethane (3 volumes) was treated with triethylamine (11.56 g, 111.4 mmol) dropwise. The reaction was monitored by observing the disappearance of compound 9 on HPLC Method 1 and was found to be complete in 10 minutes. The crude reaction mixture was diluted with 5 volumes dichloromethane and washed with saturated ammonium chloride (5 volumes) and brine (5 volumes). The organic layer was dried over sodium sulfate and concentrated to an oil. The crude oil was purified on a Teledyne ISCO Combi-Flash® purification system using a 330 g silica column. The 4-nitrophenol was eluted with 100% ethyl acetate and 10 was flushed from the column using 20% methanol/DCM resulting in a colorless oil (39 g, 81% yield). Calculated mass for C₄₂H₆₉N₅O₁₂=836.0 m/z. Found [M+H]=837.0.

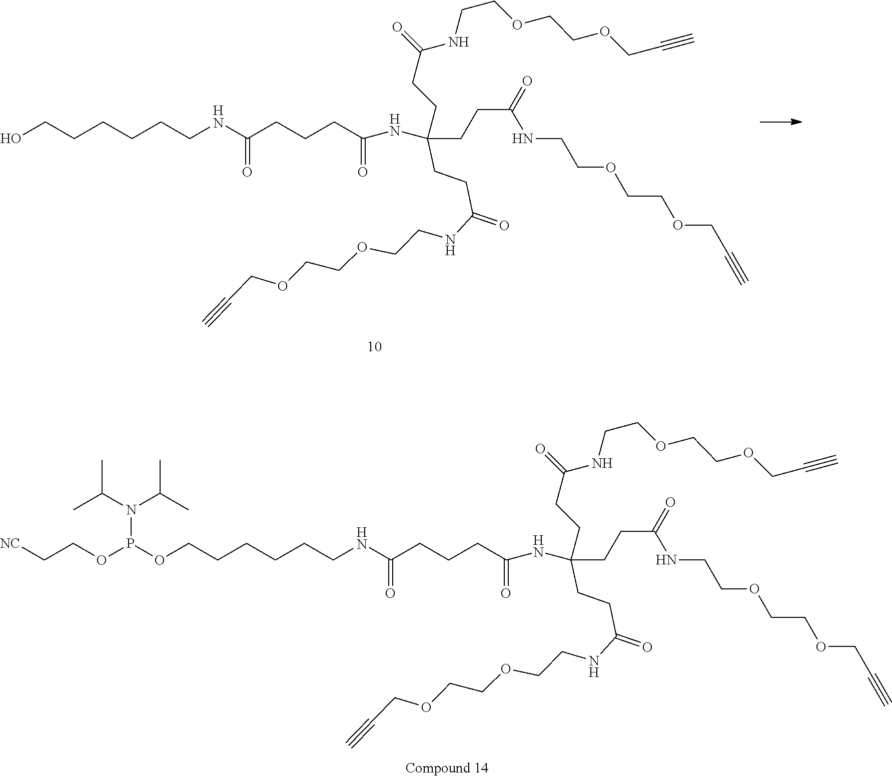 [SMILES: C#CCOCCOCCNC(=O)CCC(CCC(=O)NCCOCCOCC#C)(CCC(=O)NCCOCCOCC#C)NC(=O)CCCC(=O)NCCCCCCO ; C#CCOCCOCCNC(=O)CCC(CCC(=O)NCCOCCOCC#C)(CCC(=O)NCCOCCOCC#C)NC(=O)CCCC(=O)NCCCCCCOP(OCCC#N)N(C(C)C)C(C)C]

Alcohol 10 was co-stripped twice with 10 volumes of acetonitrile to remove any residual methanol from chromatography solvents and once more with dry dichloromethane (KF<60 ppm) to remove trace water. The alcohol 10 (2.30 g, 2.8 mmol) was dissolved in 5 volumes dry dichloromethane (KF<50 ppm) and treated with diisopropylammonium tetrazolide (188 mg, 1.1 mmol). The solution was cooled to 0° C. and treated with 2-cyanoethyl N,N,N′,N′-tetraisopropylphosphoramidite (1.00 g, 3.3 mmol) dropwise. The solution was removed from ice-bath and stirred at 20° C. The reaction was found to be complete within 3-6 hours. The reaction mixture was cooled to 0° C. and treated with 10 volumes of a 1:1 solution of saturated ammonium bicarbonate/brine and then warmed to ambient over 1 minute and allowed to stir an additional 3 minutes at 20° C. The biphasic mixture was transferred to a separatory funnel and 10 volumes of dichloromethane was added. The organic layer was separated and washed with 10 volumes of saturated sodium bicarbonate solution to hydrolyze unreacted bis-phosphorous reagent. The organic layer was dried over sodium sulfate and concentrated to an oil resulting in 3.08 g of 94 wt % Compound 14. Calculated mass for C₅₁H₈₆N₇O₁₃P=1035.6 m/z. Found [M+H]=1036.

### H. Conjugation of Targeting Ligands

Either prior to or after annealing, the 5′ or 3′ tridentate alkyne functionalized sense strand is conjugated to targeting ligands. The following example describes the conjugation of targeting ligands to the annealed duplex: Stock solutions of 0.5M Tris(3-hydroxypropyltriazolylmethyl)amine (THPTA), 0.5M of Cu(II) sulfate pentahydrate (Cu(II)SO₄·5H₂O) and 2M solution of sodium ascorbate were prepared in deionized water. A 75 mg/mL solution in DMSO of targeting ligand was made. In a 1.5 mL centrifuge tube containing tri-alkyne functionalized duplex (3 mg, 75 μL, 40 mg/mL in deionized water, ˜15,000 g/mol), 25 μL of 1M Hepes pH 8.5 buffer is added. After vortexing, 35 μL of DMSO was added and the solution is vortexed. Targeting ligand was added to the reaction (6 equivalents/duplex, 2 equivalents/alkyne, ˜15 μL) and the solution is vortexed. Using pH paper, pH was checked and confirmed to be pH ˜8. In a separate 1.5 mL centrifuge tube, 50 μL of 0.5M THPTA was mixed with 10 uL of 0.5M Cu(II)SO₄·5H₂O, vortexed, and incubated at room temp for 5 min. After 5 min, THPTA/Cu solution (7.2 μL, 6 equivalents 5:1 THPTA:Cu) was added to the reaction vial, and vortexed. Immediately afterwards, 2M ascorbate (5 μL, 50 equivalents per duplex, 16.7 per alkyne) was added to the reaction vial and vortexed. Once the reaction was complete (typically complete in 0.5-1 h), the reaction was immediately purified by non-denaturing anion exchange chromatography.

### Example 2. MMP7-SEAP Mouse Model

To assess the potency of the RNAi agents, an MMP7-SEAP mouse model was used. Six to eight week old female C57BL/6 albino mice were transiently transfected in vivo with plasmid by hydrodynamic tail vein injection, administered at least 15 days prior to administration of an MMP7 RNAi agent or control. The plasmid contains the MMP7 cDNA sequence (GenBank NM_002423.5 (SEQ ID NO:1)) inserted into the 3′ UTR of the SEAP (secreted human placental alkaline phosphatase) reporter gene. 50 μg of the plasmid containing the MMP7 cDNA sequence in Ringer's Solution in a total volume of 10% of the animal's body weight was injected into mice via the tail vein to create MMP7-SEAP model mice. The solution was injected through a 27-gauge needle in 5-7 seconds as previously described (Zhang G et al., “High levels of foreign gene expression in hepatocytes after tail vein injection of naked plasmid DNA.” Human Gene Therapy 1999 Vol. 10, p 1735-1737.). Inhibition of expression of MMP7 by an MMP7 RNAi agent results in concomitant inhibition of SEAP expression, which is measured by the Phospha-Light™ SEAP Reporter Gene Assay System (Invitrogen). Prior to treatment, SEAP expression levels in serum were measured and the mice were grouped according to average SEAP levels.

Analyses: SEAP levels may be measured at various times, both before and after administration of MMP7 RNAi agents.

i) Serum collection: Mice were anesthetized with 2-3% isoflurane and blood samples were collected from the submandibular area into serum separation tubes (Sarstedt AG & Co., Numbrecht, Germany). Blood was allowed to coagulate at ambient temperature for 20 min. The tubes were centrifuged at 8,000×g for 3 min to separate the serum and stored at 4° C.

ii) Serum SEAP levels: Serum was collected and measured by the Phospha-Light™ SEAP Reporter Gene Assay System (Invitrogen) according to the manufacturer's instructions. Serum SEAP levels for each animal was normalized to the control group of mice injected with saline in order to account for the non-treatment related decline in MMP7 expression with this model. First, the SEAP level for each animal at a time point was divided by the pre-treatment level of expression in that animal (“pre-treatment”) in order to determine the ratio of expression “normalized to pre-treatment”. Expression at a specific time point was then normalized to the control group by dividing the “normalized to pre-treatment” ratio for an individual animal by the average “normalized to pre-treatment” ratio of all mice in the normal saline control group. Alternatively, in some Examples set forth herein, the serum SEAP levels for each animal were assessed by normalizing to pre-treatment levels only.

### Example 3. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 3.0 mg/kg (mpk) of an MMP7 RNAi agent or saline without an MMP7 RNAi agent to be used as a control, according to the following Table 12.

TABLE 12
MMP7 RNAi Agent and Dosing for Example 3

| Group ID | Dosing Regimen |
|---|---|
| Group 1 (isotonic saline) | Single injection on day 1 |
| Group 2 3.0 mg/kg AD08797 | Single injection on day 1 |
| Group 3 3.0 mg/kg AD08798 | Single injection on day 1 |
| Group 4 3.0 mg/kg AD08801 | Single injection on day 1 |
| Group 5 3.0 mg/kg AD08802 | Single injection on day 1 |
| Group 6 3.0 mg/kg AD08803 | Single injection on day 1 |
| Group 7 3.0 mg/kg AD08804 | Single injection on day 1 |
| Group 8 3.0 mg/kg AD08805 | Single injection on day 1 |
| Group 9 3.0 mg/kg AD08815 | Single injection on day 1 |
| Group 10 3.0 mg/kg AD08816 | Single injection on day 1 |
| Group 11 3.0 mg/kg AD08823 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i. e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 13, with Average SEAP reflecting the normalized average value of SEAP:

TABLE 13
Average SEAP normalized to pre-treatment and saline control
in MMP7-SEAP mice from Example 3.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.509 | 1.000 | 0.414 |
| Group 2 3.0 mg/kg AD08797 | 0.217 | 0.044 | 0.187 | 0.112 |
| Group 3 3.0 mg/kg AD08798 | 0.259 | 0.070 | 0.189 | 0.048 |
| Group 4 3.0 mg/kg AD08801 | 0.265 | 0.073 | 0.401 | 0.155 |
| Group 5 3.0 mg/kg AD08802 | 0.237 | 0.036 | 0.216 | 0.082 |
| Group 6 3.0 mg/kg AD08803 | 0.125 | 0.044 | 0.104 | 0.070 |
| Group 7 3.0 mg/kg AD08804 | 0.227 | 0.103 | 0.195 | 0.063 |
| Group 8 3.0 mg/kg AD08805 | 0.145 | 0.098 | 0.129 | 0.143 |
| Group 9 3.0 mg/kg AD08815 | 0.150 | 0.014 | 0.069 | 0.028 |
| Group 10 3.0 mg/kg AD08816 | 0.643 | 0.716 | 0.288 | 0.119 |
| Group 11 3.0 mg/kg AD08823 | 0.129 | 0.026 | 0.075 | 0.030 |
| | Day 22 | | Day 29 | |
| | Avg | Std Dev | Avg | Std Dev |
| Group ID | SEAP | (+/−) | SEAP | (+/−) |
| Group 1 (Saline) | 1.000 | 0.581 | 1.000 | 0.464 |
| Group 2 3.0 mg/kg AD08797 | 0.221 | 0.021 | 0.381 | 0.082 |
| Group 3 3.0 mg/kg AD08798 | 0.227 | 0.066 | 0.377 | 0.170 |
| Group 4 3.0 mg/kg AD08801 | 0.434 | 0.109 | 0.577 | 0.108 |
| Group 5 3.0 mg/kg AD08802 | 0.220 | 0.082 | 0.279 | 0.136 |
| Group 6 3.0 mg/kg AD08803 | 0.075 | 0.050 | 0.081 | 0.039 |
| Group 7 3.0 mg/kg AD08804 | 0.169 | 0.034 | 0.299 | 0.083 |
| Group 8 3.0 mg/kg AD08805 | 0.223 | 0.317 | 0.414 | 0.568 |
| Group 9 3.0 mg/kg AD08815 | 0.057 | 0.028 | 0.111 | 0.068 |
| Group 10 3.0 mg/kg AD08816 | 0.247 | 0.075 | 0.375 | 0.212 |
| Group 11 3.0 mg/kg AD08823 | 0.092 | 0.070 | 0.093 | 0.089 |

Each of the MMP7 RNAi agents in each of the dosing groups (i.e., Groups 2 through 11) showed reduction in SEAP as compared to the saline control (Group 1) across all measured time points, which as described herein, indicates inhibition of MMP7 in the MMP7-SEAP mouse model.

### Example 4. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 3.0 mg/kg (mpk) of an MMP7 RNAi agent or saline without an MMP7 RNAi agent to be used as a control, according to the following Table 14.

TABLE 14
MMP7 RNAi Agent and Dosing for Example 4.

| Group ID | Dosing Regimen |
|---|---|
| Group 1 (isotonic saline) | Single injection on day 1 |
| Group 2 3.0 mg/kg AD08799 | Single injection on day 1 |
| Group 3 3.0 mg/kg AD08800 | Single injection on day 1 |
| Group 4 3.0 mg/kg AD08806 | Single injection on day 1 |
| Group 5 3.0 mg/kg AD08807 | Single injection on day 1 |
| Group 6 3.0 mg/kg AD08808 | Single injection on day 1 |
| Group 7 3.0 mg/kg AD08813 | Single injection on day 1 |
| Group 8 3.0 mg/kg AD08814 | Single injection on day 1 |
| Group 9 3.0 mg/kg AD08817 | Single injection on day 1 |
| Group 10 3.0 mg/kg AD08818 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i. e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 15, with Average SEAP reflecting the normalized average value of SEAP:

TABLE 15
Average SEAP normalized to pre-treatment and saline control
in MMP7-SEAP mice from Example 4.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.112 | 1.000 | 0.402 |
| Group 2 3.0 mg/kg AD08799 | 0.300 | 0.054 | 0.731 | 0.182 |
| Group 3 3.0 mg/kg AD08800 | 0.208 | 0.079 | 0.237 | 0.165 |
| Group 4 3.0 mg/kg AD08806 | 0.215 | 0.063 | 0.282 | 0.173 |
| Group 5 3.0 mg/kg AD08807 | 0.168 | 0.060 | 0.166 | 0.099 |
| Group 6 3.0 mg/kg AD08808 | 0.143 | 0.042 | 0.143 | 0.111 |
| Group 7 3.0 mg/kg AD08813 | 0.192 | 0.072 | 0.088 | 0.029 |
| Group 8 3.0 mg/kg AD08814 | 0.154 | 0.060 | 0.339 | 0.152 |
| Group 9 3.0 mg/kg AD08817 | 0.430 | 0.097 | 0.369 | 0.206 |
| Group 10 3.0 mg/kg AD08818 | 0.160 | 0.073 | 0.127 | 0.043 |
| | Day 22 | | Day 29 | |
| | Avg | Std Dev | Avg | Std Dev |
| Group ID | SEAP | (+/−) | SEAP | (+/−) |
| Group 1 (Saline) | 1.000 | 0.323 | 1.000 | 0.364 |
| Group 2 3.0 mg/kg AD08799 | 0.818 | 0.164 | 0.730 | 0.279 |
| Group 3 3.0 mg/kg AD08800 | 0.194 | 0.130 | 0.384 | 0.240 |
| Group 4 3.0 mg/kg AD08806 | 0.454 | 0.378 | 0.502 | 0.424 |
| Group 5 3.0 mg/kg AD08807 | 0.155 | 0.074 | 0.287 | 0.137 |
| Group 6 3.0 mg/kg AD08808 | 0.116 | 0.106 | 0.192 | 0.171 |
| Group 7 3.0 mg/kg AD08813 | 0.077 | 0.053 | 0.151 | 0.113 |
| Group 8 3.0 mg/kg AD08814 | 0.307 | 0.139 | 0.463 | 0.356 |
| Group 9 3.0 mg/kg AD08817 | 0.313 | 0.085 | 0.456 | 0.138 |
| Group 10 3.0 mg/kg AD08818 | 0.087 | 0.047 | 0.148 | 0.086 |

Each of the MMP7 RNAi agents in each of the dosing groups (i.e., Groups 2 through 10) showed reduction in SEAP as compared to the saline control (Group 1) across all measured time points, which as described herein, indicates inhibition of MMP7 in the MMP7-SEAP mouse model.

### Example 5. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 3.0 mg/kg (mpk) of an MMP7 RNAi agent, 1 mpk of an MMP7 RNAi agent, 0.3 mpk an MMP7 RNAi agent, or saline without an MMP7 RNAi agent to be used as a control, according to the following Table 16.

TABLE 16
MMP7 RNAi Agent and Dosing for Example 5.

| Group ID | Dosing Regimen |
|---|---|
| Group 1 (isotonic saline) | Single injection on day 1 |
| Group 2 3.0 mg/kg AD08809 | Single injection on day 1 |
| Group 3 3.0 mg/kg AD08811 | Single injection on day 1 |
| Group 4 3.0 mg/kg AD08812 | Single injection on day 1 |
| Group 5 3.0 mg/kg AD08819 | Single injection on day 1 |
| Group 6 3.0 mg/kg AD08820 | Single injection on day 1 |
| Group 7 3.0 mg/kg AD08821 | Single injection on day 1 |
| Group 8 3.0 mg/kg AD08822 | Single injection on day 1 |
| Group 9 3.0 mg/kg AD08815 | Single injection on day 1 |
| Group 10 1.0 mg/kg AD08815 | Single injection on day 1 |
| Group 11 0.3 mg/kg AD08815 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i. e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 17, with Average SEAP reflecting the normalized average value of SEAP:

TABLE 17
Average SEAP normalized to pre-treatment and saline control
in MMP7-SEAP mice from Example 5.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.264 | 1.000 | 0.478 |
| Group 2 3.0 mg/kg AD08809 | 0.807 | 0.283 | 0.853 | 0.441 |
| Group 3 3.0 mg/kg AD08811 | 0.177 | 0.046 | 0.111 | 0.076 |
| Group 4 3.0 mg/kg AD08812 | 0.704 | 0.342 | 0.567 | 0.143 |
| Group 5 3.0 mg/kg AD08819 | 0.549 | 0.079 | 0.405 | 0.083 |
| Group 6 3.0 mg/kg AD08820 | 0.133 | 0.022 | 0.046 | 0.020 |
| Group 7 3.0 mg/kg AD08821 | 0.148 | 0.061 | 0.051 | 0.028 |
| Group 8 3.0 mg/kg AD08822 | 0.111 | 0.025 | 0.048 | 0.018 |
| Group 9 3.0 mg/kg AD08815 | 0.193 | 0.043 | 0.097 | 0.043 |
| Group 10 1.0 mg/kg AD08815 | 0.630 | 0.139 | 0.299 | 0.087 |
| Group 11 0.3 mg/kg AD08815 | 0.789 | 0.185 | 0.493 | 0.157 |
| | Day 22 | | Day 29 | |
| | Avg | Std Dev | Avg | Std Dev |
| Group ID | SEAP | (+/−) | SEAP | (+/−) |
| Group 1 (Saline) | 1.000 | 0.556 | 1.000 | 0.398 |
| Group 2 3.0 mg/kg AD08809 | 0.797 | 0.396 | 0.920 | 0.517 |
| Group 3 3.0 mg/kg AD08811 | 0.118 | 0.099 | 0.202 | 0.137 |
| Group 4 3.0 mg/kg AD08812 | 0.994 | 0.384 | 0.565 | 0.079 |
| Group 5 3.0 mg/kg AD08819 | 0.375 | 0.045 | 0.652 | 0.148 |
| Group 6 3.0 mg/kg AD08820 | 0.040 | 0.022 | 0.087 | 0.065 |
| Group 7 3.0 mg/kg AD08821 | 0.038 | 0.025 | 0.050 | 0.023 |
| Group 8 3.0 mg/kg AD08822 | 0.038 | 0.016 | 0.085 | 0.030 |
| Group 9 3.0 mg/kg AD08815 | 0.078 | 0.030 | 0.156 | 0.065 |
| Group 10 1.0 mg/kg AD08815 | 0.395 | 0.074 | 0.650 | 0.179 |
| Group 11 0.3 mg/kg AD08815 | 0.625 | 0.152 | 0.819 | 0.291 |

Each of the MMP7 RNAi agents in each of the dosing groups (i.e., Groups 2 through 11) showed reduction in SEAP as compared to the saline control (Group 1) across all measured time points, which as described herein, indicates inhibition of MMP7 in the MMP7-SEAP mouse model.

### Example 6. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 2.0 mg/kg (mpk) of an MMP7 RNAi agent, 1.0 mpk of an MMP7 RNAi agent, or saline without an MMP7 RNAi agent to be used as a control, according to the following Table 18.

TABLE 18
MMP7 RNAi Agent and Dosing for Example 5.

| Group ID | Dosing Regimen |
|---|---|
| Group 1 (isotonic saline) | Single injection on day 1 |
| Group 2 2.0 mg/kg AD08815 | Single injection on day 1 |
| Group 3 1.0 mg/kg AD08815 | Single injection on day 1 |
| Group 4 2.0 mg/kg AD08823 | Single injection on day 1 |
| Group 5 1.0 mg/kg AD08823 | Single injection on day 1 |
| Group 6 2.0 mg/kg AD08803 | Single injection on day 1 |
| Group 7 1.0 mg/kg AD08803 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i. e. subcutaneous injections) into the loose skin over the neck and shoulder area. Four (4) mice in each of groups 1-6 were tested (n=4), and three (3) mice were tested in group 7 (n=3.) Serum was collected on day 8, day 15, day 22, and day 29, and SEAP expression levels were determined pursuant to the procedure set forth in Example 2, above. Data from the experiment are shown in the following Table 19, with Average SEAP reflecting the normalized average value of SEAP:

TABLE 19
Average SEAP normalized to pre-treatment and saline control
in MMP7-SEAP mice from Example 6.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.637 | 1.000 | 0.354 |
| Group 2 2.0 mg/kg AD08815 | 0.261 | 0.086 | 0.169 | 0.099 |
| Group 3 1.0 mg/kg AD08815 | 0.307 | 0.198 | 0.435 | 0.277 |
| Group 4 2.0 mg/kg AD08823 | 0.230 | 0.172 | 0.140 | 0.042 |
| Group 5 1.0 mg/kg AD08823 | 0.543 | 0.250 | 0.266 | 0.079 |
| Group 6 2.0 mg/kg AD08803 | 0.617 | 0.294 | 0.204 | 0.109 |
| Group 7 1.0 mg/kg AD08803 | 0.320 | 0.145 | 0.283 | 0.063 |
| | Day 22 | | Day 29 | |
| | Avg | Std Dev | Avg | Std Dev |
| Group ID | SEAP | (+/−) | SEAP | (+/−) |
| Group 1 (Saline) | 1.000 | 0.235 | 1.000 | 0.302 |
| Group 2 2.0 mg/kg AD08815 | 0.157 | 0.143 | 0.256 | 0.092 |
| Group 3 1.0 mg/kg AD08815 | 0.343 | 0.171 | 0.522 | 0.233 |
| Group 4 2.0 mg/kg AD08823 | 0.212 | N/A* | 0.294 | 0.155 |
| Group 5 1.0 mg/kg AD08823 | 0.291 | 0.107 | 0.502 | 0.205 |
| Group 6 2.0 mg/kg AD08803 | 0.087 | 0.042 | 0.179 | 0.092 |
| Group 7 1.0 mg/kg AD08803 | 0.309 | 0.371 | 0.211 | N/A* |

*Only one mouse from the group was tested on the indicated day.

Each of the MMP7 RNAi agents in each of the dosing groups (i.e., Groups 2 through 7) showed reduction in SEAP as compared to the saline control (Group 1) across all measured time points, which as described herein, indicates inhibition of MMP7 in the MMP7-SEAP mouse model.

### Example 7. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 2.0 mg/kg (mpk) of an MMP7 RNAi agent, 1.0 mpk of an MMP7 RNAi agent, 0.5 mpk an MMP7 RNAi agent, or saline without an MMP7 RNAi agent to be used as a control, according to the following Table 20.

TABLE 20
MMP7 RNAi Agent and Dosing for Example 7.

| Group ID | Dosing Regimen |
|---|---|
| Group 1 (isotonic saline) | Single injection on day 1 |
| Group 2 2.0 mg/kg AD08808 | Single injection on day 1 |
| Group 3 1.0 mg/kg AD08808 | Single injection on day 1 |
| Group 4 0.5 mg/kg AD08808 | Single injection on day 1 |
| Group 5 2.0 mg/kg AD08818 | Single injection on day 1 |
| Group 6 1.0 mg/kg AD08818 | Single injection on day 1 |
| Group 7 0.5 mg/kg AD08818 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i.e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 21, with Average SEAP reflecting the normalized average value of SEAP.

TABLE 21
Average SEAP normalized to pre-treatment and saline control
in MMP7-SEAP mice from Example 7.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.199 | 1.000 | 0.169 |
| Group 2 2.0 mg/kg AD08808 | 0.281 | 0.137 | 0.373 | 0.417 |
| Group 3 1.0 mg/kg AD08808 | 0.432 | 0.087 | 0.547 | 0.191 |
| Group 4 0.5 mg/kg AD08808 | 0.646 | 0.331 | 0.589 | 0.217 |
| Group 5 2.0 mg/kg AD08818 | 0.403 | 0.158 | 0.462 | 0.442 |
| Group 6 1.0 mg/kg AD08818 | 0.793 | 0.229 | 0.623 | 0.256 |
| Group 7 0.5 mg/kg AD08818 | 0.649 | 0.092 | 0.669 | 0.266 |
| | Day 22 | | Day 29 | |
| | Avg | Std Dev | Avg | Std Dev |
| Group ID | SEAP | (+/−) | SEAP | (+/−) |
| Group 1 (Saline) | 1.000 | 0.229 | 1.000 | 0.135 |
| Group 2 2.0 mg/kg AD08808 | 0.056 | 0.020 | 0.049 | N/A* |
| Group 3 1.0 mg/kg AD08808 | 0.355 | 0.176 | 0.434 | 0.080 |
| Group 4 0.5 mg/kg AD08808 | 0.649 | 0.559 | 0.639 | 0.102 |
| Group 5 2.0 mg/kg AD08818 | 0.286 | 0.294 | 0.298 | 0.313 |
| Group 6 1.0 mg/kg AD08818 | 0.485 | 0.102 | 0.404 | 0.243 |
| Group 7 0.5 mg/kg AD08818 | 0.531 | 0.271 | 0.845 | 0.635 |

*Only one mouse was tested on this day.

Each of the MMP7 RNAi agents in each of the dosing groups (i.e., Groups 2 through 7) showed reduction in SEAP as compared to the saline control (Group 1) across all measured time points, which as described herein, indicates inhibition of MMP7 in the MMP7-SEAP mouse model.

### Example 8. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 2.0 mg/kg (mpk) of an MMP7 RNAi agent, 1 mpk of an MMP7 RNAi agent, or saline without an MMP7 RNAi agent to be used as a control, according to the following Table 22.

TABLE 22
MMP7 RNAi Agent and Dosing for Example 8.

| Group ID | Dosing Regimen |
|---|---|
| Group 1 (isotonic saline) | Single injection on day 1 |
| Group 2 2.0 mg/kg AD08813 | Single injection on day 1 |
| Group 3 1.0 mg/kg AD08813 | Single injection on day 1 |
| Group 4 2.0 mg/kg AD08811 | Single injection on day 1 |
| Group 5 1.0 mg/kg AD08811 | Single injection on day 1 |
| Group 6 2.0 mg/kg AD08820 | Single injection on day 1 |
| Group 7 1.0 mg/kg AD08820 | Single injection on day 1 |
| Group 8 2.0 mg/kg AD08821 | Single injection on day 1 |
| Group 9 1.0 mg/kg AD08821 | Single injection on day 1 |
| Group 10 2.0 mg/kg AD08822 | Single injection on day 1 |
| Group 11 1.0 mg/kg AD08822 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i. e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 23, with Average SEAP reflecting the normalized average value of SEAP:

TABLE 23
Average SEAP normalized to pre-treatment and saline control
in MMP7-SEAP mice from Example 8.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.270 | 1.000 | 0.275 |
| Group 2 2.0 mg/kg AD08813 | 0.268 | 0.129 | 0.102 | 0.023 |
| Group 3 1.0 mg/kg AD08813 | 0.485 | 0.139 | 0.447 | 0.181 |
| Group 4 2.0 mg/kg AD08811 | 0.158 | 0.063 | 0.121 | 0.066 |
| Group 5 1.0 mg/kg AD08811 | 0.223 | 0.079 | 0.277 | 0.135 |
| Group 6 2.0 mg/kg AD08820 | 0.134 | 0.054 | ND** | ND** |
| Group 7 1.0 mg/kg AD08820 | 0.282 | 0.151 | 0.138 | 0.084 |
| Group 8 2.0 mg/kg AD08821 | 0.124 | 0.079 | 0.036 | N/A* |
| Group 9 1.0 mg/kg AD08821 | 0.607 | 0.226 | 0.121 | 0.061 |
| Group 10 2.0 mg/kg AD08822 | 0.860 | 0.320 | 0.058 | N/A* |
| Group 11 1.0 mg/kg AD08822 | 0.558 | 0.193 | 0.194 | 0.099 |
| | Day 22 | | Day 29 | |
| | Avg | Std Dev | Avg | Std Dev |
| Group ID | SEAP | (+/−) | SEAP | (+/−) |
| Group 1 (Saline) | 1.000 | 0.408 | 1.000 | 0.388 |
| Group 2 2.0 mg/kg AD08813 | 0.137 | 0.089 | 0.168 | 0.060 |
| Group 3 1.0 mg/kg AD08813 | 0.355 | 0.133 | 0.338 | 0.095 |
| Group 4 2.0 mg/kg AD08811 | 0.177 | 0.072 | 0.249 | 0.083 |
| Group 5 1.0 mg/kg AD08811 | 0.311 | 0.086 | 0.417 | 0.170 |
| Group 6 2.0 mg/kg AD08820 | 0.024 | 0.026 | 0.055 | 0.036 |
| Group 7 1.0 mg/kg AD08820 | 0.110 | 0.060 | 0.195 | 0.062 |
| Group 8 2.0 mg/kg AD08821 | 0.028 | N/A* | 0.084 | N/A* |
| Group 9 1.0 mg/kg AD08821 | 0.172 | 0.051 | 0.143 | 0.116 |
| Group 10 2.0 mg/kg AD08822 | 0.093 | 0.030 | 0.082 | N/A* |
| Group 11 1.0 mg/kg AD08822 | 0.131 | 0.047 | 0.319 | 0.125 |

*Only one mouse was tested on the day indicated
**No mice were tested on the day indicated

Each of the MMP7 RNAi agents in each of the dosing groups (i.e., Groups 2 througw 11) showed reduction in SEAP as compared to the saline control (Group 1) across all measured time points, which as described herein, indicates inhibition of MMP7 in the MMP7-SEAP mouse model.

### Example 9. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 1.5 mg/kg (mpk) of an MMP7 RNAi agent, or saline without an MMP7 RNAi agent to be used as a control, according to the following Table 24.

TABLE 24
MMP7 RNAi Agent and Dosing for Example 9.

| Group ID | Dosing Regimen |
|---|---|
| Group 1 (isotonic saline) | Single injection on day 1 |
| Group 2 1.5 mg/kg AD08815 | Single injection on day 1 |
| Group 3 1.5 mg/kg AD09243 | Single injection on day 1 |
| Group 4 1.5 mg/kg AD09244 | Single injection on day 1 |
| Group 5 1.5 mg/kg AD09245 | Single injection on day 1 |
| Group 6 1.5 mg/kg AD09246 | Single injection on day 1 |
| Group 7 1.5 mg/kg AD09247 | Single injection on day 1 |
| Group 8 1.5 mg/kg AD09248 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i. e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 25, with Average SEAP reflecting the normalized average value of SEAP:

TABLE 25
Average SEAP normalized to pre-treatment and saline control
in MMP7-SEAP mice from Example 8.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.440 | 1.000 | 0.145 |
| Group 2 1.5 mg/kg AD08815 | 0.569 | 0.186 | 0.152 | 0.065 |
| Group 3 1.5 mg/kg AD09243 | 0.282 | 0.154 | 0.075 | 0.049 |
| Group 4 1.5 mg/kg AD09244 | 0.535 | 0.169 | 0.270 | 0.122 |
| Group 5 1.5 mg/kg AD09245 | 0.659 | 0.190 | 0.211 | 0.140 |
| Group 6 1.5 mg/kg AD09246 | 0.528 | 0.100 | 0.199 | 0.110 |
| Group 7 1.5 mg/kg AD09247 | 0.613 | 0.227 | 0.269 | 0.086 |
| Group 8 1.5 mg/kg AD09248 | 1.047** | 1.439 | 0.259 | 0.079 |
| | Day 22 | | Day 29 | |
| | Avg | Std Dev | Avg | Std Dev |
| Group ID | SEAP | (+/−) | SEAP | (+/−) |
| Group 1 (Saline) | 1.000 | 0.436 | 1.000 | 0.530 |
| Group 2 1.5 mg/kg AD08815 | 0.195 | 0.071 | 0.576 | 0.425 |
| Group 3 1.5 mg/kg AD09243 | 0.095 | N/A* | 0.400 | 0.291 |
| Group 4 1.5 mg/kg AD09244 | 0.203 | 0.147 | 0.479 | 0.357 |
| Group 5 1.5 mg/kg AD09245 | 0.161 | 0.012 | 0.406 | 0.278 |
| Group 6 1.5 mg/kg AD09246 | 0.190 | 0.167 | 0.426 | 0.258 |
| Group 7 1.5 mg/kg AD09247 | 0.233 | 0.106 | 0.359 | 0.122 |
| Group 8 1.5 mg/kg AD09248 | 0.280 | 0.192 | 0.411 | 0.136 |

*Only one mouse was tested on the day indicated
**On Day 8, one animal in group 8 had a high signal

Each of the MMP7 RNAi agents in each of the dosing groups (i.e., Groups 2 through 8) showed reduction in SEAP as compared to the saline control (Group 1) across all measured time points (with the exception of Group 8 on day 8), which as described herein, indicates inhibition of MMP7 in the MMP7-SEAP mouse model.

### Example 10. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 2.0 mg/kg (mpk) of an MMP7 RNAi agent, 1.0 mpk of an MMP7 RNAi agent, or saline without an MMP7 RNAi agent to be used as a control, according to the following Table 26.

TABLE 26
MMP7 RNAi Agent and Dosing for Example 10.

| Group ID | Dosing Regimen |
|---|---|
| Group 1 (isotonic saline) | Single injection on day 1 |
| Group 2 2.0 mg/kg AD08815 | Single injection on day 1 |
| Group 3 1.0 mg/kg AD08815 | Single injection on day 1 |
| Group 4 2.0 mg/kg AD08821 | Single injection on day 1 |
| Group 5 1.0 mg/kg AD08821 | Single injection on day 1 |
| Group 6 2.0 mg/kg AD08808 | Single injection on day 1 |
| Group 7 1.0 mg/kg AD08808 | Single injection on day 1 |
| Group 8 2.0 mg/kg AD08813 | Single injection on day 1 |
| Group 9 1.0 mg/kg AD08813 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i.e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 27, with Average SEAP reflecting the normalized average value of SEAP:

TABLE 27
Average SEAP normalized to pre-treatment and saline
control in MMP7-SEAP mice from Example 10.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.468 | 1.000 | 0.469 |
| Group 2 2.0 mg/kg AD08815 | 0.170 | 0.102 | 0.177 | 0.195 |
| Group 3 1.0 mg/kg AD08815 | 0.365 | 0.193 | 0.330 | 0.161 |
| Group 4 2.0 mg/kg AD08821 | 0.109 | 0.054 | 0.069 | 0.061 |
| Group 5 1.0 mg/kg AD08821 | 0.134 | 0.045 | 0.115 | 0.046 |
| Group 6 2.0 mg/kg AD08808 | 0.115 | 0.034 | 0.107 | 0.023 |
| Group 7 1.0 mg/kg AD08808 | 0.178 | 0.057 | 0.209 | 0.130 |
| Group 8 2.0 mg/kg AD08813 | 0.167 | 0.047 | 0.108 | 0.065 |
| Group 9 1.0 mg/kg AD08813 | 0.224 | 0.028 | 0.190 | 0.091 |
| | Day 22 | | Day 29 | |
| | Avg | Std Dev | Avg | Std Dev |
| Group ID | SEAP | (+/−) | SEAP | (+/−) |
| Group 1 (Saline) | 1.000 | 0.300 | 1.000 | 0.297 |
| Group 2 2.0 mg/kg AD08815 | 0.158 | 0.131 | 0.226 | 0.232 |
| Group 3 1.0 mg/kg AD08815 | 0.439 | 0.277 | 0.457 | 0.383 |
| Group 4 2.0 mg/kg AD08821 | 0.095 | 0.074 | 0.110 | 0.092 |
| Group 5 1.0 mg/kg AD08821 | 0.289 | 0.259 | 0.412 | 0.331 |
| Group 6 2.0 mg/kg AD08808 | 0.179 | 0.077 | 0.373 | 0.294 |
| Group 7 1.0 mg/kg AD08808 | 0.299 | 0.124 | 0.397 | 0.201 |
| Group 8 2.0 mg/kg AD08813 | 0.133 | 0.083 | 0.244 | 0.217 |
| Group 9 1.0 mg/kg AD08813 | 0.324 | 0.149 | 0.360 | 0.134 |

Each of the MMP7 RNAi agents in each of the dosing groups (i.e., Groups 2 through 9) showed reduction in SEAP as compared to the saline control (Group 1) across all measured time points, which as described herein, indicates inhibition of MMP7 in the MMP7-SEAP mouse model.

### Example 11. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 1.0 mpk of an MMP7 RNAi agent, or saline without an MMP7 RNAi agent to be used as a control, according to the following Table 28.

TABLE 28
MMP7 RNAi Agent and Dosing for Example 11.

| Group ID | Dosing Regimen |
|---|---|
| Group 1 (isotonic saline) | Single injection on day 1 |
| Group 2 1.0 mg/kg AD08808 | Single injection on day 1 |
| Group 3 1.0 mg/kg AD09330 | Single injection on day 1 |
| Group 4 1.0 mg/kg AD09331 | Single injection on day 1 |
| Group 5 1.0 mg/kg AD09332 | Single injection on day 1 |
| Group 6 1.0 mg/kg AD09333 | Single injection on day 1 |
| Group 7 1.0 mg/kg AD08821 | Single injection on day 1 |
| Group 8 1.0 mg/kg AD09349 | Single injection on day 1 |
| Group 9 1.0 mg/kg AD09350 | Single injection on day 1 |
| Group 10 1.0 mg/kg AD09351 | Single injection on day 1 |
| Group 11 1.0 mg/kg AD09352 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i.e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 29, with Average SEAP reflecting the normalized average value of SEAP.

TABLE 29
Average SEAP normalized to pre-treatment and saline
control in MMP7-SEAP mice from Example 11.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.440 | 1.000 | 0.419 |
| Group 2 1.0 mg/kg AD08808 | 0.756 | 0.932 | 0.745 | 0.928 |
| Group 3 1.0 mg/kg AD09330 | 0.457 | 0.186 | 0.444 | 0.178 |
| Group 4 1.0 mg/kg AD09331 | 0.475 | 0.193 | 0.470 | 0.191 |
| Group 5 1.0 mg/kg AD09332 | 0.380 | 0.198 | 0.367 | 0.184 |
| Group 6 1.0 mg/kg AD09333 | 0.357 | 0.142 | 0.384 | 0.165 |
| Group 7 1.0 mg/kg AD08821 | 0.304 | 0.110 | 0.314 | 0.110 |
| Group 8 1.0 mg/kg AD09349 | 0.258 | 0.117 | 0.261 | 0.117 |
| Group 9 1.0 mg/kg AD09350 | 0.362 | 0.082 | 0.365 | 0.079 |
| Group 10 1.0 mg/kg AD09351 | 0.419 | 0.260 | 0.407 | 0.242 |
| Group 11 1.0 mg/kg AD09352 | 0.281 | 0.049 | 0.281 | 0.031 |
| | Day 22 | | Day 29 | |
| Group ID | Avg | Std Dev | Avg | Std Dev |
| | SEAP | (+/−) | SEAP | (+/−) |
| Group 1 (Saline) | 1.000 | 0.345 | 1.000 | 0.415 |
| Group 2 1.0 mg/kg AD08808 | 0.569 | 0.538 | 0.534 | 0.485 |
| Group 3 1.0 mg/kg AD09330 | 0.361 | 0.155 | 0.408 | 0.188 |
| Group 4 1.0 mg/kg AD09331 | 0.310 | 0.124 | 0.299 | 0.139 |
| Group 5 1.0 mg/kg AD09332 | 0.323 | 0.208 | 0.348 | 0.206 |
| Group 6 1.0 mg/kg AD09333 | 0.329 | 0.165 | 0.243 | 0.112 |
| Group 7 1.0 mg/kg AD08821 | 0.257 | 0.173 | 0.304 | 0.219 |
| Group 8 1.0 mg/kg AD09349 | 0.276 | 0.260 | 0.152 | 0.147 |
| Group 9 1.0 mg/kg AD09350 | 0.300 | 0.092 | 0.134 | 0.030 |
| Group 10 1.0 mg/kg AD09351 | 0.307 | 0.215 | 0.327 | 0.298 |
| Group 11 1.0 mg/kg AD09352 | 0.212 | 0.078 | 0.173 | 0.058 |

Each of the MMP7 RNAi agents in each of the dosing groups (i.e., Groups 2 through 11) showed reduction in SEAP as compared to the saline control (Group 1) across all measured time points, which as described herein, indicates inhibition of MMP7 in the MMP7-SEAP mouse model.

### Example 12. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 1.0 mpk of an MMP7 RNAi agent, or saline without an MMP7 RNAi agent to be used as a control, according to the following Table 30.

TABLE 30
MMP7 RNAi Agent and Dosing for Example 12.

| Group ID | Dosing Regimen |
|---|---|
| Group 1 (isotonic saline) | Single injection on day 1 |
| Group 2 1.0 mg/kg AD08813 | Single injection on day 1 |
| Group 3 1.0 mg/kg AD09334 | Single injection on day 1 |
| Group 4 1.0 mg/kg AD09335 | Single injection on day 1 |
| Group 5 1.0 mg/kg AD09336 | Single injection on day 1 |
| Group 6 1.0 mg/kg AD09337 | Single injection on day 1 |
| Group 7 1.0 mg/kg AD08811 | Single injection on day 1 |
| Group 8 1.0 mg/kg AD09354 | Single injection on day 1 |
| Group 9 1.0 mg/kg AD09355 | Single injection on day 1 |
| Group 10 1.0 mg/kg AD09356 | Single injection on day 1 |
| Group 11 1.0 mg/kg AD09357 | Single injection on day 1 |
| Group 12 1.0 mg/kg AD09358 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i.e. subcutaneous injections) into the loose skin over the neck and shoulder area. Four (4) mice in each group were tested (n=4). Serum was collected on day 8, day 15, and day 22, and SEAP expression levels were determined pursuant to the procedure set forth in Example 2, above. Data from the experiment are shown in the following Table 31, with Average SEAP reflecting the normalized average value of SEAP.

TABLE 31
Average SEAP normalized to pre-treatment and saline
control in MMP7-SEAP mice from Example 12.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.303 | 1.000 | 0.313 |
| Group 2 1.0 mg/kg AD08813 | 0.889 | 0.357 | 0.342 | 0.166 |
| Group 3 1.0 mg/kg AD09334 | 0.924 | 0.368 | 0.587 | 0.256 |
| Group 4 1.0 mg/kg AD09335 | 0.869 | 0.269 | 0.680 | 0.414 |
| Group 5 1.0 mg/kg AD09336 | 0.826 | 0.233 | 0.468 | 0.146 |
| Group 6 1.0 mg/kg AD09337 | 0.850 | 0.176 | 0.483 | 0.195 |
| Group 7 1.0 mg/kg AD08811 | 0.454 | 0.147 | 0.249 | 0.185 |
| Group 8 1.0 mg/kg AD09354 | 0.833 | 0.353 | 0.678 | 0.349 |
| Group 9 1.0 mg/kg AD09355 | 0.715 | 0.174 | 0.485 | 0.191 |
| Group 10 1.0 mg/kg AD09356 | 1.182 | 0.280 | 0.428 | 0.351 |
| Group 11 1.0 mg/kg AD09357 | 0.897 | 0.384 | 0.491 | 0.224 |
| Group 12 1.0 mg/kg AD09358 | 0.701 | 0.237 | 0.465 | 0.126 |

| | Day 22 | |
|---|---|---|
| | Avg | Std Dev |
| Group ID | SEAP | (+/−) |
| Group 1 (Saline) | 1.000 | 0.460 |
| Group 2 1.0 mg/kg AD08813 | 0.495 | 0.322 |
| Group 3 1.0 mg/kg AD09334 | 0.574 | 0.266 |
| Group 4 1.0 mg/kg AD09335 | 0.563 | 0.335 |
| Group 5 1.0 mg/kg AD09336 | 0.721 | 0.282 |
| Group 6 1.0 mg/kg AD09337 | 0.603 | 0.300 |
| Group 7 1.0 mg/kg AD08811 | 0.982 | 0.517 |
| Group 8 1.0 mg/kg AD09354 | 0.386 | 0.268 |
| Group 9 1.0 mg/kg AD09355 | 0.615 | 0.152 |
| Group 10 1.0 mg/kg AD09356 | 1.188 | 0.560 |
| Group 11 1.0 mg/kg AD09357 | 0.862 | 0.427 |
| Group 12 1.0 mg/kg AD09358 | 0.828 | 0.248 |

Each of the MMP7 RNAi agents in each of the dosing groups except for Group 10 (i.e., Groups 2 through 9 and 11 and 12) showed reduction in SEAP as compared to the saline control (Group 1) across all measured time points, which as described herein, indicates inhibition of MMP7 in the MMP7-SEAP mouse model.

### Example 13. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 1.0 mpk of an MMP7 RNAi agent, or saline without an MMP7 RNAi agent to be used as a control, according to the following Table 32.

TABLE 32
MMP7 RNAi Agent and Dosing for Example 13.

| Group ID | Dosing Regimen |
|---|---|
| Group 1 (isotonic saline) | Single injection on day 1 |
| Group 2 1.0 mg/kg AD08803 | Single injection on day 1 |
| Group 3 1.0 mg/kg AD09441 | Single injection on day 1 |
| Group 4 1.0 mg/kg AD09442 | Single injection on day 1 |
| Group 5 1.0 mg/kg AD09443 | Single injection on day 1 |
| Group 6 1.0 mg/kg AD09444 | Single injection on day 1 |
| Group 7 1.0 mg/kg AD08823 | Single injection on day 1 |
| Group 8 1.0 mg/kg AD09445 | Single injection on day 1 |
| Group 9 1.0 mg/kg AD09446 | Single injection on day 1 |
| Group 10 1.0 mg/kg AD09447 | Single injection on day 1 |
| Group 11 1.0 mg/kg AD09448 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i.e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 33, with Average SEAP reflecting the normalized average value of SEAP.

TABLE 33
Average SEAP normalized to pre-treatment and saline
control in MMP7-SEAP mice from Example 13.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.210 | 1.000 | 0.188 |
| Group 2 1.0 mg/kg AD08803 | 0.383 | 0.125 | 0.387 | 0.143 |
| Group 3 1.0 mg/kg AD09441 | 0.785 | 0.192 | 0.695 | 0.185 |
| Group 4 1.0 mg/kg AD09442 | 0.780 | 0.285 | 0.820 | 0.321 |
| Group 5 1.0 mg/kg AD09443 | 0.749 | 0.353 | 0.733 | 0.493 |
| Group 6 1.0 mg/kg AD09444 | 0.451 | 0.208 | 0.342 | 0.094 |
| Group 7 1.0 mg/kg AD08823 | 0.249 | 0.064 | 0.189 | 0.079 |
| Group 8 1.0 mg/kg AD09445 | 0.260 | 0.095 | 0.135 | 0.103 |
| Group 9 1.0 mg/kg AD09446 | 0.162 | 0.056 | 0.162 | 0.086 |
| Group 10 1.0 mg/kg AD09447 | 0.177 | 0.030 | 0.126 | 0.049 |
| Group 11 1.0 mg/kg AD09448 | 0.360 | 0.139 | 0.273 | 0.126 |
| | Day 22 | | Day 29 | |
| | Avg | Std Dev | Avg | Std Dev |
| Group ID | SEAP | (+/−) | SEAP | (+/−) |
| Group 1 (Saline) | 1.000 | 0.376 | 1.000 | 0.429 |
| Group 2 1.0 mg/kg AD08803 | 0.418 | 0.101 | ND* | ND* |
| Group 3 1.0 mg/kg AD09441 | 0.826 | 0.393 | ND* | ND* |
| Group 4 1.0 mg/kg AD09442 | 1.186 | 0.656 | ND* | ND* |
| Group 5 1.0 mg/kg AD09443 | 0.940 | 0.656 | ND* | ND* |
| Group 6 1.0 mg/kg AD09444 | 0.568 | 0.230 | ND* | ND* |
| Group 7 1.0 mg/kg AD08823 | 0.295 | 0.169 | 0.655 | 0.373 |
| Group 8 1.0 mg/kg AD09445 | 0.307 | 0.262 | 0.519 | 0.397 |
| Group 9 1.0 mg/kg AD09446 | 0.173 | 0.113 | 0.338 | 0.238 |
| Group 10 1.0 mg/kg AD09447 | 0.182 | 0.061 | 0.299 | 0.081 |
| Group 11 1.0 mg/kg AD09448 | 0.545 | 0.352 | 0.699 | 0.384 |

*Mice from Groups 2-6 were euthanized prior to day 29

Each of the MMP7 RNAi agents in each of the dosing groups, except for Group 4 on day 22 (i.e., Groups 2, 3 and 5 through 11), showed reduction in SEAP as compared to the saline control (Group 1) across all measured time points, which as described herein, indicates inhibition of MMP7 in the MMP7-SEAP mouse model.

### Example 14. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 3.0 mg/kg (mpk) of an MMP7 RNAi agent, 1.0 mpk of an MMP7 RNAi agent, 0.3 mpk of an MMP7 RNAi agent, or saline without an MMP7 RNAi agent to be used as a control, according to the following Table 34.

TABLE 34
MMP7 RNAi Agent and Dosing for Example 14.

| Group ID | Dosing Regimen |
|---|---|
| Group 1 (isotonic saline) | Single injection on day 1 |
| Group 2 0.3 mg/kg AD08821 | Single injection on day 1 |
| Group 3 1.0 mg/kg AD08821 | Single injection on day 1 |
| Group 4 3.0 mg/kg AD08821 | Single injection on day 1 |
| Group 5 0.3 mg/kg AD09350 | Single injection on day 1 |
| Group 6 1.0 mg/kg AD09350 | Single injection on day 1 |
| Group 7 3.0 mg/kg AD09350 | Single injection on day 1 |
| Group 8 0.3 mg/kg AD09352 | Single injection on day 1 |
| Group 9 1.0 mg/kg AD09352 | Single injection on day 1 |
| Group 10 3.0 mg/kg AD09352 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i.e. subcutaneous injections) into the loose skin over the neck and shoulder area. Four (4) mice in each group were tested (n=4). Serum was collected on day 8, day 15, day 22, and SEAP expression levels were determined pursuant to the procedure set forth in Example 2, above. Data from the experiment are shown in the following Table 35, with Average SEAP reflecting the normalized average value of SEAP.

TABLE 35
Average SEAP normalized to pre-treatment and saline
control in MMP7-SEAP mice from Example 14.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.482 | 1.000 | 0.382 |
| Group 2 0.3 mg/kg AD08821 | 0.681 | 0.235 | 0.655 | 0.419 |
| Group 3 1.0 mg/kg AD08821 | 0.291 | 0.063 | 0.251 | 0.164 |
| Group 4 3.0 mg/kg AD08821 | 0.136 | 0.051 | 0.084 | 0.076 |
| Group 5 0.3 mg/kg AD09350 | 0.507 | 0.066 | 0.685 | 0.130 |
| Group 6 1.0 mg/kg AD09350 | 0.250 | 0.090 | 0.211 | 0.093 |
| Group 7 3.0 mg/kg AD09350 | 0.098 | 0.036 | 0.044 | 0.019 |
| Group 8 0.3 mg/kg AD09352 | 0.861 | 0.180 | 0.948 | 0.401 |
| Group 9 1.0 mg/kg AD09352 | 0.295 | 0.142 | 0.203 | 0.100 |
| Group 10 3.0 mg/kg AD09352 | 0.166 | 0.043 | 0.004 | 0.005 |

| | Day 22 | |
|---|---|---|
| | Avg | Std Dev |
| Group ID | SEAP | (+/−) |
| Group 1 (Saline) | 1.000 | 0.514 |
| Group 2 0.3 mg/kg AD08821 | 0.511 | 0.302 |
| Group 3 1.0 mg/kg AD08821 | 0.168 | 0.103 |
| Group 4 3.0 mg/kg AD08821 | 0.047 | 0.024 |
| Group 5 0.3 mg/kg AD09350 | 0.426 | 0.083 |
| Group 6 1.0 mg/kg AD09350 | 0.105 | 0.073 |
| Group 7 3.0 mg/kg AD09350 | 0.026 | 0.014 |
| Group 8 0.3 mg/kg AD09352 | 0.660 | 0.103 |
| Group 9 1.0 mg/kg AD09352 | 0.154 | 0.082 |
| Group 10 3.0 mg/kg AD09352 | 0.024 | 0.006 |

Each of the MMP7 RNAi agents in each of the dosing groups (i.e., Groups 2 through 10) showed reduction in SEAP as compared to the saline control (Group 1) across all measured time points, which as described herein, indicates inhibition of MMP7 in the MMP7-SEAP mouse model.

### Example 15. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 3.0 mg/kg (mpk) of an MMP7 RNAi agent, 1.0 mpk of an MMP7 RNAi agent, 0.3 mpk of an MMP7 RNAi agent, or saline without an MMP7 RNAi agent to be used as a control, according to the following Table 36.

TABLE 36
MMP7 RNAi Agent and Dosing for Example 15.

| Group ID | Dosing Regimen |
|---|---|
| Group 1 (isotonic saline) | Single injection on day 1 |
| Group 2 0.3 mg/kg AD08808 | Single injection on day 1 |
| Group 3 1.0 mg/kg AD08808 | Single injection on day 1 |
| Group 4 3.0 mg/kg AD08808 | Single injection on day 1 |
| Group 5 0.3 mg/kg AD09332 | Single injection on day 1 |
| Group 6 1.0 mg/kg AD09332 | Single injection on day 1 |
| Group 7 3.0 mg/kg AD09332 | Single injection on day 1 |
| Group 8 0.3 mg/kg AD09333 | Single injection on day 1 |
| Group 9 1.0 mg/kg AD09333 | Single injection on day 1 |
| Group 10 3.0 mg/kg AD09333 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i. e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 37, with Average SEAP reflecting the normalized average value of SEAP:

TABLE 37
Average SEAP normalized to pre-treatment and saline
control in MMP7-SEAP mice from Example 15.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.334 | 1.000 | 0.473 |
| Group 2 0.3 mg/kg AD08808 | 0.679 | 0.506 | 0.503 | 0.164 |
| Group 3 1.0 mg/kg AD08808 | 0.365 | 0.134 | 0.316 | 0.158 |
| Group 4 3.0 mg/kg AD08808 | 0.116 | 0.069 | 0.099 | 0.084 |
| Group 5 0.3 mg/kg AD09332 | 0.482 | 0.124 | 0.337 | 0.264 |
| Group 6 1.0 mg/kg AD09332 | 0.342 | 0.177 | 0.244 | 0.091 |
| Group 7 3.0 mg/kg AD09332 | 0.139 | 0.100 | 0.066 | 0.075 |
| Group 8 0.3 mg/kg AD09333 | 0.562 | 0.377 | 0.502 | 0.250 |
| Group 9 1.0 mg/kg AD09333 | 0.305 | 0.108 | 0.191 | 0.055 |
| Group 10 3.0 mg/kg AD09333 | 0.125 | 0.020 | 0.037 | 0.020 |
| | Day 22 | | Day 29 | |
| | Avg | Std Dev | Avg | Std Dev |
| Group ID | SEAP | (+/−) | SEAP | (+/−) |
| Group 1 (Saline) | 1.000 | 0.310 | 1.000 | 0.619 |
| Group 2 0.3 mg/kg AD08808 | 1.407 | 2.130 | 0.907 | 0.778 |
| Group 3 1.0 mg/kg AD08808 | 0.308 | 0.131 | 0.333 | 0.270 |
| Group 4 3.0 mg/kg AD08808 | 0.095 | 0.062 | 0.127 | 0.083 |
| Group 5 0.3 mg/kg AD09332 | 1.652 | 2.697 | 0.471 | 0.259 |
| Group 6 1.0 mg/kg AD09332 | 0.298 | 0.121 | 0.275 | 0.313 |
| Group 7 3.0 mg/kg AD09332 | 0.065 | 0.052 | 0.070 | 0.045 |
| Group 8 0.3 mg/kg AD09333 | 0.635 | 0.240 | 0.687 | 0.321 |
| Group 9 1.0 mg/kg AD09333 | 0.236 | 0.129 | 0.289 | 0.219 |
| Group 10 3.0 mg/kg AD09333 | 0.075 | 0.052 | 0.061 | 0.039 |

Each of the MMP7 RNAi agents in each of the dosing groups, except for Groups 2 and 5 on day 22, showed reduction in SEAP as compared to the saline control (Group 1) across all measured time points, which as described herein, indicates inhibition of MMP7 in the MMP7-SEAP mouse model.

### Example 16. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 1.0 mg/kg (mpk) of an MMP7 RNAi agent, 0.5 mpk of an MMP7 RNAi agent, or saline without an MMP7 RNAi agent to be used as a control, according to the following Table 38.

TABLE 38
MMP7 RNAi Agent and Dosing for Example 16.

| Group ID | Dosing Regimen |
|---|---|
| Group 1 (isotonic saline) | Single injection on day 1 |
| Group 2 1.0 mg/kg AD08811 | Single injection on day 1 |
| Group 3 0.5 mg/kg AD08811 | Single injection on day 1 |
| Group 4 1.0 mg/kg AD08823 | Single injection on day 1 |
| Group 5 0.5 mg/kg AD08823 | Single injection on day 1 |
| Group 6 1.0 mg/kg AD09246 | Single injection on day 1 |
| Group 7 0.5 mg/kg AD09246 | Single injection on day 1 |
| Group 8 1.0 mg/kg AD09247 | Single injection on day 1 |
| Group 9 0.5 mg/kg AD09247 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i. e. subcutaneous injections) into the loose skin over the neck and shoulder area. Four (4) mice in each group were tested (n=4). Serum was collected on day 8, day 15, and day 22, and SEAP expression levels were determined pursuant to the procedure set forth in Example 2, above. Data from the experiment are shown in the following Table 39, with Average SEAP reflecting the normalized average value of SEAP:

TABLE 39
Average SEAP normalized to pre-treatment and saline
control in MMP7-SEAP mice from Example 16.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.737 | 1.000 | 0.652 |
| Group 2 1.0 mg/kg AD08811 | 0.982 | 1.098 | 1.114 | 1.386 |
| Group 3 0.5 mg/kg AD08811 | 2.107 | 1.950 | 2.015 | 1.885 |
| Group 4 1.0 mg/kg AD08823 | 0.831 | 0.728 | 0.750 | 0.643 |
| Group 5 0.5 mg/kg AD08823 | 0.818 | 0.371 | 0.802 | 0.459 |
| Group 6 1.0 mg/kg AD09246 | 0.347 | 0.223 | 0.257 | 0.173 |
| Group 7 0.5 mg/kg AD09246 | 0.592 | 0.649 | 0.535 | 0.640 |
| Group 8 1.0 mg/kg AD09247 | 0.240 | 0.265 | 0.214 | 0.225 |
| Group 9 0.5 mg/kg AD09247 | 1.108 | 0.878 | 2.426 | 1.497 |

| | Day 22 | |
|---|---|---|
| | Avg | Std Dev |
| Group ID | SEAP | (+/−) |
| Group 1 (Saline) | 1.000 | 0.663 |
| Group 2 1.0 mg/kg AD08811 | 1.699 | 2.201 |
| Group 3 0.5 mg/kg AD08811 | 3.642 | 3.831 |
| Group 4 1.0 mg/kg AD08823 | 0.918 | 0.907 |
| Group 5 0.5 mg/kg AD08823 | 0.966 | 0.480 |
| Group 6 1.0 mg/kg AD09246 | 0.441 | 0.439 |
| Group 7 0.5 mg/kg AD09246 | 0.795 | 0.983 |
| Group 8 1.0 mg/kg AD09247 | 0.285 | 0.300 |
| Group 9 0.5 mg/kg AD09247 | 0.868 | 0.630 |

Each of the MMP7 RNAi agents in each of the dosing groups 4 through 8 showed reduction in SEAP as compared to the saline control (Group 1) across all measured time points, which as described herein, indicates inhibition of MMP7 in the MMP7-SEAP mouse model.

### Example 17. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 1.0 mpk of an MMP7 RNAi agent, or saline without an MMP7 RNAi agent to be used as a control, according to the following Table 40.

TABLE 40
MMP7 RNAi Agent and Dosing for Example 17.

| Group ID | Dosing Regimen |
|---|---|
| Group 1 (isotonic saline) | Single injection on day 1 |
| Group 2 1.0 mg/kg AD08821 | Single injection on day 1 |
| Group 3 1.0 mg/kg AD09890 | Single injection on day 1 |
| Group 4 1.0 mg/kg AD09891 | Single injection on day 1 |
| Group 5 1.0 mg/kg AD09352 | Single injection on day 1 |
| Group 6 1.0 mg/kg AD09908 | Single injection on day 1 |
| Group 7 1.0 mg/kg AD09909 | Single injection on day 1 |
| Group 8 1.0 mg/kg AD09330 | Single injection on day 1 |
| Group 9 1.0 mg/kg AD09892 | Single injection on day 1 |
| Group 10 1.0 mg/kg AD09332 | Single injection on day 1 |
| Group 11 1.0 mg/kg AD09894 | Single injection on day 1 |
| Group 12 1.0 mg/kg AD09895 | Single injection on day 1 |
| Group 13 1.0 mg/kg AD08801 | Single injection on day 1 |
| Group 14 1.0 mg/kg AD09896 | Single injection on day 1 |
| Group 15 1.0 mg/kg AD09897 | Single injection on day 1 |
| Group 16 1.0 mg/kg AD09898 | Single injection on day 1 |
| Group 17 1.0 mg/kg AD09899 | Single injection on day 1 |
| Group 18 1.0 mg/kg AD08811 | Single injection on day 1 |
| Group 19 1.0 mg/kg AD08823 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i. e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 41, with Average SEAP reflecting the normalized average value of SEAP:

TABLE 41
Average SEAP normalized to pre-treatment and saline control
in MMP7-SEAP mice from Example 17.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.429 | 1.000 | 0.282 |
| Group 2 1.0 mg/kg AD08821 | 0.204 | 0.056 | 0.201 | 0.080 |
| Group 3 1.0 mg/kg AD09890 | 0.179 | 0.120 | 0.204 | 0.117 |
| Group 4 1.0 mg/kg AD09891 | 0.142 | 0.062 | 0.149 | 0.088 |
| Group 5 1.0 mg/kg AD09352 | 0.178 | 0.069 | 0.185 | 0.105 |
| Group 6 1.0 mg/kg AD09908 | 0.211 | 0.070 | 0.148 | 0.062 |
| Group 7 1.0 mg/kg AD09909 | 0.162 | 0.068 | 0.096 | 0.062 |
| Group 8 1.0 mg/kg AD09330 | 0.445 | 0.199 | 0.558 | 0.290 |
| Group 9 1.0 mg/kg AD09892 | 0.295 | 0.130 | 0.480 | 0.263 |
| Group 10 1.0 mg/kg AD09332 | 0.318 | 0.135 | 0.273 | 0.142 |
| Group 11 1.0 mg/kg AD09894 | 0.218 | 0.106 | 0.190 | 0.127 |
| Group 12 1.0 mg/kg AD09895 | 0.120 | 0.035 | 0.127 | 0.063 |
| Group 13 1.0 mg/kg AD08801 | 0.387 | 0.037 | 0.533 | 0.206 |
| Group 14 1.0 mg/kg AD09896 | 0.414 | 0.162 | 0.648 | 0.330 |
| Group 15 1.0 mg/kg AD09897 | 0.682 | 0.497 | 0.740 | 0.487 |
| Group 16 1.0 mg/kg AD09898 | 0.559 | 0.196 | 0.746 | 0.256 |
| Group 17 1.0 mg/kg AD09899 | 0.316 | 0.069 | 0.415 | 0.191 |
| Group 18 1.0 mg/kg AD08811 | 0.270 | 0.062 | 0.367 | 0.054 |
| Group 19 1.0 mg/kg AD08823 | 0.213 | 0.057 | 0.291 | 0.079 |
| | Day 22 | | Day 29 | |
| | Avg | Std Dev | Avg | Std Dev |
| Group ID | SEAP | (+/−) | SEAP | (+/−) |
| Group 1 (Saline) | 1.000 | 0.354 | 1.000 | 0.137 |
| Group 2 1.0 mg/kg AD08821 | 0.192 | 0.102 | 0.244 | 0.174 |
| Group 3 1.0 mg/kg AD09890 | 0.212 | 0.107 | 0.345 | 0.218 |
| Group 4 1.0 mg/kg AD09891 | 0.148 | 0.092 | 0.149 | 0.133 |
| Group 5 1.0 mg/kg AD09352 | 0.165 | 0.089 | 0.299 | 0.201 |
| Group 6 1.0 mg/kg AD09908 | 0.118 | 0.055 | 0.184 | 0.108 |
| Group 7 1.0 mg/kg AD09909 | 0.152 | 0.110 | 0.225 | 0.172 |
| Group 8 1.0 mg/kg AD09330 | 0.552 | 0.273 | 0.722 | 0.362 |
| Group 9 1.0 mg/kg AD09892 | 0.477 | 0.257 | 0.851 | 0.596 |
| Group 10 1.0 mg/kg AD09332 | 0.251 | 0.148 | 0.461 | 0.322 |
| Group 11 1.0 mg/kg AD09894 | 0.214 | 0.156 | 0.276 | 0.243 |
| Group 12 1.0 mg/kg AD09895 | 0.122 | 0.048 | 0.154 | 0.067 |
| Group 13 1.0 mg/kg AD08801 | 0.423 | 0.060 | 0.555 | 0.107 |
| Group 14 1.0 mg/kg AD09896 | 0.667 | 0.280 | 0.762 | 0.393 |
| Group 15 1.0 mg/kg AD09897 | 0.574 | 0.336 | 0.941 | 0.413 |
| Group 16 1.0 mg/kg AD09898 | 0.795 | 0.460 | 0.952 | 0.381 |
| Group 17 1.0 mg/kg AD09899 | 0.437 | 0.230 | 0.487 | 0.290 |
| Group 18 1.0 mg/kg AD08811 | 0.381 | 0.067 | 0.610 | 0.196 |
| Group 19 1.0 mg/kg AD08823 | 0.158 | 0.049 | 0.241 | 0.051 |

Each of the MMP7 RNAi agents in each of the dosing groups (i.e., Groups 2 through 19) showed reduction in SEAP as compared to the saline control (Group 1) across all measured time points, which as described herein, indicates inhibition of MMP7 in the MMP7-SEAP mouse model.

### Example 18. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 1.0 mpk of an MMP7 RNAi agent, or saline without an MMP7 RNAi agent to be used as a control, according to the following Table 42.

TABLE 42
MMP7 RNAi Agent and Dosing for Example 18.

| Group ID | Dosing Regimen |
|---|---|
| Group 1 (isotonic saline) | Single injection on day 1 |
| Group 2 1.0 mg/kg AD08811 | Single injection on day 1 |
| Group 3 1.0 mg/kg AD09354 | Single injection on day 1 |
| Group 4 1.0 mg/kg AD09356 | Single injection on day 1 |
| Group 5 1.0 mg/kg AD09357 | Single injection on day 1 |
| Group 6 1.0 mg/kg AD09358 | Single injection on day 1 |
| Group 7 1.0 mg/kg AD09900 | Single injection on day 1 |
| Group 8 1.0 mg/kg AD09901 | Single injection on day 1 |
| Group 9 1.0 mg/kg AD09902 | Single injection on day 1 |
| Group 10 1.0 mg/kg AD09903 | Single injection on day 1 |

Each of the MMP7 RNAi agents included N-acetyl-galactosamine targeting ligands conjugated to the 5′-terminal end of the sense strand, as shown in Tables 5 and 7B. The injections were performed between the skin and muscle (i.e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 43, with Average SEAP reflecting the normalized average value of SEAP.

TABLE 43
Average SEAP normalized to pre-treatment and saline control
in MMP7-SEAP mice from Example 18.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.177 | 1.000 | 0.343 |
| Group 2 1.0 mg/kg AD08811 | 0.322 | 0.148 | 0.341 | 0.274 |
| Group 3 1.0 mg/kg AD09354 | 0.508 | 0.197 | 0.411 | 0.144 |
| Group 4 1.0 mg/kg AD09356 | 0.561 | 0.222 | 0.528 | 0.171 |
| Group 5 1.0 mg/kg AD09357 | 0.414 | 0.150 | 0.335 | 0.167 |
| Group 6 1.0 mg/kg AD09358 | 0.298 | 0.029 | 0.207 | 0.093 |
| Group 7 1.0 mg/kg AD09900 | 0.356 | 0.208 | 0.111 | 0.096 |
| Group 8 1.0 mg/kg AD09901 | 0.581 | 0.053 | 0.360 | 0.077 |
| Group 9 1.0 mg/kg AD09902 | 0.404 | 0.242 | 0.273 | 0.174 |
| Group 10 1.0 mg/kg AD09903 | 0.492 | 0.221 | 0.317 | 0.194 |
| | Day 22 | | Day 29 | |
| | Avg | Std Dev | Avg | Std Dev |
| Group ID | SEAP | (+/−) | SEAP | (+/−) |
| Group 1 (Saline) | 1.000 | 0.356 | 1.000 | 0.328 |
| Group 2 1.0 mg/kg AD08811 | 0.493 | 0.227 | 0.623 | 0.316 |
| Group 3 1.0 mg/kg AD09354 | 0.478 | 0.244 | 0.535 | 0.241 |
| Group 4 1.0 mg/kg AD09356 | 0.654 | 0.341 | 0.622 | 0.304 |
| Group 5 1.0 mg/kg AD09357 | 0.999 | 0.550 | 0.429 | 0.295 |
| Group 6 1.0 mg/kg AD09358 | 0.398 | 0.475 | 0.241 | 0.108 |
| Group 7 1.0 mg/kg AD09900 | 0.195 | 0.155 | 0.273 | 0.213 |
| Group 8 1.0 mg/kg AD09901 | 0.358 | 0.146 | 0.354 | 0.220 |
| Group 9 1.0 mg/kg AD09902 | 0.318 | 0.248 | 0.273 | 0.149 |
| Group 10 1.0 mg/kg AD09903 | 0.375 | 0.241 | 0.399 | 0.230 |

Each of the MMP7 RNAi agents in each of the dosing groups (i.e., Groups 2 through 10) showed reduction in SEAP as compared to the saline control (Group 1) across all measured time points, which as described herein, indicates inhibition of MMP7 in the MMP7-SEAP mouse model.

### Example 19. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 0.75 mpk of an MMP7 RNAi agent, or saline without an MMP7 RNAi agent to be used as a control, according to the following Table 44.

TABLE 44
MMP7 RNAi Agent and Dosing for Example 19.

| Group ID | Dosing Regimen |
|---|---|
| Group 1 (isotonic saline) | Single injection on day 1 |
| Group 2 0.75 mg/kg AD08811 | Single injection on day 1 |
| Group 3 0.75 mg/kg AD10284 | Single injection on day 1 |
| Group 4 0.75 mg/kg AD09900 | Single injection on day 1 |
| Group 5 0.75 mg/kg AD10285 | Single injection on day 1 |
| Group 6 0.75 mg/kg AD09358 | Single injection on day 1 |
| Group 7 0.75 mg/kg AD10286 | Single injection on day 1 |
| Group 8 0.75 mg/kg AD10287 | Single injection on day 1 |
| Group 9 0.75 mg/kg AD10288 | Single injection on day 1 |
| Group 10 | Single injection on day 1 |
| 0.75 mg/kg AD10290 | |
| Group 11 | Single injection on day 1 |
| 0.75 mg/kg AD10291 | |
| Group 12 | Single injection on day 1 |
| 0.75 mg/kg AD10292 | |

Each of the MMP7 RNAi agents included N-acetyl-galactosamine targeting ligands conjugated to the 5′-terminal end of the sense strand, as shown in Tables 5 and 7B. The injections were performed between the skin and muscle (i.e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 45, with Average SEAP reflecting the normalized average value of SEAP:

TABLE 45
Average SEAP normalized to pre-treatment and saline control
in MMP7-SEAP mice from Example 19.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.149 | 1.000 | 0.109 |
| Group 2 0.75 mg/kg AD08811 | 0.439 | 0.147 | 0.447 | 0.150 |
| Group 3 0.75 mg/kg AD10284 | 0.362 | 0.124 | 0.517 | 0.229 |
| Group 4 0.75 mg/kg AD09900 | 0.473 | 0.107 | 0.363 | 0.204 |
| Group 5 0.75 mg/kg AD10285 | 0.354 | 0.117 | 0.298 | 0.143 |
| Group 6 0.75 mg/kg AD09358 | 0.573 | 0.280 | 0.315 | 0.155 |
| Group 7 0.75 mg/kg AD10286 | 0.392 | 0.198 | 0.257 | 0.213 |
| Group 8 0.75 mg/kg AD10287 | 0.448 | 0.166 | 0.259 | 0.083 |
| Group 9 0.75 mg/kg AD10288 | 0.304 | 0.126 | 0.196 | 0.086 |
| Group 10 | 0.360 | 0.138 | 0.166 | 0.074 |
| 0.75 mg/kg AD10290 | | | | |
| Group 11 | 0.354 | 0.188 | 0.273 | 0.144 |
| 0.75 mg/kg AD10291 | | | | |
| Group 12 | 0.914 | 1.247 | 0.661 | 0.865 |
| 0.75 mg/kg AD10292 | | | | |
| | Day 22 | | Day 29 | |
| | Avg | Std Dev | Avg | Std Dev |
| Group ID | SEAP | (+/−) | SEAP | (+/−) |
| Group 1 (Saline) | 1.000 | 0.273 | 1.000 | 0.300 |
| Group 2 0.75 mg/kg AD08811 | 0.445 | 0.083 | 0.357 | 0.171 |
| Group 3 0.75 mg/kg AD10284 | 0.448 | 0.173 | 0.203 | 0.192 |
| Group 4 0.75 mg/kg AD09900 | 0.330 | 0.133 | 0.263 | 0.110 |
| Group 5 0.75 mg/kg AD10285 | 0.284 | 0.086 | 0.207 | 0.095 |
| Group 6 0.75 mg/kg AD09358 | 0.512 | 0.266 | 0.403 | 0.188 |
| Group 7 0.75 mg/kg AD10286 | 0.313 | 0.133 | 0.250 | 0.178 |
| Group 8 0.75 mg/kg AD10287 | 0.310 | 0.065 | 0.224 | 0.026 |
| Group 9 0.75 mg/kg AD10288 | 0.214 | 0.183 | 0.203 | 0.161 |
| Group 10 | 0.158 | 0.073 | 0.111 | 0.059 |
| 0.75 mg/kg AD10290 | | | | |
| Group 11 | 0.274 | 0.108 | 0.331 | 0.193 |
| 0.75 mg/kg AD10291 | | | | |
| Group 12 | 0.676 | 0.892 | 0.702 | 0.726 |
| 0.75 mg/kg AD10292 | | | | |

Each of the MMP7 RNAi agents in each of the dosing groups (i.e., Groups 2 through 12) showed reduction in SEAP as compared to the saline control (Group 1) across all measured time points, which as described herein, indicates inhibition of MMP7 in the MMP7-SEAP mouse model.

### Example 20. In Vivo Testing of MMP7 RNAi Agents in MMP7-SEAP Mice

The MMP7-SEAP mouse model described in Example 2, above, was used. At day 1, each mouse was given a single subcutaneous injection of 200 μl per 20 g body weight containing either 1.0 mg/kg (mpk) of an MMP7 RNAi agent, 0.5 mpk of an MMP7 RNAi agent, or saline without an MMP7 RNAi agent to be used as a control, according to the following Table 46.

TABLE 46
MMP7 RNAi Agent and Dosing for Example 20.

| Group ID | Dosing Regimen |
|---|---|
| Group 1 (isotonic saline) | Single injection on day 1 |
| Group 2 1.0 mg/kg | Single injection on day 1 |
| AD09358 | |
| Group 3 0.5 mg/kg | Single injection on day 1 |
| AD09358 | |
| Group 4 1.0 mg/kg | Single injection on day 1 |
| AD10288 | |
| Group 5 0.5 mg/kg | Single injection on day 1 |
| AD10288 | |
| Group 6 1.0 mg/kg | Single injection on day 1 |
| AD10290 | |
| Group 7 0.5 mg/kg | Single injection on day 1 |
| AD10290 | |
| Group 8 1.0 mg/kg | Single injection on day 1 |
| AD09909 | |
| Group 9 0.5 mg/kg | Single injection on day 1 |
| AD09909 | |

Each of the MMP7 RNAi agents included N-acetyl-galactosamine targeting ligands conjugated to the 5′-terminal end of the sense strand, as shown in Tables 5 and 7B. The injections were performed between the skin and muscle (i.e. subcutaneous injections) into the loose skin over the neck and shoulder area. Four (4) mice in each group were tested (n=4). Serum was collected on day 8, day 15, and day 22, and SEAP expression levels were determined pursuant to the procedure set forth in Example 2, above. Data from the experiment are shown in the following Table 47, with Average SEAP reflecting the normalized average value of SEAP.

TABLE 47
Average SEAP normalized to pre-treatment and saline control in
MMP7-SEAP mice from Example 20.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (Saline) | 1.000 | 0.087 | 1.000 | 0.128 |
| Group 2 1.0 mg/kg AD09358 | 0.494 | 0.178 | 0.479 | 0.264 |
| Group 3 0.5 mg/kg AD09358 | 0.577 | 0.182 | 0.659 | 0.287 |
| Group 4 1.0 mg/kg AD10288 | 0.333 | 0.144 | 0.307 | 0.242 |
| Group 5 0.5 mg/kg AD10288 | 0.510 | 0.218 | 0.569 | 0.252 |
| Group 6 1.0 mg/kg AD10290 | 0.273 | 0.069 | 0.303 | 0.226 |
| Group 7 0.5 mg/kg AD10290 | 0.571 | 0.303 | 0.447 | 0.351 |
| Group 8 1.0 mg/kg AD09909 | 0.129 | 0.075 | 0.054 | 0.019 |
| Group 9 0.5 mg/kg AD09909 | 0.290 | 0.129 | 0.164 | 0.093 |

| | Day 22 | |
|---|---|---|
| Group ID | Avg SEAP | Std Dev (+/−) |
| Group 1 (Saline) | 1.000 | 0.205 |
| Group 2 1.0 mg/kg AD09358 | 0.523 | 0.296 |
| Group 3 0.5 mg/kg AD09358 | 0.963 | 0.452 |
| Group 4 1.0 mg/kg AD10288 | 0.403 | 0.392 |
| Group 5 0.5 mg/kg AD10288 | 0.471 | 0.213 |
| Group 6 1.0 mg/kg AD10290 | 0.301 | 0.155 |
| Group 7 0.5 mg/kg AD10290 | 0.517 | 0.564 |
| Group 8 1.0 mg/kg AD09909 | 0.051 | 0.011 |
| Group 9 0.5 mg/kg AD09909 | 0.198 | 0.152 |

Each of the MMP7 RNAi agents in all of the dosing groups (i.e., Groups 2 through 9) showed reduction in SEAP as compared to the saline control (Group 1) across all measured time points, which as described herein, indicates inhibition of MMP7 in the MMP7-SEAP mouse model.

### Example 21. In Vivo Inhaled Aerosolized Administration of MMP7 RNAi Agents in Cynomolgus Monkeys

On study day 1, male cynomolgus monkeys were administered a single deposited dose level of 1 mg/kg of the MMP7 RNAi agent AC001514, AC001651, or AC001516 (see Tables 8, 6, and 3 for structural information.). Twelve anesthetized male non-human primates (NHPs) were exposed to either aerosolized Isotonic Saline (control article), AC001514, AC001516 or AC001651 (test articles) using an endotracheal inhalation delivery system. All animals received a single inhalation exposure. In this study, aerosols generated using Aeroneb Solo nebulizers were delivered using a Harvard ventilation pump. The exposure duration for each of these tests was 8 minutes. Dose exposures in vivo were estimated by collecting aerosol on a filter at the end of the endotracheal tube in a separate in vitro test. One filter test was performed before and after each in vivo exposure. The filters were analyzed by gravimetric and chemical methods. A UV spectrometric method using SpectraMax i3x was deployed for the chemical analysis of the filters to determine the amount of test article administered over the course of the exposure.

The average deposited doses of saline control, AC001514, AC001516 and AC001651 were 0.0, 1.04, 1.12 and 1.21 mg/kg, respectively. Target deposited doses for each of the three test articles for this study were 1.0 mg/kg. The MMP7 RNAi agent was conjugated to a tridentate small molecule αvβ6 epithelial cell targeting ligand (Tri-SM6.1, see Table 11) at the 5′ terminal end of the sense strand, formulated in isotonic saline. The dosing groups were as follows:

TABLE 48
MMP7 RNAi Agent and Dosing for Example 21.

| | AC Duplex |
|---|---|
| Group ID | Number |
| Group 1 (isotonic saline) | N/A |
| Group 2 (1.0 mg/kg target deposited dose | AC001514 |
| Tri-SM6.1-αvß6-AD09667) | |
| Group 3 (1.0 mg/kg target deposited dose | AC001651 |
| Tri-SM6.1-αvß6-AD09887) | |
| Group 4 (1.0 mg/kg target deposited dose | AC001516 |
| Tri-SM6.1-αvß6-AD09669) | |

Three (3) monkeys were dosed per group. Bronchoalveolar lavage (BAL) and endobronchial brushings were collected at baseline and two weeks after a single inhalation exposure. All animals were euthanized just after blood and BAL sample collection to collect tissue of interest. Monkeys were sacrificed on study day 15, and total RNA was isolated from lung samples following collection and homogenization. The data in Table 49 shows mRNA expression sampled from the right cranial lobe, the right middle lobe, and the right caudal lobe. Cynomolgus monkey MMP7 mRNA expression was quantitated by probe-based quantitative PCR, normalized to Cynomolgus monkey ARL1 expression, and expressed as fraction of vehicle control group (geometric mean, +/−95% confidence interval).

TABLE 49
Average Relative Cynomolgus Monkey MMP7 mRNA Expression at
Sacrifice in Example 21.

| Hilar Region | | | Middle Region | | | Peripheral Region | | |
|---|---|---|---|---|---|---|---|---|
| | Low | High | | Low | High | | Low | High |
| Average | (error) | (error) | Average | (error) | (error) | Average | (error) | (error) |

Right Cranial Lobe

| | | | | | | | | | |
|---|---|---|---|---|---|---|---|---|---|
| Group 1 | 1.000 | 0.354 | 0.547 | 1.000 | 0.438 | 0.780 | 1.000 | 0.412 | 0.699 |
| (isotonic | | | | | | | | | |
| saline) | | | | | | | | | |
| Group 2 | 0.778 | 0.379 | 0.740 | 0.264 | 0.180 | 0.562 | 0.412 | 0.161 | 0.265 |
| (1.0 mg/kg | | | | | | | | | |
| AC001514) | | | | | | | | | |
| Group 3 | 0.115 | 0.049 | 0.085 | 0.202 | 0.093 | 0.173 | 0.461 | 0.278 | 0.703 |
| (1.0 mg/kg | | | | | | | | | |
| AC001651) | | | | | | | | | |
| Group 4 | 0.181 | 0.098 | 0.215 | 0.173 | 0.112 | 0.321 | 0.466 | 0.294 | 0.796 |
| (1.0 mg/kg | | | | | | | | | |
| AC001516) | | | | | | | | | |

Right Middle Lobe

| | | | | | | | | | |
|---|---|---|---|---|---|---|---|---|---|
| Group 1 | 1.000 | 0.227 | 0.293 | 1.000 | 0.466 | 0.874 | 1.000 | 0.366 | 0.577 |
| (isotonic | | | | | | | | | |
| saline) | | | | | | | | | |
| Group 2 | 0.537 | 0.081 | 0.096 | 0.362 | 0.172 | 0.328 | 0.548 | 0.163 | 0.231 |
| (1.0 mg/kg | | | | | | | | | |
| AC001514) | | | | | | | | | |
| Group 3 | 0.358 | 0.078 | 0.099 | 0.071 | 0.016 | 0.020 | 0.188 | 0.053 | 0.074 |
| (1.0 mg/kg | | | | | | | | | |
| AC001651) | | | | | | | | | |
| Group 4 | 0.265 | 0.158 | 0.392 | 0.574 | 0.288 | 0.579 | 0.682 | 0.372 | 0.817 |
| (1.0 mg/kg | | | | | | | | | |
| AC001516) | | | | | | | | | |

Right Caudal Lobe

| | | | | | | | | | |
|---|---|---|---|---|---|---|---|---|---|
| Group 1 | 1.000 | 0.459 | 0.848 | 1.000 | 0.425 | 0.739 | 1.000 | 0.359 | 0.561 |
| (isotonic | | | | | | | | | |
| saline) | | | | | | | | | |
| Group 2 | 1.180 | 0.184 | 0.219 | 0.565 | 0.043 | 0.047 | 0.583 | 0.136 | 0.177 |
| (1.0 mg/kg | | | | | | | | | |
| AC001514) | | | | | | | | | |
| Group 3 | 0.259 | 0.081 | 0.118 | 0.137 | 0.071 | 0.147 | 0.149 | 0.044 | 0.062 |
| (1.0 mg/kg | | | | | | | | | |
| AC001651) | | | | | | | | | |
| Group 4 | 0.580 | 0.347 | 0.863 | 0.843 | 0.358 | 0.622 | 0.664 | 0.416 | 1.111 |
| (1.0 mg/kg | | | | | | | | | |
| AC001516) | | | | | | | | | |

As shown in the data in Table 49 above, RNAi agents AC001514, AC001651 and AC001516 showed substantial inhibition across various regions and lobes of the lung demonstrating the ability to robustly silence MMP7 expression in non-human primates.

Cynomolgus monkey MMP7 protein expression in the lung tissues and BAL was quantitated by Western blot using iBright imaging system (Thermofisher), shown in the following Table 50.

TABLE 50
Average Relative Cynomolgus Monkey MMP7 Protein Expression
at Sacrifice in Example 21.

| | MMP7 Protein | | | |
|---|---|---|---|---|
| | expression in | | | |
| | lung tissue | | | |
| | (Right | | MMP7 protein | |
| | Accessory | | expression | |
| | Lobe) | | in the BAL | |
| | | Std | | Std |
| | Avg | Dev | Avg | Dev |
| Group ID | MMP7 | (+/−) | MMP7 | (+/−) |
| Group 1 (isotonic saline) | 1.00 | 0.519 | 1.00 | 0.613 |
| Group 2 (1.0 mg/kg AC001514) | N/A | | N/A | |
| Group 3 (1.0 mg/kg AC001651) | 0.181 | 0.031 | 0.171 | 0.145 |

| | | |
|---|---|---|
| Group 4 (1.0 mg/kg AC001516) | N/A | N/A |

As shown in the data in Table 50 above, RNAi agent AC001651 silences MMP7 expression, showing over 80% reduction in protein expression in both lung tissues and BAL in non-human primates.

### Example 22. AAV6.2FF-CAG-hMMP7.UTRs AAV Mouse Model

The following procedure was used to evaluate MMP7 RNAi agents in an AAV mouse model. To evaluate certain MMP7 RNAi agents, a AAV6.2FF-CAG-hMMP7.UTRs (Adeno-associated virus) mouse model was used. The transgenic sequence included human MMP7 CDS with 3′UTR. Six- to eight-week-old female C57BL/6 mice were transduced with human MMP7 using AAV with serotype 6.2FF. Mice were intratracheally administered at least 10 days prior to mutiple intracheal administration of MMP7 RNAi agents or control. Two types of AAV, AAV6.2FF-CAG-hMMP7.UTRs and AAV6.2FF-CAG-eGFP were used. The genome of the AAV6.2FF-CAG-hMMP7.UTRs construct contains the 17-1119 region of the human MMP7 cDNA sequence (GenBank NM_002423.5). AAV6.2FF-CAG-eGFP was co-dosed with AAV6.2FF-CAG-hMMP7.UTRs. eGFP was used as endogenous control to normalize human MMP7 mRNA expression by qPCR. 2E10 to 4E10 GC of the respective virus mixed in PBS in a total volume of 50 μL was intratracheally (IT) delivered into mice to create AAV-hMMP7 model mice. Lung tissues and bronchoalveolar lavage fluid (BALF) were collected 2-3 weeks after the administration of RNAi agents.

The human MMP7 mRNA and protein level expressions were measured in the lung tissues by qPCR and Western Blot. Human MMP7 protein expression in bronchoalveolar lavage fluid (BALF) was measured by ELISA.

At day 1 and Day 3, each mouse was given an intratracheal (IT) administration of 50 μL AAV solutions containing 1 GC (genome copy) of AAV6.2FF-CAG-eGFP and 2 GC of AAV6.2FF-CAG-hMMP7.UTRs in PBS or vehicle control (PBS). At Day 13, 15, and 17, each mouse was given intratracheal administration of 50 μL of different dose levels of MMP7 RNAi agents formulated in isotonic saline, or vehicle control (isotonic saline with no RNAi agent), according to the following Table 51.

TABLE 51
Targeted Positions and Dosing Groups of Example 22.

| | Targeted MMP7 Gene | | | | |
|---|---|---|---|---|---|
| | Position (within SEQ ID NO: | | RNAi Agent | | Dosing |
| Group | 1, GenBank NM_002423.5) | AAV dose | and Dose | | Regimen |
| 1 | N/A | PBS | Saline | | Mutiple IT |
| | | | (no RNAi | | doses of PBS |
| | | | agent) | | on Day 1, 3; |
| | | | | | Mutiple IT |
| | | | | | doses of |
| | | | | | saline on |
| | | | | | day 13, 15, 17 |
| 2 | N/A | 1GC of AAV6.2FF- | Saline | | Mutiple IT |
| | | CAG-eGFP and 2GC of | (no RNAi | | doses of AAV |
| | | AAV6.2FF-CAG- | agent) | | on Day 1, 3; |
| | | hMMP7.UTRs | | | Mutiple |
| | | | | | IT doses of |
| | | | | | saline on |
| | | | | | day 13, 15, 17 |
| 3 | 971 | 1GC of AAV6.2FF- | 3 | mg/kg | Mutiple IT |
| | | CAG-eGFP and 2GC of | AC001651 | | doses of AAV |
| | | AAV6.2FF-CAG- | | | on Day 1, 3; |
| | | hMMP7.UTRs | | | Mutiple IT |
| | | | | | doses of |
| | | | | | RNAi agent on |
| | | | | | day 13, 15, 17 |
| 4 | 971 | 1GC of AAV6.2FF- | 1.5 | mg/kg | Mutiple IT |
| | | CAG-eGFP and 2GC of | AC001651 | | doses of AAV |
| | | AAV6.2FF-CAG- | | | on Day 1, 3; |
| | | hMMP7.UTRs | | | Mutiple IT |
| | | | | | doses of RNAi |
| | | | | | agent on day |
| | | | | | 13, 15, 17 |
| 5 | 971 | 1GC of AAV6.2FF- | 0.75 | mg/kg | Mutiple IT |
| | | CAG-eGFP and 2GC of | AC001651 | | doses of AAV |
| | | AAV6.2FF-CAG- | | | on Day 1, 3; |
| | | hMMP7.UTRs | | | Mutiple IT |
| | | | | | doses of RNAi |
| | | | | | agent on day |
| | | | | | 13, 15, 17 |
| 6 | 735 | 1GC of AAV6.2FF- | 3 | mg/kg | Mutiple IT |
| | | CAG-eGFP and 2GC of | AC002023 | | doses of AAV |
| | | AAV6.2FF-CAG- | | | on Day 1, 3; |
| | | hMMP7.UTRs | | | Mutiple IT |
| | | | | | doses of RNAi |
| | | | | | agent on day |
| | | | | | 13, 15, 17 |
| 7 | 735 | 1GC of AAV6.2FF- | 1.5 | mg/kg | Mutiple IT |
| | | CAG-eGFP and 2GC of | AC002023 | | doses of AAV |
| | | AAV6.2FF-CAG- | | | on Day 1, 3; |
| | | hMMP7.UTRs | | | Mutiple IT |
| | | | | | doses of RNAi |
| | | | | | agent on day |
| | | | | | 13, 15, 17 |
| 8 | 735 | 1GC of AAV6.2FF- | 0.75 | mg/kg | Mutiple IT |

| | | |
|---|---|---|
| CAG-eGFP and 2GC of | AC002023 | doses of AAV |
| AAV6.2FF-CAG- | | on Day 1, 3; |
| hMMP7.UTRs | | Mutiple IT |
| | | doses of RNAi |
| | | agent on day |
| | | 13, 15, 17 |

Each of the MMP7 RNAi agents included modified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the MMP7 RNAi agents, including Tri-SM6.1-αvβ6). The MMP7 RNAi agents in Groups 3-8 each included nucleotide sequences that were designed to inhibit expression of a MMP7 gene by targeting specific positions of MMP7 mRNA as set forth in Table 50, above. (See, e.g., SEQ ID NO:1 and Table 2 for the MMP7 mRNA sequence referenced.)

Four (4) mice in each group were tested (n=4). MMP7 expression levels were determined pursuant to the procedure set forth above. Dose response of MMP7 RNAi agent AC001516 was observed in AAV6.2FF-CAG-hMMP7.UTRs mouse model. Data from the experiment are shown in the following Table 52:

TABLE 52
Average MMP7 Normalized to Control in AAV-hMMP7 Mice from Example 22.

| | Day 31 Lung tissue | | Day 31 BALF | | Day 31 | |
|---|---|---|---|---|---|---|
| | mRNA | | protein | | Lung tissue protein | |
| | Avg | Std Dev | Avg | Std Dev | Avg | Std Dev |
| Group ID | MMP7 | (+/-) | MMP7 | (+/-) | MMP7 | (+/-) |
| Group 1 (Saline vehicle) | N/A | | N/A | | N/A | |
| Group 2 (Saline vehicle) | 1.00 | 0.266 | 1.000 | 0.217 | 1.00 | 0.563 |
| Group 3 (3 mg/kg AC001651) | 0.319 | 0.159 | 0.174 | 0.061 | 0.358 | 0.077 |
| Group 4 (1.5 mg/kg AC001651) | 0.424 | 0.126 | 0.224 | 0.020 | 0.404 | 0.048 |
| Group 5 (0.75 mg/kg AC001651) | 0.606 | 0.105 | 0.440 | 0.155 | 0.524 | 0.083 |
| Group 6 (3 mg/kg AC002023) | 0.945 | 0.216 | 0.866 | 0.249 | 0.780 | 0.195 |
| Group 7 (1.5 mg/kg AC002023) | 1.100 | 0.119 | 1.046 | 0.734 | N/A | |
| Group 8 (0.75 mg/kg AC002023) | 1.100 | 0.186 | 0.753 | 0.364 | N/A | |

As shown in Table 52, above, the RNAi agent of Group 3 (targeting position 971) was active and showed reductions of approximately 68% on day 31 (0.159) at mRNA level in the lung tissues; 83% reduction of secreted human MMP7 protein and 64% reduction of human MMP7 protein in lung tissues.

### Example 23. Proof of Concept Efficacy Studies Using Rodent-Specific MMP7 RNAi Agent in Rat Bleomycin-Induced Injury Model

It has been demonstrated that MMP7 knockout mice are protected against bleomycin-mediated lung injury (Proc Natl Acad Sci USA. 2002; 99:6292-6297). To evaluate MMP7 RNAi agents, several proof of concept efficacy studies were developed using rodent-specific MMP7 RNAi agent in a rat bleomycin-induced injury model. MMP7 expression in a mouse bleomycin injury model was evaluated, and MMP7 mRNA levels were found to be not increased in lung tissues after bleomycin-induced injury. Furthermore, MMP7 expression transiently increased in rat lung tissues after bleomycin-induced injury. MMP7 expression peaked at 7-10 days after bleomycin-induced injury and decreased to baseline levels over 4-week period after injury.

One rat-specific RNAi agent was selected after screening and optimization in a rat bleomycin-induced injury model. Bleomycin was used to induce injury in rat. After injury, the RNAi agent was administered to the rat via 1.4 mg/kg single inhalation or multiple 3.0 mg/kg intratracheal doses. In the 2 to 4 weeks after the bleomycin-induced injury, the RNAi agent achieved 60-90% gene silencing of MMP7. Silencing MMP7 in the lung significantly attenuated lung injury in rat bleomycin model with reduced pulmonary fibrosis Ashcroft histology scores, reduced collagen deposition, decreased inflammatory response with reduced eosinophils and neutrophils in lavage fluid, decreased translatable fibrotic gene expression including Col1a2, Col5a1, Grem1, Cthrc1, Muc16. Furthermore, silencing MMP7 significantly improved pulmonary function with increased functional compliance, preserved blood oxygen content, less body weight loss and reduced mortality. MMP7 silencing by rodent-specific RNAi agent effectively protects lung from fibrosis development in intratracheal bleomycin injury model in rats.

### Example 24. Passive Uptake of MMP7 RNAi Agents in Human Precision Cut Lung Slices (PCLS)

Precision cut tissue slices (PCLS) represent an ex vivo model and tool for studying the structure and function of the lung in its native 3D environment, allowing for examination of the natural interactions between cells, molecules, and the extracellular matrix (ECM) ex vivo (Alsafadi H. N. et al, Am J Respir Cell Mol Biol 62(6): 681-691 (2020)). PCLS can be generated from various anatomical locations of the lung (distal and proximal), and from different species, including rodents, pigs, monkeys, and humans. However, the effectiveness of passive uptake of RNAi agents in human PCLS is still unclear. To validate the RNAi agent potency for silencing human MMP7 mRNA, fresh agarose inflated lung slices from healthy human donors were used for examination.

Saline or MMP7 RNAi agents were added to cell media, with daily media changes. The PCLS were cultured in the media from Day 1 to Day 7 and harvested at Day 8. mRNA expression of MMP7 and potential off target genes MAP3K9, MTF2, NUP107 were quantitated by qPCR normalized by endogenous control PPIA. Effective passive uptake of MMP7 RNAi agents was observed. Little off target effects was detected. MMP7 RNAi agent was administered according to the following Table 53. Data from the qPCR experiment are shown in the following Table 54.

Test Groups 11 and 12 were dosed with 1 μM AC002026. AC002026 is an RNAi agent duplex with the same modified antisense and sense strand sequences as those of AC001514. However, the AC002026 sense strand is conjugated to an inactive enantiomer of the αvβ6 integrin targeting ligand. Due to the difference in stereochemistry, this chemically modified analogue of αvβ6 integrin targeting ligand is unable to effectively bind to αvβ6 integrin, and therefore unable to effectively support cellular intake of the AC002026 RNAi agent.

TABLE 53
Dosing groups and dosing regimen of MMP7 RNAi
agents from Example 24.

| Group ID | Dosing Regimen |
|---|---|
| Group 1 (isotonic saline) | Treatment daily in the cell |
| | media for 7 days |
| Group 2 AC001514 1 μM | Treatment daily in the cell |
| | media for 7 days |
| Group 3 AC001514 0.3 μM | Treatment daily in the cell |
| | media for 7 days |
| Group 4 AC001514 0.1 μM | Treatment daily in the cell |
| | media for 7 days |
| Group 5 AC001651 1 μM | Treatment daily in the cell |
| | media for 7 days |
| Group 6 AC001651 0.3 μM | Treatment daily in the cell |
| | media for 7 days |
| Group 7 AC001651 0.1 μM | Treatment daily in the cell |
| | media for 7 days |
| Group 8 AC002023 1 μM | Treatment daily in the cell |
| | media for 7 days |
| Group 9 AC002023 0.3 μM | Treatment daily in the cell |
| | media for 7 days |
| Group 10 AC002023 0.1 μM | Treatment daily in the cell |
| | media for 7 days |
| Group 11 AC002026 1 μM | Treatment daily in the cell |
| | media for 7 days |
| Group 12 AC002026 1 μM | Treatment daily in the cell |
| | media for 7 days |

TABLE 54
mRNA Expression normalized to human endogenous control PPIA, in human PCLS from Example 24.

| | Avg | Std Dev | Avg | Std Dev | Avg | Std Dev | Avg | Std Dev |
|---|---|---|---|---|---|---|---|---|
| Group ID | MMP7 | (+/−) | MTF2 | (+/−) | MAP3K9 | (+/−) | NUP107 | (+/−) |
| Group 1 (isotonic saline) | 1.000 | 0.698 | 1.000 | 0.288 | 1.000 | 0.252 | 1.000 | 0.479 |
| Group 2 AC001514 1 μM | 0.414 | 1.132 | 1.196 | 0.610 | 1.394 | 0.631 | 0.904 | 0.779 |
| Group 3 AC001514 0.3 μM | 0.616 | 0.960 | 1.003 | 0.569 | 1.091 | 0.655 | 0.637 | 0.579 |
| Group 4 AC001514 0.1 μM | 0.470 | 0.509 | 0.680 | 1.065 | 0.847 | 0.489 | 0.466 | 1.327 |
| Group 5 AC001651 1 μM | 0.104 | 0.736 | 0.917 | 0.543 | 0.755 | 0.335 | 0.483 | 1.143 |
| Group 6 AC001651 0.3 μM | 0.102 | 0.284 | 1.575 | 0.492 | 1.221 | 0.867 | 0.675 | 0.633 |
| Group 7 AC001651 0.1 μM | 0.184 | 0.900 | 1.125 | 0.477 | 1.228 | 0.183 | 0.507 | 0.610 |
| Group 8 AC002023 1 μM | 0.769 | 1.522 | 0.888 | 0.703 | 1.500 | 0.417 | 0.601 | 0.589 |
| Group 9 AC002023 0.3 μM | 0.760 | 0.566 | 0.727 | 0.734 | 0.983 | 0.286 | 0.563 | 0.740 |
| Group 10 AC002023 0.1 μM | 0.651 | 0.961 | 0.918 | 0.408 | 1.565 | 0.505 | 0.606 | 0.473 |
| Group 11 AC002026 1 μM | 0.702 | 0.706 | 0.544 | 0.446 | 0.972 | 0.473 | 0.376 | 0.789 |
| Group 12 AC002026 1 μM | 0.603 | 1.081 | 0.863 | 0.757 | 0.972 | 0.472 | 0.713 | 1.106 |

Passive uptake of MMP7 RNAi agents was observed. As shown above in Table 54, the MMP7 RNAi agent uptake resulted in up to 82-90% MMP7 silencing from the most potent RNAi agent AC001651. This potency result is of similar rank order as the in vivo SEAP studies.

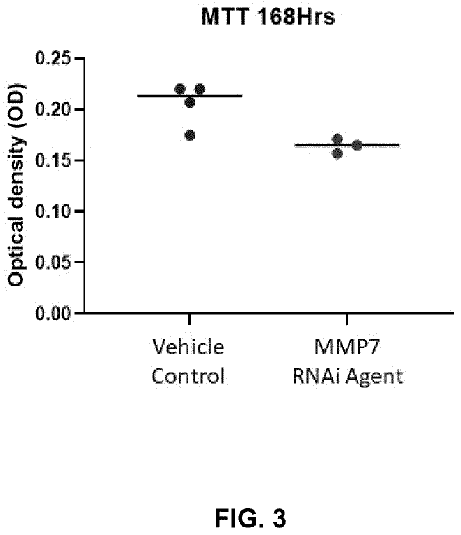

Concurrently, few off target effects were observed. The RNAi agents, upon dosing, caused only minor cytotoxic effects even at the highest dosing concentration. Such cytotoxicity and cell viability were demonstrated via MTT colorimetric assay for cell metabolic and mitochondrial activity. MTT assay showed MMP7 RNAi agent exhibiting comparable optical density (OD) as that of the control at 168 hours after dosing, as shown in FIG. 3.

### Example 25. In Vivo Inhaled Aerosolized Administration of MMP7 RNAi Agents in Cynomolgus Monkeys

On study day 1, male cynomolgus monkeys were administered a single deposited dose level of 0.24 mg/kg, 0.66 mg/kg, 1.10 mg/kg, or 1.71 mg/kg of the MMP7 RNAi agent AC001651 (see Tables 8, 6, and 3 for structural information.) or isotonic saline. Three (3) animals per group (n=3), anesthetized male non-human primates (NHPs) were exposed to either aerosolized Isotonic Saline (control article) or AC001651 (test articles) via inhalation using the face mask inhalation exposure system on Day 1. In this study, aerosols generated using a Hudson Updraft II compressed air jet nebulizer and delivered to animals by face mask inhalation. Concentration in test atmospheres were determined by gravimetric analysis of filter samples (47-mm fiber film filters, 0.5 micron, GF/A) collected during all exposures at a nominal flow. After collection, the filters were removed from the filter holders and weighed. Additionally, the contents of the filters were analyzed chemically by absorbance measurement on a SpectraMax i3x spectrophotometer. AC001651 formulation samples were analyzed for concentration at the time of filter analysis.

Target deposited doses for each of the three test articles for this study were 0.25 mg/kg, 0.5 mg/kg, 1.0 mg/kg and 2.0 mg/kg, respectively. The average deposited doses of saline control and AC001651 were 0.0, 0.24, 0.66, 1.10 and 1.71 mg/kg, respectively. The MMP7 RNAi agent was conjugated to a tridentate small molecule αvβ6 epithelial cell targeting ligand (Tri-SM6.1, see Table 11) at the 5′ terminal end of the sense strand, formulated in isotonic saline. The dosing groups were as follows:

TABLE 55
MMP7 RNAi Agent and Dosing for Example 25.

| | AC Duplex |
|---|---|
| Group ID | Number |
| Group 1 (isotonic saline) | N/A |
| Group 2 (0.25 mg/kg target deposited dose | AC001651 |
| Tri-SM6.1-αvß6-AD09887) | |
| (Average Pulmonary Deposited Dose PDD: | |
| 0.24 mg/kg) | |
| Group 3 (0.5 mg/kg target deposited dose | AC001651 |
| Tri-SM6.1-αvß6-AD09887) | |
| (Average Pulmonary Deposited Dose PDD: | |
| 0.66 mg/kg) | |
| Group 4 (1.0 mg/kg target deposited dose | AC001651 |
| Tri-SM6.1-αvß6-AD09887) | |
| (Average Pulmonary Deposited Dose PDD: | |
| 1.10 mg/kg) | |
| Group 5 (2.0 mg/kg target | AC001651 |
| deposited dose Tri-SM6.1-αvß6-AD09887) | |
| (Average Pulmonary Deposited Dose PDD: | |
| 1.71 mg/kg) | |

Three (3) monkeys were dosed per group. Bronchoalveolar lavage (BAL) fluid samples were collected at baseline on Day 1 and two weeks after a single inhalation exposure on Day 14. All animals were euthanized just after blood and BAL sample collection to collect tissue of interest. Monkeys were sacrificed on study day 14, and total RNA was isolated from lung samples following collection and homogenization. The data in Table 56 shows mRNA expression sampled from the right caudal lobe, the right cranial lobe, the right middle lobe, and the left caudal lobe. Cynomolgus monkey MMP7 mRNA expression was quantitated by probe-based quantitative PCR, normalized to Cynomolgus monkey GAPDH mRNA expression, and the vehicle control group (geometric mean, +/−geometric SD).

TABLE 56
Average Relative Cynomolgus Monkey MMP7 mRNA Expression at
Sacrifice in Example 25.

| Hilar Region | | | Middle Region | | | Peripheral Region | | |
|---|---|---|---|---|---|---|---|---|
| | Low | High | | Low | High | | Low | High |
| Average | (error) | (error) | Average | (error) | (error) | Average | (error) | (error) |

Right Caudal Lobe

| | | | | | | | | | |
|---|---|---|---|---|---|---|---|---|---|
| Group 1 | 1.000 | 0.630 | 1.700 | 1.000 | 0.540 | 1.176 | 1.000 | 0.514 | 1.060 |
| (isotonic | | | | | | | | | |
| saline) | | | | | | | | | |
| Group 2 | 1.113 | 0.269 | 0.354 | 0.609 | 0.078 | 0.089 | 1.099 | 0.238 | 0.304 |
| (0.24 | | | | | | | | | |
| mg/kg | | | | | | | | | |
| AC001651) | | | | | | | | | |
| Group 3 | 0.311 | 0.144 | 0.267 | 0.262 | 0.090 | 0.137 | 0.437 | 0.128 | 0.181 |
| (0.66 | | | | | | | | | |
| mg/kg | | | | | | | | | |
| AC001651) | | | | | | | | | |
| Group 4 | 0.259 | 0.067 | 0.091 | 0.240 | 0.105 | 0.188 | 0.310 | 0.141 | 0.258 |
| (1.10 | | | | | | | | | |
| mg/kg | | | | | | | | | |
| AC001651) | | | | | | | | | |
| Group 5 | 0.583 | 0.313 | 0.674 | 0.251 | 0.111 | 0.200 | 0.359 | 0.167 | 0.313 |
| (1.71 | | | | | | | | | |
| mg/kg | | | | | | | | | |
| AC001651) | | | | | | | | | |

Right Cranial Lobe

| | | | | | | | | | |
|---|---|---|---|---|---|---|---|---|---|
| Group 1 | 1.000 | 0.522 | 1.091 | 1.000 | 0.426 | 0.742 | 1.000 | 0.439 | 0.782 |
| (isotonic | | | | | | | | | |
| saline) | | | | | | | | | |
| Group 2 | 0.630 | 0.126 | 0.157 | 0.965 | 0.280 | 0.395 | 1.416 | 0.313 | 0.401 |
| (0.24 | | | | | | | | | |
| mg/kg | | | | | | | | | |
| AC001651) | | | | | | | | | |
| Group 3 | 0.390 | 0.150 | 0.243 | 0.448 | 0.210 | 0.395 | 0.385 | 0.077 | 0.097 |
| (0.66 | | | | | | | | | |
| mg/kg | | | | | | | | | |
| AC001651) | | | | | | | | | |
| Group 4 | 0.216 | 0.093 | 0.162 | 0.221 | 0.087 | 0.142 | 0.193 | 0.054 | 0.075 |
| (1.10 | | | | | | | | | |
| mg/kg | | | | | | | | | |
| AC001651) | | | | | | | | | |
| Group 5 | 0.222 | 0.131 | 0.318 | 0.307 | 0.133 | 0.236 | 0.559 | 0.316 | 0.728 |
| (1.71 | | | | | | | | | |
| mg/kg | | | | | | | | | |
| AC001651) | | | | | | | | | |

Right Middle Lobe

| | | | | | | | | | |
|---|---|---|---|---|---|---|---|---|---|
| Group 1 | 1.000 | 0.572 | 1.336 | 1.000 | 0.425 | 0.741 | 1.000 | 0.443 | 0.796 |
| (isotonic | | | | | | | | | |
| saline) | | | | | | | | | |
| Group 2 | 1.011 | 0.127 | 0.146 | 0.544 | 0.095 | 0.114 | 0.349 | 0.138 | 0.227 |
| (0.24 | | | | | | | | | |
| mg/kg | | | | | | | | | |
| AC001651) | | | | | | | | | |
| Group 3 | 0.337 | 0.107 | 0.157 | 0.301 | 0.066 | 0.084 | 0.217 | 0.033 | 0.039 |
| (0.66 | | | | | | | | | |
| mg/kg | | | | | | | | | |
| AC001651) | | | | | | | | | |
| Group 4 | 0.339 | 0.131 | 0.214 | 0.207 | 0.099 | 0.190 | 0.143 | 0.072 | 0.146 |
| (1.10 | | | | | | | | | |
| mg/kg | | | | | | | | | |
| AC001651) | | | | | | | | | |
| Group 5 | 0.236 | 0.118 | 0.238 | 0.224 | 0.096 | 0.169 | 0.265 | 0.097 | 0.154 |

(1.71
mg/kg
AC001651)
Left Middle Lobe

| | | | | | | | | | |
|---|---|---|---|---|---|---|---|---|---|
| Group 1 | 1.000 | 0.579 | 1.376 | 1.000 | 0.504 | 1.014 | 1.000 | 0.461 | 0.856 |
| (isotonic | | | | | | | | | |
| saline) | | | | | | | | | |
| Group 2 | 1.460 | 0.162 | 0.182 | 1.208 | 0.150 | 0.171 | 0.996 | 0.280 | 0.390 |
| (0.24 | | | | | | | | | |
| mg/kg | | | | | | | | | |
| AC001651) | | | | | | | | | |
| Group 3 | 0.464 | 0.205 | 0.366 | 0.438 | 0.131 | 0.186 | 0.513 | 0.269 | 0.564 |
| (0.66 | | | | | | | | | |
| mg/kg | | | | | | | | | |
| AC001651) | | | | | | | | | |
| Group 4 | 0.252 | 0.119 | 0.226 | 0.355 | 0.182 | 0.373 | 0.222 | 0.101 | 0.186 |
| (1.10 | | | | | | | | | |
| mg/kg | | | | | | | | | |
| AC001651) | | | | | | | | | |
| Group 5 | 0.400 | 0.190 | 0.363 | 0.366 | 0.154 | 0.265 | 0.364 | 0.164 | 0.298 |
| (1.71 | | | | | | | | | |
| mg/kg | | | | | | | | | |
| AC001651) | | | | | | | | | |

As shown in the data in Table 56 above, RNAi agent AC001651 showed substantial inhibition across various regions and lobes of the lung demonstrating the ability to robustly silence MMP7 expression in non-human primates.

Cynomolgus monkey MMP7 protein expression in the lung tissues was quantitated by Western blot using iBright imaging system (Thermo Fisher), shown in the following Table 57.

TABLE 57
Average Relative Cynomolgus Monkey MMP7 Protein Expression
at Sacrifice in Example 25.

| | MMP7 Protein expression | |
|---|---|---|
| | in lung tissue | |
| | (Right Caudal Lobe) | |
| Group ID | Avg MMP7 | Std Dev (+/−) |
| Group 1 (isotonic saline) | 1.000 | 0.772 |
| Group 2 (0.24 mg/kg PDD AC001651) | 0.778 | 0.258 |
| Group 3 (0.66 mg/kg PDD AC001651) | 0.394 | 0.166 |
| Group 4 (1.10 mg/kg PDD AC001651) | 0.309 | 0.160 |
| Group 5 (1.71 mg/kg PDD AC001651) | 0.399 | 0.206 |

As shown in the data in Table 57 above, RNAi agent AC001651 silences MMP7 expression, showing average ˜69% reduction in protein expression at 1.10 mg/kg deposited dose in lung tissues in non-human primates. PDD=pulmonary deposited dose.

MMP7 mRNA expression in BAL exosome in cynomolgus monkeys treated with single deposited dose of 0.24 mg/kg, 0.66 mg/kg, 1.10 mg/kg, or 1.71 mg/kg PDD of RNAi agent AC001651 was quantified via qPCR. The data are normalized to baseline Day −7 and GAPDH and the vehicle control group (GMEAN+/− with geometric standard deviation. The data is shown below in Table 58.

TABLE 58
MMP7 mRNA expression in BAL exosome, relative to GAPDH.

| | Baseline (Day-7) | | Day 14 | |
|---|---|---|---|---|
| | | Std | | Std |
| | Avg | Dev | Avg | Dev |
| Group ID | MMP7 | (+/−) | MMP7 | (+/−) |
| Group 1 (isotonic saline) | 1.000 | 1.045 | 2.071 | 0.717 |
| Group 2 | 1.000 | 1.416 | 1.252 | 1.110 |
| (0.24 mg/kg PDD AC001651) | | | | |
| Group 3 | 1.000 | 0.806 | 0.428 | 1.406 |
| (0.66 mg/kg PDD AC001651) | | | | |
| Group 4 | 1.000 | 0.407 | 0.358 | 0.779 |
| (1.10 mg/kg PDD AC001651) | | | | |
| Group 5 | 1.000 | 0.473 | 0.432 | 0.470 |
| (1.71 mg/kg PDD AC001651) | | | | |

As shown in the data in Table 58 above, RNAi agent AC001651 robustly silences MMP7 expression, showing average ˜64% reduction in MMP7 mRNA in BAL exosomes at 1.10 mg/kg deposited dose in non-human primates.

Cynomolgus monkey MMP7 protein expression in BAL was quantitated by Western blot using iBright imaging system (Thermo Fisher), shown in the following Table 59.

TABLE 59
MMP7 protein expression in BAL.

| | Baseline (Day-7) | | Day 14 | |
|---|---|---|---|---|
| | | Std | | Std |
| | Avg | Dev | Avg | Dev |
| Group ID | MMP7 | (+/−) | MMP7 | (+/−) |
| Group 1 (isotonic saline) | 1.000 | N/A | 0.933 | 0.698 |
| Group 2 | 1.000 | N/A | 0.617 | 0.020 |
| (0.24 mg/kg PDD AC001651) | | | | |
| Group 3 | 1.000 | N/A | 0.218 | 0.161 |
| (0.66 mg/kg PDD AC001651) | | | | |
| Group 4 | 1.000 | N/A | 0.593 | 0.552 |
| (1.10 mg/kg PDD AC001651) | | | | |
| Group 5 | 1.000 | N/A | 0.299 | 0.131 |
| (1.71 mg/kg PDD AC001651) | | | | |

As shown in the data in Table 59 above, RNAi agent AC001651 robustly silences MMP7 expression, showing average ˜78% reduction in protein expression in BAL at 0.66 mg/kg deposited dose in non-human primates.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An RNAi agent for inhibiting expression of a matrix metallopeptidase 7 gene, comprising:
an antisense strand comprising the nucleotide sequence (5′→3′) UGACAUUCAAAAACCAACU (Seq ID No. 176); and
a sense strand comprising a nucleotide sequence that is at least partially complementary to the antisense strand,
wherein at least one nucleotide of the RNAi agent is a modified nucleotide or includes a modified internucleoside linkage.

2. The RNAi agent of claim 1, wherein the antisense strand comprises the nucleotide sequence (5′→3′) UGACAUUCAAAAACCAACUGC (Seq ID No. 679).

3. (canceled)

4. (canceled)

5. (canceled)

6. The RNAi agent of claim 1, wherein the modified nucleotide is selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

7. (canceled)

8. (canceled)

9. The RNAi agent of claim 1, wherein the sense strand comprises the nucleotide sequence (5′→3′) GCAGUUGGUUUUUGAAUGUCA (Seq ID No. 726).

10. (canceled)

11. The RNAi agent of claim 1, wherein the sense strand is between 18 and 30 nucleotides in length, and the antisense strand is between 18 and 30 nucleotides in length.

12. (canceled)

13. (canceled)

14. (canceled)

15. The RNAi agent of claim 11, wherein the RNAi agent has two blunt ends.

16. The RNAi agent of claim 9, wherein the sense strand comprises one or two terminal caps.

17. The RNAi agent of claim 9, wherein the sense strand comprises one or two inverted abasic residues.

18. (canceled)

19. (canceled)

20. (canceled)

21. (canceled)

22. (canceled)

23. The RNAi agent of claim 1, comprising an antisense strand that comprises the nucleotide sequence (5′→3′):
cPrpuGfacauucAfaaAfaCfcAfacugsc (SEQ ID NO:406);
wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; cPrpu represents a 5′-cyclopropyl phosphonate-2′-O-methyl uridine; s represents a phosphorothioate linkage; and wherein all or substantially all of the nucleotides on the sense strand are modified nucleotides.

24. The RNAi agent of claim 23, wherein the sense strand comprises the nucleotide sequence (5′→3′):
gscaguuggUfuUfuUfgaauguca (SEQ ID NO:526);
wherein a, c, g, i, and u represent 2′-O-methyl adenosine, 2′-O-methyl cytidine, 2′-O-methyl guanosine, 2′-O-methyl inosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; and s represents a phosphorothioate linkage; and wherein all or substantially all of the nucleotides on the antisense strand are modified nucleotides.

25. The RNAi agent of claim 35, wherein the sense strand further includes inverted abasic residues at the 3′ terminal end of the nucleotide sequence, at the 5′ end of the nucleotide sequence, or at both.

26. The RNAi agent of claim 24, wherein the RNAi agent is linked to a targeting ligand.

27. The RNAi agent of claim 26, wherein the targeting ligand has affinity for a cell receptor expressed on an epithelial cell.

28. The RNAi agent of claim 27, wherein the targeting ligand comprises an integrin targeting ligand.

29. The RNAi agent of claim 28, wherein the integrin targeting ligand is an αvβ6 integrin targeting ligand.

30. The RNAi agent of claim 29, wherein the targeting ligand comprises the structure: 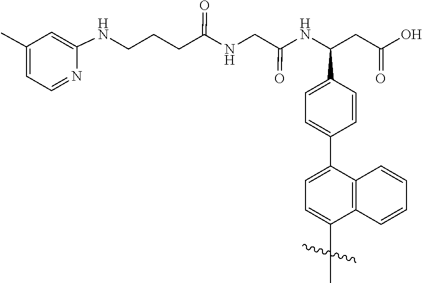 [SMILES: *c1ccc(-c2ccc([C@H](CC(=O)O)NC(=O)CNC(=O)CCCNc3cc(C)ccn3)cc2)c2ccccc12]
or a pharmaceutically acceptable salt thereof, or 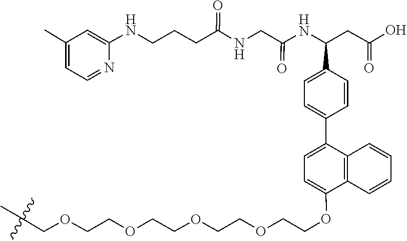 [SMILES: *COCCOCCOCCOCCOc1ccc(-c2ccc([C@H](CC(=O)O)NC(=O)CNC(=O)CCCNc3cc(C)ccn3)cc2)c2ccccc12]
or a pharmaceutically acceptable salt thereof,
wherein [symbol] indicates the point of connection to the RNAi agent.

31. The RNAi agent of claim 26, wherein the targeting ligand has a structure selected from the group consisting of: 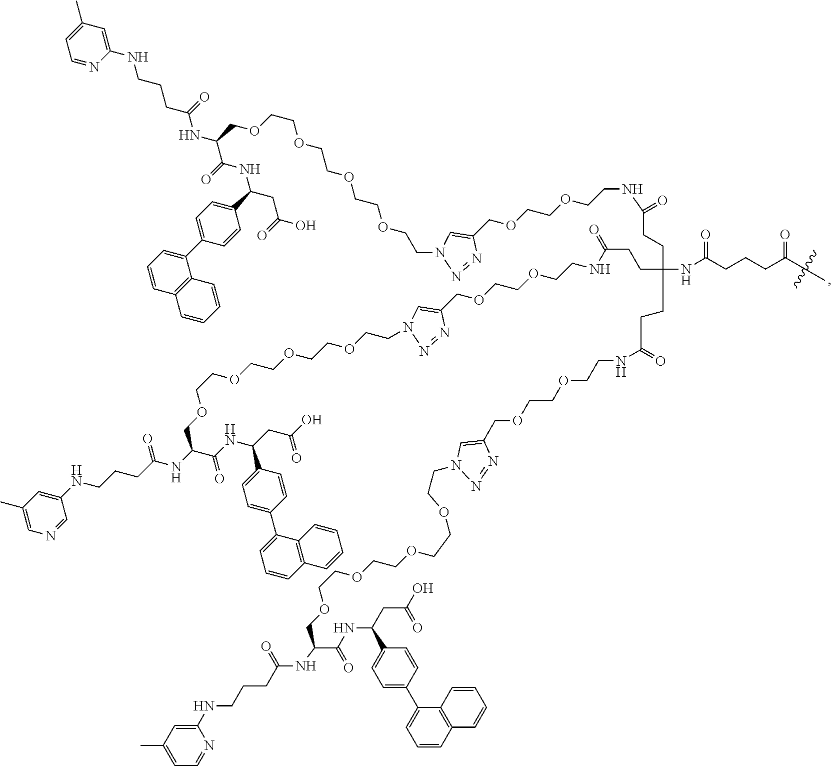 [SMILES: *C(=O)CCCC(=O)NC(CCC(=O)NCCOCCOCc1cn(CCOCCOCCOCCOC[C@H](NC(=O)CCCNc2cncc(C)c2)C(=O)N[C@@H](CC(=O)O)c2ccc(-c3cccc4ccccc34)cc2)nn1)(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1] 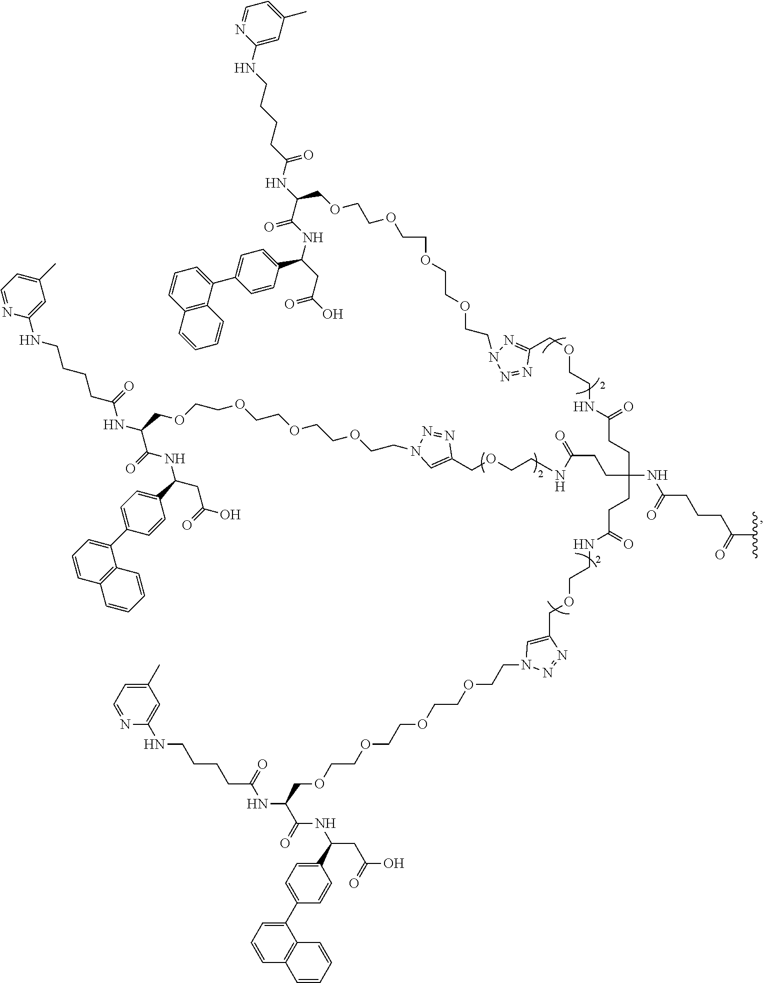 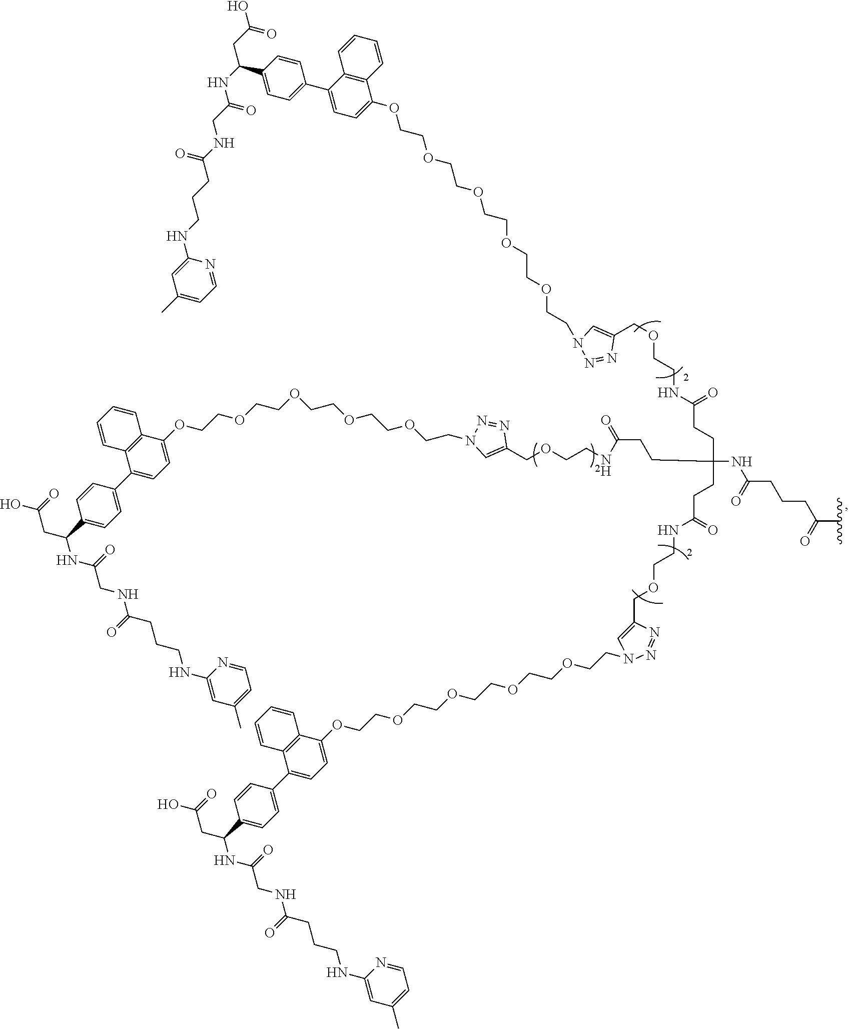 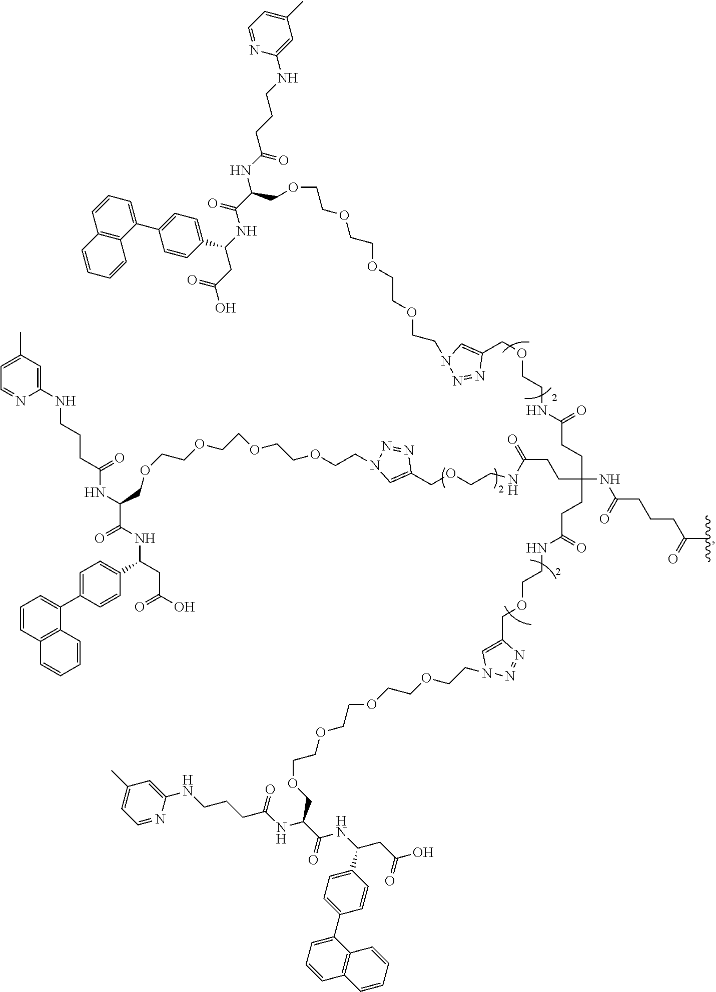 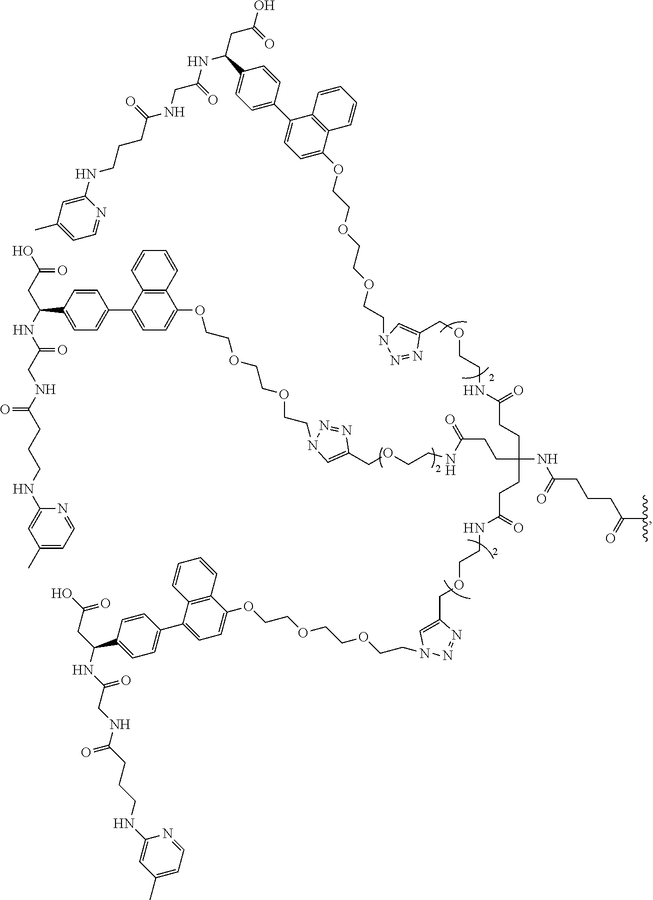 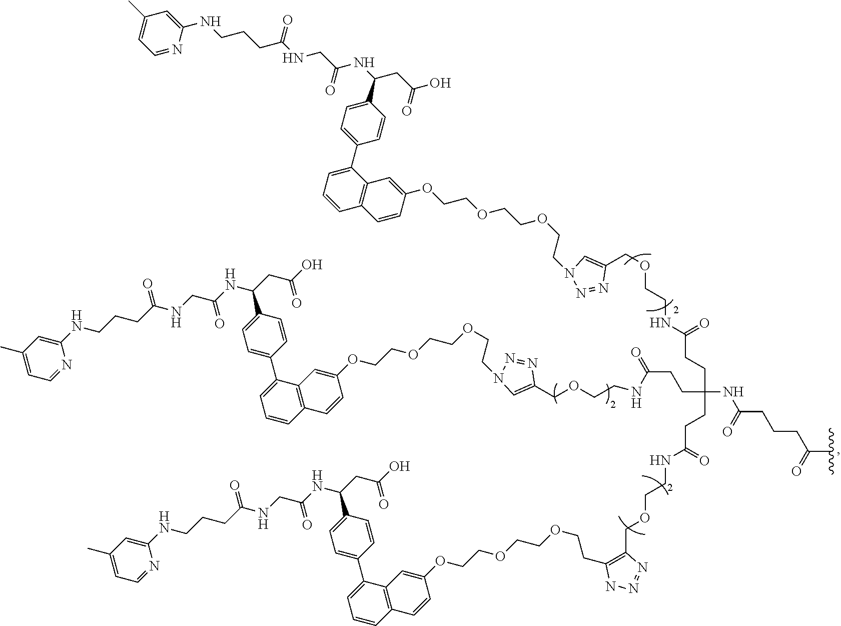 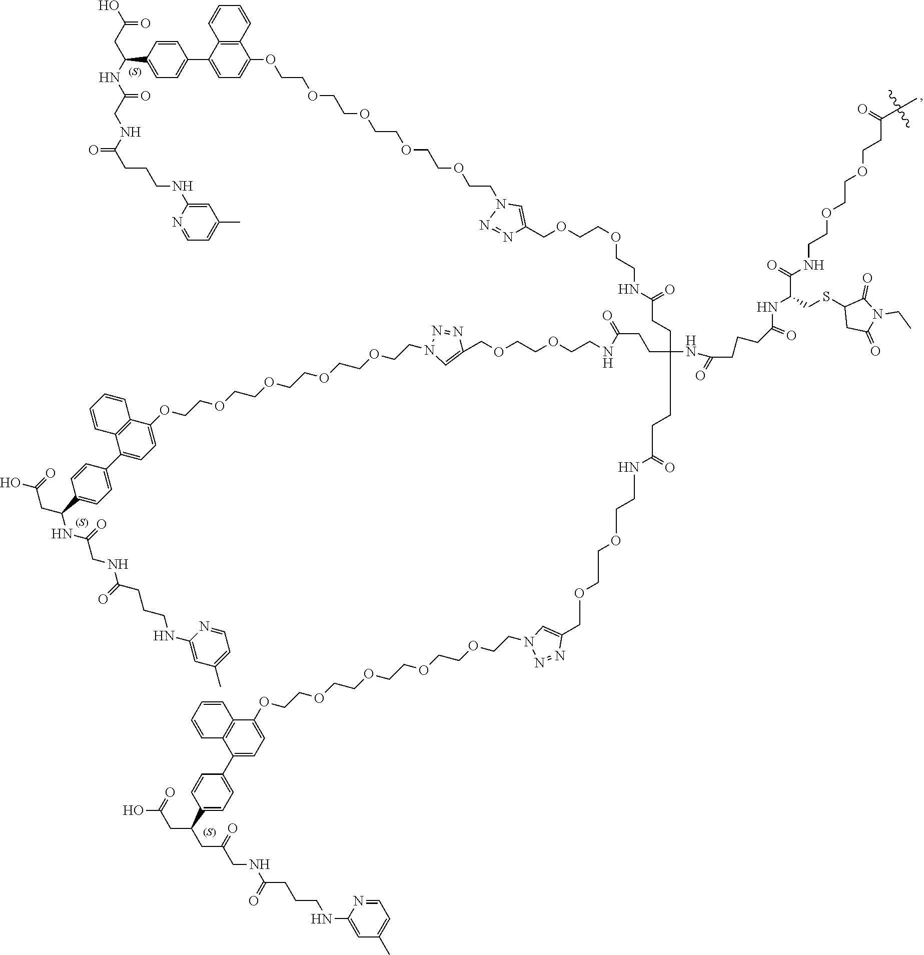 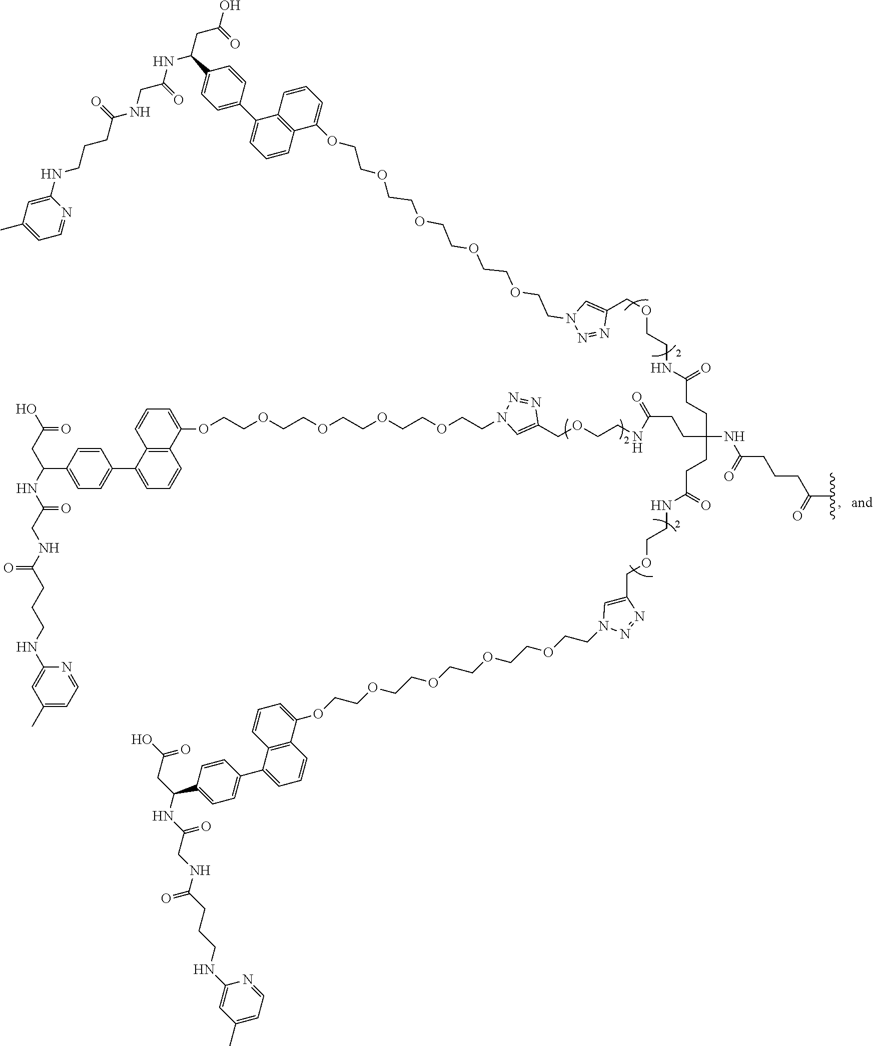 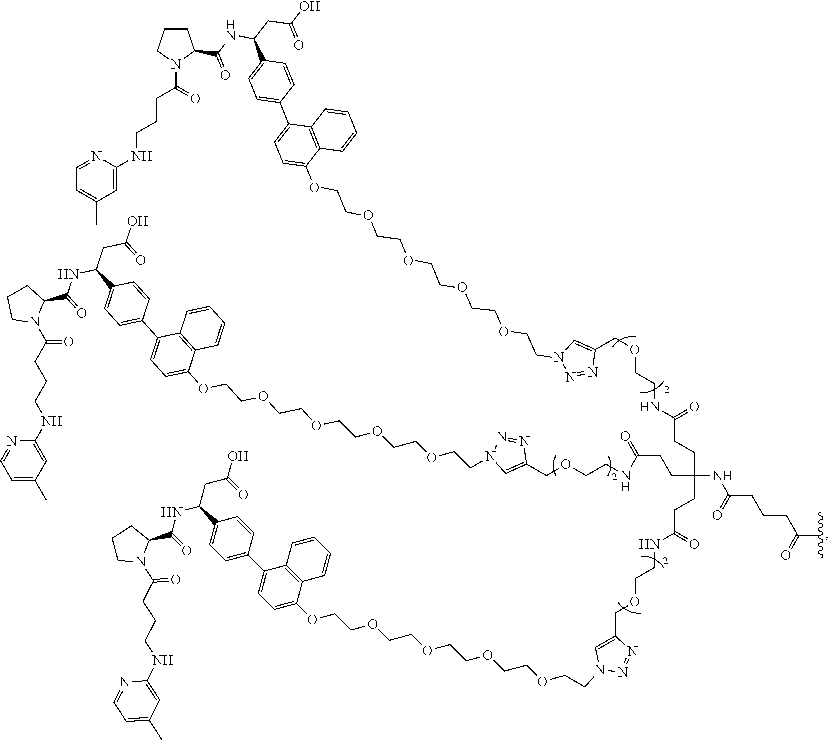
wherein [symbol] indicates the point of connection to the RNAi agent.

32. The RNAi agent of claim 31, wherein RNAi agent is conjugated to a targeting ligand having the following structure:

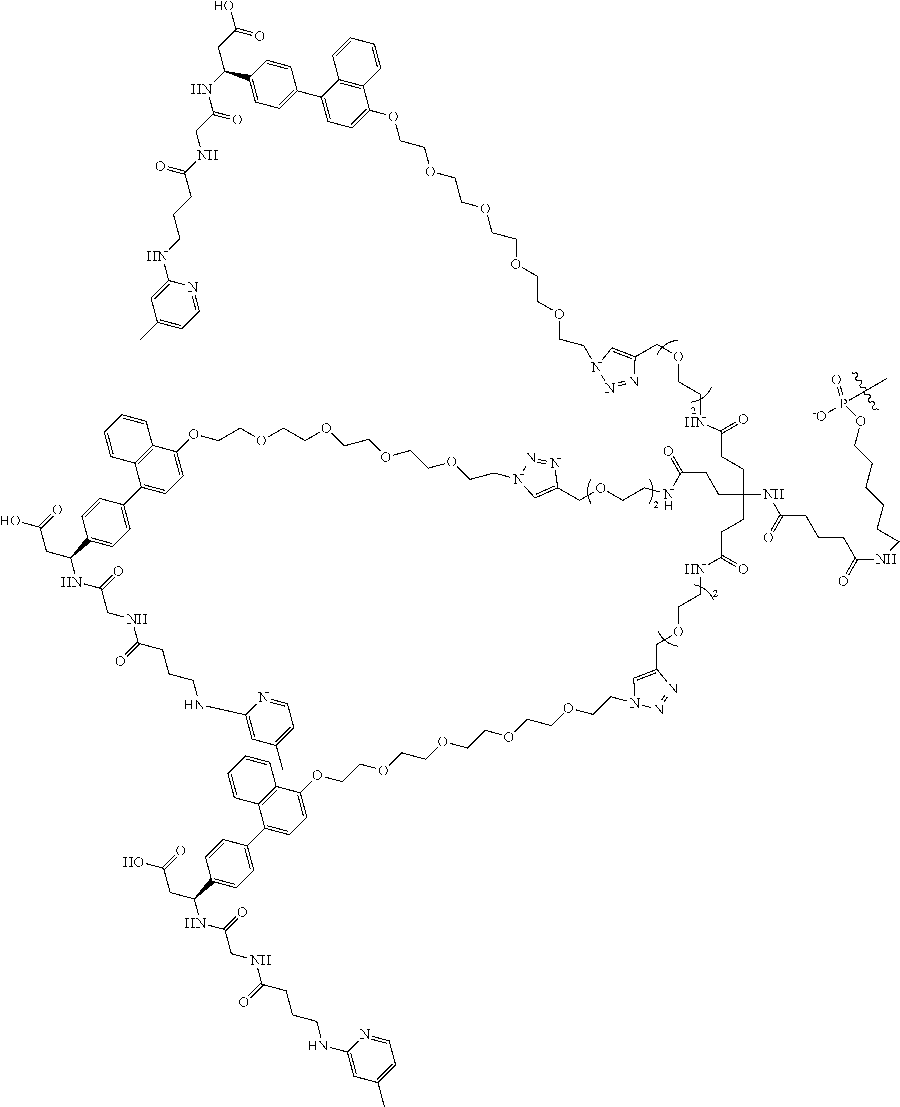

33. The RNAi agent of claim 26, wherein the targeting ligand has the following structure:

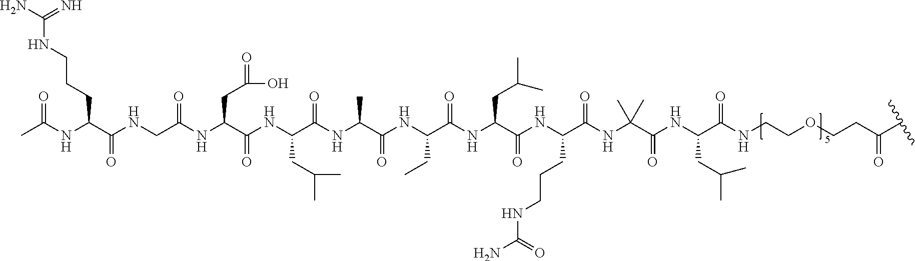

34. The RNAi agent of claim 32, wherein the targeting ligand is conjugated to the sense strand.

35. The RNAi agent of claim 34, wherein the targeting ligand is conjugated to the 5′ terminal end of the sense strand.

36. A composition comprising the RNAi agent of claim 25, wherein the composition further comprises a pharmaceutically acceptable excipient.

37. The composition of claim 36, further comprising a second RNAi agent capable of inhibiting the expression of matrix metallopeptidase 7 gene expression.

38. The composition of claim 36, further comprising one or more additional therapeutics.

39. The composition of claim 36, wherein the composition is formulated for administration by inhalation.

40. (canceled)

41. The composition of claim 36, wherein the RNAi agent is a sodium salt.

42. (canceled)

43. (canceled)

44. A method for inhibiting expression of a MMP7 gene in a cell, the method comprising introducing into a cell an effective amount of the RNAi agent of claim 25.

45. The method of claim 44, wherein the cell is within a subject.

46. The method of claim 45, wherein the subject is a human subject.

47. (canceled)

48. A method of treating one or more symptoms or diseases associated with enhanced or elevated membrane MMP7 activity levels, the method comprising administering to a human subject in need thereof a therapeutically effective amount of the composition of claim 36.

49. The method of claim 48, wherein the disease is idiopathic pulmonary fibrosis (IPF), asthma, another type of fibrosis, chronic inflammation, interstitial lung diseases (ILD), SARS-COV-2 or another type of infectious disease, acute respiratory distress syndrome (ARDS) or another type of acute lung injury, pulmonary hypertension, cancer, nonalcoholic fatty liver disease (NAFLD), nonalcoholic steatohepatitis (NASH), fatty liver disease, biliary atresia, and chronic kidney disease (CKD).

50. The method of claim 49, wherein the disease is idiopathic pulmonary fibrosis (IPF).

51. The method of claim 48, wherein the RNAi agent is administered at a deposited dose of about 0.01 mg/kg to about 5.0 mg/kg of body weight of the subject.

52. (canceled)

53. The method of claim 48, wherein the RNAi agent is administered in two or more doses.

54. (canceled)

55. (canceled)

56. (canceled)

57. (canceled)

58. (canceled)

59. (canceled)

60. (canceled)

61. A pharmaceutically acceptable salt of the RNAi agent of claim 23.

62. The pharmaceutically acceptable salt of claim 61, wherein the pharmaceutically acceptable salt is a sodium salt.

63. The RNAi agent of claim 2, wherein the sense strand comprises the nucleotide sequence (5′→3′) GCAGUUGGUUUUUGAAUGUCA (Seq ID No. 726), wherein the sense strand comprises one or two inverted abasic residues.
